(19) United States
(12) Patent Application Publication
(10) Pub. No.: US 2023/0220486 A1
(43) Pub. Date: Jul. 13, 2023

(54) Micronuclei DNA From Peripheral Red Blood Cells and Uses Thereof

(71) Applicant: Timing Biotech Technology Co. Ltd., Xiaoshan District, Hangzhou, Zhejiang (CN)

(72) Inventors: Xiaofei Gao, Hangzhou, Zhejiang (CN); Haobo Sun, Hangzhou, Zhejiang (CN); Xingyun Yao, Hangzhou, Zhejiang (CN); Ying Li, Hangzhou, Zhejiang (CN)

(21) Appl. No.: 17/998,266

(22) PCT Filed: May 14, 2021
(86) PCT No.: PCT/CN2021/093919

§ 371 Date: Nov. 9, 2022

(30) Foreign Application Priority Data
May 15, 2020 (WO) PCT/CN2020/090545

(51) Int. Cl. C12Q 1/6886

(52) U.S. Cl. CPC C12Q 1/6886; C12Q 2600/156; C12Q 1/6869

(57) ABSTRACT

Provided are peripheral red blood cell micronuclei DNA, a method for extracting the same, and the use of the micronuclei DNA in cancer screening, diagnosis, typing and/or staging.

### TECHNICAL FIELD

The present disclosure relates to the fields of biology, medicine and bioinformatics. Particularly, the present disclosure relates to peripheral red blood cell micronuclei DNA and its application in cancer detection.

### BACKGROUND

Cancer is one of the main diseases threatening human health and life. It is reported that, in 2018, there were 18.1 million new cancer cases and 9.6 million cancer deaths worldwide. Nearly half of new cancer cases and more than half of cancer deaths occurred in Asia (Global Cancer Statistics 2018: GLOBOCAN Estimates of Incidence and Mortality Worldwide for 36 Cancers in 185 Countries. Bray Freddie et al., CA: A Cancer Journal for Clinicians. 2018). Despite decades of continuous exploration, progress has been made in the diagnosis and treatment of cancer, but there is still a huge demand for cancer detection, especially the screening, diagnosis, classification and staging of cancer.

Blood continuously circulates in the body, and the total blood of normal adults accounts for about 8% of the body weight of men and about 7.5% of the body weight of women. Peripheral blood samples are easy to collect, store and transport and have high stability (Dagur, P. K. and J. J. McCoy, Collection, Storage, and Preparation of Human Blood Cells. Curr Protoc Cytom, 2015. 73: p. 5.1.1-16).

Micronuclei is usually considered as a small nuclear structure formed when chromosomes or chromosome fragments are not incorporated into one of the daughter nuclei during cell division, which is usually a sign of genotoxicity events and chromosome instability. Generally, it is a small nuclear structure formed outside the main nucleus which is independent of the main nucleus, due to the incorrect repair or unrepaired DNA breakage, or lagging asymmetric chromosome or chromatid fragment caused by chromosome non-separation (Liu, S., et al., Nuclear envelope assembly defects link mitotic errors to chromothripsis. Nature, 2018. 561(7724): p. 551-555).

Up to now, there is no report on the micronuclei DNA isolated or purified from peripheral red blood cells, and there is no report on the use of peripheral red blood cell micronuclei DNA for cancer detection.

### SUMMARY

Generally, the present disclosure relates to micronuclei DNA isolated or purified from peripheral red blood cells, its extraction method, and its application in screening, diagnosis, typing and/or staging of diseases.

The first aspect of the present disclosure relates to micronuclei DNA isolated or purified from peripheral red blood cells.

In some embodiments, the micronuclei DNA isolated or purified from peripheral red blood cells does not contain or substantially does not contain nucleated cell genomic DNA.

In some embodiments, the peripheral blood is human peripheral blood. In a specific embodiment, the peripheral blood is fresh human peripheral blood.

In some embodiments, the micronuclei DNA is used for cancer detection, such as early screening, diagnosis, typing and/or staging of cancer. In some specific embodiments, the micronuclei DNA is used for diagnosis of pan-cancer patients, including but not limited to patients suffering from colorectal cancer (also referred to as “CRC” hereinafter), hepatocellular cancer (also referred to as “HCC” hereinafter) or lung cancer (also referred to as “LC” hereinafter).

In some embodiments, the micronuclei DNA is used for early screening, diagnosis, typing and/or staging of cervical cancer.

In some embodiments, the micronuclei DNA is used for early screening, diagnosis, typing and/or staging of cervical cancer, and the micronuclei DNA comprises a gene classifier shown in Table 2, 4 or 6.

In other embodiments, the micronuclei DNA is used for early screening, diagnosis, typing and/or staging of colorectal cancer.

In a further embodiment, the micronuclei DNA is used for early screening, diagnosis, typing and/or staging of colorectal cancer, and the micronuclei DNA comprises a gene classifier shown in Table 8 or 10.

In some further embodiments, the micronuclei DNA is used for early screening, diagnosis, typing and/or staging of hepatocellular cancer.

In some even further embodiments, the micronuclei DNA is used for early screening, diagnosis, typing and/or staging of lung cancer.

In some even further embodiments, the micronuclei DNA is used for discriminating power between each of the two cancer patient groups: CRC vs. HCC, LC vs. HCC, LC vs. CRC.

In some even further embodiments, the micronuclei DNA is used for the multiclass discrimination of different types of cancers. In a specific embodiment, the micronuclei DNA is used for the multiclass discrimination of HD (“health donors”), HCC, LC and CRC.

The second aspect of the present disclosure relates to a method for isolating or purifying micronuclei DNA from peripheral red blood cells, which comprises the following steps:

a) providing peripheral blood samples;

b) isolating mononuclear cells and red blood cells from peripheral blood samples;

c) collecting red blood cells;

d) treating collected red blood cells with a red blood cell lysis buffer; and

e) extracting micronuclei DNA from the lysed red blood cells.

In a specific embodiment, the collected red blood cells are subjected to two or more sequential filtrations, e.g., filtrations by cell strainers, e.g., filtrations by 10 μm cell strainers.

In some embodiments, the red blood cell lysis buffer specifically lyses red blood cells by changing the osmotic pressure of cell suspension, but does not lyse nucleated cells.

In some embodiments, the red blood cell lysis buffer comprises NH₄Cl, NaHCO₃, EDTA or a combination thereof.

In some embodiments, micronuclei DNA is extracted from the lysed red blood cells by a DNA extraction reagent. In certain embodiments, the DNA extraction reagent comprises a protease, such as protease K. In certain specific embodiments, the DNA extraction reagent comprises protease K and EDTA.

In some embodiments, before step b), a step of diluting the peripheral blood sample is further included, for example, diluting with phosphate buffer solution in equal volume.

In some embodiments, in step b), the peripheral blood sample is subjected to density gradient centrifugation, such as Ficoll density gradient centrifugation, to obtain a mononuclear cell layer and a red blood cell layer.

A third aspect of the present disclosure relates to a method for constructing a gene classifier for cancer detection through peripheral red blood cell micronuclei DNA, which comprises:

a) providing more than one class, wherein each class represents a group of subjects with common characteristics;

b) isolating or purifying peripheral red blood cell micronuclei DNA from peripheral red blood cells of each subject of each class;

c) sequencing the whole genome of the peripheral red blood cell micronuclei DNA to obtain fragment sequence information of the micronuclei DNA;

d) comparing the fragment sequence information of micronuclei DNA from peripheral red blood cells in different classes of subjects;

e) training the characteristic DNA fragment set for specific cancers according to the differences in the distribution of fragment the sequence information of micronuclei DNA in peripheral red blood cells of different classes of subjects, thus obtaining a gene classifier for specific cancer detection.

In certain embodiments, the different classes are cancer subjects and non-cancer subjects for the same cancer.

In certain embodiments, the different classes are subjects with different types of the same cancer.

In certain embodiments, the different classes are subjects at different stages of the same cancer type.

The fourth aspect of the present disclosure relates to a gene classifier for cancer detection, which is constructed by peripheral red blood cell micronuclei DNA.

In certain embodiments, the gene classifier comprises the genes shown in Table 2, 4, 6, 8 or 10.

A fifth aspect of the present disclosure relates to a method of cancer detection for a test subject, comprising:

a) extracting micronuclei DNA in peripheral red blood cells of the test subject, wherein the extract does not contain or substantially does not contain nucleated cell genomic DNA;

b) sequencing the micronuclei DNA and sample-matched genomic DNA by whole genome sequencing to obtain signature of the micronuclei DNA from red blood cells in specific genomic elements or different bin size for the test subject;

c) comparing the sample-matched genomic DNA and micronuclei DNA in red blood cells or micronuclei DNA from different types of samples in step b) with the whole genome analysis, so as to classify the micronuclei DNA from genomic DNA and evaluate the difference of micronuclei DNA signature from types of samples;

d) comparing the signature information of the micronuclei DNA from different classes of cancer patients or healthy donors obtained in step b) with the gene classifier or other deep neural network classifier for cancer detection of the present disclosure, so as to classify the test subjects into one or more of the classes.

A sixth aspect of the present disclosure relates to a system for cancer detection of a test subject, which comprises a comparison means for comparing peripheral red blood cell micronuclei DNA from the test subject with the gene classifier of the present disclosure.

A seventh aspect of the present disclosure relates to the use of an agent for analyzing micronuclei DNA of peripheral red blood cells in the preparation of a detection device or a detection kit for cancer screening, diagnosing, typing and/or staging.

In some specific embodiments, the screening or diagnosis is early screening or diagnosis.

The eighth aspect of the present disclosure relates to peripheral red blood cell micronuclei DNA for use in cancer detection.

The ninth aspect of the present disclosure relates to a method for isolating peripheral red blood cells.

The tenth aspect of the present disclosure relates to the use of peripheral red blood cells in cancer detection.

The above content is summary in general, so it includes simplification, generalization and omission of details when necessary. Therefore, those skilled in the art will recognize that this general summary is merely illustrative and is not intended to be limiting in any way. Other aspects, features and advantages of the methods, compositions and/or devices and/or other subjects described herein will become apparent under the teachings herein. A summary is provided to simplify the introduction of some selected concepts, which will be further described in the following detailed description. This summary is not intended to identify key features or essential features of the claimed subject matter, nor is it intended to be used as an auxiliary means to determine the scope of the claimed subject matter. In addition, the contents of all references, patents and published patent applications cited throughout this application are incorporated herein by reference in their entirety.

### TECHNICAL EFFECTS OF THE INVENTION

The inventors extracted micronuclei DNA from peripheral red blood cells for the first time and performed high-throughput sequencing on the extracted micronuclei DNA. Through bioinformatics analysis, erythrocyte micronuclei DNA has been successfully used in cancer screening, diagnosis, risk ranking, typing and staging, which has important guiding significance for cancer prevention, treatment and prognosis.

The invention has achieved superior technical effects in at least the following aspects.

Abundancy in Sample Sources

According to the invention, peripheral blood is used as a sample source, and the source is abundant, stable, and easy to obtain, collect, store and transport.

Effectiveness in the Isolation of Micronuclei DNA from Red Blood Cells

By the method disclosed in the present disclosure, micronuclei DNA in red blood cells can be effectively isolated from human peripheral blood. It has not been reported in the art that micronuclei DNA in red blood cells can be effectively isolated from human peripheral blood.

Simple and Fast Operation

According to the present disclosure, only a small amount (for example, only 1 ml) of peripheral blood needs to be collected from the subject, which may relieve the psychological pressure of the subject. Particularly, for the detection of cervical cancer, there is no need to collect cervical exfoliated cells of the subjects, which is easy to operate and can effectively reduce the psychological pressure of the subjects.

In addition, by high-throughput sequencing, micronuclei DNA can be quickly sequenced to obtain genetic information.

High Sensitivity and Specificity of Cancer Detection

Using micronuclei DNA obtained from peripheral red blood cells, cancer can be detected with extremely high sensitivity and specificity by the method of the present disclosure.

### BRIEF DESCRIPTION OF THE DRAWINGS

The present invention will be more apparent to those skilled in the art through the specific embodiments and examples described in the present disclosure, combined with the following drawings.

FIG. 1 shows a schematic diagram of isolating peripheral blood cells by Ficoll density gradient centrifugation.

FIG. 2 shows that mononuclear cells and red blood cells were collected after Ficoll density gradient centrifugation.

FIG. 3 shows the flow chart of sample processing and high-throughput sequencing of peripheral blood mononuclear cell genomic DNA and erythrocyte micronuclei DNA.

FIG. 4 shows the bioinformatics analysis algorithm logic.

FIG. 5 shows hierarchical clustering of healthy individuals and cervical cancer patients.

FIG. 6 shows hierarchical clustering of patients with different types of cervical cancer (squamous cell carcinoma and adenocarcinoma).

FIG. 7 shows hierarchical clustering of cervical cancer patients at different stages.

FIG. 8 shows the risk ranking of subjects and screening of cervical cancer patients by the gene classifier of the present disclosure.

FIG. 9 shows the risk ranking of subjects by the gene classifier of the present disclosure, and patients with cervical squamous cell carcinoma from patients with cervical adenocarcinoma were distinguished.

FIG. 10 shows hierarchical clustering of healthy individuals and patients with colorectal cancer.

FIG. 11 shows hierarchical clustering of patients with different types of colorectal cancer (colon cancer and rectal cancer).

FIG. 12 shows the risk ranking of subjects by the gene classifier of the present disclosure to screen colorectal cancer patients.

FIG. 13 shows the risk ranking of subjects by the gene classifier of the present disclosure to differentiate colon cancer patients from rectal cancer patients.

FIG. 14 shows the multiclass discrimination of HD, HCC, LC and CRC samples in a training cohort (left), validation cohort (middle) and test cohort (right).

FIG. 15 (FIG. 15A-D) shows the characterization the profile of the red blood cell micronuclei DNA (i.e., a rbcDNA signature) in healthy donors and cancer patients.

### DETAILED DESCRIPTION

While the present invention can be embodied in many different manners, specific illustrative embodiments thereof which demonstrate the principles of the invention are disclosed herein. It should be emphasized that the present invention is not limited to the specific embodiments illustrated. In addition, any chapter titles used herein are for organizational purposes only and should not be interpreted as limiting the described subject matter.

Unless otherwise defined herein, scientific and technical terms used in connection with the present invention will have the meanings commonly understood by those of ordinary skill in the art. In addition, unless the context requires otherwise, the singular term shall include the plural, and the plural term shall include the singular. More specifically, as used in this specification and the appended claims, unless the context clearly indicates otherwise, the singular forms “a,” “an” and “the” include plural referents. Therefore, for example, reference to “a protein” may include a variety of proteins; and reference to “a cell” includes a mixture of cells, etc. In this application, unless otherwise stated, the use of the expression “or” refers to “and/or.” In addition, the use of the term “comprising” and other forms such as “comprise” and “comprises” is not limiting. Furthermore, the ranges provided in the description and the appended claims include all values between endpoints and breakpoints.

Generally, the terms related to cell and tissue culture, molecular biology, immunology, microbiology, genetics, and protein as well as nucleic acid chemistry and hybridization described herein, and their techniques are well-known and commonly used in the art. Unless otherwise stated, the methods and techniques of the present invention are generally carried out according to conventional methods known in the art, as described in various general and more specific references cited and discussed throughout this specification. See, for example, Abbas et al., Cellular and Molecular Immunology, 6th ed., W. B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). Terms related to analytical chemistry, synthetic organic chemistry and drugs and pharmaceutical chemistry described herein, as well as laboratory procedures and techniques, are well-known and commonly used terms in the field. In addition, any chapter titles used herein are for organizational purposes only and are not to be interpreted as limiting the described subject matter.

### Definition

In order to better understand the present invention, definitions and interpretations of related terms are provided as follows.

In the context of the present disclosure, the term “DNA” refers to deoxyribonucleic acid.

In the context of the present disclosure, the term “micronuclei” is intended to refer to a small nuclear structure containing DNA in a specific cell other than the nucleus. There is no nucleus in peripheral red blood cells, so there is only micronuclei structure.

In the context of the present disclosure, the term “cervical cells” include cells located at any part of the cervix and cells detached from any part of the cervix that can be diseased. In one embodiment, cervical cells are cells isolated from tissues exfoliated from that inner wall of the cervix in a natural or artificial way, also called “cervical exfoliated cells.”

In the context of the present disclosure, a “subject” refers to a subject to be tested. In certain embodiments, the “subject” is a human subject.

In the context of the present disclosure, a “patient” refers to a subject suffering from a certain disease, such as cervical cancer.

In the context of the present disclosure, “cancer” is a general term for malignant tumors. “Tumor” refers to the abnormal proliferation of cells in local tissues under the influence of various tumorigenic factors.

In the context of the present disclosure, a “cancer subject” or a “cancer patient” are used interchangeably, referring to a subject suffering from a certain cancer, such as cervical cancer or colorectal cancer.

In the context of the present disclosure, a “non-cancer subject” refers to a subject who does not suffer a certain cancer. For example, “non-cervical cancer subject” refers to a subject without cervical cancer. In the specific embodiments and examples of the present disclosure, a “non-cancer subject” is also referred to as a “healthy individual,” and likewise, it refers to that the individual or subject does not have such cancer.

In the context of the present disclosure, the term “cancer detection” refers to detecting the condition of a subject suffering from cancer. “Detecting” includes but is not limited to screening, diagnosis, typing and staging. “Screening” refers to preliminarily detecting whether there is cancer or the risk of cancer. “Diagnosis” or “medical diagnosis” refers to assessing the patient's condition from a medical point of view. “Typing” refers to further dividing the same kind of cancer into specific subtypes. For example, cervical cancer can be classified into cervical squamous cell carcinoma and cervical adenocarcinoma. “Staging” refers to predicting, assessing or dividing the stage of a cancer. For example, cervical cancer (squamous cell carcinoma) can be divided into three stages: low differentiation, low-medium differentiation, medium differentiation and high differentiation.

In the context of the present disclosure, the term “nucleated cell” refers to a cell in which a nucleus exists. For peripheral blood, the term “nucleated cell” is the general term for a granulocyte, a monocyte and a lymphocyte.

In the context of the present disclosure, the term “genome” refers to the sum of all genetic information in a cell, especially a complete set of haploid genetic material in a cell.

In the context of the present disclosure, the term “nucleated cell genomic DNA,” “nucleated cell nucleus genome,” or “nucleated cell nucleus genomic DNA” are used interchangeably, meaning all genetic information contained in nuclear chromosomes.

In the context of the present disclosure, the term “gene classifier” or “classifier” can be used interchangeably, referring to a group of DNA fragments or a group of genes in genomic DNA or micronuclei DNA that are specific for a specific disease.

In the context of the present disclosure, the term “DNA fragment library” or “DNA library” can be used interchangeably, which refers to double-stranded DNA obtained by completing the ends of a sample DNA fragment, adding a phosphate group at the 5′ end, adding an adenine nucleotide (A) at the 3′ end, and connecting an adapter and a sample barcode at both ends.

In the context of the present disclosure, the term “micronuclei DNA from red blood cells” and “erythrocyte micronuclei DNA” are used interchangeably, and is intends to refer micronuclei DNA isolated from red blood cells. In a specific embodiment, the red blood cells are peripheral red blood cells. Accordingly, in the context of the present disclosure, “peripheral red blood cells micronuclei DNA, “peripheral erythrocyte micronuclei DNA”, and “micronuclei DNA from peripheral red blood cells” are used interchangeably. In a specific embodiment, micronuclei DNA is isolated or purified from peripheral red blood cells.

In the context of the present disclosure, the term “high-throughput sequencing” (also known as Next-Generation Sequencing (NGS)) refers to DNA sequencing technology that simultaneously sequences thousands (even millions) of DNA templates in a single chemical reaction.

In the context of the present disclosure, the term “reads” refers to the sequence of a sample DNA fragment in a DNA fragment library measured by high-throughput sequencing, with the sequence linked in the library preparation stage removed.

In the context of the present disclosure, the term “coverage depth” refers to an effective nucleic acid sequencing fragment for base recognition in a specific region, also known as the number of reads.

In the context of the present disclosure, the term “sequence alignment” refers to the alignment of reads to a reference genome (e.g., a human reference genome) by the principle of sequence identity.

In the context of the present disclosure, the term “reference genome” is the whole genome sequence of an organism of the same species as the sample DNA, which can be obtained from a public database. In one embodiment, the reference genome is a human reference genome. The public database is not particularly limited. In some embodiments, the public database is GenBank database of NCBI.

In the context of the present disclosure, the term “sensitivity” refers to the percentage of samples with positive tests in the total number of patients. In medical diagnosis, sensitivity can be expressed by the following formula, reflecting the ratio of correctly diagnosing patients:

Sensitivity=true positive number/(true positive number+false negative number) ×100%.

In short, if “true positive,” “false positive,” “true negative” and “false negative” are represented by “a”, “b”, “c” and “d”, respectively, the relationship among sensitivity, specificity, missed diagnosis rate, misdiagnosis rate and accuracy can be shown as follows.

| | Pathological diagnosis (gold standard) | | |
|---|---|---|---|
| Screening method | Diseased | Non-diseased | Sum |
| Positive (+) | True positive (a) | False positive (b) | a + b |
| Negative (−) | False negative (c) | True negative (d) | c + d |
| Sum | a + c | b + d | a + b + c + d |

Among the cases with positive screening results by this method, “true positive (a)” refers to the number of cases diagnosed as diseased by pathology, and the result of a method is also positive; “false positive (b)” refers to the number of cases diagnosed as non-diseased by pathology, and the result of a method is positive; “false negative (c)” refers to the number of cases diagnosed as diseased by pathology and the result of a method is negative; and “true negative (d)” refers to the number of cases diagnosed as non-diseased by pathology and the result of a method is negative.

Sensitivity(sen)=a/(a+c);

Specificity(sep)=d/(b+d);

Missed diagnosis rate=c/(a+c);

Misdiagnosis rate=b/(b+d);

Accuracy=(a+d)/(a+b+c+d)

As known by those skilled in the art, the higher the value of sensitivity and specificity, the better; and the lower the missed diagnosis rate and misdiagnosis rate, the better.

In the context of the present disclosure, the term “specificity” refers to the percentage of samples with negative tests in healthy people in the total number of healthy people. In medical diagnosis, “specificity” can be expressed by the following formula, which reflects the ratio of correct diagnosis of non-patients:

Specificity=true negative number/(true negative number+false positive number)×100%.

In the context of the present disclosure, the term “missed diagnosis rate,” also known as false negative rate, refers to the percentage of patients who are actually diseased when screening or diagnosing a disease in a population, but are determined as non-patients according to the diagnostic criteria. In medical diagnosis, the missed diagnosis rate can be expressed by the following formula:

Missed diagnosis rate=false negative number/(true positive number+false negative number)×100%.

In the context of the present disclosure, the term “misdiagnosis rate,” also known as false positive rate, refers to the percentage of subjects who do not actually suffer from a disease when screening or diagnosing a disease in a population, but are determined as patients with such a disease according to the diagnostic criteria. In medical diagnosis, the misdiagnosis rate can be expressed by the following formula:

Misdiagnosis rate=false positive number/(true negative number+false positive number)×100%.

In the context of the present disclosure, the expression “about” refers to that the deviation does not exceed plus or minus 10% of a specified value or range.

Peripheral Blood

In the present disclosure, “peripheral blood” refers to blood released into the circulatory system by hematopoietic organs and participating in the circulation. “Peripheral blood” is different from immature blood cells in hematopoietic organs such as bone marrow. In the present disclosure, peripheral blood can be collected by reference to known methods in the art such as venous blood collection, fingertip blood collection or earlobe blood collection.

Generally, peripheral blood consists of plasma and blood cells, wherein the blood cells further include white blood cells (also called “leukocytes”), red blood cells and platelets. By volume, red blood cells account for about 45%, plasma accounts for about 54.3%, and white blood cells account for about 0.7% of the total peripheral blood. Leukocytes are nucleated cells, which are the general term of granulocytes, monocytes and lymphocytes. Normal red blood cells have no nucleus, no genomic DNA, and are nuclear-free cells.

In the context of the present disclosure, a “peripheral blood mononuclear cell” (PBMC) refers to a cell with a single nucleus in peripheral blood, including monocytes and lymphocytes.

Separation of Peripheral Blood Cells

The separation methods of peripheral blood cells include natural sedimentation, differential sedimentation, sodium chloride separation, density gradient centrifugation and so on.

Different components of peripheral blood can be separated by using the density difference between different components of peripheral blood. For example, different components of peripheral blood can be separated by Ficoll density gradient centrifugation or Percoll method.

In a specific embodiment of the present disclosure, peripheral blood is separated by Ficoll density gradient centrifugation. Specifically, it is carried out in the following ways:

1. Peripheral Blood Collection and Sample Preparation

Peripheral blood is obtained from a subject and diluted appropriately. For example, it can be diluted by adding phosphate buffer solution (PBS). In certain embodiments, about 1-5 ml of fresh peripheral blood is obtained from a subject and diluted by adding an equal volume of PBS to obtain a diluted blood sample. In a specific embodiment, 1 ml of fresh peripheral blood is obtained from a subject, and 1×PBS is added for equal volume dilution to obtain diluted peripheral blood samples.

2. Density Gradient Centrifugation of Peripheral Blood Samples

Initially, an appropriate amount of Ficoll density gradient centrifuge is added into the density gradient centrifuge tube, and then the diluted peripheral blood sample as described above is add thereto. In certain embodiments, an appropriate amount of Ficoll density gradient centrifuge is added to the density gradient centrifuge tube in a ratio of the volume of peripheral blood collected from the subject to the volume of Ficoll density gradient centrifuge of about 1:3 to 1:10. For example, in a specific embodiment, 1 ml of fresh peripheral blood is obtained from a subject, and 5 ml of Ficoll density gradient centrifuge (Stemcell, Lymphoprep™ 07801) is added to the density gradient centrifuge tube.

Then, the diluted peripheral blood sample was slowly added onto the Ficoll density gradient centrifuge in the Ficoll density gradient centrifuge tube for density gradient centrifugation. Density gradient centrifugation can be carried out for about 10-15 minutes at about 15-25° C. and at about 1000-1500 g g. In a specific embodiment, the density gradient centrifugation is performed by 1200 g centrifugation at 18° C. for 15 minutes.

After density gradient centrifugation, it is divided into three layers: the upper layer is plasma, the middle layer is PBMC layer, and the bottom layer is RBC layer.

Collection of PBMC and RBC respectively. For example, the middle and upper layer liquid in the density gradient centrifuge tube is sucked by a suction means (such as a straw), and PBMC is separated and collected. An extraction means (such as a needle tube) is used to extract bottom red blood cells from the bottom of the density gradient centrifuge tube, and RBCs are separated and collected. In a specific embodiment, the bottom red blood cells are extracted from the bottom of the density gradient centrifuge tube by using a needle tube to a 1.5 ml centrifuge tube, with 1×PBS added up to a volume of 1 ml. Centrifugation is conducted at room temperature for 10 min at 300 g, and red blood cells at the bottom of the tube are collected. The collected RBCs were then subject to two sequential filtrations by 10 μm cell strainers to remove potential contamination of nucleated cells.

Isolation of Micronuclei DNA from Peripheral Red Blood Cells

According to the inventor's knowledge, there is no report on isolating micronuclei DNA from human peripheral red blood cells in the prior art. Unexpectedly, the inventors found that the micronuclei DNA of peripheral red blood cells can be separated simply and efficiently by the method of the present disclosure. In certain embodiments, the collected red blood cells are first lysed and then centrifuged. Thereafter, micronuclei DNA was extracted from the supernatant after centrifugation. In certain embodiments of the present disclosure, “peripheral red blood cell micronuclei DNA” includes all DNA present in peripheral red blood cells. In a specific embodiment of the present disclosure, the isolated “peripheral red blood cell Micronuclei DNA” does not contain nucleated cell genomic DNA. In another specific embodiment of the present disclosure, isolated “peripheral red blood cell micronuclei DNA” substantially does not contain nucleated cell genomic DNA.

The inventors also unexpectedly found that micronuclei DNA isolated from peripheral red blood cells can be used to detect various cancers.

Lysis of Red Blood Cells

In some embodiments, the collected red blood cells are lysed by adding a red blood cell lysis buffer. Erythrocyte lysis buffer can lyse erythrocytes while hardly damaging nucleated cells (such as PBMC). It can lyse erythrocytes effectively by slightly changing the osmotic pressure of cell suspension without affecting all nucleated cells. The red blood cell lysis buffer commonly used in the art contains NH₄Cl, NaHCO₃, EDTA or other combinations, for example, NH₄Cl, NaHCO₃ and EDTA. For example, every 1000 ml of red blood cell lysis buffer contains 8.3 g NH₄Cl, 1.0 g NaHCO₃, 1.8 ml of 5% EDTA and ultra-pure water.

The red blood cell lysis buffer can be, for example, a red blood cell lysis buffer (Biosharp, Cat No./ID: BL503B), a red blood cell lysis buffer (Solarbio, Cat No./ID: R1010) or a BD FACS Lysing Solution red blood cell lysis buffer (BD, Cat No./1 D: 349202). In a specific embodiment, 10 ml of red blood cell lysis buffer (Biosharp, Cat No./ID: BL503B) is added to the collected red blood cells, and the collected red blood cells are lysed for 20 minutes at room temperature in the dark.

Centrifugation

Thereafter, supernatant and precipitate (cell debris) are separated by centrifugation. In a specific embodiment, centrifugation is performed at 3000 g at room temperature for 10 minutes, and then the supernatant is taken.

Isolation of Micronuclei DNA

Then, micronuclei DNA is extracted from the supernatant. In certain embodiments, the DNA contained in the supernatant is pretreated by adding EDTA and protease K. EDTA is added in the digestion process with protease K to inhibit the influence of Mg²⁺-dependent nuclease. In a specific embodiment, the supernatant is incubated with 10 mm EDTA (Solarbio Cat No./ID: E1170), 200 ug/ul protease K (ProteinaseK, Ambion, Cat No./ID: AM2548) at 56° C. for 8 hours.

After incubation, commercial kits or reagents are used to extract micronuclei DNA. Examples of commercial kits include but are not limited to QIAamp DNA Blood Mini Kit, DNAzol reagent, PureLink™ Pro 96 Genomic DNA Purification Kit (Thermo, Cat No./ID: K182104A), blood genomic DNA extraction system (0.1-20 ml) (TIANGEND, Cat No./ID: P349), HiPure Blood DNA Midi Kit III(Magen, Cat No./ID: D3114). In a specific embodiment, erythrocyte micronuclei DNA is extracted using QIAamp DNA Blood Mini Kit (Qiagen, Cat No./ID: 51106).

Extraction of Genomic DNA from Peripheral Blood Mononuclear Cells

The genomic DNA of peripheral blood mononuclear cells can be extracted by commercial kits. In a specific embodiment, for peripheral blood mononuclear cell samples obtained after density gradient centrifugation, genomic DNA is extracted using QIAamp DNA Blood Mini Kit (Qiagen, Cat No./ID: 51106).

Whole Genome Amplification

Whole-genome amplification (WGA) is non-selective amplification of the whole genome sequence. Its main purpose is to maximize the amount of DNA on the basis of faithfully reflecting the whole genome, and to amplify the whole genome DNA of micro tissues and single cells without sequence bias.

Whole-genome amplification methods are mainly divided into the following types: first, amplification technology based on thermal cycles and PCR; second, amplification technology based on isothermal reaction and not based on PCR; and the third is MALBAC (Multiple Annealing and Looping-based Amplification Cycles). The WGA technology based on PCR includes degenerate oligonucleotide primer PCR (DOP-PCR), linker-adapter PCR (LA-PCR), interspersed repeat sequence PCR (IRS-PCR), tagged random primer PCR (T-PCR), primer extension preamplification PCR (PEP-PCR), among others. WGA based on isothermal reaction includes multiple displacement amplification (MDA), primase-based whole genome amplification (pWGA) and so on.

The methods of amplifying the whole genome DNA of a single cell mainly include MDA, MALBAC and DOP-PCR. These amplification methods can amplify pg-level or fg-level DNA in cells to μg-level which can satisfy sequencing.

Multiple Displacement Amplification (MDA)

Multiple displacement amplification (MDA) was first proposed by Dr. Lizardi of Yale University in 1998. This method is a constant temperature amplification method based on the principle of strand displacement amplification. Phage 129 DNA polymerase was used in multiple displacement amplification. PhageΦ29 DNA polymerase has a strong binding ability to DNA template, which can continuously amplify 100 Kb DNA template without dissociation from the template. At the same time, the enzyme has 3′-5′ exonuclease activity and low amplification error rate.

Multiple displacement amplification has the following advantages:
samples need not be purified;
stable yield;
uniform amplification of the genome;
amplification with high fidelity;
simple operation, independent of PCR reaction.

Commercial kits for MDA include REPLI-g series kits (Qiagen Inc), GenomiPhi series kits (GE Healthcare Inc), among others.

MALBAC (Multiple Annealing and Looping-Based Amplification Cycles)

MALBAC is different from non-linear or exponential amplification, but uses special primers to make the ends of amplicon complementary to each other. In this technique, the unique DNA polymerase with strand displacement activity is used for quasi-linear whole genome pre-amplification, and then exponential amplification is performed by PCR technology, which provides sufficient experimental materials for downstream analysis. In 2012, Science magazine published two articles related to this technology (C. Zong et al., Science 2012: 1622-1626; S. Lu et al., Science: 1627-1630).

MALBAC has the following advantages:
high amplification success rate;
good uniformity;
high coverage.

Commercial kits for MALBAC include MALBAC® single cell amplification kit from YiKon.

Degenerate Oligonucleotide Primer PCR(DOP-PCR)

The difference between DOP-PCR and conventional PCR is that it uses a single semi-degenerate primer and low renaturation temperature, has no species specificity, has no relation with the complexity of DNA, and can uniformly amplify the whole genome.

Commercial kits for DOP-PCR include PicoPlex series kits (Rubicon Genomics Inc), GenomePlex series kits (Sigma Aldrich Inc), SurePlex series kits (BlueGnome, which has been acquired by Illumina) and so on.

In the present disclosure, PBMC genomic DNA and RBC micronuclei DNA can be amplified by the whole genome amplification methods known in the art. In a specific embodiment, PBMC genomic DNA and RBC micronuclei DNA are amplified by MDA. Specifically, for PBMC genomic DNA and RBC micronuclei DNA extracted by QIAamp DNA Blood Mini Kit (Qiagen, Cat No./ID: 51106), MDA was performed by using REPLI-g Single Cell Kit (Qiagen, Cat No./ID: 150345), respectively. And the amplified DNA sample is obtained.

The REPLI-g Single Cell Kit adopts multiple displacement amplification (MDA) technology, which can uniformly amplify single cell or purified genomic DNA, and can cover all loci of genome. All buffers and reagents are produced through a strictly controlled process to avoid DNA contamination and ensure reliable results for each experiment.

Library Construction

A library is constructed by fragmenting the genomic DNA into short DNA molecules, then connecting the fragmented genomic DNA to universal adaptors, and then generating millions or even more single-molecule multi-copy PCR clone arrays.

In the present disclosure, any conventional method in the field can be used to fragment the amplified DNA and construct a DNA fragment library. For example, a commercially available kit can be used to fragment genomic DNA and construct a library of DNA fragments.

In certain embodiments, the process of fragmenting genomic DNA and constructing a DNA fragment library by using a kit may include:

(i) performing fragmentation on genomic DNA;
(ii) carrying out terminal modification on the obtained DNA fragments:
End Repair,
adding a phosphate group to the 5′ end of the DNA fragment repaired as above, and
adding an adenine nucleotide (A) to the 3′ end of the DNA fragment repaired as above (A-tailing);
   (iii) ligating an adapter and a sample barcode at the ends of the DNA fragment modified as above;
   (iv) Fragment Selection: agarose gel electrophoresis is performed on the ligation products as above, and the DNA fragments (i.e., DNA fragment library) correctly ligated with the adaptor and the sample barcode were recovered by using any commercially available kit.
   (v) Library Amplification: the DNA fragments with the adaptor and the sample barcode correctly ligated as above is amplified by polymerase chain reaction (PCR).

In a specific embodiment of the present disclosure, after MDA, the amplified DNA samples are subjected to secondary sequencing library construction using TruePrep DNA Library Prep Kit V2 for Illumina (Vazyme, TD503).

High Throughput Sequencing

In the present disclosure, as long as the high-throughput sequencing of the DNA fragment library can be realized, there is no special restriction on the sequencing method and apparatus adopted. In certain embodiments, the library of DNA fragments is high-throughput sequenced using a commercially available sequencer. For example, the high-throughput sequencing of DNA fragment library can be performed by using a sequencer from Illumina, a sequencer from Apply Biosystems (ABI), a sequencer from Roche, a sequencer from Helicos, or a sequencer from Complete Genomics.

In a specific embodiment, the genomic DNA of peripheral blood mononuclear cells and erythrocyte micronuclei DNA are sequenced by Novo-seq platform (NovaSeq 6000, from Novogene, Beijing), with 10×sequencing depth and 30 G data volume.

In the specific embodiment of the present disclosure, the original sequencing files for sequencing the erythrocyte micronuclei DNA and the genomic DNA of peripheral blood mononuclear cells are stored in FASTQ files. FASTQ is a standard text-based format to save biological sequences (usually nucleic acid sequences) and their sequencing quality information.

Bioinformatics Analysis

After high-throughput sequencing, bioinformatics analysis of the obtained sequencing results generally includes quality control, data comparison, post-alignment processing, among others.

In certain embodiments of the present disclosure, quality control is performed on the original sequencing files of erythrocyte micronuclei DNA, and the sequencing data passing the quality control is compared with the reference genome, and then post-processing is performed.

In a further embodiment of the present disclosure, quality control is performed on genomic DNA of peripheral blood mononuclear cells, and sequencing data passing the quality control is compared with a reference genome.

Quality Control

The sequencing data is quality controlled by data quality control software. The process of quality control includes adapter removal, filtering of low-quality reads, removal of low-quality 3′ and 5′ ends, removal of reads with more N, inspection of data quality, etc. Commonly used data software includes FastQC, Fastx_toolkit, Trimmomaic and so on.

As the most classic quality control software, FastQC can make quick statistics on gene information of high-throughput sequencing data and give corresponding chart reports. The software can be obtained at the following website: http://www.bioinformatics.babraham.ac.uk/projects/fastqc/.

In addition, Fastx_toolkit software can be obtained at the following website: http://hannonlab.cshl.edu/fastx_toolkit/; and Trimmomaic software can be obtained through the following web site: http://www.usadellab.org/cms/?page=trimmomatic.

In a specific embodiment of the present disclosure, the original sequencing files of erythrocyte micronuclei DNA and genomic DNA of peripheral blood mononuclear cells are subjected to adaptor removal by cutadapter software (Kong, Y., Btrim: a fast, lightweight adapter and quality trimming program for next-generation sequencing technologies. Genomics, 2011. 98(2): p. 152-3), and the quality control is carried out by FastQC software.

Data Comparison

After quality control, the data passed the quality control will be compared to the reference genome by a software. Sequencing data comparison software commonly used in this field includes BWA, Bowtie, Maq, Novoalign, etc., which can be obtained from the following website:

BMA: http://bio-bwa.sourceforge.net

Bowtie: http://bowtie-bio.sourceforge.net

Maq: http://maq.sourceforge.net

Novoalign: http://www.novocraft.com/products/novoalign/

In certain embodiments of the present disclosure, the sequencing data of erythrocyte micronuclei DNA and genomic DNA of peripheral blood mononuclear cells can be compared to reference genomes, such as human genomes, respectively, through data comparison software in the field. In a specific embodiment of the present disclosure, the sequencing data of erythrocyte micronuclei DNA and peripheral blood mononuclear cell genome DNA were compared to human genome (GenBank) by BWA software.

Post-Alignment Processing of Data

Post-alignment processing may include the following situations, such as removing duplicate reads, Indel local re-alignment, re-proofreading of base mass values, and so on. Whether or not to carry out post-alignment processing is determined according to actual needs. The commonly used post-alignment processing includes removing duplicate reads. Different reads aligned onto the same position of the reference genome may be considered as duplication due to quality problems, sequencing errors, alignment errors, alleles, among others.

In some embodiments of the present disclosure, post-alignment processing is performed by removing duplicate reads. In a specific embodiment of the present disclosure, improper alignment and repeated reads are removed by Picard software (Weisenfeld, N. I., et al., Direct determination of diploid genome sequences. Genome Res, 2017. 27(5): p. 757-767). Picard software can be obtained from the following website:

http://broadinstitute.github.io/picard/

Data Analysis

After data processing, the sequencing data obtained are analyzed.

Comparison and Counting of Reads

In certain embodiments of the present disclosure, whether there are significant differences in the fragmentation degree of DNA fragments in red blood cells of different types of subjects is compared. For example, the reads of sequencing fragments existing in micronuclei DNA of samples can be counted by software for reads counting (such as HTseq-count, featureCounts, BEDTools, Qualimap, Rsubread, GenomicRanges, etc.). Variance analysis (such as ANOVA test) is applied to judge whether there is a significant difference therebetween.

In certain specific embodiments of the present disclosure, the reads of small sequencing fragments existing in erythrocytes micronuclei DNA are counted corresponding to the gene regions of human genome by HTseq-count software (Anders, S., P. T. Pyl and W. Huber, HTSeq-a Python framework to work with high-throughput sequencing data. Bioinformatics, 2015. 31(2): p. 166-9).

In a specific embodiment of the present disclosure, one class is peripheral red blood cell micronuclei DNA from cervical cancer patients and the other class is peripheral red blood cell micronuclei DNA from healthy individuals.

In another specific embodiment of the present disclosure, one class is peripheral red blood cell micronuclei DNA from patients with cervical adenocarcinoma and the other class is peripheral red blood cell micronuclei DNA from cervical squamous cell carcinoma.

In another specific embodiment of the present disclosure, one class is peripheral red blood cell micronuclei DNA from medium-differentiated patients in cervical squamous cell carcinoma, and the other class is peripheral red blood cell micronuclei DNA from low-medium differentiated or low differentiated patients in cervical squamous cell carcinoma.

In a further embodiment of the present disclosure, one class is peripheral red blood cell micronuclei DNA from colorectal cancer patients and the other class is peripheral red blood cell micronuclei DNA from healthy individuals.

In a further embodiment of the present disclosure, one class is peripheral red blood cell micronuclei DNA from colon cancer patients and the other class is peripheral red blood cell micronuclei DNA from rectal cancer.

Data Classification and Classifier Construction

Classification is an important method of data mining. Based on the existing data, a classification function is learned or a classification model is constructed, which also called a classifier. Classifiers can map data records in the database to a given class, which can be applied to data prediction. Classification methods include decision tree, selection tree, logistic regression, Naive Bayes and deep neural network.

In certain embodiments of the present disclosure, genes with significant differences are selected as features, and a classifier is constructed for known classified samples based on support vector machine (SVM) to predict the specific disease classification of unknown samples (Huang, M. W., et al., SVM and SVM Ensembles in Breast Cancer Prediction. PLoS One, 2017. 12(1): p. e0161501). In some specific embodiments of the present disclosure, through the hierarchical clustering-based support vector machine algorithm, a classifier composed of a group of genes corresponding to DNA fragments is constructed. In a specific embodiment of the present disclosure, two types of samples are randomly clustered according to Pearson correlation to construct a classifier composed of a group of genes.

In certain embodiments of the present disclosure, specific regions of erythrocyte micronuclei DNA are further selected before constructing the classifier.

In certain embodiments of the present disclosure, macs2 software is used to search for the fragments of erythrocyte micronuclei DNA which are mainly enriched in a specific region relative to the genome DNA sequencing reads of peripheral blood mononuclear cells, and to remove the peak areas which are more enriched by peripheral blood mononuclear cells relative to peripheral blood mononuclear cells per se as a whole. Compared with peripheral blood mononuclear cells, genome information annotation and pathway enrichment (KEGG, gene ontology) were performed on red blood cell-specific fragments (Chen, L., et al., Gene Ontology and KEGG Pathway Enrichment Analysis of a Drug Target-Based Classification System. PLoS One, 2015. 10(5): p. e0126492.).

Application of Classifier

On the basis of the classifier constructed in the present disclosure, the present invention can be widely used in biological research, medical research, clinical diagnosis and other fields by isolating peripheral blood micronuclei DNA from subjects in the manner described in the present disclosure and performing biological analysis. The invention has important value in scientific research and medical fields.

### Application of the Invention

The inventors have successfully isolated erythrocyte micronuclei DNA from peripheral blood and applied it to cancer detection for the first time, including screening, diagnosis, typing and staging of cancer.

Among cancers, cervical cancer and colorectal cancer account for a large proportion of new cases and fatal cases.

Cervical Cancer

Cervical cancer is one of the most common gynecological tumors, and its incidence is increasing year by year. According to the statistics of the World Health Organization (WHO), there are an average of 530,000 new cases of cervical cancer every year, and about 250,000 women die from cervical cancer, among which developing countries account for 80% of the global total cases (Schiffman, M., et al., Carcinogenic human papillomavirus infection. Nat Rev Dis Primers, 2016. 2: p. 16086). In China, there are about 140,000 new cases of cervical cancer and about 37,000 deaths every year. Therefore, early screening and clinical staging of cervical cancer patients are of great significance to the treatment of cervical cancer.

Pathogenic Factors of Cervical Cancer

Pathogenic factors of cervical cancer include but are not limited to the following aspects:

Virus Infection

HPV infection is the main pathogenic factor of cervical cancer. There are many subtypes of HPV, about 40 of which are related to reproductive tract infection. Continuous infection by high-risk HPV subtypes (subtypes 16, 18, 31, 33, 35, 39, 45, 51, 52, 56, 58, 59 and 69), especially HPV subtypes 16 and 18 can cause cervical cancer.

Sexual Behavior and Number of Deliveries

Other Biological Factors

Chlamydia trachomatis, herpes simplex virus type II, trichomoniasis and other pathogens have synergistic effects in the pathogenesis of cervical cancer caused by high-risk HPV infection.

Other Behavioral Factors

Smoking as a synergistic factor of HPV infection can increase the risk of cervical cancer. In addition, malnutrition and poor sanitation can also affect the occurrence of diseases.

Early Screening Method for Cervical Cancer in the Prior Art

At present, the early screening of cervical cancer is mainly carried out by virus detection and cytological detection. Among them, virus detection is mainly human papillomavirus (HPV) detection, while cytological detection mainly includes Pap smear and TCT detection.

(1) HPV Detection

HPV can cause squamous epithelial proliferation of human skin mucosa. According to its pathogenicity, it can be divided into low-risk type and high-risk type. Low risk infection can cause common warts, genital warts (condyloma acuminatum) and other symptoms. Persistent high-risk human papillomavirus (HPV) infection is the main cause of cervical cancer. Molecular epidemiological analysis shows that some types of human papillomavirus (HPV) are the main causes of invasive cervical cancer and cervical intraepithelial neoplasia. At present, more than 80 types of HPV have been found, and about 40 of them can infect reproductive tract [Schiffman, M., et al., Carcinogenic human papillomavirus infection. Nat Rev Dis Primers, 2016. 2: p. 16086; Munoz, N., et al., Epidemiologic classification of human papillomavirus types associated with cervical cancer. N Engl J Med, 2003. 348(6): p. 518-27.]. Among them, high-risk HPV (such as HPV 16 and HPV 18) is often associated with invasive cervical cancer. The detection methods of high-risk HPV mainly include morphological observation, immunohistochemistry, dot-blot hybridization, in situ blotting hybridization, PCR/RFLP, PCR/Southern and so on.

Screening cervical cancer by HPV virus detection can identify more than 95% of precancerous cervical lesions, but it is mainly aimed at patients with cervical intraepithelial neoplasia (CIN) grade 2 or more, while the specificity for CIN2 negative patients is relatively low, because most women have spontaneous clearance after transient HPV infection, and hardly progress to C1N3 and cancer (Cook, D. A., et al., Evaluation of a validated methylation triage signature for human papillomavirus positive women in the HPV FOCAL cervical cancer screening trial. Int J Cancer, 2018). HPV detection can only determine whether women are infected with carcinogenic HPV, but cannot determine the risk of individual cancer, and there are still few HPV negative cervical cancer patients. Therefore, there may be false positives in HPV testing. On the basis of HPV detection, it is usually necessary to combine other clinical detection indications for subsequent diagnosis.

(2) Pap Smear

Pap Smear, also known as cervical smear or Pap test, is a traditional and most commonly used screening method for cervical cancer. In this method, the cervical exfoliated cells are collected, stained and microscopically observed to test whether there are precancerous cells or cancer cells on the cervix, which has always been regarded as the “gold standard” for cervical cancer detection (Rodriguez, A. C. and J. Salmeron, Cervical cancer prevention in upper middle-income countries. Prev Med, 2017. 98: p. 36-38).

Combined with pathological observation, Pap smear can clearly identify the development of cervical cancer, but this method can only detect about 50% of cervical precancerous lesions. The difference in sample collection quality, insufficient cell collection, fewer abnormal cells, and the shielding of abnormal cells by blood or inflammatory cells will affect smear observation, resulting in poor detection sensitivity (Cook, D. A., et al., Evaluation of a validated methylation triage signature for human papillomavirus positive women in the HPV FOCAL cervical cancer screening trial. Int J Cancer, 2018). At the same time, due to the limitation of sampling, it is difficult to have regular detection and trace cases.

(3) TCT Detection

TCT test, also named as liquid-based thin-layer cytology test, collects cervical cell samples through a special sampler, but does not directly carry out smear observation, and instead puts the collector into a culture bottle filled with cell preservation solution for rinsing to obtain enough cell samples (Massad, L. S., et al., 2012 updated consensus guidelines for the management of abnormal cervical cancer screening tests and cancer precursors. Obstet Gynecol, 2013. 121(4): p. 829-46). After that, the cell sample bottles were sent for laboratory inspection, and the cell samples were dispersed and filtered by automatic cell detector, so as to reduce the interference of blood, mucus and inflammatory tissues and obtain a thin cervical cell layer for further microscopic detection and diagnosis.

TCT detection is an optimized detection scheme for pap smear of cervical cancer developed in recent decades. Compared with the traditional Pap smear of cervical cancer, TCT detection significantly improved the satisfaction of specimens and the detection rate of abnormal cells of cervical cancer. The detection rate of cervical cancer cells by TCT was 100%, and some precancerous lesions could also be found (Andy, C., L. F. Turner and J. O. Neher, Clinical inquiries. Is the ThinPrep better than conventional Pap smear at detecting cervical cancer? J Fam Pract, 2004. 53(4): p. 313-5). However, the detection rate of TCT for cervical precancerous lesions is still low, and the sensitivity for early screening and detection of cervical cancer is low, and there are still many atypical squamous cells (ASC-US) and atypical glandular cells (AGC) with unknown meanings.

The above methods still have some limitations. First of all, for the above-mentioned methods, it is often necessary to use combined screening methods in clinical use (Zigras, T., et al., Early Cervical Cancer: Current Dilemmas of Staging and Surgery. Curr Oncol Rep, 2017. 19(8): p. 51). Secondly, at present, the samples used for cervical cancer screening by the above method are cervical exfoliated cells, and the sampling method will inevitably cause damage and psychological burden to patients, and at the same time, there are certain restrictions on sampling requirements, and the quality of samples is difficult to control. In addition, screening for cervical cancer often requires regular detection. According to FDA standards, for women over 21 years old, regular detection should be conducted every 3 years to assess the risks. The large fluctuation of sampling quality stability may lead to the loss of long-term regression testing. Therefore, a more reliable and stable sample source is needed to provide a more dynamic, accurate and instructive monitoring method and system for cervical cancer screening.

In the context of the present disclosure, “cervical cancer” includes any type of cervical cancer.

Classification and Staging of Cervical Cancer

The occurrence and development of cervical cancer has a gradual evolution process, which can last from several years to several decades. It is generally considered that the evolution can be divided into several stages: mild intraepithelial neoplasia (CINI), moderate intraepithelial neoplasia (CINII), severe intraepithelial neoplasia (CINIII) and invasive cancer.

Cervical cancer can be classified into different types according to different standards.

According to whether cancer has metastasized or not, cervical cancer can be divided into cancer in situ and invasive cancer. Cancer in situ is more common in women aged 30-35, while invasive cancer is more common in women aged 45-55. Lymphatic metastasis may occur in patients with severe cervical cancer. After local infiltration, the cancer invaded lymphatic vessels to form tumor plugs, which were drained into local lymph nodes with lymph fluid and spread in lymphatic vessels.

According to pathological types, cervical cancer can be divided into three types: squamous cell carcinoma, adenocarcinoma and adeno-squamous carcinoma.

Cervical squamous cell carcinoma is the main type of cervical cancer. According to histological differentiation, it can be divided into three grades: Grade I is highly differentiated squamous cell carcinoma, Grade II is medium differentiated squamous cell carcinoma (non-keratinized large cell type), and Grade III is low-medium differentiated and low differentiated squamous cell carcinoma (small cell type).

Cervical adenocarcinoma includes mucinous adenocarcinoma type and malignant adenoma type. Mucinous adenocarcinoma originates from columnar mucous cells of cervical canal, and the glandular structure can be seen under microscope. The hyperplasia of glandular epithelial cells is multilayer, the dysplasia is obvious, and mitosis is seen. The cancer cells protrude into the glandular cavity in mastoid shape. Malignant adenoma is a highly differentiated adenocarcinoma of cervical canal mucosa. There are many cancerous glands with different sizes and varied shapes, which extend into the deep cervical stroma in a punctate way. The glandular epithelial cells are atypical and often have lymph node metastasis.

Unexpectedly, the inventors found that peripheral red blood cell micronuclei DNA can be used for screening and diagnosing cervical cancer. The inventors further unexpectedly found that the micronuclei DNA of peripheral red blood cells can be used to distinguish the types of cervical cancer, which can be divided into squamous cell carcinoma and adenocarcinoma. The inventor further unexpectedly found that, peripheral red blood cell micronuclei DNA can stage cervical cancer, for example, cervical squamous cell carcinoma can be divided into high-differentiated type, medium-differentiated type, and low-medium-differentiated and low-differentiated type. It is of great significance for the early diagnosis, screening, classification and staging of cervical cancer.

Colorectal Cancer

Colorectal cancer (CRC) is a cancer that arises from the colon or rectum. It is one of the most common malignant tumors in the gastrointestinal tract. The early symptoms are not obvious. The symptoms and signs shown with the increase of the cancer can include blood in stools, weight loss, and constant fatigue (General Information About Colon Cancer. NCI. May 12, 2014. Archived from the original on Jul. 4, 2014. Retrieved Jun. 29, 2014).

There are approximately 1.4 million new cases of colorectal cancer each year. Colorectal cancer ranks third among newly diagnosed cancers, and it is also the fourth cause of death from cancer. Studies have shown that by 2030, the number of global colorectal cancer cases is expected to increase by 60%, with more than 2.2 million new cases per year and approximately 1.1 million deaths per year (Global patterns and trends in colorectal cancer incidence and mortality. M, et al. Gut. 2017; 66:683-91).

Globally, colorectal cancer is the third most common cancer, accounting for about 10% of all cancer cases. It is especially common in developed countries, where more than 65% of cases are found to be CRC, and it is usually less common in women than in men (Forman D, Ferlay J (2014). “Chapter 1.1: The global and regional burden of cancer”. In Stewart B W, Wild C P (eds.). World Cancer Report. the International Agency for Research on Cancer, World Health Organization. pp. 16-53. ISBN 978-92-832-0443-5).

With the improvement of people's living standards in China, the incidence of colorectal cancer is on the rise. The latest statistics show that the incidence and mortality of colorectal cancer (CRC) in China have maintained an upward trend. Cancer statistics in China in 2015 show that the incidence and mortality of colorectal cancer in my country rank fifth among all malignant tumors, with 376,000 new cases and 191,000 deaths. Among them, the amount in urban areas is much higher than that in rural areas, and the incidence of colon cancer has increased significantly. Most patients are already in the middle and late stages when found. Early diagnosis of colorectal cancer is extremely important, and early diagnosis can significantly increase the possibility of successful treatment (5. Standards for Diagnosis and Treatment of Colorectal Cancer in China (2017 Edition) [J]. Chinese Journal of Medical Frontiers (Electronic Edition), 2018, 10(3): 1-21).

Causes of Disease

Most colorectal cancers are caused by factors like aging and lifestyle, and only a few cases are caused by potential hereditary diseases. Risk factors include diet, obesity, smoking and lack of physical activity. Another risk factor is inflammatory bowel disease, including Crohn's disease and ulcerative colitis. Some hereditary diseases lead to colorectal cancer, including familial adenomatous polyposis and hereditary nonpolyposis colon cancer. CRC usually begins with benign tumor and appears as polyp, which may become cancerous with time.

Classification

Classification According to Causes

According to the causes, colorectal cancer can be divided into three classes, two of which have genetic factors:
sporadic colorectal cancer (50% to 60%);
familial colorectal cancer (30% to 40%);
hereditary colorectal cancer (4% to 6%).

Sporadic colorectal cancer: Sporadic colorectal cancer is the most common type, with 90% of patients diagnosed at the age of 50 and above. It is not directly related to genetics or family history. About one in every 20 Americans has this type of CRC.

Familial colorectal cancer: Some families are prone to CRC. If more than one person in the family suffers from CRC, especially before the age of 50, attention must be paid to it. If immediate family members (parents, siblings or children) have colorectal cancer, the risk of such family members will double.

Hereditary colorectal cancer: At present, many hereditary diseases have been found to be related to CRC, including hereditary nonpolyposis colon cancer (HNPCC), also known as Lynch syndrome; Familial adenomatous polyposis (FAP); Attenuated familial adenomatous polyposis (AFAP); APCI 1307K; Potts-Jaggers syndrome; MYH-associated polyposis (MAP); Juvenile polyposis; Hereditary polyposis.

Classification According to Focus of Cancer

According to the focus of cancer, colorectal cancer can be divided into colon cancer and rectal cancer.

Importance of Early Screening

Lifestyles such as high-fat diet, smoking and alcoholism may increase the risk of colorectal cancer. More than 90% of colorectal cancer patients are over 50 years old. Usually, the best treatment period is missed because of neglecting the early symptoms of the disease, including bloody feces or changes in defecation habits. Early diagnosis can significantly increase the possibility of successful treatment.

In recent years, in the United States, the incidence rate and mortality rate of CRC are gradually decreasing. The microscopic simulation model MISCAN-Colon suggests that the observed mortality rate of CRC is decreasing, and about 53% of it may benefit from CRC screening. In 2012, 65.1% of adults at the age of 50-75 in the United States had been screened for CRC, and 27.7% had never been screened. Colonoscopy is the most frequently used screening examination (nearly 62%). From 2002 to 2010, the screening rate increased from 52.3% to 65.4%. With the improvement of screening rate, early treatment and intervention for risk individuals significantly reduced the incidence rate and mortality rate of CRC (Cronin K A, Lake A J, Scott S, et al. Annual Report to the Nation on the Status of Cancer, part I: National cancer statistics. Cancer 2018; 124:2785).

Early Screening and Diagnosis Methods of Colorectal Cancer in the Prior Art

Early screening and diagnosis of colorectal cancer mainly include the following ways:

(1) Colonoscopy

Colonoscopy is the most accurate and universal diagnostic examination of CRC, which can locate lesions in the whole large intestine and perform biopsy to find simultaneous tumors and remove polyps. Observed under endoscope, most colon cancer and rectal cancer are intraluminal masses which originate from mucosa and protrude into the lumen. Tumors can be exophytic or polypoid. Bleeding (blood oozing or obvious bleeding) may be observed in fragile, necrotic or ulcerated lesions. A few gastrointestinal tumor lesions (both asymptomatic and symptomatic individuals) are non-polypoid. A study found that, non-polypoid colorectal tumors are more prone to carcinogenesis than polypoid tumors. Compared with polypoid lesions, cancer caused by non-polypoid adenoma may be more difficult to be detected under colonoscopy, but colonoscopy is more sensitive to this situation than barium enema or CT colonography. When experienced endoscopy operators use colonoscopy to examine asymptomatic patients, the missed diagnosis rate of CRC is 2%-6%.

(2) Flexible Sigmoidoscopy

It has been observed that in the past 50 years, the proportion of right colon cancer or proximal colon cancer in the United States and around the world is gradually increasing, and the incidence of tumors originating from cecum is increasing with the fastest speed. In view of this, and considering the high incidence of simultaneous CRC, for patients with suspected CRC, flexible sigmoidoscopy is generally considered not to be an appropriate diagnostic examination, unless the tumor is palpable in rectum. In this case, total colonoscopy is still needed to assess whether the rest of the colon has simultaneous polyps and cancer. However, flexible sigmoidoscopy is used to screen CRC. It is one of the few methods that have been proved by randomized controlled trials to reduce the incidence and morbidity of CRC.

(3) CT Colonography

CT colonography, also known as virtual colonoscopy or CT colonography, can provide a computer-simulated intraluminal perspective for the inflated colon. This technology uses traditional spiral CT scanning or MRI to obtain a large number of continuous data, and uses complex post-processing software to generate images, which can enable operators to walk and pass in any selected direction in the clean colon cavity. CT colonography needs mechanical bowel preparation similar to barium enema, because feces can be similar to polyps in image, causing interference. CT colonography can also detect extracolonic lesions, which can provide information on the causes of symptoms and tumor staging, but it may also lead to anxiety and increase costs due to unnecessary examinations. And its detection rate for clinically important lesions may also be low.

Compared with colonoscopy, CT colonography is an alternative with similar sensitivity and less trauma for patients with CRC. However, considering that colonoscopy can remove/biopsy the lesions and simultaneous cancers or polyps seen during the operation, colonoscopy is still considered as the gold standard for CRC symptoms. When the use of colonoscopy is limited, CT colonography is preferred over barium enema (Mulder S A, Kranse R, Damhuis R A, et al. Prevalence and prognosis of synchronous colorectal cancer: a Dutch population-based study. Cancer Epidemiol 2011; 35:442).

However, due to the particularity of sampling and testing methods, the above screening methods will inevitably lead to psychological burden and local injury of some screeners, which is also an influencing factor that limits the long-term and large-scale screening, and it is necessary to consider the adaptability of patients' age and screening methods.

(4) Guaiac-Based Faecal Occult Blood Test (gFOBT)

This test detects whether there is blood in a patient's stool sample. But blood stool test is not 100% accurate, because not all cancers cause bleeding, or they may not bleed all the time. Therefore, this test can give false negative results. Blood may also be present due to other diseases or conditions, such as hemorrhoids. The method of detecting fecal hemoglobin by guaiac is an indirect method for detecting peroxidase activity. There are non-hemoglobin peroxidase catalytic components in various foods, which may cause false positive, thus limiting the application of this method. Its advantage lies in the convenience and rapidity of initial detection and screening, which has certain guiding significance for further detection and diagnosis, but its accuracy is relatively low.

(5) Immunochemical Test (Faecal Immunochemical Test, FIT)

This test uses antibodies to detect fecal occult blood. FIT uses monoclonal or polyclonal antibodies to directly detect hemoglobin in human feces, which is not affected by dietary. In qualitative FIT, color change is visible after the hemoglobin content in feces exceeds a certain threshold. While quantitative FIT can measure the value: when it exceeds a certain normal range, it is defined as positive. Compared with gFOBT, immunochemical test requires less stool samples, and there is no dietary restriction before collecting stool samples, but only one or two stool samples are collected each time (Mettle Kalager, et al. Overdiagnosis in Colorectal Cancer Screening: Time to Acknowledge a Blind Spot[J]. Gastroenterology, 2018 Aug. 1). Even if there is merely occult blood in a sample, occult blood can also be detected. Occult blood in a sample indicates intestinal bleeding. This method has relatively high specificity, but poor sensitivity, and there may also be false positive or negative results due to interference from other diseases, which makes it impossible to make a definite diagnosis.

(6) Fecal DNA Test

Colorectal cancer generally occurs in colorectal epithelial tissue, and first grows into intestinal cavity. During its growth, tumor cells are constantly shed into intestinal cavity and discharged with feces. The shed tumor cells in feces contain special components (such as mutated and methylated human genes), which can be used as tumor markers. Fecal DNA test analyzed several DNA markers of colon cancer or precancerous polyp cells flowing into feces. Patients can be provided with a kit containing instructions on how to collect stool samples at home, and then send it to the laboratory for detection and analysis. This test is more accurate for detecting colon cancer than polyp, but it cannot detect all DNA mutations that indicate the existence of tumor. The value of fecal gene detection lies in early diagnosis, which can prompt the occurrence of colorectal cancer, find precancerous adenoma and help patients to find colorectal cancer at an earlier stage (Imperiale, T. F., et al., Multitarget Stool DNA Testing for Colorectal-Cancer Screening. New England Journal of Medicine, 2014. 370(14): p. 1287-1297). However, fecal genetic testing can only be used as an auxiliary diagnostic method. If there is a positive result, it must be confirmed and intervened by colonoscopy. However, due to the complexity of fecal DNA, its low specificity and low success rate of fecal DNA preparation will lead to insufficient cost-effectiveness, which greatly hinders its practical application.

The above methods are relatively convenient for sampling, and non-invasive. Non-invasive detection is more acceptable to patients, which may be used as an indicator of CRC screening. However, due to the specificity and sensitivity of the methods, most of them can only be used as an auxiliary means of diagnosis, and other means such as colonoscopy are still needed for diagnosis and intervention. Meanwhile, for stool sampling and treatment, the psychological burden to a certain extent, as well as the complexity and pollution of stool samples, also cause problems in the stability and repeatability of sample detection (Brenner, H., et al., Prevention, Early Detection, and Overdiagnosis of Colorectal Cancer Within 10 Years of Screening Colonoscopy in Germany. Clinical Gastroenterology and Hepatology, 2015. 13(4): p. 717-723). Therefore, a more reliable and stable sample source is needed to provide a more dynamic, accurate and instructive monitoring system for CRC screening.

Surprisingly, the inventors found that peripheral red blood cell micronuclei DNA can be used to screen and diagnose colorectal cancer. The inventors further surprisingly found that micronuclei DNA of peripheral red blood cells can be used to distinguish the types of colorectal cancer, which can be divided into colon cancer and rectal cancer. It is of great significance for early diagnosis, screening and risk ranking of colorectal cancer.

Lung Cancer

Lung cancer is the most common cancer type worldwide in terms of both incidence and mortality. The key cause of lung cancer is tobacco smoking, which is responsible for 63% of overall global deaths from lung cancer and for more than 90% of lung cancer deaths in countries where smoking is prevalent in both sexes. Causes of lung cancer also including: secondhand smoke, family history of lung cancer, exposed to asbestos, arsenic, chromium, beryllium, nickel, soot, or tar in the workplace, air population, etc.

Classification according to causes:

According to the causes, lung cancer can be divided into two main classes are small-cell lung carcinoma (SCLC) and non-small-cell lung carcinoma (NSCLC).

SCLC (10%-15%): This type of lung cancer is the most aggressive and rapidly growing of all the types. SCLC is strongly related to cigarette smoking. SCLCs metastasize rapidly to many sites within the body and are most often discovered after they have spread extensively.

NSCLC (85%): NSCLC has three main types designated by the type of cells found in the tumor. They are:
Adenocarcinomas (40%), while adenocarcinomas are associated with smoking like other lung cancers, this type is also seen in non-smokers—especially women—who develop lung cancer. Adenocarcinoma in situ (previously called bronchioloalveolar carcinoma) is a subtype of adenocarcinoma that frequently develops at multiple sites in the lungs and spreads along the preexisting alveolar walls. It may also look like pneumonia on a chest X-ray. It is increasing in frequency and is more common in women. People with this type of lung cancer tend to have a better prognosis than those with other types of lung cancer;
Squamous cell carcinomas (25%-30%), squamous cell cancers arise most frequently in the central chest area in the bronchi. This type of lung cancer most often stays within the lung, spreads to lymph nodes, and grows quite large, forming a cavity;
Large cell carcinomas (10%-15%), this type of cancer has a high tendency to spread to the lymph nodes and distant sites.
   Other types of cancers can arise in the lung; these types are much less common than NSCLC and SCLC and together comprise only 5%-10% of lung cancers.

The Diagnosis of Lung Cancer is Mainly Focused on Imaging Examination:

(1) X-ray inspection: X-ray examination can understand the location and size of lung cancer, and may see local emphysema, atelectasis, or infiltrating lesions or pulmonary inflammation near the lesion due to bronchial obstruction. (2) Bronchoscopy: the bronchoscope can directly observe the pathological conditions of the bronchial lining and lumen. Tumor tissue can be taken for pathological examination, or bronchial secretions can be drawn for cytological examination to confirm the diagnosis and determine the histological type. (3) Cytological examination: sputum cytology is a simple and effective method for general screening and diagnosis of lung cancer. Most patients with primary lung cancer can find shed cancer cells in the sputum. The positive rate of sputum cytology for central lung cancer can reach 70% to 90%, while that for peripheral lung cancer is only about 50%. (4) ECT inspection:ECT bone imaging can detect bone metastases earlier. Both X-ray film and bone imaging have positive findings. If the osteogenesis reaction of the lesion is static and the metabolism is not active, the bone imaging is negative and the X-ray film is positive. The two complement each other and can improve the diagnosis rate. (6) Mediastinoscopy: mediastinoscopy is mainly used for patients with mediastinal lymph node metastasis, which is not suitable for surgical treatment, and other methods cannot obtain pathological diagnosis.

Surprisingly, the inventors found that peripheral red blood cell micronuclei DNA (rbcDNA) can be used to screen and diagnose lung cancer. The inventors further surprisingly found that rbcDNA signature is of great significance for early diagnosis, screening and risk ranking of lung cancer.

Hepatocellular Cancer

Hepatocellular cancer (HCC) is the fifth most common cause of cancer, and the incidence is increasing globally due to the spread of hepatitis B and C virus infections, causes also including: cirrhosis, heavy drinking, obesity and diabetes, abusive anabolic steroids, iron storage disease and aflatoxin. If caught early, it can sometimes be cured by surgery or transplantation. In more severe cases, it cannot be cured.

Detection of Serum Bio-Markers for Hepatocellular Cancer

(1) The determination of serum alpha-fetoprotein (AFP) is relatively specific for the diagnosis of this disease. Immunoassay measures continuous serum AFP≥400 μg/L, and can rule out pregnancy, active liver disease, etc., then the diagnosis of liver cancer can be considered. However, approximately 30% of patients with liver cancer are clinically negative for AFP. (2) Blood enzymology and other tumor marker examinations. The levels of γ-glutamyl transpeptidase and its isoenzymes, abnormal prothrombin, alkaline phosphatase, and lactate dehydrogenase isoenzymes in the serum of patients with liver cancer may be higher than normal, but it lacks specificity.

Imaging Examination

(1) Ultrasound examination can show the size, shape, location of the tumor and whether there are tumor thrombi in the hepatic vein or portal vein, and the diagnostic coincidence rate can reach 90%. (2) CT examination has a high resolution, and the diagnostic coincidence rate for liver cancer can reach more than 90%, and it can detect small cancer foci with a diameter of about 1.0 cm. (3) The diagnostic value of MRI is similar to that of CT. It is better than CT in distinguishing benign and malignant intrahepatic lesions, especially from hemangioma. (4) Selective angiography of celiac artery or hepatic arteriography. For cancers with abundant blood vessels, but the low-resolution limit for small liver cancers tumor volume less than 2.0 cm, the positive rate can reach 90%. (5) Needle aspiration cytology for liver puncture, needle aspiration under the guidance of B-mode ultrasound can help increase the positive rate in cancer diagnosis, but with invasive tissue damage.

Surprisingly, the inventors found that peripheral red blood cell micronuclei DNA (rbcDNA) can be used to screen and diagnose hepatocellular cancer. The inventors further surprisingly found that rbcDNA signature is of great significance for early diagnosis, screening and risk ranking of hepatocellular cancer.

Combined Application of the Invention and Other Methods

In certain embodiments, the methods of the present disclosure can also be combined with other methods for screening, diagnosing or risk ranking of cancer. Those skilled in the art can select suitable other methods in the prior art as required.

In certain embodiments, methods related to cervical cancer that can be combined with the methods of the present disclosure include, for example, detection of high-risk HPV and cytological examination of cervical exfoliated cells. In an embodiment, the detection methods for high-risk HPV include morphological observation method, immunohistochemistry method, dot-blot hybridization method, blotting hybridization in situ, PCR/RFLP method, PCR/Southern method and the like. In one embodiment, the cytological examination of cervical exfoliated cells includes TCT, Pap smear, etc.

In certain embodiments, methods related to colorectal cancer that can be combined with the methods of the present disclosure include, for example, colonoscopy, flexible sigmoidoscopy, CT colonography, fecal occult blood test, immunochemical test, fecal DNA test, and the like.

### EXAMPLES

In the following section, the present invention is further illustrated by examples. Examples are provided by way of illustration, but the present invention is not limited to the following examples. In the following examples, the subjects are all human subjects.

### Example 1: Density Gradient Centrifugation of Peripheral Blood

Through the following steps, the peripheral blood samples of each subject were subjected to density gradient centrifugation.

Step 1. 1 ml fresh peripheral blood was obtained from a subject, and 1×PBS was added in equal volume to prepare a diluted blood sample.

Step 2. 5 ml Ficoll density gradient centrifuge (Stemcell, Lymphoprep™ 07801) was added into the density gradient centrifuge tube.

Step 3. The diluted blood sample prepared in step 1 was slowly added to the density gradient centrifuge tube in Step 2. Density gradient centrifugation was performed at 1200 g at 18° C. for 15 minutes.

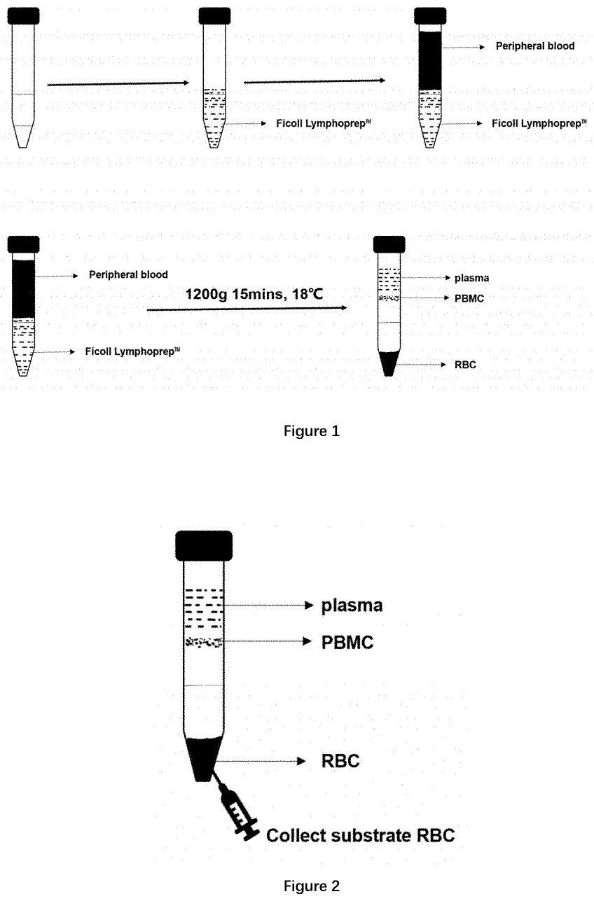

After density gradient centrifugation, the sample was divided into three layers: the upper layer was plasma, the middle layer was peripheral blood mononuclear cells (PBMC), and the bottom layer was red blood cells (as shown in FIG. 1).

### Example 2: Separation of Blood Cells

After density gradient centrifugation in Example 1, peripheral blood mononuclear cells and red blood cells were separated.

Specifically, as shown in FIG. 2, the middle-upper layer liquid in the density gradient centrifuge tube was sucked through a straw, and the peripheral blood mononuclear cell sample was separated and collected. Red blood cells were extracted from the bottom of density gradient centrifuge tube via a needle tube, added into a 1.5 ml centrifuge tube. 1×PBS was added into the centrifuge tube to a volume of 1 ml liquid. Centrifugation was proceeded at room temperature for 10 min at 300 g, and red blood cells at the bottom of the tube were collected. The collected RBCs were then subject to two sequential filtrations by 10 μm cell strainers to remove potential contamination of nucleated cells.

### Example 3: DNA Extraction

In this example, the genome of peripheral blood mononuclear cells and erythrocyte micronuclei DNA were extracted, respectively.

3.1 Extraction of Genomic DNA from Peripheral Blood Mononuclear Cells

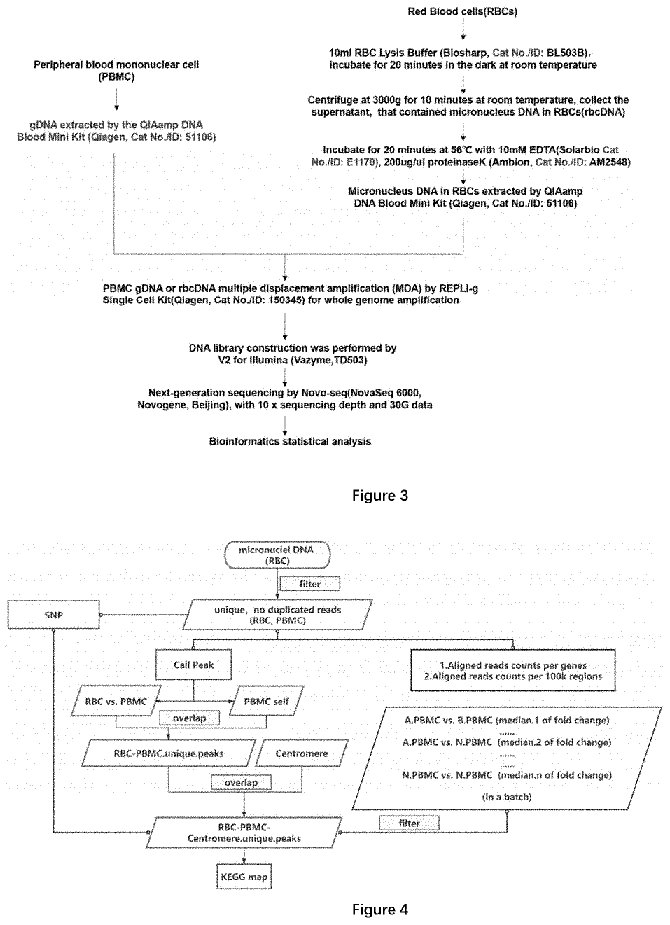

Genomic DNA was extracted from the peripheral blood mononuclear cell sample obtained in Example 2 using QIAamp DNA Blood Mini Kit (Qiagen, Cat No./ID: 51106), as shown in FIG. 3.

3.2 Extraction of Erythrocyte Micronuclei DNA

Red blood cells obtained in Example 2 were lysed by a red blood cell lysis buffer. Specifically, 10 ml of red blood cell lysis buffer (Biosharp, Cat No./ID: BL503B) was added to the red blood cells collected in Example 2, lysed for 20 minutes at room temperature in the dark, and centrifuged at 3000 g at room temperature for 10 minutes. Supernatant was taken and incubated with 10 mm EDTA (Solarbio Cat No./ID: E1170) and 200 ug/ul protease K (Ambion, Cat No./ID: AM2548) at 56° C. for 8 hours. Erythrocyte micronuclei DNA was extracted using QIAamp DNA Blood Mini Kit (Qiagen, Cat No./ID: 51106).

### Example 4: DNA Amplification, Library Construction and Sequencing

Genomic DNA of peripheral blood mononuclear cells and erythrocyte micronuclei DNA extracted in Example 3 were amplified, library constructed and sequenced, respectively.

4.1 DNA Amplification

Genomic DNA of peripheral blood mononuclear cells and erythrocyte Micronuclei DNA prepared in Example 3 were subjected to multiple displacement amplification (MDA) using REPLI-g Single Cell Kit(Qiagen, Cat No./ID: 150345), to obtain amplified DNA samples.

4.2 Library Construction

After MDA, the amplified DNA samples were subjected to second-generation sequencing library construction using TruePrep DNA Library Prep Kit V2 for Illumina (Vazyme, TD503).

4.3 High-Throughput Sequencing

Genomic DNA of peripheral blood mononuclear cells and erythrocyte micronuclei DNA were sequenced by Novo-seq platform, with 10×sequencing depth and 30 G data.

### Example 5: Bioinformatics Analysis of Erythrocyte Micronuclei DNA and Genomic DNA of Peripheral Blood Mononuclear Cells

Bioinformatics analysis was made on micronuclei DNA information in red blood cells by the following steps (see FIG. 4 for the logic of bioinformatics analysis algorithm):

1. Quality control. Quality control on the original sequencing files of double-ended sequencing of erythrocyte micronuclei DNA and peripheral blood mononuclear cell genome DNA respectively through FastQC software.

2. Adaptor removal. Adaptor removal in the original sequencing file through cutadapter software (Kong, Y., Btrim: a fast, lightweight adapter and quality trimming program for next-generation sequencing technologies. Genomics, 2011. 98(2): p. 152-3). According to sequencing quality, the reads of small fragments with appropriate length and accurate pairing were reserved.

3. Data alignment. Sequenced fragments of red blood cell micronuclei DNA and peripheral blood mononuclear cell genome DNA were analyzed by bwa software (http://bio-bwa.sourceforge.net) was aligned to the human genome, and inappropriate and repeated reads were removed by Picard (Weisenfeld, N. I., et al., Direct determination of diploid genome sequences. Genome Res, 2017. 27(5): p. 757-767).

4. Comparison and counting of reads. The reads of sequenced small fragments in erythrocyte micronuclei DNA were counted corresponding to the gene regions of human genome using htseq-count software (Anders, S., P. T. Pyl and W. Huber, HTSeq-a Python framework to work with high-throughput sequencing data. Bioinformatics, 2015. 31(2): p. 166-9), to compare whether there were significant differences in the degree of DNA fragmentation in red blood cells of healthy individuals and cancer patients.

5. Peak Calling. Searching for the fragments of red blood cell micronuclei DNA which were mainly enriched in a specific genetic region relative to the genome DNA sequencing reads of peripheral blood mononuclear cells through macs2 software, and removing the peak areas which were more enriched by peripheral blood mononuclear cells relative to PBMC per se as a whole.

6. Genome information annotation and pathway enrichment of specific broken fragments in erythrocyte micronuclei DNA. Compared with peripheral blood mononuclear cells, genome information annotation and pathway enrichment (KEGG, Gene Ontology) were performed on specific broken fragments of erythrocytes (Chen, L., et al., Gene Ontology and KEGG Pathway Enrichment Analysis of a Drug Target-Based Classification System. PLoS One, 2015. 10(5): p. e0126492), the specific broken genes in erythrocyte micronuclei DNA were obtained.

7. Data classification and classifier construction. Differentiated genes were select as features to construct classifiers for known classified samples based on support vector machine (SVM), and to predict unknown samples (Huang, M. W., et al., SVM and SVM Ensembles in Breast Cancer Prediction. PLoS One, 2017. 12(1): p. e0161501).

7.1 Data Classification

Specifically, the reads count in gene regions of “n” experimental samples and “m” control samples were selected every time, wherein “n” and “m” refers to the number of samples). The differentiated genes (also called “characteristic genes”) were screened out by ANOVA test to distinguish the two types of samples.

7.2 Classifier Construction

Classifier parameter adjustment. Based on the characteristic genes screened in step 7.1, the training group (n=100) was determined by using the algorithm SVM/LOOCV (leave-one-out cross-validation support vector machine). First, the true labels of all samples were set (e.g., the sample in the experimental group was recorded as 1, and the sample in the control group was recorded as 0). One sample was picked out at a time as a test set, and all other samples (n−1) were used to build a model and test the “test set”. The test set traversed all samples to complete n rounds of cross-validation, and the test results for each sample were obtained. Based on the whole test results and the real label of each sample, the accuracy, sensitivity and specificity were calculated, so as to adjust the best parameters of the model and construct the training model. In this study, the parameters of SVM were set as C=100 and gamma=10⁻⁴, wherein C is the penalty coefficient, that is, the tolerance of errors; gamma is a default parameter when RBF function is selected as kernel.

7.3 Prediction on Unknown Samples

Based on the training model obtained in the previous step, unknown samples (i.e., test sets) that did not participate in the training were used to predict the test set samples through the classifier constructed in the previous step, to obtain the prediction results of the test set and the real labels of the samples, and to present the proportion of each prediction result in the two classes (i.e., risk assessment index). Unknown samples were predicted and to show the results of binary classification.

### Example 6: Construction of a Classifier for Clustering Healthy Individuals and Cervical Cancer Patients Using Erythrocyte Micronuclei DNA

In this example, there were 15 subjects, including:

Experimental group: 9 patients diagnosed with cervical cancer by other methods

Control group: 6 healthy individuals (non-cervical disease individuals).

The peripheral blood samples from patients with cervical cancer were expressed in the form of “P” plus patient number. For example, “P1” represented a peripheral blood sample from the first cervical cancer patient (“Patient 1”), “P2” represented a peripheral blood sample from the second cervical cancer patient (“Patient 2”), and so on. In addition, peripheral blood samples from healthy individuals were expressed in the form of “H” plus individual number. For example, “H1” represents the peripheral blood sample from the first healthy individual, “H2” represents the peripheral blood sample from the second healthy individual, and so on.

The basic information of 9 patients with cervical cancer is shown in Table 1. “cervical cancer type” refers to the type of cervical cancer diagnosed by other methods.

TABLE 1

| | | HPV | Maximum | | |
|---|---|---|---|---|---|
| Patient | | Detected | diameter of | | Existence of lymph |
| number | Age | value | tumor (cm) | Type of cervical cancer | node metastasis or not |
| 1 | 65 | 25.55 | 3.3 | Medium differentiated | No |
| | | | | squamous cell carcinoma | |
| 2 | 36 | 50.13 | 4.2 | Low-medium differentiated | No |
| | | | | adenocarcinoma | |
| 3 | 36 | 916.-3 | 4.0 | CINIII budding growth | Not dissected |
| | | | | (early invasion of cancer) | |
| 4 | 43 | 240.09 | 4.1 | Low differentiated | Yes (without surgery) |
| | | | | squamous cell carcinoma | |
| 5 | 5 | 3.67 | 3.7 | Non-invasive carcinoma | Not dissected |
| 6 | 56 | 503.61 | 3.2 | Low-medium differentiated | No |
| | | | | squamous cell carcinoma | |
| 7 | 71 | 1212.8 | 2.6 | Medium differentiated | Yes (without surgery) |
| | | | | squamous cell carcinoma | |
| 8* | 37 | No | 5.4 | Low-medium differentiated | Yes |
| | | | | squamous cell carcinoma | |
| 9 | 44 | 68.36 | 2.1 | Medium-high differentiated | No |
| | | | | adenocarcinoma | |

*Patient 8 is HPV negative.

Erythrocyte micronuclei DNA and genomic DNA of peripheral blood mononuclear cells of each subject were obtained as described in Examples 1-4, and bioinformatics analysis was carried out as described in Example 5.

Specifically, 9 samples of primary cervical cancer and 6 samples of healthy women were selected for the reads counting, and 2306 differential genes were screened out by ANOVA test to distinguish the two classes of samples. Then, according to Pearson correlation, the two classes of samples were clustered in unsupervised hierarchy, showing that there were significant differences between the two classes of samples.

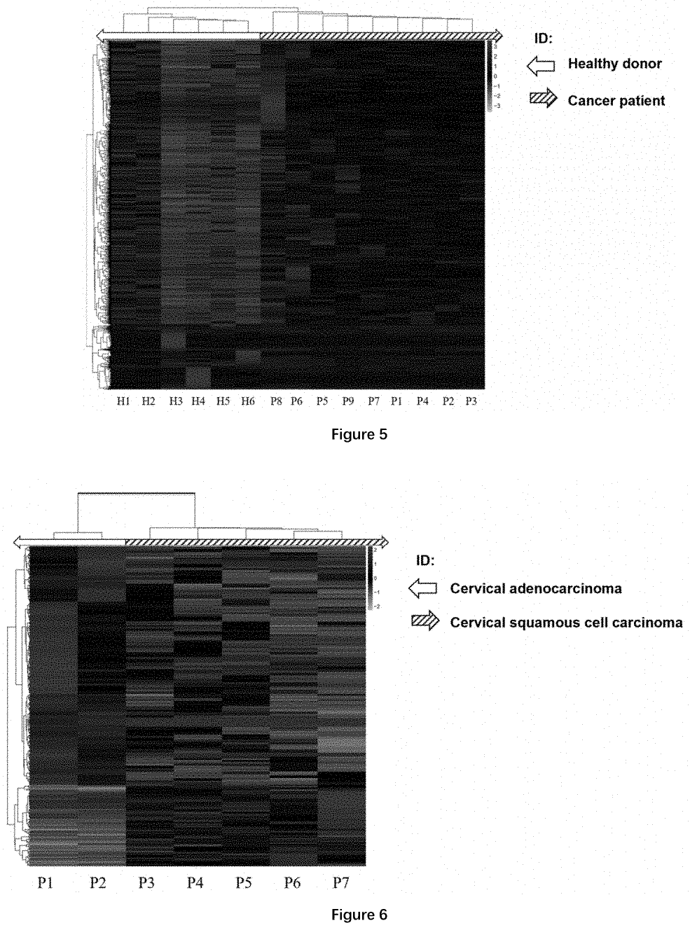

As shown in FIG. 5, the erythrocyte micronuclei DNA from the peripheral blood samples of cervical cancer patients and the erythrocyte micronuclei DNA from the peripheral blood samples of healthy individuals were clustered to obtain 2,306 differential genes (forming a classifier for distinguishing healthy individuals from cervical cancer patients). In FIG. 5, each row represents a differential gene, and each column represents a patient.

The list of 2,306 differential genes is shown in Table 2. Each gene corresponds to each row from top to bottom in FIG. 5.

TABLE 2

| | |
|---|---|
| 1 | A2MP1 |
| 2 | ABCA11P |
| 3 | ABCA9-AS1 |
| 4 | ABCB7 |
| 5 | ABCG4 |
| 6 | ABCG5 |
| 7 | ABHD3 |
| 8 | ABRAXAS1 |
| 9 | ACADM |
| 10 | ACAN |
| 11 | ACAP2 |
| 12 | ACAT2 |
| 13 | ACKR3 |
| 14 | ACKR4 |
| 15 | ACOD1 |
| 16 | ACP7 |
| 17 | ACSM5 |
| 18 | ACTR1B |
| 19 | ACTR3C |
| 20 | ACTR6 |
| 21 | ADAM21 |
| 22 | ADAM30 |
| 23 | ADAT1 |
| 24 | ADCY2 |
| 25 | ADCY6 |
| 26 | ADCY9 |
| 27 | ADD3-AS1 |
| 28 | ADPGK |
| 29 | ADRB1 |
| 30 | AFDN-DT |
| 31 | AFG1L |
| 32 | AFTPH |
| 33 | AGAP4 |
| 34 | AGFG1 |
| 35 | AGO2 |
| 36 | AGPAT1 |
| 37 | AGPAT3 |
| 38 | AIMP1 |
| 39 | AIPL1 |
| 40 | AK4 |
| 41 | AKAIN1 |
| 42 | AKAP11 |
| 43 | AKAP5 |
| 44 | AKIRIN1 |
| 45 | AKR1C4 |
| 46 | AKTIP |
| 47 | ALDH3A2 |
| 48 | ALKBH8 |
| 49 | AMMECR1 |
| 50 | AMPH |
| 51 | ANGPT2 |
| 52 | ANKRD13C |
| 53 | ANKRD18DP |
| 54 | ANKRD20A19P |
| 55 | ANKRD36BP1 |
| 56 | ANKRD62P1-PARP4P3 |
| 57 | ANKS4B |
| 58 | ANXA2P1 |
| 59 | AP3D1 |
| 60 | AP3M2 |
| 61 | AP4S1 |
| 62 | APOBEC1 |
| 63 | APOBEC4 |
| 64 | APOL4 |
| 65 | APOL6 |
| 66 | APOLD1 |
| 67 | ARAP1 |
| 68 | ARF4-AS1 |
| 69 | AREGAP2 |
| 70 | ARFGEF1 |
| 71 | ARHGAP22-IT1 |
| 72 | ARHGAP26-AS1 |
| 73 | ARHGAP36 |
| 74 | ARHGEF11 |
| 75 | ARHGEF7 |
| 76 | ARID1B |
| 77 | ARL11 |
| 78 | ARL2BP |
| 79 | ARL6IP1 |
| 80 | ARMC6 |
| 81 | ARMCX5 |
| 82 | ARPC1B |
| 83 | ARPP19 |
| 84 | ARSJ |
| 85 | ASB10 |
| 86 | ASB3 |
| 87 | ASIC4 |
| 88 | ATP13A3 |
| 89 | ATP2B1-AS1 |
| 90 | ATP5F1C |
| 91 | ATP5PO |
| 92 | ATP6V0A4 |
| 93 | ATP7A |
| 94 | ATXN7L3B |
| 95 | AUNIP |
| 96 | AWAT1 |
| 97 | AXIN2 |
| 98 | B3GALNT1 |
| 99 | B3GNT5 |
| 100 | BAG2 |
| 101 | BBS1 |
| 102 | BBS2 |
| 103 | BBS9 |
| 104 | BEX1 |
| 105 | BEX5 |
| 106 | BFSP1 |
| 107 | BHLHE40-AS1 |
| 108 | BICRAL |
| 109 | BIRC6-AS2 |
| 110 | BLOC1S2 |
| 111 | BLVRB |
| 112 | BMP15 |
| 113 | BMS1 |
| 114 | BORA |
| 115 | BRMS1L |
| 116 | BRWD1-AS1 |
| 117 | BSND |
| 118 | BTBD3 |
| 119 | BTBD9-AS1 |
| 120 | BTG1 |
| 121 | BTG2 |
| 122 | BTG4 |
| 123 | BTN2A2 |
| 124 | BTN3A2 |
| 125 | BTNL2 |
| 126 | BUD31 |
| 127 | C10orf143 |
| 128 | C11orf44 |
| 129 | C11orf53 |
| 130 | C11orf54 |
| 131 | C12orf10 |
| 132 | C12orf45 |
| 133 | C12orf71 |
| 134 | C12orf75 |
| 135 | C14orf39 |
| 136 | C16orf89 |
| 137 | C17orf75 |
| 138 | C18orf54 |
| 139 | C19orf66 |
| 140 | C1GALT1 |
| 141 | C1QTNF6 |
| 142 | C1S |
| 143 | C1orf68 |
| 144 | C21orf59 |
| 145 | C2orf48 |
| 146 | C2orf49 |
| 147 | C2orf74 |
| 148 | C2orf78 |
| 149 | C3orf18 |
| 150 | C3orf38 |
| 151 | C4orf45 |
| 152 | C4orf47 |
| 153 | C5orf24 |
| 154 | C5orf64 |
| 155 | C5orf66 |
| 156 | C6orf99 |
| 157 | C7orf13 |
| 158 | C7orf26 |
| 159 | C8orf31 |
| 160 | C8orf48 |
| 161 | C9orf170 |
| 162 | C9orf40 |
| 163 | C9orf43 |
| 164 | C9orf66 |
| 165 | C9orf92 |
| 166 | CA2 |
| 167 | CA8 |
| 168 | CACNA1I |
| 169 | CACNB3 |
| 170 | CADM4 |
| 171 | CALCRL |
| 172 | CAMK1G |
| 173 | CASC1 |
| 174 | CASC3 |
| 175 | CASC9 |
| 176 | CAVIN4 |
| 177 | CBWD5 |
| 178 | CBX2 |
| 179 | CBX3 |
| 180 | CBX5 |
| 181 | CC2D1B |
| 182 | CCDC120 |
| 183 | CCDC121 |
| 184 | CCDC152 |
| 185 | CCDC17 |
| 186 | CCDC18-AS1 |
| 187 | CCDC186 |
| 188 | CCDC89 |
| 189 | CCEPR |
| 190 | CCIN |
| 191 | CCL11 |
| 192 | CCL22 |
| 193 | CCL5 |
| 194 | CCNG1 |
| 195 | CCNI2 |
| 196 | CCNT2 |
| 197 | CCNT2-AS1 |
| 198 | CCR5 |
| 199 | CCT2 |
| 200 | CCT6A |
| 201 | CCT8 |
| 202 | CD160 |
| 203 | CD28 |
| 204 | CD300LB |
| 205 | CD33 |
| 206 | CD37 |
| 207 | CD3E |
| 208 | CD40 |
| 209 | CD40LG |
| 210 | CD58 |
| 211 | CD72 |
| 212 | CDC40 |
| 213 | CDCA3 |
| 214 | CDCP1 |
| 215 | CDH5 |
| 216 | CDK13 |
| 217 | CDKN2B |
| 218 | CDSN |
| 219 | CDV3 |
| 220 | CENPL |
| 221 | CEP78 |
| 222 | CEP83-AS1 |
| 223 | CETN1 |
| 224 | CFAP126 |
| 225 | CFAP77 |
| 226 | CFAP97 |
| 227 | CFDP1 |
| 228 | CFHR4 |
| 229 | CHAMP1 |
| 230 | CHIC1 |
| 231 | CHMP4B |
| 232 | CHRNA10 |
| 233 | CKAP5 |
| 234 | CLDN19 |
| 235 | CLDN22 |
| 236 | CLDN7 |
| 237 | CLDND1 |
| 238 | CLEC1B |
| 239 | CLEC2B |
| 240 | CLEC5A |
| 241 | CLK2P1 |
| 242 | CLRN1 |
| 243 | CLRN3 |
| 244 | CLSTN2-AS1 |
| 245 | CLTA |
| 246 | CLTB |
| 247 | CLUL1 |
| 248 | CMC1 |
| 249 | CMC2 |
| 250 | CNBP |
| 251 | CNDP2 |
| 252 | CNGA1 |
| 253 | CNGA4 |
| 254 | CNIH4 |
| 255 | CNKSR3 |
| 256 | CNOT6L |
| 257 | CNP |
| 258 | COG4 |
| 259 | COG8 |
| 260 | COL6A4P1 |
| 261 | COMMD6 |
| 262 | COPB2 |
| 263 | COX7A2L |
| 264 | CPB1 |
| 265 | CPEB1 |
| 266 | CPHXL |
| 267 | CPLX2 |
| 268 | CPNE6 |
| 269 | CREB1 |
| 270 | CRH |
| 271 | CRIPT |
| 272 | CRISPLD1 |
| 273 | CRNN |
| 274 | CRYBG2 |
| 275 | CRYM |
| 276 | CSF1R |
| 277 | CST1 |
| 278 | CST3 |
| 279 | CST9 |
| 280 | CT62 |
| 281 | CTHRC1 |
| 282 | CTIF |
| 283 | CTSO |
| 284 | CTSV |
| 285 | CTXND1 |
| 286 | CTXND2 |
| 287 | CUL7 |
| 288 | CXCL3 |
| 289 | CXCL5 |
| 290 | CXCL6 |
| 291 | CXCL9 |
| 292 | CXCR4 |
| 293 | CYB5D2 |
| 294 | CYP11B1 |
| 295 | CYP27C1 |
| 296 | CYP4V2 |
| 297 | CYP4Z1 |
| 298 | CYP4Z2P |
| 299 | CYP8B1 |
| 300 | CYTH3 |
| 301 | CYTH4 |
| 302 | CYYR1 |
| 303 | DACT1 |
| 304 | DACT3-AS1 |
| 305 | DAGLB |
| 306 | DARS-AS1 |
| 307 | DAZL |
| 308 | DBF4 |
| 309 | DBT |
| 310 | DBX2 |
| 311 | DCAF12L1 |
| 312 | DCAF12L2 |
| 313 | DCK |
| 314 | DCLRE1A |
| 315 | DCPS |
| 316 | DCTN1-AS1 |
| 317 | DDB2 |
| 318 | DDI1 |
| 319 | DDX42 |
| 320 | DEFA5 |
| 321 | DEFA9P |
| 322 | DENND1B |
| 323 | DENND1C |
| 324 | DENND2A |
| 325 | DEPDC1-AS1 |
| 326 | DEPDC1B |
| 327 | DGCR5 |
| 328 | DGUOK |
| 329 | DHRS4-AS1 |
| 330 | DIRC3 |
| 331 | DIS3 |
| 332 | DKFZP586I1420 |
| 333 | DKK2 |
| 334 | DLG3 |
| 335 | DMWD |
| 336 | DNAAF3 |
| 337 | DNAI1 |
| 338 | DNAJA3 |
| 339 | DNAJC27 |
| 340 | DNAJC5G |
| 341 | DNAJC6 |
| 342 | DNM1P46 |
| 343 | DNM3-IT1 |
| 344 | DNMT1 |
| 345 | DNPH1 |
| 346 | DPM1 |
| 347 | DPP10-AS1 |
| 348 | DPPA2P3 |
| 349 | DPRXP4 |
| 350 | DPY19L2P1 |
| 351 | DPYD-AS2 |
| 352 | DPYS |
| 353 | DR1 |
| 354 | DRAIC |
| 355 | DSTYK |
| 356 | DTX3L |
| 357 | DUOXA1 |
| 358 | DUSP18 |
| 359 | DUSP21 |
| 360 | DUSP5 |
| 361 | DYNC1LI2 |
| 362 | DYNLRB2 |
| 363 | DYRK2 |
| 364 | EAF2 |
| 365 | ECHDC2 |
| 366 | EDRF1 |
| 367 | EFNA2 |
| 368 | EIF2D |
| 369 | EIF2S2 |
| 370 | EIF2S3L |
| 371 | EIF3B |
| 372 | EIF3J |
| 373 | EIF5A2 |
| 374 | EIF6 |
| 375 | ELAVL1 |
| 376 | ELMOD2 |
| 377 | ELOA2 |
| 378 | ELSPBP1 |
| 379 | EMB |
| 380 | EML6 |
| 381 | EMP1 |
| 382 | EMX1 |
| 383 | ENG |
| 384 | ENOPH1 |
| 385 | ENTPD3-AS1 |
| 386 | ENTPD6 |
| 387 | EPB41L4A-AS1 |
| 388 | EPB41L4B |
| 389 | EPB41L5 |
| 390 | EPCAM |
| 391 | EPHA10 |
| 392 | EPN2 |
| 393 | ERCC1 |
| 394 | ERGIC2 |
| 395 | ERICH2 |
| 396 | ERICH3-AS1 |
| 397 | ERN2 |
| 398 | ERVV-1 |
| 399 | ERVW-1 |
| 400 | ESCO2 |
| 401 | ESX1 |
| 402 | ETFBKMT |
| 403 | ETNPPL |
| 404 | ETS2 |
| 405 | ETV4 |
| 406 | EWSAT1 |
| 407 | EXOC5 |
| 408 | EXOC6B |
| 409 | EXOSC7 |
| 410 | EXPH5 |
| 411 | EXTL1 |
| 412 | EXTL3 |
| 413 | EZR |
| 414 | FABP4 |
| 415 | FAM105A |
| 416 | FAM111A-DT |
| 417 | FAM117B |
| 418 | FAM124B |
| 419 | FAM131A |
| 420 | FAM161B |
| 421 | FAM162B |
| 422 | FAM183A |
| 423 | FAM196B |
| 424 | FAM199X |
| 425 | FAM19A3 |
| 426 | FAM200B |
| 427 | FAM201A |
| 428 | FAM205C |
| 429 | FAM212B |
| 430 | FAM217A |
| 431 | FAM228B |
| 432 | FAM238B |
| 433 | FAM238C |
| 434 | FAM24A |
| 435 | FAM45BP |
| 436 | FAM47A |
| 437 | FAM69A |
| 438 | FAM76A |
| 439 | FAM83C |
| 440 | FAM87B |
| 441 | FAM90A1 |
| 442 | FAM92A |
| 443 | FAM98B |
| 444 | FAM9C |
| 445 | FASTKD5 |
| 446 | FBXL14 |
| 447 | FBXL2 |
| 448 | FBXL4 |
| 449 | FBXO15 |
| 450 | FBXO4 |
| 451 | FBXO40 |
| 452 | FBXO45 |
| 453 | FBXW2 |
| 454 | FCER1G |
| 455 | FCN3 |
| 456 | FCRL2 |
| 457 | FCRL3 |
| 458 | FCRLA |
| 459 | FCRLB |
| 460 | FECH |
| 461 | FEZ2 |
| 462 | FEZF1-AS1 |
| 463 | FGD3 |
| 464 | FGF12-AS2 |
| 465 | FGF9 |
| 466 | FGFBP3 |
| 467 | FGFR1OP |
| 468 | FGG |
| 469 | FGL2 |
| 470 | FHAD1 |
| 471 | FHL5 |
| 472 | FILIP1L |
| 473 | FJX1 |
| 474 | FLG2 |
| 475 | FLJ12825 |
| 476 | FLJ31104 |
| 477 | FLJ37201 |
| 478 | FLJ42393 |
| 479 | FOLR2 |
| 480 | FOXP1-AS1 |
| 481 | FRZB |
| 482 | FSHB |
| 483 | FSTL5 |
| 484 | FTSJ3 |
| 485 | FTX |
| 486 | FUNDC2P2 |
| 487 | FUT8-AS1 |
| 488 | FUT9 |
| 489 | FXN |
| 490 | FZD3 |
| 491 | FZD4 |
| 492 | FZD7 |
| 493 | GABARAPL3 |
| 494 | GABRA5 |
| 495 | GART |
| 496 | GAS1 |
| 497 | GATA1 |
| 498 | GATA6 |
| 499 | GBA2 |
| 500 | GBP1 |
| 501 | GBP6 |
| 502 | GCH1 |
| 503 | GDPD2 |
| 504 | GEN1 |
| 505 | GGPS1 |
| 506 | GGT7 |
| 507 | GHRLOS |
| 508 | GID8 |
| 509 | GIMAP1 |
| 510 | GIMAP7 |
| 511 | GIMAP8 |
| 512 | GINM1 |
| 513 | GIPC1 |
| 514 | GJA10 |
| 515 | GJA3 |
| 516 | GJD3 |
| 517 | GKN2 |
| 518 | GLI3 |
| 519 | GLS |
| 520 | GLT8D1 |
| 521 | GLYR1 |
| 522 | GMFB |
| 523 | GMPPA |
| 524 | GNAI3 |
| 525 | GNPDA1 |
| 526 | GOLGA8J |
| 527 | GOLIM4 |
| 528 | GOLPH3L |
| 529 | GON7 |
| 530 | GORASP2 |
| 531 | GOT2 |
| 532 | GP1BA |
| 533 | GP6 |
| 534 | GPALPP1 |
| 535 | GPR1-AS |
| 536 | GPR141 |
| 537 | GPR142 |
| 538 | GPR149 |
| 539 | GPR52 |
| 540 | GPR6 |
| 541 | GPR75 |
| 542 | GPR82 |
| 543 | GRHL1 |
| 544 | GRIN2D |
| 545 | GRIPAP1 |
| 546 | GSK3B |
| 547 | GSN |
| 548 | GSN-AS1 |
| 549 | GTF3C3 |
| 550 | GTSE1-AS1 |
| 551 | GUCA1C |
| 552 | GYPE |
| 553 | GZF1 |
| 554 | H1FNT |
| 555 | H3F3B |
| 556 | HAAO |
| 557 | HAND1 |
| 558 | HAS3 |
| 559 | HBB |
| 560 | HBP1 |
| 561 | HCCS |
| 562 | HCFC2 |
| 563 | HCG24 |
| 564 | HCG9 |
| 565 | HCRTR2 |
| 566 | HEATR1 |
| 567 | HEYL |
| 568 | HHAT |
| 569 | HIF1A-AS1 |
| 570 | HIGD1B |
| 571 | HIKESHI |
| 572 | HINT2 |
| 573 | HINT3 |
| 574 | HIST1H2AL |
| 575 | HIST1H2BA |
| 576 | HIST1H2BD |
| 577 | HIST1H4F |
| 578 | HLA-DOA |
| 579 | HLA-DOB |
| 580 | HLA-DPB1 |
| 581 | HLA-DQB2 |
| 582 | HLA-F |
| 583 | HLTF |
| 584 | HMGA1P7 |
| 585 | HMGN5 |
| 586 | HMGXB3 |
| 587 | HNF1A |
| 588 | HNRNPA1P10 |
| 589 | HNRNPA3 |
| 590 | HNRNPD |
| 591 | HNRNPDL |
| 592 | HNRNPL |
| 593 | HNRNPU |
| 594 | HOOK3 |
| 595 | HOTAIRM1 |
| 596 | HOXC8 |
| 597 | HPCAL4 |
| 598 | HPS6 |
| 599 | HRASLS |
| 600 | HRASLS2 |
| 601 | HRC |
| 602 | HS3ST3B1 |
| 603 | HSD3BP4 |
| 604 | HSD52 |
| 605 | HSDL1 |
| 606 | HSDL2 |
| 607 | HSF2 |
| 608 | HSPA14 |
| 609 | HSPA9 |
| 610 | HTATIP2 |
| 611 | HTR1A |
| 612 | HTR1D |
| 613 | HTR3D |
| 614 | HYALP1 |
| 615 | HYKK |
| 616 | IAPP |
| 617 | ICE1 |
| 618 | IDS |
| 619 | IFT20 |
| 620 | IFT88 |
| 621 | IGFBP1 |
| 622 | IGFL3 |
| 623 | IGLL5 |
| 624 | IGSF9B |
| 625 | IL10RB-AS1 |
| 626 | IL13 |
| 627 | IL17RC |
| 628 | IL17RE |
| 629 | IL20RB |
| 630 | IL21 |
| 631 | IL33 |
| 632 | IL4R |
| 633 | IL6 |
| 634 | IL6R |
| 635 | INE2 |
| 636 | ING4 |
| 637 | INHBA |
| 638 | IPO8 |
| 639 | IQCA1 |
| 640 | IQCF2 |
| 641 | IQGAP2 |
| 642 | IRAK1BP1 |
| 643 | IRS2 |
| 644 | ISLR2 |
| 645 | ISM1 |
| 646 | ISOC1 |
| 647 | ITFG1-AS1 |
| 648 | ITFG2 |
| 649 | ITGAL |
| 650 | ITGAV |
| 651 | ITGB3BP |
| 652 | IWS1 |
| 653 | JADE3 |
| 654 | JAM3 |
| 655 | JMJD1C-AS1 |
| 656 | JRK |
| 657 | KANSL3 |
| 658 | KATNBL1P6 |
| 659 | KBTBD13 |
| 660 | KC6 |
| 661 | KCNA3 |
| 662 | KCNH1-IT1 |
| 663 | KCNJ1 |
| 664 | KCNJ13 |
| 665 | KCNK3 |
| 666 | KCNK5 |
| 667 | KCNMB3 |
| 668 | KCNS1 |
| 669 | KCTD21 |
| 670 | KCTD9 |
| 671 | KDELC1 |
| 672 | KDM4A-AS1 |
| 673 | KIAA0754 |
| 674 | KIAA0895L |
| 675 | KIAA2026 |
| 676 | KIF20A |
| 677 | KIF4B |
| 678 | KIF6 |
| 679 | KIRREL3-AS3 |
| 680 | KLF11 |
| 681 | KLF12 |
| 682 | KLF13 |
| 683 | KLF3 |
| 684 | KLHDC1 |
| 685 | KLHL34 |
| 686 | KLK11 |
| 687 | KLK2 |
| 688 | KLLN |
| 689 | KLRB1 |
| 690 | KLRC1 |
| 691 | KLRF2 |
| 692 | KLRG1 |
| 693 | KMT2E-AS1 |
| 694 | KPNA6 |
| 695 | KPRP |
| 696 | KRBA2 |
| 697 | KRCC1 |
| 698 | KRIT1 |
| 699 | KRT10 |
| 700 | KRT13 |
| 701 | KRT25 |
| 702 | KRT4 |
| 703 | KRT6B |
| 704 | KRT83 |
| 705 | KRTAP12-2 |
| 706 | KRTAP17-1 |
| 707 | KRTAP19-2 |
| 708 | KRTAP20-1 |
| 709 | KRTAP4-5 |
| 710 | KRTAP5-2 |
| 711 | KRTAP5-AS1 |
| 712 | KRTAP9-4 |
| 713 | KRTDAP |
| 714 | LAMP5 |
| 715 | LAMP5-AS1 |
| 716 | LAMTOR5-AS1 |
| 717 | LAPTM4B |
| 718 | LATS1 |
| 719 | LCORL |
| 720 | LCP2 |
| 721 | LDLRAD4-AS1 |
| 722 | LEF1-AS1 |
| 723 | LGALS13 |
| 724 | LGALS17A |
| 725 | LGALS3 |
| 726 | LGALS8-AS1 |
| 727 | LIMD1-AS1 |
| 728 | LIMK2 |
| 729 | LINC00161 |
| 730 | LINC00216 |
| 731 | LINC00240 |
| 732 | LINC00308 |
| 733 | LINC00309 |
| 734 | LINC00317 |
| 735 | LINC00331 |
| 736 | LINC00339 |
| 737 | LINC00363 |
| 738 | LINC00392 |
| 739 | LINC00440 |
| 740 | LINC00467 |
| 741 | LINC00491 |
| 742 | LINC00520 |
| 743 | LINC00539 |
| 744 | LINC00548 |
| 745 | LINC00615 |
| 746 | LINC00620 |
| 747 | LINC00626 |
| 748 | LINC00628 |
| 749 | LINC00641 |
| 750 | LINC00668 |
| 751 | LINC00674 |
| 752 | LINC00837 |
| 753 | LINC00844 |
| 754 | LINC00862 |
| 755 | LINC00889 |
| 756 | LINC00901 |
| 757 | LINC00924 |
| 758 | LINC00926 |
| 759 | LINC00970 |
| 760 | LINC00987 |
| 761 | LINC00999 |
| 762 | LINC01012 |
| 763 | LINC01016 |
| 764 | LINC01036 |
| 765 | LINC01037 |
| 766 | LINC01039 |
| 767 | LINC01049 |
| 768 | LINC01099 |
| 769 | LINC01121 |
| 770 | LINC01135 |
| 771 | LINC01140 |
| 772 | LINC01191 |
| 773 | LINC01202 |
| 774 | LINC01209 |
| 775 | LINC01224 |
| 776 | LINC01243 |
| 777 | LINC01305 |
| 778 | LINC01340 |
| 779 | LINC01370 |
| 780 | LINC01416 |
| 781 | LINC01432 |
| 782 | LINC01461 |
| 783 | LINC01474 |
| 784 | LINC01476 |
| 785 | LINC01478 |
| 786 | LINC01486 |
| 787 | LINC01499 |
| 788 | LINC01564 |
| 789 | LINC01565 |
| 790 | LINC01580 |
| 791 | LINC01586 |
| 792 | LINC01594 |
| 793 | LINC01600 |
| 794 | LINC01623 |
| 795 | LINC01628 |
| 796 | LINC01657 |
| 797 | LINC01682 |
| 798 | LINC01683 |
| 799 | LINC01695 |
| 800 | LINC01698 |
| 801 | LINC01721 |
| 802 | LINC01723 |
| 803 | LINC01725 |
| 804 | LINC01737 |
| 805 | LINC01846 |
| 806 | LINC01851 |
| 807 | LINC01877 |
| 808 | LINC01878 |
| 809 | LINC01907 |
| 810 | LINC01915 |
| 811 | LINC01933 |
| 812 | LINC01934 |
| 813 | LINC01939 |
| 814 | LINC01959 |
| 815 | LINC01975 |
| 816 | LINC01985 |
| 817 | LINC01986 |
| 818 | LINC01990 |
| 819 | LINC01991 |
| 820 | LINC02009 |
| 821 | LINC02010 |
| 822 | LINC02020 |
| 823 | LINC02041 |
| 824 | LINC02049 |
| 825 | LINC02071 |
| 826 | LINC02106 |
| 827 | LINC02120 |
| 828 | LINC02135 |
| 829 | LINC02149 |
| 830 | LINC02158 |
| 831 | LINC02167 |
| 832 | LINC02185 |
| 833 | LINC02210 |
| 834 | LINC02217 |
| 835 | LINC02226 |
| 836 | LINC02233 |
| 837 | LINC02278 |
| 838 | LINC02287 |
| 839 | LINC02292 |
| 840 | LINC02303 |
| 841 | LINC02314 |
| 842 | LINC02324 |
| 843 | LINC02339 |
| 844 | LINC02363 |
| 845 | LINC02365 |
| 845 | LINC02376 |
| 847 | LINC02380 |
| 848 | LINC02382 |
| 849 | LINC02387 |
| 850 | LINC02414 |
| 851 | LINC02431 |
| 852 | LINC02454 |
| 853 | LINC02485 |
| 854 | LINC02488 |
| 855 | LINC02523 |
| 856 | LINC02542 |
| 857 | LINC02544 |
| 858 | LINC02565 |
| 859 | LIPJ |
| 860 | LIVAR |
| 861 | LMCD1-AS1 |
| 862 | LMNTD1 |
| 863 | LMO4 |
| 864 | LNX1-AS1 |
| 865 | LOC100128594 |
| 866 | LOC100129138 |
| 867 | LOC100129148 |
| 868 | LOC100129297 |
| 869 | LOC100129636 |
| 870 | LOC100129935 |
| 871 | LOCIOO130075 |
| 872 | LOC100130298 |
| 873 | LOC100132831 |
| 874 | LOC100192426 |
| 875 | LOC100240728 |
| 876 | LOC100270746 |
| 877 | LOC100270804 |
| 878 | LOC100272217 |
| 879 | LOC100287467 |
| 880 | LOC100287497 |
| 881 | LOC100288637 |
| 882 | LOC100288842 |
| 883 | LOC100289230 |
| 884 | LOC100289495 |
| 885 | LOC100335030 |
| 886 | LOC100500773 |
| 887 | LOC100505978 |
| 888 | LOC100506098 |
| 889 | LOC100506302 |
| 890 | LOC100506358 |
| 891 | LOC100506497 |
| 892 | LOC100506548 |
| 893 | LOC100506682 |
| 894 | LOC100507002 |
| 895 | LOC100507388 |
| 896 | LOC100508631 |
| 897 | LOC100996635 |
| 898 | LOC100996664 |
| 899 | LOC101805491 |
| 900 | LOC101926943 |
| 901 | LOC101927045 |
| 902 | LOC101927142 |
| 903 | LOC101927196 |
| 904 | LOC101927314 |
| 905 | LOC101927391 |
| 906 | LOC101927437 |
| 907 | LOC101927516 |
| 908 | LOC101927550 |
| 909 | LOC101927560 |
| 910 | LOC101927661 |
| 911 | LOC101927708 |
| 912 | LOC101927919 |
| 913 | LOC101927972 |
| 914 | LOC101928131 |
| 915 | LOC101928134 |
| 916 | LOC101928140 |
| 917 | LOC101928143 |
| 918 | LOC101928253 |
| 919 | LOC101928307 |
| 920 | LOC101928336 |
| 921 | LOC101928370 |
| 922 | LOC101928429 |
| 923 | LOC101928443 |
| 924 | LOC101928495 |
| 925 | LOC101928517 |
| 926 | LOC101928530 |
| 927 | LOC101928535 |
| 928 | LOC101928622 |
| 929 | LOC101928797 |
| 930 | LOC101928847 |
| 931 | LOC101928858 |
| 932 | LOC101928936 |
| 933 | LOC101928940 |
| 934 | LOC101929353 |
| 935 | LOC101929372 |
| 936 | LOC101929517 |
| 937 | LOC101929529 |
| 938 | LOC101929541 |
| 939 | LOC101929577 |
| 940 | LOC101929586 |
| 941 | LOC101929633 |
| 942 | LOC101929662 |
| 943 | LOC101929709 |
| 944 | LOC101929753 |
| 945 | LOC101930085 |
| 946 | LOC102467217 |
| 947 | LOC102723313 |
| 948 | LOC102723335 |
| 949 | LOC102723373 |
| 950 | LOC102723439 |
| 951 | LOC102723544 |
| 952 | LOC102723833 |
| 953 | LOC102724153 |
| 954 | LOC102724264 |
| 955 | LOC102724297 |
| 956 | LOC102724601 |
| 957 | LOC102725080 |
| 958 | LOC105372795 |
| 959 | LOC105373352 |
| 960 | LOC105373876 |
| 961 | LOC105376365 |
| 962 | LOC105377342 |
| 963 | LOC105377651 |
| 964 | LOC105377967 |
| 965 | LOC105378098 |
| 966 | LOC105378146 |
| 967 | LOC105378330 |
| 968 | LOC105378385 |
| 969 | LOC105378853 |
| 970 | LOC106699570 |
| 971 | LOC148709 |
| 972 | LOC283177 |
| 973 | LOC283387 |
| 974 | LOC283683 |
| 975 | LOC284581 |
| 976 | LOC286059 |
| 977 | LOC339529 |
| 978 | LOC339666 |
| 979 | LOC392364 |
| 980 | LOC399716 |
| 981 | LOC400655 |
| 982 | LOC401127 |
| 983 | LOC401442 |
| 984 | LOC403312 |
| 985 | LOC439933 |
| 986 | LOC440311 |
| 987 | LOC574538 |
| 988 | LOC643201 |
| 989 | LOC643406 |
| 990 | LOC643733 |
| 991 | LOC645513 |
| 992 | LOC645752 |
| 993 | LOC646762 |
| 994 | LOC728158 |
| 995 | LOC728327 |
| 996 | LOC728392 |
| 997 | LOC729224 |
| 998 | LOC729603 |
| 999 | LOC729870 |
| 1000 | LOC730202 |
| 1001 | LOC730234 |
| 1002 | LOC91548 |
| 1003 | LOXL3 |
| 1004 | LPAR5 |
| 1005 | LPAR6 |
| 1006 | LPP-AS2 |
| 1007 | LRFN1 |
| 1008 | LRIT2 |
| 1009 | LRP8 |
| 1010 | LRRC32 |
| 1011 | LRRC57 |
| 1012 | LRRC58 |
| 1013 | LRRC61 |
| 1014 | LRRC66 |
| 1015 | LRRC75A |
| 1016 | LRRIQ1 |
| 1017 | LRRIQ4 |
| 1018 | LSAMP-AS1 |
| 1019 | LSM14B |
| 1020 | LSMEM1 |
| 1021 | LUC7L |
| 1022 | LURAP1 |
| 1023 | LUZP4 |
| 1024 | LYPD5 |
| 1025 | LYRM9 |
| 1026 | LYVE1 |
| 1027 | MAGEA11 |
| 1028 | MAGEA8 |
| 1029 | MAGEA8-AS1 |
| 1030 | MAGEB6 |
| 1031 | MAGEE2 |
| 1032 | MAGEL2 |
| 1033 | MAGIX |
| 1034 | MAGOH2P |
| 1035 | MALSU1 |
| 1036 | MAN1C1 |
| 1037 | MAP1A |
| 1038 | MAP2K4P1 |
| 1039 | MAP3K21 |
| 1040 | MAP3K8 |
| 1041 | MAPRE1 |
| 1042 | MAPT |
| 1043 | MAPT-IT1 |
| 1044 | MASP1 |
| 1045 | MATN3 |
| 1046 | MBL2 |
| 1047 | MBLAC2 |
| 1048 | MCCC1 |
| 1049 | MCF2L |
| 1050 | MCM6 |
| 1051 | MCPH1-AS1 |
| 1052 | MCRS1 |
| 1053 | MDM2 |
| 1054 | MEAT6 |
| 1055 | MED13 |
| 1056 | MED20 |
| 1057 | MED28 |
| 1058 | MED8 |
| 1059 | MEDAG |
| 1060 | METTL11B |
| 1061 | METTL25 |
| 1062 | MFAP4 |
| 1063 | MFGE8 |
| 1064 | MFN2 |
| 1065 | MGAT4D |
| 1066 | MGC15885 |
| 1067 | MGC16275 |
| 1068 | MGC34796 |
| 1069 | MGEA5 |
| 1070 | MGME1 |
| 1071 | MGST3 |
| 1072 | MICALCL |
| 1073 | MICU2 |
| 1074 | MICU3 |
| 1075 | MIER2 |
| 1076 | MINDY1 |
| 1077 | MIR1243 |
| 1078 | MIR1298 |
| 1079 | MIR1343 |
| 1080 | MIR23C |
| 1081 | MIR2681 |
| 1082 | MIR302D |
| 1083 | MIR3121 |
| 1084 | MIR3146 |
| 1085 | MIR4306 |
| 1086 | MIR4439 |
| 1087 | MIR4447 |
| 1088 | MIR4464 |
| 1089 | MIR4501 |
| 1090 | MIR548F3 |
| 1091 | MIR548X |
| 1092 | MIR562 |
| 1093 | MIR5689HG |
| 1094 | MIR571 |
| 1095 | MIR6130 |
| 1096 | MIR620 |
| 1097 | MIR624 |
| 1098 | MIR8062 |
| 1099 | MKRN2 |
| 1100 | MKX-AS1 |
| 1101 | MLLT3 |
| 1102 | MMAA |
| 1103 | MMP1 |
| 1104 | MMP12 |
| 1105 | MNAT1 |
| 1106 | MOB1B |
| 1107 | MORF4L1 |
| 1108 | MORF4L2 |
| 1109 | MORN1 |
| 1110 | MPZ |
| 1111 | MRAS |
| 1112 | MRFAP1 |
| 1113 | MRGPRX1 |
| 1114 | MRGPRX3 |
| 1115 | MRPL17 |
| 1116 | MRPL19 |
| 1117 | MRPL30 |
| 1118 | MRPL37 |
| 1119 | MRPL45P2 |
| 1120 | MRPL52 |
| 1121 | MRPL9 |
| 1122 | MRPS18C |
| 1123 | MRPS22 |
| 1124 | MRS2P2 |
| 1125 | MS4A14 |
| 1126 | MS4A4E |
| 1127 | MSANTD4 |
| 1128 | MSC-AS1 |
| 1129 | MSH6 |
| 1130 | MSL3 |
| 1131 | MSL3P1 |
| 1132 | MSRB2 |
| 1133 | MTERF2 |
| 1134 | MTRNR2L3 |
| 1135 | MTRNR2L4 |
| 1136 | MTRNR2L5 |
| 1137 | MTX3 |
| 1138 | MUC21 |
| 1139 | MUM1L1 |
| 1140 | MUTYH |
| 1141 | MXD4 |
| 1142 | MXRA5 |
| 1143 | MYC |
| 1144 | MYF5 |
| 1145 | MYH16 |
| 1146 | MYL1 |
| 1147 | MYLK2 |
| 1148 | MYSM1 |
| 1149 | NAA30 |
| 1150 | NAA35 |
| 1151 | NAALAD2 |
| 1152 | NAGA |
| 1153 | NANOS1 |
| 1154 | NANP |
| 1155 | NAT1 |
| 1156 | NAT16 |
| 1157 | NAV2-AS4 |
| 1158 | NBPF13P |
| 1159 | NCALD |
| 1160 | NCAPD3 |
| 1161 | NCAPG |
| 1162 | NCBP2L |
| 1163 | NCK1-AS1 |
| 1164 | NCKIPSD |
| 1165 | NCL |
| 1166 | NDST3 |
| 1167 | NDUFA4 |
| 1168 | NDUFB8 |
| 1169 | NDUFV2-AS1 |
| 1170 | NECTIN3-AS1 |
| 1171 | NEDD9 |
| 1172 | NEFM |
| 1173 | NEGR1-IT1 |
| 1174 | NEK8 |
| 1175 | NELFCD |
| 1176 | NETO2 |
| 1177 | NEUROD6 |
| 1178 | NFAT5 |
| 1179 | NFE2L3 |
| 1180 | NFIA-AS2 |
| 1181 | NFIB |
| 1182 | NFIC |
| 1183 | NHLH1 |
| 1184 | NHLRC3 |
| 1185 | NIFK |
| 1186 | NIPBL-AS1 |
| 1187 | NIPSNAP3A |
| 1188 | NIPSNAP3B |
| 1189 | NKAIN3 |
| 1190 | NKAPL |
| 1191 | NKRF |
| 1192 | NKX1-2 |
| 1193 | NLGN2 |
| 1194 | NLRP2B |
| 1195 | NLRP7 |
| 1196 | NNT-AS1 |
| 1197 | NOL3 |
| 1198 | NOS2 |
| 1199 | NOV |
| 1200 | NPTN |
| 1201 | NPVF |
| 1202 | NR2F1-AS1 |
| 1203 | NRG3-AS1 |
| 1204 | NRIP2 |
| 1205 | NRN1 |
| 1206 | NSMCE3 |
| 1207 | NT5E |
| 1208 | NTM-AS1 |
| 1209 | NTS |
| 1210 | NUBP1 |
| 1211 | NUDT5 |
| 1212 | NUFIP1 |
| 1213 | NUGGC |
| 1214 | NUF188 |
| 1215 | NXF4 |
| 1216 | NXFE3 |
| 1217 | OCLM |
| 1218 | ODAM |
| 1219 | ODF4 |
| 1220 | OGT |
| 1221 | OLFM5P |
| 1222 | OLMALINC |
| 1223 | OMG |
| 1224 | OR10A7 |
| 1225 | OR10AD1 |
| 1226 | OR10AG1 |
| 1227 | OR10H2 |
| 1228 | OR10Q1 |
| 1229 | OR10R2 |
| 1230 | OR10S1 |
| 1231 | OR10T2 |
| 1232 | OR11H6 |
| 1233 | OR11H7 |
| 1234 | OR12D2 |
| 1235 | OR14J1 |
| 1236 | OR1A2 |
| 1237 | OR1E3 |
| 1238 | OR1F2P |
| 1239 | OR1J1 |
| 1240 | OR1K1 |
| 1241 | OR1S1 |
| 1242 | OR2A25 |
| 1243 | OR2AG1 |
| 1244 | OR2AG2 |
| 1245 | OR2AJ1 |
| 1246 | OR2C3 |
| 1247 | OR2D2 |
| 1248 | OR2F1 |
| 1249 | OR2M1P |
| 1250 | OR2T27 |
| 1251 | OR2T7 |
| 1252 | OR2V1 |
| 1253 | OR2V2 |
| 1254 | OR2W5 |
| 1255 | OR4C12 |
| 1256 | OR4C13 |
| 1257 | OR4C3 |
| 1258 | OR4E2 |
| 1259 | OR4F6 |
| 1260 | OR4K13 |
| 1261 | OR4Q2 |
| 1262 | OR4S1 |
| 1263 | OR51E1 |
| 1264 | OR51I2 |
| 1265 | OR51T1 |
| 1266 | OR52B6 |
| 1267 | OR52E2 |
| 1268 | OR52E4 |
| 1269 | OR52N2 |
| 1270 | OR52N5 |
| 1271 | OR52W1 |
| 1272 | OR52Z1 |
| 1273 | OR5AC2 |
| 1274 | OR5B3 |
| 1275 | OR5K3 |
| 1276 | OR5M8 |
| 1277 | OR6B1 |
| 1278 | OR6C2 |
| 1279 | OR6C68 |
| 1280 | OR6C74 |
| 1281 | OR6K3 |
| 1282 | OR6S1 |
| 1283 | OR7A5 |
| 1284 | OR7C1 |
| 1285 | OR7C2 |
| 1286 | OR7D2 |
| 1287 | OR7E24 |
| 1288 | OR7G1 |
| 1289 | OR8A1 |
| 1290 | OR8B4 |
| 1291 | OR8J3 |
| 1292 | OR9A4 |
| 1293 | OSTF1 |
| 1294 | OSTM1 |
| 1295 | OTOR |
| 1296 | OTUB2 |
| 1297 | OTUD1 |
| 1298 | OTUD6B-AS1 |
| 1299 | OVAAL |
| 1300 | PABPC5-AS1 |
| 1301 | PACRG-AS1 |
| 1302 | PACS1 |
| 1303 | PACSIN2 |
| 1304 | PADI2 |
| 1305 | PAFAH2 |
| 1306 | PAGE2 |
| 1307 | PAK1IP1 |
| 1308 | PAPD7 |
| 1309 | PAPSS2 |
| 1310 | PARP3 |
| 1311 | PARP8 |
| 1312 | PATE3 |
| 1313 | PAWR |
| 1314 | PAX2 |
| 1315 | PAX6 |
| 1316 | PAXBP1-AS1 |
| 1317 | PBDC1 |
| 1318 | PBX1 |
| 1319 | PCDH9-AS3 |
| 1320 | PCDHA11 |
| 1321 | PCDHA13 |
| 1322 | PCDHA2 |
| 1323 | PCDHA5 |
| 1324 | PCDHAC1 |
| 1325 | PCDHAC2 |
| 1326 | PCDHB1 |
| 1327 | PCDHB12 |
| 1328 | PCDHB15 |
| 1329 | PCDHB16 |
| 1330 | PCDHB4 |
| 1331 | PCDHB8 |
| 1332 | PCDHGA12 |
| 1333 | PCDHGA2 |
| 1334 | PCDHGA5 |
| 1335 | PCDHGA8 |
| 1336 | PCDHGA9 |
| 1337 | PCDHGB4 |
| 1338 | PCDHGB5 |
| 1339 | PCDHGB6 |
| 1340 | PCDHGB8P |
| 1341 | PCDHGC4 |
| 1342 | PCDHGC5 |
| 1343 | PCGF3 |
| 1344 | PCK2 |
| 1345 | PCMTD1 |
| 1346 | PCOTH |
| 1347 | PCTP |
| 1348 | PDAP1 |
| 1349 | PDC |
| 1350 | PDE6D |
| 1351 | PDK4 |
| 1352 | PDLIM1 |
| 1353 | PDPK1 |
| 1354 | PDZD8 |
| 1355 | PECR |
| 1356 | PELO |
| 1357 | PEX2 |
| 1358 | PEX5 |
| 1359 | PFDN4 |
| 1360 | PFKFB1 |
| 1361 | PFN4 |
| 1362 | PGBD2 |
| 1363 | PGK1 |
| 1364 | PGLYRP1 |
| 1365 | PGM2L1 |
| 1366 | PGM5P2 |
| 1367 | PGR-AS1 |
| 1368 | PGRMC2 |
| 1369 | PHC1 |
| 1370 | PHF20L1 |
| 1371 | PHF24 |
| 1372 | PHLDA3 |
| 1373 | PHYHD1 |
| 1374 | PI4K2B |
| 1375 | PIGBOS1 |
| 1376 | PIGZ |
| 1377 | PIN1P1 |
| 1378 | PIN4P1 |
| 1379 | PISRT1 |
| 1380 | PITRM1 |
| 1381 | PITRM1-AS1 |
| 1382 | PITX2 |
| 1383 | PKD2L1 |
| 1384 | PLA2G4F |
| 1385 | PLAGL1 |
| 1386 | PLCL2 |
| 1387 | PLEKHA1 |
| 1388 | PLEKHA4 |
| 1389 | PLEKHB2 |
| 1390 | PLIN1 |
| 1391 | PLPP1 |
| 1392 | PLPP3 |
| 1393 | PLPPR1 |
| 1394 | PLPPR2 |
| 1395 | PLRG1 |
| 1396 | PLSCR2 |
| 1397 | PLUT |
| 1398 | PMCH |
| 1399 | PMS2P1 |
| 1400 | PNLIPRP3 |
| 1401 | PNMA8B |
| 1402 | PNRC2 |
| 1403 | POLG2 |
| 1404 | POLN |
| 1405 | POM121L12 |
| 1406 | POM121L2 |
| 1407 | POMP |
| 1408 | POPDC3 |
| 1409 | POU2F3 |
| 1410 | PPARG |
| 1411 | PPBPP2 |
| 1412 | PPIE |
| 1413 | PPM1E |
| 1414 | PPM1F |
| 1415 | PPM1N |
| 1416 | PPP1R10 |
| 1417 | PPP1R14D |
| 1418 | PPP1R15A |
| 1419 | PPP1R7 |
| 1420 | PPP1R9B |
| 1421 | PPP4R3C |
| 1422 | PPWD1 |
| 1423 | PRDM13 |
| 1424 | PRKAB1 |
| 1425 | PRKAR2A-AS1 |
| 1426 | PRKX-AS1 |
| 1427 | PRLHR |
| 1428 | PRMT6 |
| 1429 | PRMT9 |
| 1430 | PROK2 |
| 1431 | PROKR2 |
| 1432 | PROSER1 |
| 1433 | PRPF38A |
| 1434 | PRPS1 |
| 1435 | PRR15L |
| 1436 | PRR23B |
| 1437 | PRR23C |
| 1438 | PRR26 |
| 1439 | PRRG4 |
| 1440 | PRRT4 |
| 1441 | PRSS22 |
| 1442 | PRSS30P |
| 1443 | PRSS46P |
| 1444 | PSD2-AS1 |
| 1445 | PSG3 |
| 1446 | PSG8 |
| 1447 | PSMA3 |
| 1448 | PSMC2 |
| 1449 | PSMD10 |
| 1450 | PSMD12 |
| 1451 | PSMD9 |
| 1452 | PSTK |
| 1453 | PTAFR |
| 1454 | PTBP3 |
| 1455 | PTGDR |
| 1456 | PTPN2 |
| 1457 | PTPRA |
| 1458 | PTPRG-AS1 |
| 1459 | PTTG1 |
| 1460 | PURG |
| 1461 | PUS3 |
| 1462 | PXN |
| 1463 | PYCARD-AS1 |
| 1464 | QKI |
| 1465 | RAB23 |
| 1466 | RABIF |
| 1467 | RAD51 |
| 1468 | RAD51AP1 |
| 1469 | RAET1G |
| 1470 | RAG1 |
| 1471 | RAP1B |
| 1472 | RAPGEF3 |
| 1473 | RASL11B |
| 1474 | RASSF10 |
| 1475 | RASSF4 |
| 1476 | RASSF6 |
| 1477 | RBM11 |
| 1478 | RBM15B |
| 1479 | RBM26-AS1 |
| 1480 | RBM3 |
| 1481 | RBM44 |
| 1482 | RBM46 |
| 1483 | RBM48 |
| 1484 | RBMS3-AS3 |
| 1485 | RBMX |
| 1486 | RBMXL2 |
| 1487 | RBPJL |
| 1488 | RC3H2 |
| 1489 | RCAN1 |
| 1490 | REG1CP |
| 1491 | RERG |
| 1492 | RFC3 |
| 1493 | RFESD |
| 1494 | RFPL1 |
| 1495 | RFPL1S |
| 1496 | RGR |
| 1497 | RGS12 |
| 1498 | RGS13 |
| 1499 | RGS18 |
| 1500 | RHOBTB1 |
| 1501 | RHOJ |
| 1502 | RHOU |
| 1503 | RIF1 |
| 1504 | RIOK2 |
| 1505 | RLIM |
| 1506 | RLN2 |
| 1507 | RNASE11 |
| 1508 | RNASE2 |
| 1509 | RNASE3 |
| 1510 | RNASE4 |
| 1511 | RNASE7 |
| 1512 | RNF103 |
| 1513 | RNF135 |
| 1514 | RNF152 |
| 1515 | RNF157-AS1 |
| 1516 | RNF167 |
| 1517 | RNF168 |
| 1518 | RNF24 |
| 1519 | RNF31 |
| 1520 | RNF34 |
| 1521 | RNF39 |
| 1522 | RNF5 |
| 1523 | RPAP1 |
| 1524 | RPL13AP17 |
| 1525 | RPL22L1 |
| 1526 | RPL31 |
| 1527 | RPL6 |
| 1528 | RPS20 |
| 1529 | RPS3A |
| 1530 | RPTN |
| 1531 | RRM1 |
| 1532 | RRP15 |
| 1533 | RRP1B |
| 1534 | RRP8 |
| 1535 | RRP9 |
| 1536 | RRS1-AS1 |
| 1537 | RSPH6A |
| 1538 | RTCA-AS1 |
| 1539 | RTL9 |
| 1540 | RUNX1-IT1 |
| 1541 | S1PR3 |
| 1542 | SALL1 |
| 1543 | SALL4 |
| 1544 | SALRNA2 |
| 1545 | SAMD9L |
| 1546 | SAP30 |
| 1547 | SASS6 |
| 1548 | SATB2-AS1 |
| 1549 | SCAF8 |
| 1550 | SCARF1 |
| 1551 | SCART1 |
| 1552 | SCG2 |
| 1553 | SCGB1B2P |
| 1554 | SCGB1D1 |
| 1555 | SCGB1D2 |
| 1556 | SCGB1D4 |
| 1557 | SCGB2B2 |
| 1558 | SCGB3A2 |
| 1559 | SCML1 |
| 1560 | SCO1 |
| 1561 | SCUBE3 |
| 1562 | SCYL3 |
| 1563 | SDR39U1 |
| 1564 | SDR9C7 |
| 1565 | SECISBP2 |
| 1566 | SELENOP |
| 1557 | SELPLG |
| 1568 | SEMA4A |
| 1569 | SEPSECS-AS1 |
| 1570 | SERBP1 |
| 1571 | SERPINA7 |
| 1572 | SERPINB4 |
| 1573 | SERTAD3 |
| 1574 | SERTM2 |
| 1575 | SETD1B |
| 1576 | SGCD |
| 1577 | SGO2 |
| 1578 | SH2D3C |
| 1579 | SH2D5 |
| 1580 | SH3D21 |
| 1581 | SH3GLB1 |
| 1582 | SHANK3 |
| 1583 | SHB |
| 1584 | SHCBP1 |
| 1585 | SHISA7 |
| 1586 | SHISAL2A |
| 1587 | SHOC2 |
| 1588 | SHOX2 |
| 1589 | SIAH1 |
| 1590 | SIKE1 |
| 1591 | SIRPA |
| 1592 | SLAIN2 |
| 1593 | SLC10A5 |
| 1594 | SLC11A2 |
| 1595 | SLC12A4 |
| 1596 | SLC16A10 |
| 1597 | SLC16A12 |
| 1598 | SLC16A13 |
| 1599 | SLC16A9 |
| 1600 | SLC17A2 |
| 1601 | SLC22A10 |
| 1602 | SLC22A3 |
| 1603 | SLC22A4 |
| 1604 | SLC22A7 |
| 1605 | SLC22A8 |
| 1606 | SLC23A1 |
| 1607 | SLC25A16 |
| 1608 | SLC25A19 |
| 1609 | SLC25A32 |
| 1610 | SLC25A35 |
| 1611 | SLC25A36 |
| 1612 | SLC25A4 |
| 1613 | SLC25A42 |
| 1614 | SLC30A7 |
| 1615 | SLC30A8 |
| 1616 | SLC31A1 |
| 1617 | SLC35B4 |
| 1618 | SLC35E4 |
| 1619 | SLC35G1 |
| 1620 | SLC35G3 |
| 1621 | SLC36A1 |
| 1622 | SLC37A2 |
| 1623 | SLC37A3 |
| 1624 | SLC46A1 |
| 1625 | SLC51A |
| 1626 | SLC52A3 |
| 1627 | SLC5A10 |
| 1628 | SLC5A3 |
| 1629 | SLC5A6 |
| 1630 | SLC5A8 |
| 1631 | SLC6A11 |
| 1632 | SLC6A4 |
| 1633 | SLC6A7 |
| 1634 | SLC7A6OS |
| 1635 | SLC7A9 |
| 1636 | SLC8B1 |
| 1637 | SLFNL1 |
| 1638 | SLITRK5 |
| 1639 | SMAD1-AS2 |
| 1640 | SMAD5-AS1 |
| 1641 | SMIM10L2B |
| 1642 | SMIM12 |
| 1643 | SMIM18 |
| 1644 | SMIM27 |
| 1645 | SMLR1 |
| 1646 | SMO |
| 1647 | SNAI2 |
| 1648 | SNAPC5 |
| 1649 | SNAR-I |
| 1650 | SNHG6 |
| 1651 | SNIP1 |
| 1652 | SNORA20B |
| 1653 | SNORA32 |
| 1654 | SNORA8 |
| 1655 | SNORA80A |
| 1656 | SNORA87 |
| 1657 | SNORD114-29 |
| 1658 | SNORD114-30 |
| 1659 | SNORD115-45 |
| 1660 | SNORD38D |
| 1661 | SNORD3G |
| 1662 | SNORD89 |
| 1663 | SNORD91B |
| 1664 | SNRNP27 |
| 1665 | SNX10 |
| 1666 | SNX18 |
| 1667 | SNX21 |
| 1668 | SOST |
| 1669 | SOWAHB |
| 1670 | SOWAHC |
| 1671 | SP1 |
| 1672 | SP2 |
| 1673 | SPACA1 |
| 1674 | SPACA3 |
| 1675 | SPATA3-AS1 |
| 1676 | SPATA31D1 |
| 1677 | SPATA31E1 |
| 1678 | SPATA33 |
| 1679 | SPATA41 |
| 1680 | SPC25 |
| 1681 | SPEN |
| 1682 | SPINK7 |
| 1683 | SPINK9 |
| 1684 | SPINT4 |
| 1685 | SPRR1A |
| 1686 | SPRR2E |
| 1687 | SPRY2 |
| 1688 | SPRYD4 |
| 1689 | SRP14-AS1 |
| 1690 | SRR |
| 1691 | SRSF4 |
| 1692 | SRSF7 |
| 1693 | SSFA2 |
| 1694 | SSR1 |
| 1695 | SSR3 |
| 1696 | SSX9 |
| 1697 | ST13P4 |
| 1698 | ST8SIA6 |
| 1699 | STARD5 |
| 1700 | STEAP4 |
| 1701 | STK4-AS1 |
| 1702 | STN1 |
| 1703 | STOM |
| 1704 | STON1 |
| 1705 | STPG1 |
| 1706 | STX16 |
| 1707 | STX1B |
| 1708 | STXBP3 |
| 1709 | STYK1 |
| 1710 | SUGT1 |
| 1711 | SUMF1 |
| 1712 | SUMO1P3 |
| 1713 | SUPT20HL2 |
| 1714 | SUPT3H |
| 1715 | SURF4 |
| 1716 | SUV39H1 |
| 1717 | SVBP |
| 1718 | SYCE1L |
| 1719 | SYDE2 |
| 1720 | SYNE1-AS1 |
| 1721 | SYPL1 |
| 1722 | SYT4 |
| 1723 | TAAR9 |
| 1724 | TAB2-AS1 |
| 1725 | TACSTD2 |
| 1726 | TAF1L |
| 1727 | TAS1R1 |
| 1728 | TAS2R1 |
| 1729 | TAS2R3 |
| 1730 | TAS2R30 |
| 1731 | TAS2R39 |
| 1732 | TAS2R42 |
| 1733 | TATDN2 |
| 1734 | TBPL1 |
| 1735 | TBR1 |
| 1736 | TCAM1P |
| 1737 | TCEAL3 |
| 1738 | TCEAL4 |
| 1739 | TCEANC |
| 1740 | TCF24 |
| 1741 | TCHH |
| 1742 | TCHHL1 |
| 1743 | TCOF1 |
| 1744 | TCP11L1 |
| 1745 | TCP11L2 |
| 1746 | TDRD6 |
| 1747 | TDRKH-AS1 |
| 1748 | TEAD3 |
| 1749 | TEAD4 |
| 1750 | TEFM |
| 1751 | TET2 |
| 1752 | TEX101 |
| 1753 | TFB2M |
| 1754 | TFE3 |
| 1755 | TFF2 |
| 1756 | TGFA |
| 1757 | TGFB3 |
| 1758 | THAP2 |
| 1759 | THEM4 |
| 1760 | TIGD4 |
| 1761 | TIMM21 |
| 1762 | TIMM8A |
| 1763 | TIMM9 |
| 1764 | TKTL2 |
| 1765 | TLCD2 |
| 1766 | TLE3 |
| 1767 | TLR1 |
| 1768 | TLR4 |
| 1769 | TLX1NB |
| 1770 | TM6SF1 |
| 1771 | TMCO1 |
| 1772 | TMEM128 |
| 1773 | TMEM132E |
| 1774 | TMEM14EP |
| 1775 | TMEM155 |
| 1776 | TMEM169 |
| 1777 | TMEM170A |
| 1778 | TMEM177 |
| 1779 | TMEM200C |
| 1780 | TMEM209 |
| 1781 | TMEM220 |
| 1782 | TMEM225 |
| 1783 | TMEM229B |
| 1784 | TMEM235 |
| 1785 | TMEM254 |
| 1786 | TMEM255B |
| 1787 | TMEM53 |
| 1788 | TMEM62 |
| 1789 | TMEM65 |
| 1790 | TMEM81 |
| 1791 | TMEM86A |
| 1792 | TMEM9 |
| 1793 | TMEM99 |
| 1794 | TMIGD3 |
| 1795 | TMOD4 |
| 1796 | TMPPE |
| 1797 | TMPRSS4 |
| 1798 | TMSB15A |
| 1799 | TNFSF13B |
| 1800 | TNK2-AS1 |
| 1801 | TNPO1 |
| 1802 | TNRC17 |
| 1803 | TNRC6C |
| 1804 | TOMM34 |
| 1805 | TOP1P2 |
| 1806 | TOPORS |
| 1807 | TP53 |
| 1808 | TPCN2 |
| 1809 | TPD52L3 |
| 1810 | TPI1P2 |
| 1811 | TPRG1-AS1 |
| 1812 | TRAF6 |
| 1813 | TRAM1 |
| 1814 | TRAM1L1 |
| 1815 | TRDN-AS1 |
| 1816 | TRIB1 |
| 1817 | TRIB2 |
| 1818 | TRIM23 |
| 1819 | TRIM3 |
| 1820 | TRIM32 |
| 1821 | TRIM51 |
| 1822 | TRIM58 |
| 1823 | TRIM69 |
| 1824 | TRIQK |
| 1825 | TRMT5 |
| 1826 | TRNT1 |
| 1827 | TRPC7-AS2 |
| 1828 | TSC22D1 |
| 1829 | TSGA13 |
| 1830 | TSHR |
| 1831 | TSPAN2 |
| 1832 | TSPYL4 |
| 1833 | TSR1 |
| 1834 | TSSK1B |
| 1835 | TSSK4 |
| 1836 | TTBK1 |
| 1837 | TTC8 |
| 1838 | TTF1 |
| 1839 | TTL |
| 1840 | TTPA |
| 1841 | TUBB4A |
| 1842 | TUBG1 |
| 1843 | TWF1 |
| 1844 | TXNDC2 |
| 1845 | TYMS |
| 1846 | TYMSOS |
| 1847 | UBA5 |
| 1848 | UBA6 |
| 1849 | UBA7 |
| 1850 | UBE2D1 |
| 1851 | UBE2G1 |
| 1852 | UBE2L6 |
| 1853 | UBE2MP1 |
| 1854 | UBE2N |
| 1855 | UBE2Q2 |
| 1856 | UBR7 |
| 1857 | UCA1 |
| 1858 | UGDH |
| 1859 | UGT1A1 |
| 1860 | UGT1A10 |
| 1861 | UNC45B |
| 1862 | UOX |
| 1863 | UPF1 |
| 1864 | UPF3B |
| 1865 | USP15 |
| 1866 | USP20 |
| 1867 | USP30 |
| 1868 | USP40 |
| 1869 | USP51 |
| 1870 | UTP14C |
| 1871 | UTP3 |
| 1872 | VARS |
| 1873 | VCAM1 |
| 1874 | VGLL1 |
| 1875 | VMA21 |
| 1876 | VPS37C |
| 1877 | VPS51 |
| 1878 | VPS53 |
| 1879 | VSIG1 |
| 1880 | VSIG10L |
| 1881 | VSTM2L |
| 1882 | VSTM5 |
| 1883 | VSX1 |
| 1884 | VWA5B2 |
| 1885 | VWC2 |
| 1886 | WASF3 |
| 1887 | WBP11 |
| 1888 | WDFY4 |
| 1889 | WDR45 |
| 1890 | WDR55 |
| 1891 | WDR92 |
| 1892 | WFDC2 |
| 1893 | WFDC5 |
| 1894 | WFIKKN2 |
| 1895 | WISP3 |
| 1896 | WNT10B |
| 1897 | WNT9B |
| 1898 | WSB2 |
| 1899 | WTAP |
| 1900 | XCR1 |
| 1901 | XK |
| 1902 | XKR4 |
| 1903 | XKR6 |
| 1904 | XKR9 |
| 1905 | XXYLT1-AS2 |
| 1906 | XYLT2 |
| 1907 | YBEY |
| 1908 | YBX1 |
| 1909 | YBX3P1 |
| 1910 | YES1 |
| 1911 | YIPF4 |
| 1912 | YIPF7 |
| 1913 | YTHDF1 |
| 1914 | ZBED2 |
| 1915 | ZBED3 |
| 1916 | ZBED6CL |
| 1917 | ZBTB24 |
| 1918 | ZBTB41 |
| 1919 | ZBTB8B |
| 1920 | ZC2HC1A |
| 1921 | ZC3H15 |
| 1922 | ZC3HAV1L |
| 1923 | ZCCHC10 |
| 1924 | ZCCHC12 |
| 1925 | ZDHHC2 |
| 1926 | ZDHHC9 |
| 1927 | ZFAND2B |
| 1928 | ZFP14 |
| 1929 | ZFP91 |
| 1930 | ZNF155 |
| 1931 | ZNF19 |
| 1932 | ZNF195 |
| 1933 | ZNF207 |
| 1934 | ZNF211 |
| 1935 | ZNF234 |
| 1936 | ZNF235 |
| 1937 | ZNF254 |
| 1938 | ZNF260 |
| 1939 | ZNF275 |
| 1940 | ZNF280A |
| 1941 | ZNF280C |
| 1942 | ZNF284 |
| 1943 | ZNF30-AS1 |
| 1944 | ZNF33BP1 |
| 1945 | ZNF34 |
| 1946 | ZNF362 |
| 1947 | ZNF384 |
| 1948 | ZNF398 |
| 1949 | ZNF432 |
| 1950 | ZNF44 |
| 1951 | ZNF460-AS1 |
| 1952 | ZNF503 |
| 1953 | ZNF529-AS1 |
| 1954 | ZNF559 |
| 1955 | ZNF57 |
| 1956 | ZNF571 |
| 1957 | ZNF577 |
| 1958 | ZNF595 |
| 1959 | ZNF611 |
| 1960 | ZNF619 |
| 1961 | ZNF665 |
| 1962 | ZNF691 |
| 1963 | ZNF692 |
| 1964 | ZNF70 |
| 1965 | ZNF710 |
| 1966 | ZNF770 |
| 1967 | ZNF804A |
| 1968 | ZNF804B |
| 1969 | ZNF805 |
| 1970 | ZNF823 |
| 1971 | ZNF843 |
| 1972 | ZNF847P |
| 1973 | ZNF850 |
| 1974 | ZRANB1 |
| 1975 | ZSCAN2 |
| 1976 | ZSCAN20 |
| 1977 | ZSWIM9 |
| 1978 | ZXDA |
| 1979 | ZYG11A |
| 1980 | AAMP |
| 1981 | ABCC5-AS1 |
| 1982 | ADGRE5 |
| 1983 | ADH1A |
| 1984 | AJAP1 |
| 1985 | ALAD |
| 1986 | ALG1 |
| 1987 | ALG14 |
| 1988 | ANKRD39 |
| 1989 | APOA5 |
| 1990 | AQP3 |
| 1991 | ARHGEF25 |
| 1992 | ARID5B |
| 1993 | ARL4D |
| 1994 | ATE1-AS1 |
| 1995 | ATP10B |
| 1996 | ATXN7L1 |
| 1997 | B3GNT2 |
| 1998 | B9D2 |
| 1999 | BLK |
| 2000 | BRD9 |
| 2001 | BRINP2 |
| 2002 | BYSL |
| 2003 | C12orf43 |
| 2004 | C12orf50 |
| 2005 | C12orf77 |
| 2006 | C14orf144 |
| 2007 | C16orf90 |
| 2008 | C22orf24 |
| 2009 | C4orf3 |
| 2010 | C8orf74 |
| 2011 | CADM2-AS2 |
| 2012 | CCAR1 |
| 2013 | CCDC9 |
| 2014 | CD2 |
| 2015 | CD68 |
| 2016 | CD82 |
| 2017 | CD8A |
| 2018 | CEACAM19 |
| 2019 | CFB |
| 2020 | CHCHD5 |
| 2021 | CHRNB2 |
| 2022 | CIDECP |
| 2023 | CIT |
| 2024 | CLDN11 |
| 2025 | CMTM7 |
| 2026 | CNGB1 |
| 2027 | CNPPD1 |
| 2028 | COL9A3 |
| 2029 | CPA2 |
| 2030 | CSTA |
| 2031 | CWC25 |
| 2032 | DDX47 |
| 2033 | DEFB115 |
| 2034 | DGKK |
| 2035 | DIAPH2-AS1 |
| 2036 | DISC1-IT1 |
| 2037 | DNAH17-AS1 |
| 2038 | DNAJC12 |
| 2039 | DPH3P1 |
| 2040 | DPRX |
| 2041 | DPYD-AS1 |
| 2042 | DYNC1I2 |
| 2043 | EEF1AKMT2 |
| 2044 | EIF2B2 |
| 2045 | EIF4E2 |
| 2046 | F3 |
| 2047 | FADS2 |
| 2048 | FAM214B |
| 2049 | FBXO9 |
| 2050 | FBXW4 |
| 2051 | FCF1P2 |
| 2052 | FRG1DP |
| 2053 | FRY-AS1 |
| 2054 | FXYD5 |
| 2055 | GABARAPL1 |
| 2056 | GDF11 |
| 2057 | GGA3 |
| 2058 | GID4 |
| 2059 | GNG7 |
| 2060 | GPATCH1 |
| 2061 | GPATCH4 |
| 2062 | GPC6 |
| 2063 | GPR153 |
| 2064 | GPX3 |
| 2065 | GRAMD2A |
| 2066 | GRB10 |
| 2067 | GRM7 |
| 2068 | HAX1 |
| 2069 | HMGB1 |
| 2070 | HNRNPH1 |
| 2071 | HSD17B2 |
| 2072 | IDH3B |
| 2073 | INO80D |
| 2074 | INTS14 |
| 2075 | IVD |
| 2076 | JAKMIP1 |
| 2077 | KANSL1-AS1 |
| 2078 | KATNAL1 |
| 2079 | KCNA4 |
| 2080 | KCNK10 |
| 2081 | KCTD2 |
| 2082 | KDM4D |
| 2083 | KLHL29 |
| 2084 | KRBOX4 |
| 2085 | KRT18 |
| 2086 | LAIR1 |
| 2087 | LHFPL3-AS1 |
| 2088 | LIN9 |
| 2089 | LINC00114 |
| 2090 | LINC00342 |
| 2091 | LINC00347 |
| 2092 | LINC00433 |
| 2093 | LINC00443 |
| 2094 | LINC00536 |
| 2095 | LINC00599 |
| 2096 | LINC00694 |
| 2097 | LINC00704 |
| 2098 | LINC00705 |
| 2099 | LINC00864 |
| 2100 | LINC01005 |
| 2101 | LINC01048 |
| 2102 | LINC01102 |
| 2103 | LINC01118 |
| 2104 | LINC01220 |
| 2105 | LINC01231 |
| 2106 | LINC01307 |
| 2107 | LINC01354 |
| 2108 | LINC01470 |
| 2109 | LINC01482 |
| 2110 | LINC01555 |
| 2111 | LINC01611 |
| 2112 | LINC01750 |
| 2113 | LINC01910 |
| 2114 | LINC01930 |
| 2115 | LINC02118 |
| 2116 | LINC02214 |
| 2117 | LINC02219 |
| 2118 | LINC02274 |
| 2119 | LINC02280 |
| 2120 | LINC02351 |
| 2121 | LOC100653233 |
| 2122 | LOC101926933 |
| 2123 | LOC101927278 |
| 2124 | LOC101927365 |
| 2125 | LOC101927822 |
| 2126 | LOC101928087 |
| 2127 | LOC101928445 |
| 2128 | LOC101929124 |
| 2129 | LOC102725126 |
| 2130 | LOC105370473 |
| 2131 | LOC105375843 |
| 2132 | LOC105376351 |
| 2133 | LOC339685 |
| 2134 | LOC399886 |
| 2135 | LOC401176 |
| 2136 | LSP1P3 |
| 2137 | LIB4R |
| 2138 | MAEL |
| 2139 | MAGEB5 |
| 2140 | MAN2B1 |
| 2141 | MAP6 |
| 2142 | MARF1 |
| 2143 | MCHR2-AS1 |
| 2144 | MED13L |
| 2145 | METTL5 |
| 2146 | MGAT1 |
| 2147 | MIATNB |
| 2148 | MIR1305 |
| 2149 | MIR205HG |
| 2150 | MIR3936 |
| 2151 | MIR3937 |
| 2152 | MIR4423 |
| 2153 | MIR4477A |
| 2154 | MIR548BA |
| 2155 | MIR576 |
| 2156 | MIR585 |
| 2157 | MIR6082 |
| 2158 | MIR8074 |
| 2159 | MIR8076 |
| 2160 | MIXL1 |
| 2161 | MLX |
| 2162 | MOBP |
| 2163 | MPV17L |
| 2164 | MRPS10 |
| 2165 | MRPS11 |
| 2166 | MSH2 |
| 2167 | MYBPHL |
| 2168 | MYZAP |
| 2169 | NAA10 |
| 2170 | NECAB2 |
| 2171 | NEDD1 |
| 2172 | NELFE |
| 2173 | NEU2 |
| 2174 | NFKB1 |
| 2175 | NIPAL2 |
| 2176 | NNMT |
| 2177 | NPL |
| 2178 | NREP |
| 2179 | NTRK3 |
| 2180 | ODC1 |
| 2181 | OOEP |
| 2182 | OR4B1 |
| 2183 | OR4C11 |
| 2184 | OSER1-AS1 |
| 2185 | OVCH2 |
| 2186 | PCAT19 |
| 2187 | PCDH9-AS4 |
| 2188 | PCP4L1 |
| 2189 | PHOSPHO1 |
| 2190 | PIGL |
| 2191 | PIGW |
| 2192 | PIP5K1C |
| 2193 | PLA2G10 |
| 2194 | PMS2 |
| 2195 | POLR2J4 |
| 2196 | PPM1D |
| 2197 | PPP2R2B |
| 2198 | PPP2R2C |
| 2199 | PSG5 |
| 2200 | PSMA6 |
| 2201 | PTEN |
| 2202 | RALGDS |
| 2203 | RASSF5 |
| 2204 | RBFADN |
| 2205 | RBM15 |
| 2206 | RET |
| 2207 | RGS10 |
| 2208 | RIMBP2 |
| 2209 | RMST |
| 2210 | RNF207 |
| 2211 | RNR1 |
| 2212 | RNR2 |
| 2213 | RUBCNL |
| 2214 | RXFP2 |
| 2215 | RYR3 |
| 2216 | S100A2 |
| 2217 | SAMD7 |
| 2218 | SCARNA3 |
| 2219 | SEMA4F |
| 2220 | SERP2 |
| 2221 | SERPINB11 |
| 2222 | SFMBT2 |
| 2223 | SFPQ |
| 2224 | SHISA6 |
| 2225 | SIM1 |
| 2226 | SIRT5 |
| 2227 | SKIV2L |
| 2228 | SLAMF8 |
| 2229 | SLC17A4 |
| 2230 | SLC22A5 |
| 2231 | SLC23A3 |
| 2232 | SLC5A4 |
| 2233 | SLC6A16 |
| 2234 | SMIM15-AS1 |
| 2235 | SMUG1 |
| 2236 | SNORD114-14 |
| 2237 | SNORD114-15 |
| 2238 | SNORD114-19 |
| 2239 | SPACA6P-AS |
| 2240 | SPANXN5 |
| 2241 | SPIRE2 |
| 2242 | SRRM1 |
| 2243 | STK3 |
| 2244 | STK35 |
| 2245 | STT3B |
| 2246 | STX4 |
| 2247 | SYAP1 |
| 2248 | SYT17 |
| 2249 | TARDBP |
| 2250 | TAS2R16 |
| 2251 | TBC1D26 |
| 2252 | TBL2 |
| 2253 | TCFL5 |
| 2254 | THRA |
| 2255 | THY1 |
| 2256 | TLR8 |
| 2257 | TMBIM1 |
| 2258 | TMCC1 |
| 2259 | TMEM139 |
| 2260 | TMEM42 |
| 2261 | TMEM5-AS1 |
| 2262 | TMEM98 |
| 2263 | TNFRSF1A |
| 2264 | TOX4 |
| 2265 | TRNP1 |
| 2266 | TTC32 |
| 2267 | TXN2 |
| 2268 | TXNL4A |
| 2269 | TYR |
| 2270 | UBE4B |
| 2271 | UBXN10-AS1 |
| 2272 | UGT2A1 |
| 2273 | UTP4 |
| 2274 | VIM |
| 2275 | VTRNA1-1 |
| 2276 | VWC2L |
| 2277 | WDR11-AS1 |
| 2278 | WDR61 |
| 2279 | WDR91 |
| 2280 | WLS |
| 2281 | YEATS2 |
| 2282 | YEATS2-AS1 |
| 2283 | ZC3H12C |
| 2284 | ZDHHC11B |
| 2285 | ZFP2 |
| 2286 | ZMYND10 |
| 2287 | ZNF14 |
| 2288 | ZNF142 |
| 2289 | ZNF157 |
| 2290 | ZNF160 |
| 2291 | ZNF182 |
| 2292 | ZNF230 |
| 2293 | ZNF346 |
| 2294 | ZNF441 |
| 2295 | ZNF462 |
| 2296 | ZNF534 |
| 2297 | ZNF544 |
| 2298 | ZNF56 |
| 2299 | ZNF569 |
| 2300 | ZNF613 |
| 2301 | ZNF627 |
| 2302 | ZNF684 |
| 2303 | ZNF707 |
| 2304 | ZNF883 |
| 2305 | ZNF98 |
| 2306 | ZRSR2 |

### Example 7: Construction of a Classifier for Typing Cervical Cancer Patients Using Erythrocyte Micronuclei DNA

In this example, there were 8 subjects, including 2 patients diagnosed as cervical adenocarcinoma and 5 patients diagnosed as cervical squamous cell carcinoma by other methods.

The peripheral blood samples from patients with cervical cancer were expressed in the form of “P” plus patient number. For example, “P1” represents a peripheral blood sample from the first cervical cancer patient (“Patient 1”), “P2” represents a peripheral blood sample from the second cervical cancer patient (“Patient 2”), and so on.

The basic information of 7 patients with cervical cancer is shown in Table 3. “Cervical cancer type” refers to the type of cervical cancer diagnosed by other methods.

TABLE 3

| | | HPV | Maximum | | |
|---|---|---|---|---|---|
| Patient | | Detected | diameter of | | Existence of lymph |
| number | Age | value | tumor (cm) | Type of cervical cancer | node metastasis or not |
| 1 | 44 | 68.36 | 2.1 | Medium-high differentiated | No |
| | | | | adenocarcinoma | |
| 2 | 36 | 50.13 | 4.2 | Low-medium differentiated | No |
| | | | | adenocarcinoma | |
| 3 | 71 | 1212.8 | 2.6 | Medium differentiated | Yes (without surgery) |
| | | | | squamous cell carcinoma | |
| 4 | 43 | 240.09 | 4.1 | Low differentiated | Yes (without surgery) |
| | | | | squamous cell carcinoma | |
| 5 | 65 | 25.55 | 3.3 | Medium differentiated | No |
| | | | | squamous cell carcinoma | |
| 6 | 56 | 503.61 | 3.2 | Low-medium differentiated | No |
| | | | | squamous cell carcinoma | |
| 7* | 37 | No | 5.4 | Low-medium differentiated | Yes |
| | | | | squamous cell carcinoma | |

*Patient 7 is an HPV negative patient.

Erythrocyte micronuclei DNA and genomic DNA of peripheral blood mononuclear cells of each subject were obtained as described in Examples 1-4, and bioinformatics analysis was carried out as described in Example 5.

Specifically, 2 adenocarcinoma samples and 6 squamous cell carcinoma (including one HPV-negative sample) in primary cervical cancer samples were selected for the reads counting, and 360 differential genes were screened out by ANOVA test to distinguish the two classes of samples. Then, according to Pearson correlation, the two classes of samples were clustered in unsupervised hierarchy, showing that there were significant differences between the two classes of samples.

As shown in FIG. 6, 360 differential genes (forming a classifier for distinguishing cervical adenocarcinoma from cervical squamous cell carcinoma patients) were clustered. In FIG. 6, each row represents a differential gene, and each column represents a patient.

The list of 360 differential genes is shown in Table 4. Each gene corresponds to each row from top to bottom in FIG. 6.

TABLE 4

| | |
|---|---|
| 1 | ACAD10 |
| 2 | ACOD1 |
| 3 | ACTR1A |
| 4 | ANP32A-IT1 |
| 5 | ARL5C |
| 6 | ATP13A2 |
| 7 | B2M |
| 8 | C11orf70 |
| 9 | C16orf70 |
| 10 | C19orf66 |
| 11 | C1orf194 |
| 12 | CABYR |
| 13 | CADM2-AS2 |
| 14 | CALB1 |
| 15 | CAPSL |
| 16 | CCT8 |
| 17 | CNFN |
| 18 | CROCC |
| 19 | CYP27C1 |
| 20 | DNAAF1 |
| 21 | DYNLRB2 |
| 22 | ESCO1 |
| 23 | ESYT3 |
| 24 | FAM199X |
| 25 | FAM19A1 |
| 26 | FGFBP3 |
| 27 | FITM2 |
| 28 | FLJ37201 |
| 29 | GLA |
| 30 | GPN1 |
| 31 | GTF2H1 |
| 32 | HAS2-AS1 |
| 33 | HOXD1 |
| 34 | IP6K3 |
| 35 | KCNH3 |
| 36 | KRTAP22-2 |
| 37 | LINC00605 |
| 38 | LINC01393 |
| 39 | LINC01602 |
| 40 | LINC01803 |
| 41 | LINC01845 |
| 42 | LINC01991 |
| 43 | LOC101928211 |
| 44 | LOC101929130 |
| 45 | LOC101929524 |
| 46 | LOC102467081 |
| 47 | LOC107133515 |
| 48 | LRRC17 |
| 49 | LUC7L2 |
| 50 | LYPLA1 |
| 51 | MARCH4 |
| 52 | MGAT4EP |
| 53 | MINDY4B |
| 54 | MIR3663HG |
| 55 | MIR553 |
| 56 | MKRN3 |
| 57 | MRPS18C |
| 58 | NBPF9 |
| 59 | NDUFV2 |
| 60 | NKRF |
| 61 | NUDT5 |
| 62 | OMA1 |
| 63 | OR10J1 |
| 64 | OR10Z1 |
| 65 | OR1S1 |
| 66 | OR4C11 |
| 67 | OR4X1 |
| 68 | OR51B5 |
| 69 | OR5AN1 |
| 70 | P4HA1 |
| 71 | PCED1B |
| 72 | SLC22A7 |
| 73 | SLC35B2 |
| 74 | SLC35F3 |
| 75 | SMS |
| 76 | SNORD3K |
| 77 | SVIP |
| 78 | TAF6 |
| 79 | TAS2R3 |
| 80 | TCAM1P |
| 81 | TGM1 |
| 82 | TGOLN2 |
| 83 | TMCO2 |
| 84 | TMEM132A |
| 85 | TMEM14EP |
| 86 | TREML4 |
| 87 | TSPAN31 |
| 88 | UBE2N |
| 89 | VAMP4 |
| 90 | XIAP |
| 91 | ZNF718 |
| 92 | ADAM30 |
| 93 | AMER3 |
| 94 | ANKRD62P1-PARP4P3 |
| 95 | ANP32A |
| 96 | APOF |
| 97 | APOOP5 |
| 98 | ARHGEF25 |
| 99 | ASAP1-IT2 |
| 100 | ASF1B |
| 101 | ASIC2 |
| 102 | ASNS |
| 103 | ATP6V0B |
| 104 | BANCR |
| 105 | BMS1P21 |
| 106 | BOD1L2 |
| 107 | BRWD1-AS2 |
| 108 | BTNL8 |
| 109 | C12orf71 |
| 110 | C2orf88 |
| 111 | C6orf222 |
| 112 | C9orf3 |
| 113 | CAHM |
| 114 | CD24 |
| 115 | CDKN2AIP |
| 116 | CEP170 |
| 117 | CERS3 |
| 118 | CFL2 |
| 119 | CHAMP1 |
| 120 | CHERP |
| 121 | CHRNE |
| 122 | CLLU1 |
| 123 | CNOT8 |
| 124 | COL23A1 |
| 125 | COL4A1 |
| 126 | CREB3L3 |
| 127 | CTAGE9 |
| 128 | CTRC |
| 129 | CYB5R1 |
| 130 | CYMP-AS1 |
| 131 | CYP4F29P |
| 132 | DCAF8L1 |
| 133 | DCK |
| 134 | DEFA11P |
| 135 | DGCR11 |
| 136 | DGCR9 |
| 137 | DICER1-AS1 |
| 138 | DLGAP3 |
| 139 | DPPA3 |
| 140 | DPY19L1P2 |
| 141 | DSCR9 |
| 142 | EDAR |
| 143 | EIF3IP1 |
| 144 | EIF5A |
| 145 | ESRP2 |
| 146 | FABP3 |
| 147 | FAM129A |
| 148 | FAM49A |
| 149 | FBXL19-AS1 |
| 150 | FFAR2 |
| 151 | FKBP1A |
| 152 | FRMD1 |
| 153 | GABPB1-AS1 |
| 154 | GAGE1 |
| 155 | GALNT16 |
| 156 | GCK |
| 157 | GNAI3 |
| 158 | GPR83 |
| 159 | GRIK1-AS1 |
| 160 | GSX1 |
| 161 | GTF2H5 |
| 162 | GTPBP2 |
| 163 | H19 |
| 164 | HLA-DQB1 |
| 165 | HOXC4 |
| 166 | HRAT5 |
| 167 | HSD17B1 |
| 168 | IFITM10 |
| 169 | IFNA1 |
| 170 | IGLL1 |
| 171 | INSL4 |
| 172 | KAZN-AS1 |
| 173 | KCNG1 |
| 174 | KCNIP3 |
| 175 | KCNJ9 |
| 176 | KCNQ4 |
| 177 | KIRREL2 |
| 178 | KLF4 |
| 179 | KRTAP20-2 |
| 180 | LAMB2P1 |
| 181 | LCE1E |
| 182 | LINC00312 |
| 183 | LINC00449 |
| 184 | LINC00469 |
| 185 | LINC00547 |
| 186 | LINC00561 |
| 187 | LINC00656 |
| 188 | LINC00680 |
| 189 | LINC00851 |
| 190 | LINC00857 |
| 191 | LINC00887 |
| 192 | LINC01058 |
| 193 | LINC01118 |
| 194 | LINC01496 |
| 195 | LINC01565 |
| 196 | LINC01579 |
| 197 | LINC01621 |
| 198 | LINC01658 |
| 199 | LINC01674 |
| 200 | LINC01737 |
| 201 | LINC01752 |
| 202 | LINC01833 |
| 203 | LINC01872 |
| 204 | LINC01894 |
| 205 | LINC01953 |
| 206 | LINC01964 |
| 207 | LINC01982 |
| 208 | LINC02145 |
| 209 | LINC02214 |
| 210 | LINC02226 |
| 211 | LINC02283 |
| 212 | LINC02313 |
| 213 | LINC02468 |
| 214 | LOC100129129 |
| 215 | LOC100130872 |
| 216 | LOC100190986 |
| 217 | LOC100505824 |
| 218 | LOC100506585 |
| 219 | LOC100996415 |
| 220 | LOC101593348 |
| 221 | LOC101927342 |
| 222 | LOC101927497 |
| 223 | LOC101927588 |
| 224 | LOC101927762 |
| 225 | LOC101928307 |
| 226 | LOC101928489 |
| 227 | LOC103611081 |
| 228 | LOC105369632 |
| 229 | LOC105371880 |
| 230 | LOC105372493 |
| 231 | LOC105373876 |
| 232 | LOC105377967 |
| 233 | LOC105378385 |
| 234 | LOC105378663 |
| 235 | LOC285556 |
| 236 | LOC400682 |
| 237 | LOC441666 |
| 238 | LOC643802 |
| 239 | LOC646903 |
| 240 | LOC729681 |
| 241 | LRRC3-AS1 |
| 242 | LRRC4B |
| 243 | LY6K |
| 244 | LYZL2 |
| 245 | MAGEB6 |
| 246 | MECOM |
| 247 | MESTIT1 |
| 248 | MGAT1 |
| 249 | MIR205HG |
| 250 | MIR30B |
| 251 | MIR4677 |
| 252 | MIR502 |
| 253 | MIR503HG |
| 254 | MIR513A1 |
| 255 | MIR7852 |
| 256 | MIRLET7F2 |
| 257 | MKLN1-AS |
| 258 | MN1 |
| 259 | MRGPRX2 |
| 260 | MRPL33 |
| 261 | MT1IP |
| 262 | MTCH1 |
| 263 | MXRA5 |
| 264 | NANOGP8 |
| 265 | NCF4 |
| 266 | NDRG4 |
| 267 | NDST1-AS1 |
| 268 | NDUFC2 |
| 269 | NECAP1 |
| 270 | NEIL1 |
| 271 | NKD1 |
| 272 | NMNAT1 |
| 273 | NUDT16 |
| 274 | NUS1 |
| 275 | OR13H1 |
| 276 | OR2B6 |
| 277 | OR4K2 |
| 278 | OR51D1 |
| 279 | OR5M11 |
| 280 | OTOF |
| 281 | OTUB2 |
| 282 | P3H2-AS1 |
| 283 | PACERR |
| 284 | PADI4 |
| 285 | PCDHGA12 |
| 286 | PDLIM2 |
| 287 | PGAM4 |
| 288 | PHGR1 |
| 289 | PIGW |
| 290 | PIP5KL1 |
| 291 | PLEC |
| 292 | PMCHL1 |
| 293 | POLH |
| 294 | POU5F1 |
| 295 | PRAMEF12 |
| 296 | PRAMEF2 |
| 297 | PRSS16 |
| 298 | PSG9 |
| 299 | PSMA6 |
| 300 | PTPN12 |
| 301 | RBM15 |
| 302 | RGS1 |
| 303 | RPUSD2 |
| 304 | SALL3 |
| 305 | SCG5 |
| 306 | SELL |
| 307 | SERPINE1 |
| 308 | SEZ6L2 |
| 309 | SHISA2 |
| 310 | SIGLEC14 |
| 311 | SLC25A21-AS1 |
| 312 | SLC25A3P1 |
| 313 | SLC39A1 |
| 314 | SLC5A2 |
| 315 | SMAD5-AS1 |
| 316 | SMARCD2 |
| 317 | SNHG20 |
| 318 | SNORA120 |
| 319 | SNORA70I |
| 320 | SNORD113-5 |
| 321 | SOX4 |
| 322 | SPATA19 |
| 323 | SPON2 |
| 324 | SPSB4 |
| 325 | SSX8 |
| 326 | SUCLG1 |
| 327 | SYNJ2BP |
| 328 | TARP |
| 329 | TAS2R14 |
| 330 | TCEAL1 |
| 331 | TIGIT |
| 332 | TMEM134 |
| 333 | TMEM14A |
| 334 | TMEM45B |
| 335 | TMEM80 |
| 336 | TNK1 |
| 337 | TRAPPC2 |
| 338 | TRIM6 |
| 339 | TRIP13 |
| 340 | TSTD3 |
| 341 | TTPAL |
| 342 | TUBA3FP |
| 343 | TUSC8 |
| 344 | UBE2E1 |
| 345 | UBE2G2 |
| 346 | UBE2V2 |
| 347 | UBXN1 |
| 348 | UG0898H09 |
| 349 | UPK3A |
| 350 | VAMP5 |
| 351 | WDR83 |
| 352 | ZCCHC10 |
| 353 | ZFHX4-AS1 |
| 354 | ZFP57 |
| 355 | ZFP69B |
| 356 | ZNF217 |
| 357 | ZNF319 |
| 358 | ZNF76 |
| 359 | ZNF792 |
| 360 | ZWINT |

### Example 8: Construction of a Classifier for Staging Cervical Cancer Patients Using Erythrocyte Micronuclei DNA

In this example, there were 5 subjects, including 2 patients diagnosed as medium differentiated cervical squamous cell carcinoma by other methods and 3 patients diagnosed as low differentiated and low-medium differentiated cervical squamous cell carcinoma.

The peripheral blood samples from patients with cervical cancer were expressed in the form of “P” plus patient number. For example, “P1” represents a peripheral blood sample from the first cervical cancer patient (“Patient 1”), “P2” represents a peripheral blood sample from the second cervical cancer patient (“Patient 2”), and so on.

The basic information of 5 patients with cervical cancer is shown in Table 5. “Cervical cancer type” refers to the type of cervical cancer diagnosed by other methods.

TABLE 5

| | | HPV | Maximum | | |
|---|---|---|---|---|---|
| Patient | | Detected | diameter of | | Existence of lymph |
| number | Age | value | tumor (cm) | Type of cervical cancer | node metastasis or not |
| 1′ | 65 | 25.55 | 3.3 | Medium differentiated | No |
| | | | | squamous cell | |
| | | | | carcinoma | |
| 2′ | 71 | 1212.8 | 2.6 | Medium differentiated | Yes (without surgery) |
| | | | | squamous cell | |
| | | | | carcinoma | |
| 3′ | 43 | 240.09 | 4.1 | low differentiated | Yes (without surgery) |
| | | | | squamous cell | |
| | | | | carcinoma | |
| 4′* | 37 | No | 5.4 | Low-medium | Yes |
| | | | | differentiated | |
| | | | | squamous cell | |
| | | | | carcinoma | |
| 5′ | 56 | 503.61 | 3.2 | Low-medium | No |
| | | | | differentiated | |
| | | | | squamous cell | |
| | | | | carcinoma | |

*Patient 4 was HPV negative.

Erythrocyte micronuclei DNA and genomic DNA of peripheral blood mononuclear cells of each subject were obtained as described in Examples 1-4, and bioinformatics analysis was carried out as described in Example 5.

Specifically, 2 medium differentiated cervical squamous cell carcinoma samples and 3 low differentiated and low-medium differentiated squamous cell carcinoma samples in primary cervical squamous cell carcinoma samples were selected for the reads counting, and 466 differential genes were screened out by ANOVA test to distinguish the two classes of samples. Then, according to Pearson correlation, the two classes of samples were clustered in unsupervised hierarchy, showing that there were significant differences between the two classes of samples.

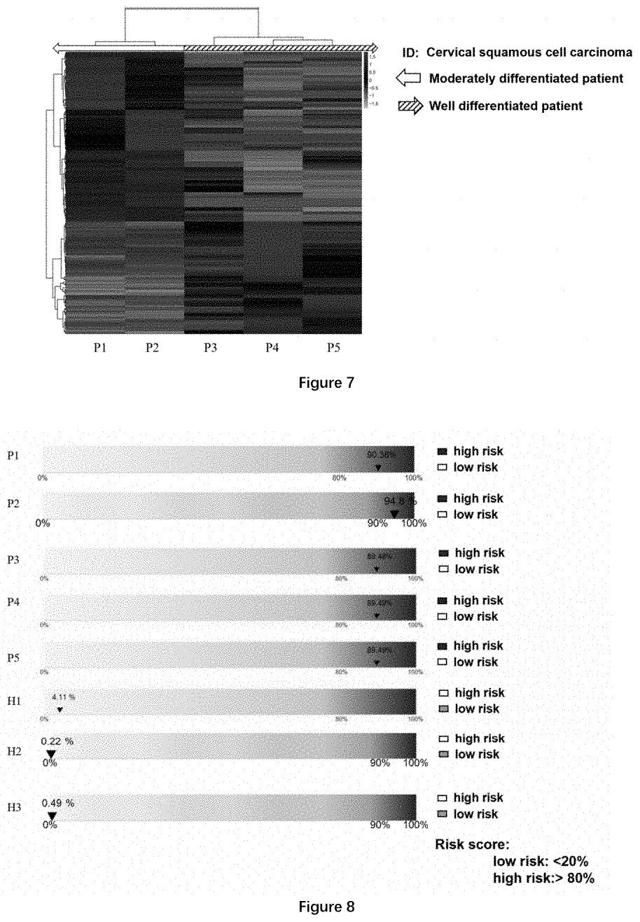

As shown in FIG. 7, 466 differential genes (forming a classifier for distinguishing medium differentiated cervical squamous cell carcinoma from cervical low differentiated and low-medium differentiated squamous cell carcinoma patients) were clustered. In FIG. 7, each row represents a differential gene, and each column represents a patient.

The list of 466 differential genes is shown in Table 6. Each gene corresponds to each row from top to bottom in FIG. 7.

TABLE 6

| | |
|---|---|
| 1 | ACP1 |
| 2 | ADAD1 |
| 3 | AGAP1-IT1 |
| 4 | AKAP7 |
| 5 | AKR7A2 |
| 6 | ANKRD27 |
| 7 | ANXA9 |
| 8 | ARMC12 |
| 9 | ASCL4 |
| 10 | AZIN1 |
| 11 | B2M |
| 12 | BACE1 |
| 13 | BANF2 |
| 14 | C12orf74 |
| 15 | C15orf48 |
| 16 | C15orf59-AS1 |
| 17 | C18orf61 |
| 18 | C19orf66 |
| 19 | C21orf62 |
| 20 | C2orf69 |
| 21 | C2orf81 |
| 22 | C3orf56 |
| 23 | C8orf34-AS1 |
| 24 | C9orf66 |
| 25 | CARD18 |
| 26 | CASC11 |
| 27 | CASP14 |
| 28 | CCR5 |
| 29 | CDIP1 |
| 30 | CDK9 |
| 31 | CENPVL3 |
| 32 | CERNA2 |
| 33 | CFTR-AS1 |
| 34 | CIITA |
| 35 | CLEC2B |
| 36 | CLRN1-AS1 |
| 37 | COA5 |
| 38 | CPHXL |
| 39 | CPNE6 |
| 40 | CPXCR1 |
| 41 | CRYBG3 |
| 42 | CYP4V2 |
| 43 | DBF4B |
| 44 | DBIL5P |
| 45 | DDX39B-AS1 |
| 46 | DPYS |
| 47 | DUSP1 |
| 48 | EBNA1BP2 |
| 49 | EDEM1 |
| 50 | EEF1E1 |
| 51 | ELOA2 |
| 52 | EMC1 |
| 53 | FAM111A-DT |
| 54 | FAM185A |
| 55 | FAM201B |
| 56 | FAM219A |
| 57 | FAM237A |
| 58 | FAM24A |
| 59 | FAR2P2 |
| 60 | FGFBP3 |
| 61 | FLJ36000 |
| 62 | FLJ37201 |
| 63 | FOXP1-AS1 |
| 64 | FTSJ1 |
| 65 | GASAL1 |
| 66 | GKN2 |
| 67 | GNG5 |
| 68 | GOLM1 |
| 69 | GPR55 |
| 70 | GSN-AS1 |
| 71 | HACD4 |
| 72 | HHEX |
| 73 | HIP1 |
| 74 | HIST1H3H |
| 75 | HIST4H4 |
| 76 | HNRNPA1P10 |
| 77 | HSPA1L |
| 78 | IKZF4 |
| 79 | IL10RB-AS1 |
| 80 | IL1R1 |
| 81 | IL26 |
| 82 | INAFM1 |
| 83 | ITPK1 |
| 84 | KAZN |
| 85 | KCNAB1-AS2 |
| 86 | KDM4A-AS1 |
| 87 | KDM8 |
| 88 | KIAA1456 |
| 89 | KLF12 |
| 90 | KLHL15 |
| 91 | KRR1 |
| 92 | KRT77 |
| 93 | KRTAP16-1 |
| 94 | KRTAP29-1 |
| 95 | KRTAP6-1 |
| 96 | LGALS12 |
| 97 | LHFPL6 |
| 98 | LHX4-AS1 |
| 99 | LINC00299 |
| 100 | LINC00317 |
| 101 | LINC00415 |
| 102 | LINC00485 |
| 103 | LINC00563 |
| 104 | LINC00676 |
| 105 | LINC00919 |
| 106 | LINC01114 |
| 107 | LINC01203 |
| 108 | LINC01644 |
| 109 | LINC01876 |
| 110 | LINC02041 |
| 111 | LINC02120 |
| 112 | LINC02210 |
| 113 | LINC02308 |
| 114 | LINC02310 |
| 115 | LOC100128288 |
| 116 | LOC100131496 |
| 117 | LOC100505635 |
| 118 | LOC100505715 |
| 119 | LOC101927402 |
| 120 | LOC101927523 |
| 121 | LOC101927884 |
| 122 | LOC101929718 |
| 123 | LOC102723883 |
| 124 | LOC105370954 |
| 125 | LOC105373064 |
| 126 | LOC105377348 |
| 127 | LOC105378052 |
| 128 | LOC643406 |
| 129 | LOC646548 |
| 130 | LOC727896 |
| 131 | LPCAT3 |
| 132 | LRRC17 |
| 133 | LRRC42 |
| 134 | LY9 |
| 135 | MAGEA11 |
| 136 | MBIP |
| 137 | MCM3 |
| 138 | MED28 |
| 139 | METTL18 |
| 140 | MIR1322 |
| 141 | MIR3663HG |
| 142 | MIR4303 |
| 143 | MIR4699 |
| 144 | MIR570HG |
| 145 | MLIP-AS1 |
| 146 | MORF4L1 |
| 147 | MSANTD2 |
| 148 | MYH2 |
| 149 | MYO9B |
| 150 | MYOZ3 |
| 151 | NAA30 |
| 152 | NDNF |
| 153 | NEU2 |
| 154 | NFYC-AS1 |
| 155 | NHS-AS1 |
| 156 | NKILA |
| 157 | NKX3-1 |
| 158 | NPTN-IT1 |
| 159 | NRG1-IT1 |
| 160 | NRN1 |
| 161 | NTM-IT |
| 162 | OR10Q1 |
| 163 | OR10Z1 |
| 164 | OR13C3 |
| 165 | OR1Q1 |
| 166 | OR2K2 |
| 167 | OR8K3 |
| 168 | OR9G4 |
| 169 | OTUD7B |
| 170 | OTX2 |
| 171 | PCDHA10 |
| 172 | PCDHB14 |
| 173 | PCDHGA1 |
| 174 | PCDHGC4 |
| 175 | PCDHGC5 |
| 176 | PCED1B |
| 177 | PFAS |
| 178 | PICART1 |
| 179 | PLP1 |
| 180 | PNISR |
| 181 | PRKAB2 |
| 182 | PRKAR1A |
| 183 | PROKR2 |
| 184 | PSMD3 |
| 185 | PSTK |
| 186 | PTTG2 |
| 187 | QARS |
| 188 | RAB11FIP4 |
| 189 | RAB31 |
| 190 | RBMXL2 |
| 191 | RBPMS-AS1 |
| 192 | RMDN3 |
| 193 | RNF152 |
| 194 | RPS17 |
| 195 | RRAS |
| 196 | SAMD9L |
| 197 | SCARNA17 |
| 198 | SCARNA6 |
| 199 | SCRT2 |
| 200 | SET |
| 201 | SLC39A2 |
| 202 | SLC3A2 |
| 203 | SLC9A9-AS1 |
| 204 | SMARCA5-AS1 |
| 205 | SNAP25-AS1 |
| 206 | SNORD3K |
| 207 | SNRNP35 |
| 208 | SPATA7 |
| 209 | SPRR4 |
| 210 | SRGAP3-AS2 |
| 211 | SYNPR-AS1 |
| 212 | TBCEL |
| 213 | TENM4 |
| 214 | TEX53 |
| 215 | TIGD2 |
| 216 | TMEM140 |
| 217 | TMEM214 |
| 218 | TRIM3 |
| 219 | TXNDC2 |
| 220 | TXNL1 |
| 221 | TYW5 |
| 222 | UBN1 |
| 223 | UBQLN2 |
| 224 | UCK2 |
| 225 | UPP1 |
| 226 | UQCRQ |
| 227 | VPS35 |
| 228 | VPS37A |
| 229 | VPS45 |
| 230 | VTI1A |
| 231 | XAF1 |
| 232 | XPC |
| 233 | ZBTB33 |
| 234 | ZFP37 |
| 235 | ZMYM3 |
| 236 | ZNF212 |
| 237 | ZNF329 |
| 238 | ZNF492 |
| 239 | ZNF550 |
| 240 | ZNF70 |
| 241 | ZNF740 |
| 242 | ZNF770 |
| 243 | ZNF827 |
| 244 | ZPBP2 |
| 245 | ZSCAN29 |
| 246 | AARD |
| 247 | ABCB8 |
| 248 | ACOXL-AS1 |
| 249 | ACTN1-AS1 |
| 250 | ADAMTS2 |
| 251 | AHSG |
| 252 | AP2A1 |
| 253 | ARHGEF7-AS2 |
| 254 | ARMS2 |
| 255 | BAALC-AS2 |
| 256 | BMP4 |
| 257 | BOLA1 |
| 258 | C11orf42 |
| 259 | C16orf52 |
| 260 | C17orf102 |
| 261 | C17orf62 |
| 262 | C19orf48 |
| 263 | C19orf57 |
| 264 | C5orf66-AS2 |
| 265 | C6orf120 |
| 266 | CAPNS2 |
| 267 | CASC17 |
| 268 | CATIP-AS2 |
| 269 | CCL3L1 |
| 270 | CD274 |
| 271 | CDC42EP2 |
| 272 | CDKN2A-AS1 |
| 273 | CELSR3 |
| 274 | CEP19 |
| 275 | CEP85 |
| 276 | CLLU1 |
| 277 | COX16 |
| 278 | CSE1L |
| 279 | CSPG4 |
| 280 | CSRP2 |
| 281 | CSTA |
| 282 | DCP2 |
| 283 | DCUN1D1 |
| 284 | DDIT4L |
| 285 | DDX21 |
| 286 | DGCR9 |
| 287 | DHH |
| 288 | DOHH |
| 289 | DPYD-AS2 |
| 290 | DSCAM-AS1 |
| 291 | EAF1 |
| 292 | EGFLAM-AS4 |
| 293 | EHBP1L1 |
| 294 | EPOR |
| 295 | FAM189B |
| 296 | FAM32A |
| 297 | FAM43A |
| 298 | FAM90A27P |
| 299 | FBRS |
| 300 | FBXL15 |
| 301 | FLNB-AS1 |
| 302 | FOXD1 |
| 303 | FOXO4 |
| 304 | FPR1 |
| 305 | FPR2 |
| 306 | FRY-AS1 |
| 307 | FSCN3 |
| 308 | GCSAM |
| 309 | GDF6 |
| 310 | GFRA4 |
| 311 | GJB4 |
| 312 | GPR179 |
| 313 | GPRC5C |
| 314 | GPSM3 |
| 315 | GRM7-AS1 |
| 316 | GSG1L |
| 317 | H3F3AP4 |
| 318 | HCG4B |
| 319 | HCP5 |
| 320 | HHATL |
| 321 | HIST1H2AB |
| 322 | HLA-DMB |
| 323 | HLA-F |
| 324 | HLA-H |
| 325 | HNRNPUL2 |
| 326 | HULC |
| 327 | HYAL4 |
| 328 | IER2 |
| 329 | IFIT5 |
| 330 | IGLL1 |
| 331 | ING4 |
| 332 | INTS5 |
| 333 | IQCE |
| 334 | IRX6 |
| 335 | KCNN1 |
| 336 | KCNS2 |
| 337 | KIAA1147 |
| 338 | KLHL11 |
| 339 | KLK1 |
| 340 | KRT7 |
| 341 | LAMTOR5 |
| 342 | LCE1E |
| 343 | LGI4 |
| 344 | LILRB1 |
| 345 | LINC00174 |
| 346 | LINC00459 |
| 347 | LINC00513 |
| 348 | LINC00526 |
| 349 | LINC00547 |
| 350 | LINC00550 |
| 351 | LINC00552 |
| 352 | LINC00578 |
| 353 | LINC01143 |
| 354 | LINC01204 |
| 355 | LINC01502 |
| 356 | LINC01732 |
| 357 | LINC01782 |
| 358 | LINC01833 |
| 359 | LINC01855 |
| 360 | LINC01867 |
| 361 | LINC01884 |
| 362 | LINC01960 |
| 363 | LINC02043 |
| 364 | LINC02441 |
| 365 | LINC02530 |
| 366 | LIPE-AS1 |
| 367 | LMO1 |
| 368 | LNX1-AS2 |
| 369 | LOC100128361 |
| 370 | LOC100130476 |
| 371 | LOC100131303 |
| 372 | LOC100421746 |
| 373 | LOC100506178 |
| 374 | LOC100507507 |
| 375 | LOC101926963 |
| 376 | LOC101927138 |
| 377 | LOC101927143 |
| 378 | LOC101928177 |
| 379 | LOC101928725 |
| 380 | LOC101928791 |
| 381 | LOC101928887 |
| 382 | LOC101929549 |
| 383 | LOC101929721 |
| 384 | LOC105371083 |
| 385 | LOC105373609 |
| 386 | LOC105374952 |
| 387 | LOC105377621 |
| 388 | LOC105378397 |
| 389 | LOC105379393 |
| 390 | LOC107984703 |
| 391 | LOC148413 |
| 392 | LOC257396 |
| 393 | LOC387810 |
| 394 | LOC401320 |
| 395 | LOC644936 |
| 396 | LOC646903 |
| 397 | LOC729224 |
| 398 | LOC729305 |
| 399 | LRG1 |
| 400 | LRP12 |
| 401 | LSR |
| 402 | LUM |
| 403 | LUNAR1 |
| 404 | MAGEB2 |
| 405 | MAGEF1 |
| 406 | MAGIX |
| 407 | MALL |
| 408 | MAPK13 |
| 409 | MAPKAPK5-AS1 |
| 410 | MESP1 |
| 411 | METTL3 |
| 412 | MGC4859 |
| 413 | MIP |
| 414 | MIR1255A |
| 415 | MIR1265 |
| 416 | MIR1291 |
| 417 | MIR17HG |
| 418 | MIR2682 |
| 419 | MIR3145 |
| 420 | MIR31HG |
| 421 | MIR4255 |
| 422 | MIR548U |
| 423 | MIR5692C2 |
| 424 | MIR643 |
| 425 | MIR7-3 |
| 426 | MIR891B |
| 427 | MOB3A |
| 428 | MRAP |
| 429 | MRPL32 |
| 430 | MRPS30-DT |
| 431 | MSGN1 |
| 432 | MUC22 |
| 433 | NDUFAB1 |
| 434 | NELFA |
| 435 | NHLRC1 |
| 436 | OLFML3 |
| 437 | OR12D2 |
| 438 | OR2AK2 |
| 439 | OR2H1 |
| 440 | OR51F1 |
| 441 | OR51I2 |
| 442 | OR5AP2 |
| 443 | OR6C65 |
| 444 | OR6J1 |
| 445 | OR6V1 |
| 446 | OR8K1 |
| 447 | OSCAR |
| 448 | OXA1L |
| 449 | PAX6-AS1 |
| 450 | PCDHA11 |
| 451 | PLAC9 |
| 452 | PLCH1-AS2 |
| 453 | PLVAP |
| 454 | PPP1R3A |
| 455 | PPP1R3D |
| 456 | PRC1-AS1 |
| 457 | PRR15 |
| 458 | PRR23C |
| 459 | PRRT2 |
| 460 | PRSS41 |
| 461 | PRSS57 |
| 462 | PTGS2 |
| 463 | RAB6C-AS1 |
| 464 | RAI2 |
| 465 | RASGRP4 |
| 466 | RBMXL3 |

### Example 9. Classification of Healthy Individuals and Cervical Cancer Patients Using Erythrocyte Micronuclei DNA

Using the classifier (2,306 genes) constructed in Example 6 for clustering healthy individuals and cervical cancer patients, 8 unknown samples from 8 subjects were predicted.

Erythrocyte micronuclei DNA and genomic DNA of peripheral blood mononuclear cells of each subject were obtained as described in Examples 1-4, and bioinformatics analysis was carried out as described in Example 5.

After testing, it was found that 5 of the 8 samples were at high risk of cervical cancer (the risk probabilities were all over 85%), and 3 were at low risk of cervical cancer (the risk probabilities were all less than 5%). Tracing back the sample sources of subjects predicted to be high risk and subjects predicted to be low risk, it was found that 5 samples with high risk of cervical cancer were obtained from patients who were diagnosed as cervical cancer by other diagnostic methods. Three samples with low risk of cervical cancer were obtained from healthy individuals detected by other diagnostic methods.

The result is shown in FIG. 8. In FIGS. 8, P1, P2, P3, P4 and P5 were 5 cervical cancer patients, P3, P4 and P5 were 3 of 9 cervical cancer samples in training set, and P1 and P2 were cervical cancer samples not in model training set; H1, H2 and H3 were all samples of non-cervical cancer healthy individuals.

Therefore, the method and the gene classifier of the present disclosure can effectively distinguish cervical cancer patients from healthy individuals.

### Example 10: Typing of Cervical Cancer Patients Using Erythrocyte Micronuclei DNA

Erythrocyte micronuclei DNA and genomic DNA of peripheral blood mononuclear cells of each subject were obtained as described in Examples 1-4, and bioinformatics analysis was carried out as described in Example 5.

Using the classifier (360 genes) constructed in Example 7 for clustering patients with cervical squamous cell carcinoma and cervical adenocarcinoma, three cervical cancer samples with unknown classification were predicted.

After testing, it was found that two of the three samples were of high risk (the risk probabilities were all over 85%) and one was of low risk (the risk probability was less than 5%). Tracing back the sample sources of subjects with high risk of cervical squamous cell carcinoma and subjects with low risk of cervical squamous cell carcinoma, it was found that two samples with high risk of cervical squamous cell carcinoma were obtained from patients with cervical squamous cell carcinoma detected by other diagnostic methods, and one sample with low risk of cervical squamous cell carcinoma was obtained from healthy individuals as detected by other diagnostic methods.

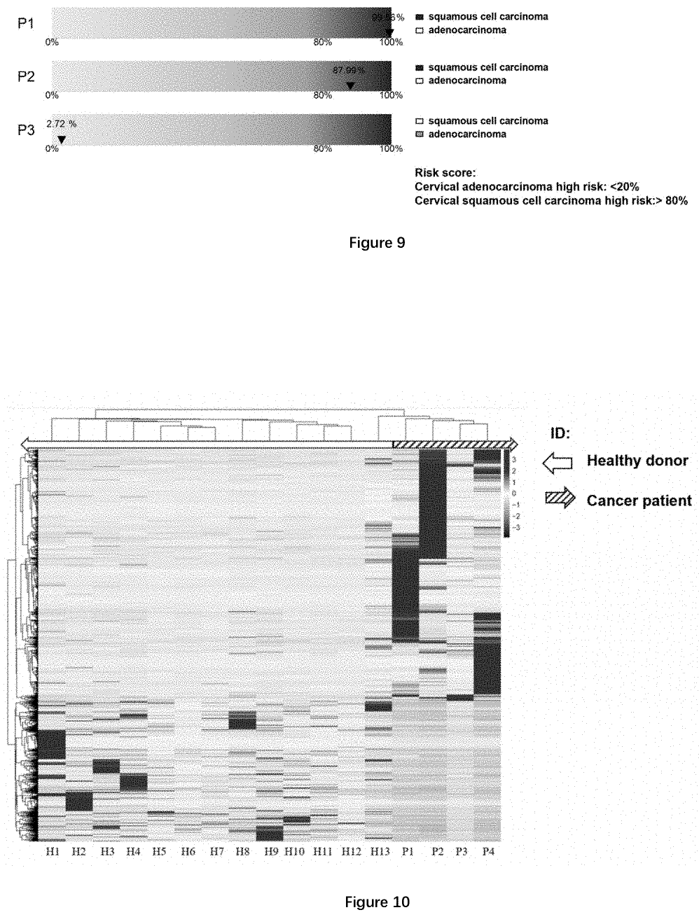

The result is shown in FIG. 9. In FIG. 9, P1 was a patient with cervical adenocarcinoma, and P2 and P3 were patients with cervical squamous cell carcinoma.

Therefore, the method and gene classifier of the present disclosure can effectively classify cervical cancer patients and distinguish cervical squamous cell carcinoma from cervical adenocarcinoma.

### Example 11: Construction of a Classifier for Classifying Healthy Individuals and Colorectal Cancer Patients Using Erythrocyte Micronuclei DNA

In this Example, there were 17 subjects, including:

Experimental group: 4 patients diagnosed as colorectal cancer by other methods

Control group: 13 healthy individuals (non-colorectal cancer individuals).

The peripheral blood samples from patients with colorectal cancer were expressed in the form of “P” plus patient number. For example, “P1” represented a peripheral blood sample from the first colorectal cancer patient (“Patient 1”), “P2” represented a peripheral blood sample from the second colorectal cancer patient (“Patient 2”), and so on. In addition, peripheral blood samples from healthy individuals were expressed in the form of “H” plus individual number. For example, “H1” represents the peripheral blood sample from the first healthy individual, “H2” represents the peripheral blood sample from the second healthy individual, and so on.

The basic information of 4 patients with colorectal cancer is shown in Table 7. Colorectal cancer type, e.g., “adenocarcinoma”, refers to the type of colorectal cancer diagnosed by other methods.

TABLE 7

| Patient | | | Enteroscopy |
|---|---|---|---|
| number | Gender | Complaint | pathology |
| 1 | Female | Increased stool frequency | (sigmoid colon) |
| | | with bloody stool for | low differentiated |
| | | more than 2 months | adenocarcinoma |
| 2 | Female | Black stool for more | Adenocarcinoma |
| | | than 2 months | (65 cm from anus) |
| 3 | Male | Rectal cancer was | Adenocarcinoma |
| | | diagnosed for | (rectum) |
| | | more than 1 month | |
| 4 | Male | Latent pain in the left | (40 cm from anus) villous |
| | | lower abdomen for 1 | adenoma, high-grade |
| | | week | intraepithelial |
| | | | neoplasia, and focal |
| | | | Cancerization cannot |
| | | | be excluded |

Erythrocyte micronuclei DNA and genomic DNA of peripheral blood mononuclear cells of each subject were obtained as described in Examples 1-4, and bioinformatics analysis was carried out as described in Example 5.

Specifically, the reads counts of the gene regions of 4 primary colorectal cancer samples and 13 healthy female samples were selected, and 903 differential genes were screened out by ANOVA test to distinguish the two classes of samples. Then, the unsupervised hierarchical clustering of the two classes of samples was carried out according to Pearson correlation, and it was found that there were significant differences between the two classes of samples.

As shown in FIG. 10, red cell micronuclei DNA from peripheral blood samples of colorectal cancer patients and red cell micronuclei DNA from peripheral blood samples of healthy individuals were clustered to obtain 903 differential genes (forming a classifier for distinguishing healthy individuals from colorectal cancer patients). In FIG. 10, each row represents a differential gene, and each column represents a patient.

The list of 903 differential genes is shown in Table 8. Each gene corresponds to each row from top to bottom in FIG. 10.

TABLE 8

| | |
|---|---|
| 1 | FXYD3 |
| 2 | CD9 |
| 3 | DYNLL1 |
| 4 | CTSA |
| 5 | PDCD6IP |
| 6 | IDH2 |
| 7 | RBMX |
| 8 | CORO1B |
| 9 | SLC12A2 |
| 10 | RCC2 |
| 11 | SETD3 |
| 12 | PAPOLA |
| 13 | PLEC |
| 14 | DRG1 |
| 15 | DTYMK |
| 16 | RBM47 |
| 17 | NFU1 |
| 18 | DNAJB6 |
| 19 | CDC42SE2 |
| 20 | CCND2 |
| 21 | EPHA2 |
| 22 | STT3B |
| 23 | PITRM1 |
| 24 | NARF |
| 25 | WDR45 |
| 26 | EXT2 |
| 27 | LTBP4 |
| 28 | HSPG2 |
| 29 | OTUD5 |
| 30 | COG4 |
| 31 | NEK6 |
| 32 | CCNK |
| 33 | UBE3C |
| 34 | VDR |
| 35 | IGF2R |
| 36 | FOXA3 |
| 37 | SH3BP4 |
| 38 | LAMC1 |
| 39 | SPPL2A |
| 40 | GBP2 |
| 41 | KREMEN1 |
| 42 | PIP5K1B |
| 43 | FAF1 |
| 44 | RAB9A |
| 45 | ALAD |
| 46 | PDE9A |
| 47 | CORO7 |
| 48 | TFE3 |
| 49 | NEDD9 |
| 50 | CCDC107 |
| 51 | TMEM181 |
| 52 | SLC1A4 |
| 53 | HDAC7 |
| 54 | MEMO1 |
| 55 | ATRN |
| 56 | VWF |
| 57 | MXI1 |
| 58 | FOXK2 |
| 59 | MCCC1 |
| 60 | IPO8 |
| 61 | EPS15 |
| 62 | CCDC80 |
| 63 | ACOT1 |
| 64 | ABCB7 |
| 65 | RANBP2 |
| 66 | HS6ST1 |
| 67 | PIP5K1C |
| 68 | PLXND1 |
| 69 | MED27 |
| 70 | SLC25A29 |
| 71 | XIAP |
| 72 | CCDC85C |
| 73 | ATP11A |
| 74 | AGFG1 |
| 75 | BNIP2 |
| 76 | LRRC32 |
| 77 | WDR4 |
| 78 | ISY1 |
| 79 | RBM19 |
| 80 | CYBB |
| 81 | PARVA |
| 82 | UPF2 |
| 83 | RECQL |
| 84 | UBIAD1 |
| 85 | MICALL1 |
| 86 | CRTC3 |
| 87 | PEX1 |
| 88 | PPP1R13B |
| 89 | GBP4 |
| 90 | MCF2L |
| 91 | DCUN1D1 |
| 92 | CBWD6 |
| 93 | IFNAR2 |
| 94 | HAUS2 |
| 95 | IGSF9 |
| 96 | PTPRE |
| 97 | CCDC120 |
| 98 | JMJD1C |
| 99 | RLIM |
| 100 | TRAPPC2 |
| 101 | MSRA |
| 102 | TRAF3IP1 |
| 103 | MZF1 |
| 104 | RAI1 |
| 105 | C11orf80 |
| 106 | SHF |
| 107 | LZTFL1 |
| 108 | ASH1L |
| 109 | PKD1 |
| 110 | NFAT5 |
| 111 | POFUT2 |
| 112 | CD300A |
| 113 | CEMP1 |
| 114 | SDR16C5 |
| 115 | PTPRD |
| 116 | ENOX2 |
| 117 | ZSWIM7 |
| 118 | CABLES1 |
| 119 | ZNF74 |
| 120 | MYPOP |
| 121 | ZNF91 |
| 122 | CREB1 |
| 123 | CHRDL1 |
| 124 | LIMS1 |
| 125 | NOX4 |
| 126 | CELSR1 |
| 127 | SLA |
| 128 | PRKCH |
| 129 | ARID2 |
| 130 | PKD2 |
| 131 | INVS |
| 132 | PASK |
| 133 | MSX1 |
| 134 | FAM129A |
| 135 | TCF4 |
| 136 | ABTB2 |
| 137 | ZAP70 |
| 138 | RALGAPA1 |
| 139 | HIVEP2 |
| 140 | RIC8B |
| 141 | ORAI2 |
| 142 | DUSP28 |
| 143 | CSGALNACT1 |
| 144 | CDADC1 |
| 145 | SEMA3A |
| 146 | ZBP1 |
| 147 | ABI3BP |
| 148 | GAMT |
| 149 | KLHL13 |
| 150 | EME1 |
| 151 | ANO7 |
| 152 | PNMA5 |
| 153 | ZFHX3 |
| 154 | SLC13A3 |
| 155 | SLC16A2 |
| 156 | BDKRB1 |
| 157 | TOR1AIP2 |
| 158 | USP45 |
| 159 | LDLRAD2 |
| 160 | ACTL8 |
| 161 | TNNI3 |
| 162 | SLIT3 |
| 163 | TFAP2A |
| 164 | TNRC6C |
| 165 | SPTLC3 |
| 166 | ITPR1 |
| 167 | ZNF222 |
| 168 | EPHB1 |
| 169 | PRICKLE2 |
| 170 | DMD |
| 171 | RASGEF1A |
| 172 | DTNA |
| 173 | ATAD3C |
| 174 | HHIPL1 |
| 175 | ADAM33 |
| 176 | CHAD |
| 177 | WDR31 |
| 178 | NRXN3 |
| 179 | GPM6A |
| 180 | C22orf23 |
| 181 | TBXA2R |
| 182 | SETBP1 |
| 183 | CREB5 |
| 184 | SIRPB2 |
| 185 | HECW2 |
| 186 | GDPD1 |
| 187 | ADAMTS5 |
| 188 | TIAM1 |
| 189 | PI15 |
| 190 | TCEANC |
| 191 | PDE10A |
| 192 | DYNC1I1 |
| 193 | SNAP25 |
| 194 | FASLG |
| 195 | GRIP1 |
| 196 | MAP3K9 |
| 197 | LSAMP |
| 198 | SOX6 |
| 199 | SCG3 |
| 200 | CDKL3 |
| 201 | CACNA2D1 |
| 202 | CCDC157 |
| 203 | FAM155A |
| 204 | ADAMTS17 |
| 205 | OPRL1 |
| 206 | FUNDC2P2 |
| 207 | ACSM1 |
| 208 | ALPK2 |
| 209 | SEMA5B |
| 210 | PHACTR1 |
| 211 | KRT12 |
| 212 | UNC5A |
| 213 | JAKMIP1 |
| 214 | DGKB |
| 215 | GRIN2B |
| 216 | CCDC17 |
| 217 | C8orf48 |
| 218 | FAM163A |
| 219 | MYLK4 |
| 220 | UGT2B10 |
| 221 | ANKRD53 |
| 222 | SH2D5 |
| 223 | KCNA2 |
| 224 | CFC1B |
| 225 | OPCML |
| 226 | ANKS1B |
| 227 | ABCA4 |
| 228 | RAB3B |
| 229 | ZNF732 |
| 230 | CTNND2 |
| 231 | TTC24 |
| 232 | PPM1E |
| 233 | GPC5 |
| 234 | FBXO15 |
| 235 | AR |
| 236 | LY6G6F |
| 237 | GRIK4 |
| 238 | CTNNA3 |
| 239 | DDX53 |
| 240 | TECTA |
| 241 | CCL16 |
| 242 | TMC3 |
| 243 | EPO |
| 244 | PTPRT |
| 245 | KCTD8 |
| 246 | CDH12 |
| 247 | KCNQ3 |
| 248 | CADM2 |
| 249 | DAZL |
| 250 | GRM1 |
| 251 | SLC12A5 |
| 252 | LRP1B |
| 253 | MED12L |
| 254 | OR14I1 |
| 255 | CDH10 |
| 256 | CNTN5 |
| 257 | TMC1 |
| 258 | TRDN |
| 259 | ZNF716 |
| 260 | MYT1L |
| 261 | GSX1 |
| 262 | CSMD3 |
| 263 | UNC13C |
| 264 | C20orf173 |
| 265 | KCNU1 |
| 266 | DKFZp434L192 |
| 267 | VSX2 |
| 268 | C4orf22 |
| 269 | KCNK16 |
| 270 | FSD2 |
| 271 | CPXCR1 |
| 272 | GRID2 |
| 273 | ATP13A5 |
| 274 | H1FOO |
| 275 | SPINK8 |
| 276 | LEUTX |
| 277 | GFRA4 |
| 278 | IQCJ |
| 279 | KRTAP19-8 |
| 280 | HTN3 |
| 281 | TTC24 |
| 282 | PPP4R3C |
| 283 | SYNDIG1 |
| 284 | TRAPPC2 |
| 285 | OTUD5 |
| 286 | RBMX |
| 287 | PDE10A |
| 288 | LINC00871 |
| 289 | DNAJB6 |
| 290 | LOC101928254 |
| 291 | GMNC |
| 292 | CSGALNACT1 |
| 293 | LINC01581 |
| 294 | AGFG1 |
| 295 | PLS3-AS1 |
| 296 | LINC00972 |
| 297 | FAF1 |
| 298 | PPP4R3C |
| 299 | DMD |
| 300 | DAZL |
| 301 | TNNI3 |
| 302 | DMD |
| 303 | TNNI3 |
| 304 | PIP5K1B |
| 305 | MEMO1 |
| 306 | MZF1 |
| 307 | PPM1E |
| 308 | ADAMTS5 |
| 309 | MED27 |
| 310 | WDR45 |
| 311 | PPP4R3C |
| 312 | MAP3K9 |
| 313 | CDH12 |
| 314 | DYNC1I1 |
| 315 | MAP3K9 |
| 316 | LINC01019 |
| 317 | GBP2 |
| 318 | CADM2 |
| 319 | SLA |
| 320 | SPTLC3 |
| 321 | KCTD8 |
| 322 | SEMA3A |
| 323 | UNC13C |
| 324 | TFE3 |
| 325 | XRCC6P5 |
| 326 | RALGAPA1 |
| 327 | CPXCR1 |
| 328 | ALPK2 |
| 329 | XRCC6P5 |
| 330 | LOC102724908 |
| 331 | CCDC80 |
| 332 | ALPK2 |
| 333 | KCTD8 |
| 334 | DTNA |
| 335 | FAM129A |
| 336 | PDE9A |
| 337 | GRM1 |
| 338 | CYBB |
| 339 | ZNF91 |
| 340 | SYNDIG1 |
| 341 | CPXCR1 |
| 342 | CPXCR1 |
| 343 | PLCB1-IT1 |
| 344 | RNA5-8SN5 |
| 345 | GRM1 |
| 346 | PNMA5 |
| 347 | SYNDIG1 |
| 348 | CADM2 |
| 349 | SYNDIG1 |
| 350 | LINC00548 |
| 351 | LOC101928012 |
| 352 | LINC00972 |
| 353 | KLHL13 |
| 354 | KCTD8 |
| 355 | MIR5011 |
| 356 | EPS15 |
| 357 | GBP4 |
| 358 | LEUTX |
| 359 | CCDC120 |
| 360 | DKFZp434L192 |
| 361 | MAP3K21 |
| 362 | NFU1 |
| 363 | DMD |
| 364 | MAP3K9 |
| 365 | LOC728739 |
| 366 | CPXCR1 |
| 367 | PHACTR1 |
| 368 | PPM1E |
| 369 | LINC00871 |
| 370 | LOC101928012 |
| 371 | PDE10A |
| 372 | SLC16A2 |
| 373 | XRCC6P5 |
| 374 | SDR16C5 |
| 375 | CSGALNACT1 |
| 376 | LINC01317 |
| 377 | GBP4 |
| 378 | RALGAPA1 |
| 379 | GBP2 |
| 380 | LINC00871 |
| 381 | CPXCR1 |
| 382 | ALPK2 |
| 383 | PPM1E |
| 384 | ATP13A5 |
| 385 | CYBB |
| 386 | DMD |
| 387 | GRIP1 |
| 388 | RLIM |
| 389 | LINC01467 |
| 390 | AR |
| 391 | ANKS1B |
| 392 | LINC00871 |
| 393 | SDR16C5 |
| 394 | MIR4675 |
| 395 | ZFPM2-AS1 |
| 396 | LINC00972 |
| 397 | RAB9A |
| 398 | ZFPM2-AS1 |
| 399 | UNC13C |
| 400 | DMD |
| 401 | RALGAPA1 |
| 402 | CBWD6 |
| 403 | ANKS1B |
| 404 | NFU1 |
| 405 | PDE10A |
| 406 | CBWD6 |
| 407 | SYNDIG1 |
| 408 | NFU1 |
| 409 | CSGALNACT1 |
| 410 | PDE10A |
| 411 | NXPE4 |
| 412 | SLC12A2 |
| 413 | CPXCR1 |
| 414 | MAP3K21 |
| 415 | TCEANC |
| 416 | SLC16A2 |
| 417 | MAP3K21 |
| 418 | GBP2 |
| 419 | ZFPM2-AS1 |
| 420 | ABI3BP |
| 421 | ANKS1B |
| 422 | TECTA |
| 423 | ALPK2 |
| 424 | NRXN3 |
| 425 | ABI3BP |
| 426 | JAKMIP1 |
| 427 | ALB |
| 428 | APOA2 |
| 429 | HP |
| 430 | FTL |
| 431 | APOC1 |
| 432 | APOA1 |
| 433 | APOC3 |
| 434 | ORM1 |
| 435 | SERPINA1 |
| 436 | APOE |
| 437 | RBP4 |
| 438 | FGA |
| 439 | AMBP |
| 440 | FGG |
| 441 | VTN |
| 442 | APOC2 |
| 443 | APOH |
| 444 | TF |
| 445 | TTR |
| 446 | B2M |
| 447 | FGB |
| 448 | C3 |
| 449 | SAA1 |
| 450 | FABP1 |
| 451 | AHSG |
| 452 | SERPINA3 |
| 453 | RPLP1 |
| 454 | CYP2E1 |
| 455 | IFITM3 |
| 456 | SERPINC1 |
| 457 | ALDOB |
| 458 | GAPDH |
| 459 | FTH1 |
| 460 | MT2A |
| 461 | RPL8 |
| 462 | HPX |
| 463 | ORM2 |
| 464 | EPHX1 |
| 465 | CLU |
| 466 | SAA2 |
| 467 | ACTB |
| 468 | H19 |
| 469 | GC |
| 470 | CES1 |
| 471 | RPL30 |
| 472 | SERPING1 |
| 473 | RPL37A |
| 474 | PLG |
| 475 | RPS11 |
| 476 | FGL1 |
| 477 | HRG |
| 478 | RPL41 |
| 479 | RPL19 |
| 480 | SEPP1 |
| 481 | CRP |
| 482 | AGT |
| 483 | RARRES2 |
| 484 | RPS12 |
| 485 | KNG1 |
| 486 | RPS16 |
| 487 | RPS6 |
| 488 | CFH |
| 489 | RPS24 |
| 490 | RPL3 |
| 491 | TMSB10 |
| 492 | P4HB |
| 493 | APCS |
| 494 | RPLP2 |
| 495 | RPS20 |
| 496 | UBB |
| 497 | A1BG |
| 498 | MT1G |
| 499 | TMEM176B |
| 500 | CTSD |
| 501 | PEBP1 |
| 502 | RPS8 |
| 503 | RPS9 |
| 504 | RPLP0 |
| 505 | ADH1B |
| 506 | RPS4X |
| 507 | RPL27 |
| 508 | CD74 |
| 509 | RPL11 |
| 510 | RPS19 |
| 511 | DCXR |
| 512 | ACTG1 |
| 513 | RPL23 |
| 514 | RPL35 |
| 515 | AGXT |
| 516 | C1S |
| 517 | AZGP1 |
| 518 | RPL38 |
| 519 | RPL36 |
| 520 | CALR |
| 521 | RPS14 |
| 522 | RPS21 |
| 523 | GLUL |
| 524 | IL32 |
| 525 | ITIH2 |
| 526 | GNB2L1 |
| 527 | F2 |
| 528 | MGST1 |
| 529 | PPIB |
| 530 | ITIH4 |
| 531 | EEF1A1 |
| 532 | GSTA1 |
| 533 | PRAP1 |
| 534 | RPL18 |
| 535 | TMEM176A |
| 536 | HPD |
| 537 | RPS5 |
| 538 | RPL24 |
| 539 | A2M |
| 540 | SOD1 |
| 541 | SOD2 |
| 542 | RPL4 |
| 543 | IGF2 |
| 544 | MYL6 |
| 545 | RPL35A |
| 546 | RPS15A |
| 547 | AKR1C1 |
| 548 | RPS25 |
| 549 | RPL31 |
| 550 | RPL29 |
| 551 | FAU |
| 552 | HSPB1 |
| 553 | MT1X |
| 554 | CYP2A6 |
| 555 | SERPINF2 |
| 556 | RPL5 |
| 557 | MIF |
| 558 | RPL7A |
| 559 | HMGCS2 |
| 560 | CYP3A4 |
| 561 | ATP5E |
| 562 | CFHR1 |
| 563 | ITIH1 |
| 564 | COX7C |
| 565 | REG3A |
| 566 | RPL13A |
| 567 | SERPINF1 |
| 568 | ASS1 |
| 569 | CD63 |
| 570 | C4BPA |
| 571 | RPL26 |
| 572 | C1R |
| 573 | RPS13 |
| 574 | ITIH3 |
| 575 | EEF2 |
| 576 | FN1 |
| 577 | SQSTM1 |
| 578 | ALDH2 |
| 579 | KRT18 |
| 580 | HINT1 |
| 581 | ALDH1A1 |
| 582 | RPL12 |
| 583 | RPL10A |
| 584 | UBC |
| 585 | TMBIM6 |
| 586 | CYB5A |
| 587 | IGFBP1 |
| 588 | PSAP |
| 589 | SPP1 |
| 590 | PLA2G2A |
| 591 | TPT1 |
| 592 | GPX3 |
| 593 | PTMS |
| 594 | IFI27 |
| 595 | UGT2B4 |
| 596 | SPINK1 |
| 597 | ENO1 |
| 598 | IGFBP4 |
| 599 | AKR1B10 |
| 600 | SERPIND1 |
| 601 | RPS10 |
| 602 | ECHS1 |
| 603 | ADH4 |
| 604 | ANG |
| 605 | RPS2 |
| 606 | ASGR2 |
| 607 | HSP90B1 |
| 608 | COX4I1 |
| 609 | CFL1 |
| 610 | RPL14 |
| 611 | EDF1 |
| 612 | COX6B1 |
| 613 | EIF1 |
| 614 | GATM |
| 615 | ADH1A |
| 616 | HULC |
| 617 | HSP90AB1 |
| 618 | LBP |
| 619 | RPL37 |
| 620 | MT1E |
| 621 | SAA4 |
| 622 | RPS7 |
| 623 | IGFBP2 |
| 624 | CFHR2 |
| 625 | KRT8 |
| 626 | RPS27A |
| 627 | APOC4 |
| 628 | PRDX1 |
| 629 | RPS15 |
| 630 | ASGR1 |
| 631 | NNMT |
| 632 | RPL32 |
| 633 | PTMA |
| 634 | AKR1C3 |
| 635 | SCD |
| 636 | APOB |
| 637 | RPS17 |
| 638 | ATP5B |
| 639 | CD81 |
| 640 | EEF1G |
| 641 | NME2 |
| 642 | CPS1 |
| 643 | LASS2 |
| 644 | OAZ1 |
| 645 | PCK1 |
| 646 | HPR |
| 647 | SDS |
| 648 | TM4SF4 |
| 649 | TIMP1 |
| 650 | NDUFB9 |
| 651 | TST |
| 652 | IFITM2 |
| 653 | DHCR24 |
| 654 | SERPINA6 |
| 655 | LYZ |
| 656 | PFN1 |
| 657 | EEF1D |
| 658 | CPB2 |
| 659 | PRDX6 |
| 660 | NDUFA13 |
| 661 | ATP5H |
| 662 | PSMB4 |
| 663 | CP |
| 664 | COX6A1 |
| 665 | ADH1C |
| 666 | ETFB |
| 667 | NUPR1 |
| 668 | ATP5I |
| 669 | HSPA8 |
| 670 | ATF5 |
| 671 | RPS18 |
| 672 | ECH1 |
| 673 | GPX4 |
| 674 | RPL17 |
| 675 | RPL34 |
| 676 | PFDN5 |
| 677 | GPX2 |
| 678 | PABPC1 |
| 679 | TXN |
| 680 | TAGLN2 |
| 681 | LRG1 |
| 682 | MYL12B |
| 683 | RPL6 |
| 684 | HRSP12 |
| 685 | DDT |
| 686 | COX5B |
| 687 | HSD17B6 |
| 688 | COX7B |
| 689 | COX8A |
| 690 | CHI3L1 |
| 691 | GSTK1 |
| 692 | TAT |
| 693 | RPS3 |
| 694 | MAT1A |
| 695 | SULT2A1 |
| 696 | AFP |
| 697 | CTSB |
| 698 | ALDOA |
| 699 | PAH |
| 700 | RPL15 |
| 701 | GAMT |
| 702 | ATOX1 |
| 703 | XBP1 |
| 704 | BTF3 |
| 705 | GRHPR |
| 706 | PRDX5 |
| 707 | CYP2C9 |
| 708 | GABARAP |
| 709 | DBI |
| 710 | C8G |
| 711 | TPI1 |
| 712 | KRTCAP2 |
| 713 | GPX1 |
| 714 | SHFM1 |
| 715 | ADI1 |
| 716 | SDC1 |
| 717 | CYP27A1 |
| 718 | TFR2 |
| 719 | APOA4 |
| 720 | RPL36AL |
| 721 | F12 |
| 722 | HSPD1 |
| 723 | NDUFA1 |
| 724 | RPL10 |
| 725 | MASP2 |
| 726 | LDHA |
| 727 | ARG1 |
| 728 | TUBA1B |
| 729 | ARF1 |
| 730 | TM4SF5 |
| 731 | CD14 |
| 732 | BSG |
| 733 | SSR2 |
| 734 | CYP2D6 |
| 735 | GSTO1 |
| 736 | COX6C |
| 737 | DEFB1 |
| 738 | ATP5J2 |
| 739 | POR |
| 740 | SAT1 |
| 741 | PNKD |
| 742 | GJB1 |
| 743 | GRINA |
| 744 | LGALS4 |
| 745 | SLC25A5 |
| 746 | PSMB3 |
| 747 | HSPA5 |
| 748 | HSD11B1 |
| 749 | HAMP |
| 750 | CHCHD2 |
| 751 | RHOB |
| 752 | GNAS |
| 753 | TFPI |
| 754 | ITM2B |
| 755 | C4BPB |
| 756 | OST4 |
| 757 | SLC38A3 |
| 758 | BAAT |
| 759 | LCN2 |
| 760 | REEP6 |
| 761 | PTGR1 |
| 762 | SERPINA5 |
| 763 | CFB |
| 764 | SEPHS2 |
| 765 | SLC25A3 |
| 766 | GPC3 |
| 767 | UBL5 |
| 768 | PTGDS |
| 769 | PGRMC1 |
| 770 | CTSZ |
| 771 | ACADVL |
| 772 | CYP2C8 |
| 773 | EIF5A |
| 774 | EEF1B2 |
| 775 | FBP1 |
| 776 | AKR1C4 |
| 777 | PARK7 |
| 778 | EFNA1 |
| 779 | CALM2 |
| 780 | FDPS |
| 781 | SEC61B |
| 782 | LGALS1 |
| 783 | MPST |
| 784 | RPSAP58 |
| 785 | SLPI |
| 786 | INSIG1 |
| 787 | SLC27A5 |
| 788 | NDUFB7 |
| 789 | IGFBP7 |
| 790 | PRDX2 |
| 791 | TCEB2 |
| 792 | MYL12A |
| 793 | ATP5A1 |
| 794 | NPM1 |
| 795 | ALDH1L1 |
| 796 | LGALS3BP |
| 797 | HSPE1 |
| 798 | RPSA |
| 799 | PSME1 |
| 800 | PCBP1 |
| 801 | ATP5G2 |
| 802 | EBP |
| 803 | ANXA2 |
| 804 | HPN |
| 805 | PSME2 |
| 806 | PCK2 |
| 807 | GUK1 |
| 808 | HSP90AA1 |
| 809 | PLIN2 |
| 810 | PSMD4 |
| 811 | CYP4A11 |
| 812 | TGM2 |
| 813 | NDUFS5 |
| 814 | PDIA3 |
| 815 | BLVRB |
| 816 | GSTA2 |
| 817 | ATP5C1 |
| 818 | ACAA2 |
| 819 | CFI |
| 820 | IFI6 |
| 821 | LMAN2 |
| 822 | TMED9 |
| 823 | PON1 |
| 824 | NUCB1 |
| 825 | PHYH |
| 826 | ADAM6 |
| 827 | FMO3 |
| 828 | ADH6 |
| 829 | ERBB3 |
| 830 | UGT2B7 |
| 831 | ATPIF1 |
| 832 | HIST1H1C |
| 833 | BCAP31 |
| 834 | SSR4 |
| 835 | HGD |
| 836 | UQCRQ |
| 837 | AADAC |
| 838 | OS9 |
| 839 | NDUFB2 |
| 840 | ATP5O |
| 841 | IFI30 |
| 842 | EIF4A1 |
| 843 | DAD1 |
| 844 | TALDO1 |
| 845 | CHCHD10 |
| 846 | ANGPTL4 |
| 847 | EIF3K |
| 848 | FIS1 |
| 849 | UQCRC1 |
| 850 | BST2 |
| 851 | PROC |
| 852 | RPL23A |
| 853 | DECR1 |
| 854 | CAT |
| 855 | UGT1A6 |
| 856 | ATP6V0E1 |
| 857 | COL18A1 |
| 858 | CANX |
| 859 | ANPEP |
| 860 | C1orf43 |
| 861 | DYNLL1 |
| 862 | YBX1 |
| 863 | AOX1 |
| 864 | SCP2 |
| 865 | PDIA6 |
| 866 | BHMT |
| 867 | CES2 |
| 868 | PSMB1 |
| 869 | AQP9 |
| 870 | HDGF |
| 871 | ACSL1 |
| 872 | CYC1 |
| 873 | FTCD |
| 874 | EIF6 |
| 875 | PCBD1 |
| 876 | NDUFS6 |
| 877 | NACA |
| 878 | STARD10 |
| 879 | RPN1 |
| 880 | LAMP1 |
| 881 | BRI3 |
| 882 | C11orf10 |
| 883 | RHOA |
| 884 | CD151 |
| 885 | TMEM59 |
| 886 | RNASE4 |
| 887 | CCT3 |
| 888 | NCRNA00188 |
| 889 | HLA-B |
| 890 | ACAA1 |
| 891 | CAPNS1 |
| 892 | APLP2 |
| 893 | SDC4 |
| 894 | HIST1H2AC |
| 895 | RPS29 |
| 896 | ATP5D |
| 897 | SELENBP1 |
| 898 | RPN2 |
| 899 | GLUD1 |
| 900 | PSMA7 |
| 901 | ST6GAL1 |
| 902 | SNRPD2 |
| 903 | MLF2 |

### Example 12: Construction of a Classifier for the Typing of Colorectal Cancer Patients Using Erythrocyte Micronuclei DNA

In this Example, there were 10 patients with colorectal cancer, including 5 patients diagnosed with colon cancer and 5 patients diagnosed with rectal cancer by other methods.

The peripheral blood samples from the above patients are expressed in the form of “P” plus patient number. For example, “P1” represents a peripheral blood sample from the first colorectal cancer patient (“Patient 1”), “P2” represents a peripheral blood sample from the second colorectal cancer patient (“Patient 2”), and so on.

The basic information of 10 colorectal cancer patients is shown in Table 9. Colorectal cancer type, e.g., “adenocarcinoma”, refers to the type of colorectal cancer diagnosed by other methods.

TABLE 9

| Patient | | | |
|---|---|---|---|
| number | Gender | Complaint | Enteroscopy pathology |
| 1 | Male | Rectal cancer was | Adenocarcinoma (rectum) |
| | | diagnosed for more | |
| | | than 1 month | |
| 2 | Female | The stool habit | (rectal) villous adenoma with low-grade |
| | | changed for more | intraepithelial neoplasia, local high-grade |
| | | than half a year | intraepithelial neoplasia, the lesion was confined |
| | | | to the lamina propria, and no submucosal |
| | | | infiltration was found. It was recommended to |
| | | | biopsy again to exclude Cancerization. |
| 3 | Male | Changes in stool | Medium differentiated adenocarcinoma (rectum) |
| | | characteristics with | |
| | | hematochezia for more | |
| | | than 3 months | |
| 4 | Female | Rectal cancer was | Medium differentiated adenocarcinoma (rectum) |
| | | diagnosed for | |
| | | more than 3 months | |
| 5 | Male | CEA gradually | Adenocarcinoma (rectum) |
| | | increased for | |
| | | more than 9 months | |
| 6 | Male | After more than 1 | Medium differentiated adenocarcinoma |
| | | month of hematochezia, | (sigmoid colon) |
| | | colon cancer was | |
| | | diagnosed for 4 days. | |
| 7 | Male | Blood in stool for 1 | (sigmoid colon) The broken mucosa showed |
| | | week | high-grade intraepithelial neoplasia. |
| | | | Cancerization cannot be excluded. |
| 8 | Female | Repeated abdominal | Adenocarcinoma of colon |
| | | pain for more than 2 | |
| | | months | |
| 9 | Female | Diarrhea and | (colon biopsy) low differentiated |
| | | hematochezia for three | adenocarcinoma |
| | | months, and fatigue for | |
| | | one month | |
| 10 | Female | Colonic tumor was | Considered as adenocarcinoma (liver curvature |
| | | found for 1 week | of colon). |

Erythrocyte micronuclei DNA and genomic DNA of peripheral blood mononuclear cells of each subject were obtained as described in Examples 1-4, and bioinformatics analysis was carried out as described in Example 5.

Specifically, 97 different genes were screened out by ANOVA test from the reads count of gene regions of 5 colon cancer samples and 5 rectal cancer samples, and then unsupervised hierarchical clustering of the two types of samples according to Pearson correlation showed that there were significant differences between the two classes of samples.

As shown in FIG. 11, a total of 97 genes from colon cancer and rectal cancer samples were clustered. Each row represents a differential gene, and each column represents a patient.

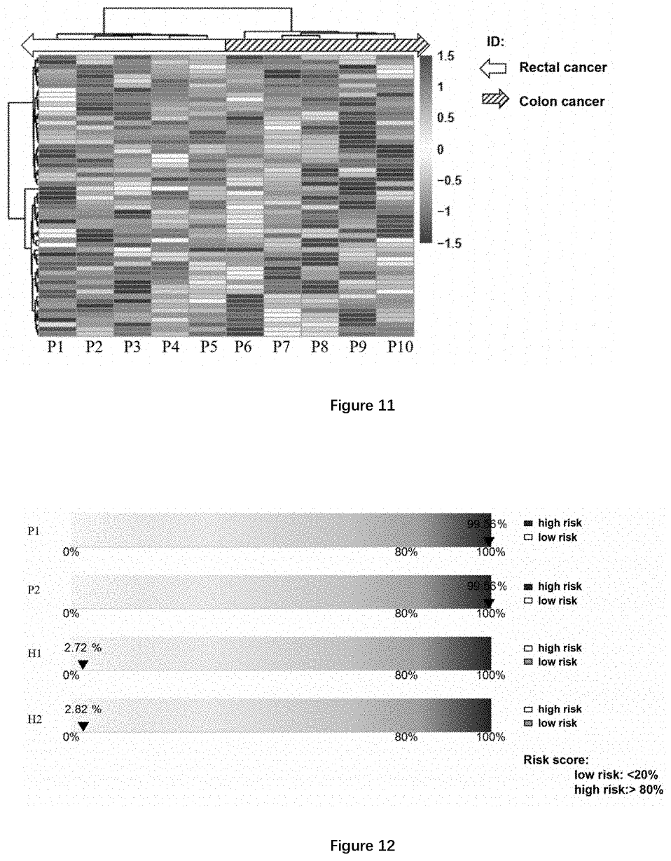

The list of 97 differential genes is shown in Table 10. Each gene corresponds to each row from top to bottom in FIG. 12.

TABLE 10

| | |
|---|---|
| 1 | FXYD3 |
| 2 | CD9 |
| 3 | DYNLL1 |
| 4 | CTSA |
| 5 | CCDC107 |
| 6 | TMEM181 |
| 7 | SLC1A4 |
| 8 | HDAC7 |
| 9 | MEMO1 |
| 10 | ATRN |
| 11 | VWF |
| 12 | MXI1 |
| 13 | FOXK2 |
| 14 | MCCC1 |
| 15 | PAPOLA |
| 16 | PLEC |
| 17 | DRG1 |
| 18 | DTYMK |
| 19 | RBM47 |
| 20 | NFU1 |
| 21 | DNAJB6 |
| 22 | CDC42SE2 |
| 23 | CCND2 |
| 24 | EPHA2 |
| 25 | STT3B |
| 26 | PITRM1 |
| 27 | LTBP4 |
| 28 | HSPG2 |
| 29 | OTUD5 |
| 30 | COG4 |
| 31 | NEK6 |
| 32 | CCNK |
| 33 | UBE3C |
| 34 | VDR |
| 35 | IGF2R |
| 36 | FOXA3 |
| 37 | SH3BP4 |
| 38 | LAMC1 |
| 39 | SPPL2A |
| 40 | GBP2 |
| 41 | KREMEN1 |
| 42 | PIP5K1B |
| 43 | FAF1 |
| 44 | RAB9A |
| 45 | ALAD |
| 46 | PDE9A |
| 47 | CORO7 |
| 48 | TFE3 |
| 49 | NEDD9 |
| 50 | PDCD6IP |
| 51 | IDH2 |
| 52 | RBMX |
| 53 | CORO1B |
| 54 | SLC12A2 |
| 55 | RCC2 |
| 56 | SETD3 |
| 57 | NARF |
| 58 | WDR45 |
| 59 | EXT2 |
| 60 | IPO8 |
| 61 | EPS15 |
| 62 | CCDC80 |
| 63 | ACOT1 |
| 64 | ABCB7 |
| 65 | RANBP2 |
| 66 | HS6ST1 |
| 67 | PIP5K1C |
| 68 | PLXND1 |
| 69 | MED27 |
| 70 | SLC25A29 |
| 71 | XIAP |
| 72 | CCDC85C |
| 73 | ATP11A |
| 74 | AGFG1 |
| 75 | BNIP2 |
| 76 | LRRC32 |
| 77 | WDR4 |
| 78 | ISY1 |
| 79 | RBM19 |
| 80 | CYBB |
| 81 | PARVA |
| 82 | UPF2 |
| 83 | RECQL |
| 84 | UBIAD1 |
| 85 | MICALL1 |
| 86 | CREB1 |
| 87 | CHRDL1 |
| 88 | LIMS1 |
| 89 | NOX4 |
| 90 | CELSR1 |
| 91 | SLA |
| 92 | PRKCH |
| 93 | ARID2 |
| 94 | PKD2 |
| 95 | INVS |
| 96 | PASK |
| 97 | MSX1 |

### Example 13. Classification of Healthy Individuals and Colorectal Cancer Patients Using Erythrocyte Micronuclei DNA

Using the classifier (903 genes) constructed in Example 11 for clustering healthy individuals and colorectal cancer patients, four unknown samples from four subjects were predicted.

Erythrocyte micronuclei DNA and genomic DNA of peripheral blood mononuclear cells of each subject were obtained as described in Examples 1-4, and bioinformatics analysis was carried out as described in Example 5.

After testing, it was found that two of the four samples were at high risk of colorectal cancer (the risk probabilities were all over 90%) and two were at low risk of colorectal cancer (the risk probabilities were all less than 5%). Tracing back the sample sources of subjects predicted to be high risk and subjects predicted to be low risk, it was found that the two samples with high risk of colorectal cancer were obtained from patients who were diagnosed as colorectal cancer by other diagnostic methods, and the two samples with low risk of colorectal cancer were obtained from healthy individuals detected by other diagnostic methods.

The result is shown in FIG. 12. In FIGS. 12, P1 and P2 were two patients with colorectal cancer, and H1 and H2 were samples of non-colorectal cancer healthy individuals.

Therefore, the method and gene classifier of the present disclosure can effectively distinguish colorectal cancer patients from healthy individuals.

### Example 14. Typing of Colorectal Cancer Patients Using Erythrocyte Micronuclei DNA

Erythrocyte micronuclei DNA and genomic DNA of peripheral blood mononuclear cells of each subject were obtained as described in Examples 1-4, and bioinformatics analysis was carried out as described in Example 5.

Using the classifier (97 genes) constructed in Example 12 for clustering colon cancer and rectal cancer patients, four colorectal cancer samples with unknown classification were predicted.

After testing, it was found that two of the four samples were at high risk of colon cancer (the risk probabilities were all over 85%) and two were at low risk of colon cancer (the risk probabilities were all less than 5%). Tracing back the sample sources of subjects with high risk of colon cancer and subjects with low risk of colon cancer, it was found that the two samples with high risk of colon cancer were obtained from patients with colon cancer detected by other diagnostic methods, and the two samples with low risk of colon cancer came from subjects who were diagnosed as rectal cancer by other diagnostic methods.

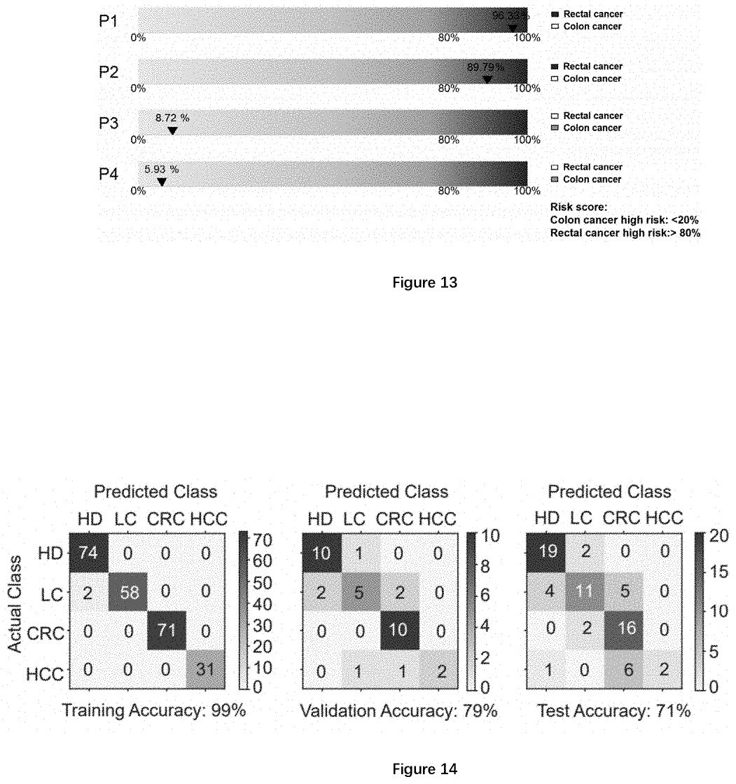

The result is shown in FIG. 13. In FIGS. 13, P1 and P2 are colon cancer patients, and P2 and P3 are rectal cancer patients.

Therefore, the method and gene classifier of the present disclosure can effectively classify colorectal cancer patients and distinguish colon cancer from rectal cancer.

### Example 15. Discriminative Performance of the rbcDNA Signature in Cancer Patients

We randomly assigned HD and cancer samples to a training set (70%, n=236) for model development, a validation set (10%, n=34) for hyper-parameter selection and a test set (20%, n=68) for model validation. Our results showed that 91% (95% confidence interval 84-100%) of cancer patients including 85% LC, 100% CRC and 90% HCC were detected with 99% specificity. This includes 86% patients with stage I, 92% of patients with stage II and 100% of patients with stage III cancers (Table 14). This data suggests the presence of specific rbcDNA signatures that can differentiate between healthy donors and cancer patients. We next tested the efficacy of rbcDNA in differentiating among specific cancer types. rbcDNA signatures exhibit high discriminatory performance in pairwise comparisons of healthy and cancer groups, our results showed that 90% (95% confidence interval 68-100%) of HCC patients, 100% (95% confidence interval 100-100%) of CRC patients and 85% (95% confidence interval 70-100%) of LC patients, all were detected with 95% specificity (Table 15). Moreover, pairwise and multiclass tests showed overall high accuracy in detecting specific cancers indicating significant discriminative power of rbcDNA profiles (FIG. 14).

The result is shown in Table 14, the list of differential rbcDNA signature is shown in Table 16.

The result is shown in Table 15, the list of differential rbcDNA signature is shown in Table 17 for HD vs. LC, Table 18 for HD vs. CRC, Table 19 for HD vs. HCC.

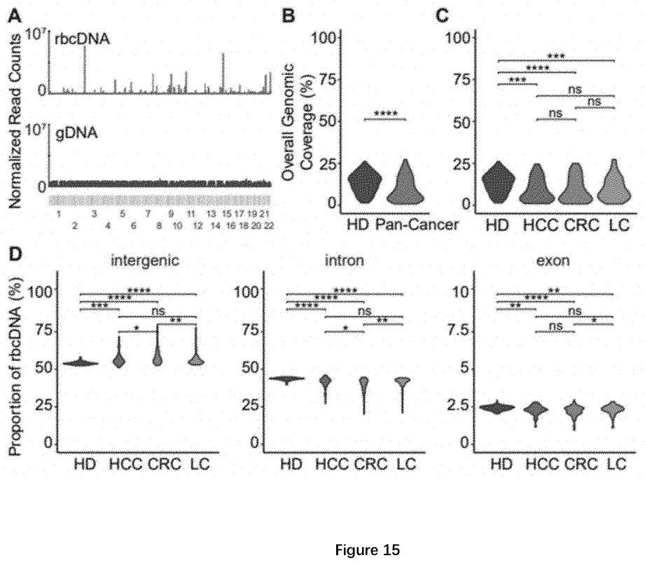

The result is shown in FIG. 15, the list of differential rbcDNA signature is shown in Table 20.

TABLE 14

| Pan-Cancer analysed | | | | 99% specificity | | |
|---|---|---|---|---|---|---|
| | | Test | | Individual | Sensitivity | 95% CI |
| | | (Total) | | detected | (%) | (%) |
| HD | | 21 | (106) | 0 | | |
| Cancer | | 47 | (232) | 43 | 91 | 84-100 |
| Type | HCC | 9 | (44) | 8 | 90 | 68-100 |
| | CRC | 18 | (99) | 18 | 100 | 100-100 |
| | LC | 20 | (89) | 17 | 85 | 69-100 |
| Stage | I | 22 | (106) | 19 | 86 | 72-100 |
| | II | 12 | (61) | 11 | 92 | 76-100 |
| | III | 13 | (65) | 13 | 100 | 100-100 |

TABLE 15

95% specificity

| | Test | Individual | | |
|---|---|---|---|---|
| Cancer Type | (Total) | detected | Sensitivity | 95% CI |
| HCC | 9 | 8 | 90 | 68-100 |
| CRC | 18 | 18 | 100 | 100-100 |
| LC | 20 | 17 | 85 | 70-100 |

Table 14 shows accuracy ratios of pan-cancer deep neural network classification in the test set for each cancer type, including corresponding sensitivity with 99% specificity (CI, Confidence Interval).

Table 15 shows accuracy ratios of each cancer type deep neural network classification in the test set for each cancer type, including corresponding sensitivity with 95% specificity (CI, Confidence Interval).

TABLE 16

| | |
|---|---|
| 1 | chr1: 500000-1000000, chr1: 1500000-2000000, chr1: 2500000-3000000, chr1: 8000000-8500000, |
| | chr1: 8500000-9000000, chr1: 9500000-10000000, chr1: 10000000-10500000, chr1: 11500000- |
| | 12000000, chr1: 12000000-12500000, chr1: 13000000-13500000, chr1: 15500000-16000000, |
| | chr1: 16000000-16500000, chr1: 16500000-17000000, chr1: 19000000-19500000, chr1: 19500000- |
| | 20000000, chr1: 20000000-20500000, chr1: 20500000-21000000, chr1: 21000000-21500000, |
| | chr1: 23000000-23500000, chr1: 24500000-25000000, chr1: 25500000-26000000, chr1: 26000000- |
| | 26500000, chr1: 26500000-27000000, chr1: 27000000-27500000, chr1: 27500000-28000000, |
| | chr1: 28000000-28500000, chr1: 28500000-29000000, chr1: 30500000-31000000, chr1: 31500000- |
| | 32000000, chr1: 32000000-32500000, chr1: 32500000-33000000, chr1: 33000000-33500000, |
| | chr1: 33500000-34000000, chr1: 35500000-36000000, chr1: 36000000-36500000, chr1: 36500000- |
| | 37000000, chr1: 37500000-38000000, chr1: 38500000-39000000, chr1: 39000000-39500000, |
| | chr1: 40000000-40500000, chr1: 40500000-41000000, chr1: 42000000-42500000, chr1: 44500000- |
| | 45000000, chr1: 45500000-46000000, chr1: 51500000-52000000, chr1: 52500000-53000000 |
| | chr1: 53500000-54000000, chr1: 54000000-54500000, chr1: 58500000-59000000, chr1: 59000000- |
| | 59500000, chr1: 61000000-61500000, chr1: 63500000-64000000, chr1: 64500000-65000000, |
| | chr1: 65000000-65500000, chr1: 66000000-66500000, chr1: 77000000-77500000, chr1: 77500000- |
| | 78000000, chr1: 83000000-83500000, chr1: 88500000-89000000, chr1: 91500000-92000000, |
| | chr1: 94500000-95000000, chr1: 98500000-99000000, chr1: 101000000-101500000, |
| | chr1: 107500000-108000000, chr1: 108500000-109000000, chr1: 109000000-109500000, |
| | chr1: 109500000-110000000, chr1: 110500000-111000000, chr1: 111500000-112000000, |
| | chr1: 112500000-113000000, chr1: 114000000-114500000, chr1: 116000000-116500000, |
| | chr1: 116500000-117000000, chr1: 120000000-120500000, chr1: 120500000-121000000, |
| | chr1: 121000000-121500000, chr1: 125000000-125500000, chr1: 143000000-143500000, |
| | chr1: 143500000-144000000, chr1: 144000000-144500000, chr1: 144500000-145000000, |
| | chr1: 145000000-145500000, chr1: 146000000-146500000, chr1: 146500000-147000000, |
| | chr1: 147500000-148000000, chr1: 148000000-148500000, chr1: 148500000-149000000, |
| | chr1: 149000000-149500000, chr1: 149500000-150000000, chr1: 150000000-150500000, |
| | chr1: 150500000-151000000, chr1: 151000000-151500000, chr1: 151500000-152000000, |
| | chr1: 153500000-154000000, chr1: 154000000-154500000, chr1: 154500000-155000000, |
| | chr1: 155000000-155500000, chr1: 155500000-156000000, chr1: 156000000-156500000, |
| | chr1: 156500000-157000000, chr1: 158000000-158500000, chr1: 159000000-159500000, |
| | chr1: 160000000-160500000, chr1: 160500000-161000000, chr1: 161000000-161500000, |
| | chr1: 162000000-162500000, chr1: 163500000-164000000, chr1: 165500000-166000000, |
| | chr1: 167000000-167500000, chr1: 169500000-170000000, chr1: 171000000-171500000, |
| | chr1: 171500000-172000000, chr1: 172500000-173000000, chr1: 173500000-174000000, |
| | chr1: 177500000-178000000, chr1: 181000000-181500000, chr1: 182000000-182500000, |
| | chr1: 182500000-183000000, chr1: 183000000-183500000, chr1: 193000000-193500000, |
| | chr1: 197000000-197500000, chr1: 198500000-199000000, chr1: 199000000-199500000, |
| | chr1: 200000000-200500000, chr1: 200500000-201000000, chr1: 202500000-203000000, |
| | chr1: 203000000-203500000, chr1: 203500000-204000000, chr1: 204000000-204500000, |
| | chr1: 205000000-205500000, chr1: 206500000-207000000, chr1: 208000000-208500000, |
| | chr1: 211500000-212000000, chr1: 212500000-213000000, chr1: 214000000-214500000, |
| | chr1: 221500000-222000000, chr1: 222500000-223000000, chr1: 225500000-226000000, |
| | chr1: 226000000-226500000, chr1: 230500000-231000000, chr1: 235000000-235500000, |
| | chr1: 235500000-236000000, chr1: 242000000-242500000, chr1: 242500000-243000000, |
| | chr1: 243500000-244000000, chr1: 246500000-247000000, chr1: 247000000-247500000 |
| 2 | chr2: 24500000-25000000, chr2: 25000000-25500000, chr2: 25500000-26000000, chr2: 26000000- |
| | 26500000, chr2: 27000000-27500000, chr2: 27500000-28000000, chr2: 28500000-29000000, |
| | chr2: 36500000-37000000, chr2: 37000000-37500000, chr2: 37500000-38000000, chr2: 38500000- |
| | 39000000, chr2: 39000000-39500000, chr2: 42000000-42500000, chr2: 45500000-46000000, |
| | chr2: 48000000-48500000, chr2: 60500000-61000000, chr2: 61000000-61500000, chr2: 61500000- |
| | 62000000, chr2: 64000000-64500000, chr2: 64500000-65000000, chr2: 65000000-65500000, |
| | chr2: 68000000-68500000, chr2: 69500000-70000000, chr2: 70000000-70500000, chr2: 70500000- |
| | 71000000, chr2: 71000000-71500000, chr2: 72500000-73000000, chr2: 73500000-74000000, |
| | chr2: 74500000-75000000, chr2: 76000000-76500000, chr2: 79000000-79500000, chr2: 84500000- |
| | 85000000, chr2: 87000000-87500000, chr2: 87500000-88000000, chr2: 89000000-89500000, |
| | chr2: 89500000-90000000, chr2: 90000000-90500000, chr2: 91000000-91500000, chr2: 91500000- |
| | 92000000, chr2: 92000000-92500000, chr2: 94500000-95000000, chr2: 95500000-96000000, |
| | chr2: 96000000-96500000, chr2: 97000000-97500000, chr2: 102000000-102500000, |
| | chr2: 107500000-108000000, chr2: 109500000-110000000, chr2: 110000000-110500000, |
| | chr2: 112500000-113000000, chr2: 113500000-114000000, chr2: 116000000-116500000, |
| | chr2: 119500000-120000000, chr2: 122000000-122500000, chr2: 126500000-127000000, |
| | chr2: 129000000-129500000, chr2: 130000000-130500000, chr2: 131500000-132000000, |
| | chr2: 132000000-132500000, chr2: 132500000-133000000, chr2: 135500000-136000000, |
| | chr2: 136000000-136500000, chr2: 143000000-143500000, chr2: 144000000-144500000, |
| | chr2: 144500000-145000000, chr2: 148500000-149000000, chr2: 150000000-150500000, |
| | chr2: 156000000-156500000, chr2: 158000000-158500000, chr2: 162000000-162500000, |
| | chr2: 165500000-166000000, chr2: 168000000-168500000, chr2: 168500000-169000000, |
| | chr2: 171500000-172000000, chr2: 174500000-175000000, chr2: 177000000-177500000, |
| | chr2: 178000000-178500000, chr2: 181000000-181500000, chr2: 181500000-182000000, |
| | chr2: 190000000-190500000, chr2: 190500000-191000000, chr2: 191000000-191500000, |
| | chr2: 196000000-196500000, chr2: 197000000-197500000, chr2: 200500000-201000000, |
| | chr2: 201000000-201500000, chr2: 201500000-202000000, chr2: 203000000-203500000, |
| | chr2: 207000000-207500000, chr2: 207500000-208000000, chr2: 208500000-209000000, |
| | chr2: 210000000-210500000, chr2: 216000000-216500000, chr2: 217500000-218000000, |
| | chr2: 218000000-218500000, chr2: 218500000-219000000, chr2: 219000000-219500000, |
| | chr2: 224500000-225000000, chr2: 225000000-225500000, chr2: 228000000-228500000, |
| | chr2: 230000000-230500000, chr2: 230500000-231000000, chr2: 235500000-236000000, |
| | chr2: 238500000-239000000, chr2: 240500000-241000000 |
| 3 | chr3: 500000-1000000, chr3: 9000000-9500000, chr3: 9500000-10000000, chr3: 10000000- |
| | 10500000, chr3: 12500000-13000000, chr3: 13500000-14000000, chr3: 14000000-14500000, |
| | chr3: 17000000-17500000, chr3: 17500000-18000000, chr3: 23500000-24000000, chr3: 30500000- |
| | 31000000, chr3: 31500000-32000000, chr3: 32000000-32500000, chr3: 32500000-33000000, |
| | chr3: 36000000-36500000, chr3: 39000000-39500000, chr3: 43500000-44000000, chr3: 47000000- |
| | 47500000, chr3: 47500000-48000000, chr3: 48500000-49000000, chr3: 49000000-49500000, |
| | chr3: 49500000-50000000, chr3: 51500000-52000000, chr3: 52000000-52500000, chr3: 56500000- |
| | 57000000, chr3: 57500000-58000000, chr3: 59000000-59500000, chr3: 64500000-65000000, |
| | chr3: 66000000-66500000, chr3: 69000000-69500000, chr3: 70000000-70500000, chr3: 71000000- |
| | 71500000, chr3: 72000000-72500000, chr3: 75000000-75500000, chr3: 75500000-76000000, |
| | chr3: 76500000-77000000, chr3: 88000000-88500000, chr3: 91000000-91500000, chr3: 101500000- |
| | 102000000, chr3: 107500000-108000000, chr3: 119500000-120000000, chr3: 122000000- |
| | 122500000, chr3: 127000000-127500000, chr3: 129500000-130000000, chr3: 135500000- |
| | 136000000, chr3: 136500000-137000000, chr3: 141500000-142000000, chr3: 143000000- |
| | 143500000, chr3: 143500000-144000000, chr3: 150000000-150500000, chr3: 151000000- |
| | 151500000, chr3: 156500000-157000000, chr3: 157000000-157500000, chr3: 161000000- |
| | 161500000, chr3: 169500000-170000000, chr3: 170000000-170500000, chr3: 171000000 |
| | 171500000, chr3: 172000000-172500000, chr3: 179500000-180000000, chr3: 184000000- |
| | 184500000, chr3: 184500000-185000000, chr3: 185500000-186000000, chr3: 186000000- |
| | 186500000, chr3: 186500000-187000000, chr3: 188000000-188500000, chr3: 189000000- |
| | 189500000, chr3: 194500000-195000000, chr3: 198000000-198295559 |
| 4 | chr4: 2500000-3000000, chr4: 8500000-9000000, chr4: 9000000-9500000, chr4: 15000000- |
| | 15500000, chr4: 21000000-21500000, chr4: 26000000-26500000, chr4: 36000000-36500000, |
| | chr4: 37500000-38000000, chr4: 38000000-38500000, chr4: 38500000-39000000, chr4: 39500000- |
| | 40000000, chr4: 40000000-40500000, chr4: 40500000-41000000, chr4: 45000000-45500000, |
| | chr4: 48500000-49000000, chr4: 49000000-49500000, chr4: 49500000-50000000, chr4: 61500000- |
| | 62000000, chr4: 70500000-71000000, chr4: 77500000-78000000, chr4: 78500000-79000000, |
| | chr4: 80000000-80500000, chr4: 81500000-82000000, chr4: 83000000-83500000, chr4: 88500000- |
| | 89000000, chr4: 98500000-99000000, chr4: 99500000-100000000, chr4: 102000000-102500000, |
| | chr4: 108000000-108500000, chr4: 111000000-111500000, chr4: 113500000-114000000, |
| | chr4: 121500000-122000000, chr4: 126500000-127000000, chr4: 131500000-132000000, |
| | chr4: 139000000-139500000, chr4: 142000000-142500000, chr4: 146000000-146500000, |
| | chr4: 152000000-152500000, chr4: 152500000-153000000, chr4: 153000000-153500000, |
| | chr4: 153500000-154000000, chr4: 158500000-159000000, chr4: 167000000-167500000, |
| | chr4: 172500000-173000000, chr4: 184500000-185000000, chr4: 187000000-187500000, |
| | chr4: 189500000-190000000, chr4: 190000000-190214555 |
| 5 | chr5: 7000000-7500000, chr5: 9000000-9500000, chr5: 9500000-10000000, chr5: 10500000- |
| | 11000000, chr5: 17500000-18000000, chr5: 21000000-21500000, chr5: 26000000-26500000, |
| | chr5: 34000000-34500000, chr5: 36500000-37000000, chr5: 39000000-39500000, chr5: 50500000- |
| | 51000000, chr5: 51000000-51500000, chr5: 56000000-56500000, chr5: 56500000-57000000, |
| | chr5: 59000000-59500000, chr5: 59500000-60000000, chr5: 68000000-68500000, chr5: 69500000- |
| | 70000000, chr5: 70000000-70500000, chr5: 70500000-71000000, chr5: 71000000-71500000, |
| | chr5: 80000000-80500000, chr5: 80500000-81000000, chr5: 87000000-87500000, chr5: 91000000- |
| | 91500000, chr5: 94500000-95000000, chr5: 96500000-97000000, chr5: 102500000-103000000, |
| | chr5: 109500000-110000000, chr5: 112000000-112500000, chr5: 112500000-113000000, |
| | chr5: 115500000-116000000, chr5: 119000000-119500000, chr5: 124000000-124500000, |
| | chr5: 125000000-125500000, chr5: 126500000-127000000, chr5: 131000000-131500000, |
| | chr5: 132000000-132500000, chr5: 133500000-134000000, chr5: 136500000-137000000, |
| | chr5: 138000000-138500000, chr5: 138500000-139000000, chr5: 139500000-140000000, |
| | chr5: 140500000-141000000, chr5: 141000000-141500000, chr5: 141500000-142000000, |
| | chr5: 142000000-142500000, chr5: 142500000-143000000, chr5: 143000000-143500000, |
| | chr5: 146000000-146500000, chr5: 148500000-149000000, chr5: 151500000-152000000, |
| | chr5: 157000000-157500000, chr5: 157500000-158000000, chr5: 158500000-159000000, |
| | chr5: 159000000-159500000, chr5: 160000000-160500000, chr5: 166500000-167000000, |
| | chr5: 169500000-170000000, chr5: 170000000-170500000, chr5: 172000000-172500000, |
| | chr5: 172500000-173000000, chr5: 177000000-177500000, chr5: 181000000-181500000 |
| 6 | chr6: 2500000-3000000, chr6: 6500000-7000000, chr6: 8500000-9000000, chr6: 11000000- |
| | 11500000, chr6: 13000000-13500000, chr6: 13500000-14000000, chr6: 14000000-14500000, |
| | chr6: 14500000-15000000, chr6: 15000000-15500000, chr6: 16000000-16500000, chr6: 17500000- |
| | 18000000, chr6: 25500000-26000000, chr6: 26000000-26500000, chr6: 29500000-30000000, |
| | chr6: 30000000-30500000, chr6: 30500000-31000000, chr6: 31000000-31500000, chr6: 31500000- |
| | 32000000, chr6: 32000000-32500000, chr6: 32500000-33000000, chr6: 33000000-33500000, |
| | chr6: 33500000-34000000, chr6: 34500000-35000000, chr6: 36000000-36500000, chr6: 36500000- |
| | 37000000, chr6: 37000000-37500000, chr6: 37500000-38000000, chr6: 40500000-41000000, |
| | chr6: 41000000-41500000, chr6: 41500000-42000000, chr6: 42500000-43000000, chr6: 43000000- |
| | 43500000, chr6: 44000000-44500000, chr6: 53000000-53500000, chr6: 53500000-54000000, |
| | chr6: 57500000-58000000, chr6: 58000000-58500000, chr6: 58500000-59000000, chr6: 60000000- |
| | 60500000, chr6: 60500000-61000000, chr6: 61000000-61500000, chr6: 73000000-73500000, |
| | chr6: 77500000-78000000, chr6: 86000000-86500000, chr6: 87000000-87500000, chr6: 87500000- |
| | 88000000, chr6: 88500000-89000000, chr6: 89000000-89500000, chr6: 106000000-106500000, |
| | chr6: 108500000-109000000, chr6: 110500000-111000000, chr6: 111500000-112000000, |
| | chr6: 112000000-112500000, chr6: 112500000-113000000, chr6: 119000000-119500000, |
| | chr6: 119500000-120000000, chr6: 128000000-128500000, chr6: 129500000-130000000, |
| | chr6: 130500000-131000000, chr6: 135000000-135500000, chr6: 135500000-136000000, |
| | chr6: 137000000-137500000, chr6: 137500000-138000000, chr6: 138000000-138500000, |
| | chr6: 139000000-139500000, chr6: 142000000-142500000, chr6: 143500000-144000000, |
| | chr6: 144000000-144500000, chr6: 144500000-145000000, chr6: 149000000-149500000, |
| | chr6: 149500000-150000000, chr6: 154000000-154500000, chr6: 159500000-160000000 |
| 7 | chr7: 3000000-3500000, chr7: 4000000-4500000, chr7: 6000000-6500000, chr7: 7500000-8000000, |
| | chr7: 26000000-26500000, chr7: 27000000-27500000, chr7: 30000000-30500000, chr7: 37000000- |
| | 37500000, chr7: 39500000-40000000, chr7: 44500000-45000000, chr7: 45500000-46000000, |
| | chr7: 46000000-46500000, chr7: 51500000-52000000, chr7: 54000000-54500000, chr7: 56500000- |
| | 57000000, chr7: 57000000-57500000, chr7: 57500000-58000000, chr7: 58000000-58500000, |
| | chr7: 63000000-63500000, chr7: 63500000-64000000, chr7: 65000000-65500000, chr7: 66500000- |
| | 67000000, chr7: 72000000-72500000, chr7: 72500000-73000000, chr7: 73500000-74000000, |
| | chr7: 74000000-74500000, chr7: 74500000-75000000, chr7: 75500000-76000000, chr7: 76500000- |
| | 77000000, chr7: 77500000-78000000, chr7: 92500000-93000000, chr7: 97500000-98000000, |
| | chr7: 98000000-98500000, chr7: 99000000-99500000, chr7: 100000000-100500000, |
| | chr7: 100500000-101000000, chr7: 101000000-101500000, chr7: 102500000-103000000, |
| | chr7: 105000000-105500000, chr7: 106000000-106500000, chr7: 107000000-107500000, |
| | chr7: 112000000-112500000, chr7: 127500000-128000000, chr7: 128000000-128500000, |
| | chr7: 129000000-129500000, chr7: 129500000-130000000, chr7: 130000000-130500000, |
| | chr7: 133000000-133500000, chr7: 135500000-136000000, chr7: 139000000-139500000, |
| | chr7: 141500000-142000000, chr7: 143000000-143500000, chr7: 148500000-149000000, |
| | chr7: 151000000-151500000 |
| 8 | chr8: 0-500000, chr8: 3000000-3500000, chr8: 7000000-7500000, chr8: 7500000-8000000, |
| | chr8: 8000000-8500000, chr8: 12000000-12500000, chr8: 12500000-13000000, chr8: 17500000- |
| | 18000000, chr8: 20000000-20500000, chr8: 20500000-21000000, chr8: 22500000-23000000, |
| | chr8: 27000000-27500000, chr8: 29000000-29500000, chr8: 30000000-30500000, chr8: 30500000- |
| | 31000000, chr8: 35500000-36000000, chr8: 37500000-38000000, chr8: 42000000-42500000, |
| | chr8: 55000000-55500000, chr8: 58500000-59000000, chr8: 60500000-61000000, chr8: 66500000- |
| | 67000000, chr8: 69000000-69500000, chr8: 70000000-70500000, chr8: 72000000-72500000, |
| | chr8: 73500000-74000000, chr8: 74500000-75000000, chr8: 85500000-86000000, chr8: 91500000- |
| | 92000000, chr8: 94500000-95000000, chr8: 96500000-97000000, chr8: 100000000-100500000, |
| | chr8: 103500000-104000000, chr8: 104000000-104500000, chr8: 120500000-121000000, |
| | chr8: 122500000-123000000, chr8: 124000000-124500000, chr8: 125000000-125500000, |
| | chr8: 130000000-130500000, chr8: 133000000-133500000, chr8: 139500000-140000000, |
| | chr8: 140000000-140500000, chr8: 140500000-141000000 |
| 9 | chr9: 4500000-5000000, chr9: 5500000-6000000, chr9: 6500000-7000000, chr9: 15000000- |
| | 15500000, chr9: 17000000-17500000, chr9: 19000000-19500000, chr9: 21000000-21500000, |
| | chr9: 21500000-22000000, chr9: 33000000-33500000, chr9: 34000000-34500000, chr9: 34500000- |
| | 35000000, chr9: 35000000-35500000, chr9: 35500000-36000000, chr9: 36000000-36500000, |
| | chr9: 37500000-38000000, chr9: 39000000-39500000, chr9: 39500000-40000000, chr9: 40000000- |
| | 40500000, chr9: 40500000-41000000, chr9: 41000000-41500000, chr9: 41500000-42000000, |
| | chr9: 42000000-42500000, chr9: 42500000-43000000, chr9: 43000000-43500000, chr9: 60500000- |
| | 61000000, chr9: 61000000-61500000, chr9: 61500000-62000000, chr9: 62000000-62500000, |
| | chr9: 62500000-63000000, chr9: 63000000-63500000, chr9: 63500000-64000000, chr9: 64000000- |
| | 64500000, chr9: 64500000-65000000, chr9: 65000000-65500000, chr9: 65500000-66000000, |
| | chr9: 66000000-66500000, chr9: 66500000-67000000, chr9: 67000000-67500000, chr9: 67500000- |
| | 68000000, chr9: 77500000-78000000, chr9: 78500000-79000000, chr9: 80500000-81000000, |
| | chr9: 86500000-87000000, chr9: 89000000-89500000, chr9: 92500000-93000000, chr9: 99000000- |
| | 99500000, chr9: 105000000-105500000, chr9: 114500000-115000000, chr9: 124500000- |
| | 125000000, chr9: 125000000-125500000, chr9: 126000000-126500000, chr9: 127500000- |
| | 128000000, chr9: 128000000-128500000, chr9: 128500000-129000000, chr9: 129000000- |
| | 129500000, chr9: 129500000-130000000, chr9: 132000000-132500000, chr9: 135000000- |
| | 135500000 |
| 10 | chr10: 3000000-3500000, chr10: 3500000-4000000, chr10: 4000000-4500000, chr10: 6000000- |
| | 6500000, chr10: 8000000-8500000, chr10: 9000000-9500000, chr10: 13500000-14000000, |
| | chr10: 20500000-21000000, chr10: 21500000-22000000, chr10: 27500000-28000000, |
| | chr10: 28000000-28500000, chr10: 36500000-37000000, chr10: 37000000-37500000, |
| | chr10: 38000000-38500000, chr10: 38500000-39000000, chr10: 42000000-42500000, |
| | chr10: 42500000-43000000, chr10: 43000000-43500000, chr10: 44000000-44500000, |
| | chr10: 46000000-46500000, chr10: 46500000-47000000, chr10: 47500000-48000000, |
| | chr10: 52000000-52500000, chr10: 53500000-54000000, chr10: 55500000-56000000, |
| | chr10: 58000000-58500000, chr10: 59000000-59500000, chr10: 62000000-62500000, |
| | chr10: 63000000-63500000, chr10: 68000000-68500000, chr10: 68500000-69000000, |
| | chr10: 69000000-69500000, chr10: 72000000-72500000, chr10: 73000000-73500000, |
| | chr10: 79000000-79500000, chr10: 84500000-85000000, chr10: 87000000-87500000, |
| | chr10: 88000000-88500000, chr10: 92500000-93000000, chr10: 94500000-95000000, |
| | chr10: 95000000-95500000, chr10: 97000000-97500000, chr10: 97500000-98000000, |
| | chr10: 100000000-100500000, chr10: 100500000-101000000, chr10: 101000000-101500000, |
| | chr10: 101500000-102000000, chr10: 102000000-102500000, chr10: 102500000-103000000, |
| | chr10: 104000000-104500000, chr10: 108000000-108500000, chr10: 109000000-109500000, |
| | chr10: 109500000-110000000, chr10: 110500000-111000000, chr10: 111500000-112000000, |
| | chr10: 119000000-119500000, chr10: 125500000-126000000, chr10: 126500000-127000000, |
| | chr10: 127000000-127500000, chr10: 128500000-129000000, chr10: 129500000-130000000, |
| | chr10: 130000000-130500000, chr10: 130500000-131000000, chr10: 133500000-133797422 |
| 11 | chr11: 0-500000, chr11: 1500000-2000000, chr11: 3500000-4000000, chr11: 6000000-6500000, |
| | chr11: 6500000-7000000, chr11: 12000000-12500000, chr11: 13000000-13500000, |
| | chr11: 18000000-18500000, chr11: 33000000-33500000, chr11: 34500000-35000000, |
| | chr11: 39500000-40000000, chr11: 40000000-40500000, chr11: 44500000-45000000, |
| | chr11: 46000000-46500000, chr11: 47000000-47500000, chr11: 47500000-48000000, |
| | chr11: 48500000-49000000, chr11: 49000000-49500000, chr11: 49500000-50000000, |
| | chr11: 50000000-50500000, chr11: 55500000-56000000, chr11: 56500000-57000000, |
| | chr11: 57500000-58000000, chr11: 59500000-60000000, chr11: 61000000-61500000, |
| | chr11: 62000000-62500000, chr11: 62500000-63000000, chr11: 63500000-64000000, |
| | chr11: 64500000-65000000, chr11: 65000000-65500000, chr11: 65500000-66000000, |
| | chr11: 66000000-66500000, chr11: 67000000-67500000, chr11: 72000000-72500000, |
| | chr11: 72500000-73000000, chr11: 73000000-73500000, chr11: 73500000-74000000, |
| | chr11: 77500000-78000000, chr11: 78000000-78500000, chr11: 86000000-86500000, |
| | chr11: 89000000-89500000, chr11: 89500000-90000000, chr11: 92000000-92500000, |
| | chr11: 93000000-93500000, chr11: 95000000-95500000, chr11: 95500000-96000000, |
| | chr11: 99500000-100000000, chr11: 100000000-100500000, chr11: 101000000-101500000, |
| | chr11: 102000000-102500000, chr11: 104000000-104500000, chr11: 104500000-105000000, |
| | chr11: 109000000-109500000, chr11: 111500000-112000000, chr11: 113500000-114000000, |
| | chr11: 114000000-114500000, chr11: 118000000-118500000, chr11: 119000000-119500000, |
| | chr11: 121000000-121500000, chr11: 122500000-123000000, chr11: 128500000-129000000, |
| | chr11: 135000000-135086622 |
| 12 | chr12: 500000-1000000, chr12: 1500000-2000000, chr12: 6000000-6500000, chr12: 6500000- |
| | 7000000, chr12: 8500000-9000000, chr12: 12000000-12500000, chr12: 12500000-13000000, |
| | chr12: 14500000-15000000, chr12: 25500000-26000000, chr12: 29500000-30000000, |
| | chr12: 34500000-35000000, chr12: 37000000-37500000, chr12: 38000000-38500000, |
| | chr12: 42000000-42500000, chr12: 47500000-48000000, chr12: 48500000-49000000, |
| | chr12: 49000000-49500000, chr12: 49500000-50000000, chr12: 50000000-50500000, |
| | chr12: 50500000-51000000, chr12: 51000000-51500000, chr12: 53000000-53500000, |
| | chr12: 53500000-54000000, chr12: 54000000-54500000, chr12: 55500000-56000000, |
| | chr12: 56000000-56500000, chr12: 57000000-57500000, chr12: 57500000-58000000, |
| | chr12: 60500000-61000000, chr12: 61000000-61500000, chr12: 62000000-62500000, |
| | chr12: 64500000-65000000, chr12: 66000000-66500000, chr12: 68000000-68500000, |
| | chr12: 73000000-73500000, chr12: 75500000-76000000, chr12: 76000000-76500000, |
| | chr12: 91500000-92000000, chr12: 93000000-93500000, chr12: 93500000-94000000, |
| | chr12: 96000000-96500000, chr12: 98500000-99000000, chr12: 101500000-102000000, |
| | chr12: 102500000-103000000, chr12: 104000000-104500000, chr12: 104500000-105000000, |
| | chr12: 105000000-105500000, chr12: 106500000-107000000, chr12: 108500000-109000000, |
| | chr12: 109500000-110000000, chr12: 110000000-110500000, chr12: 110500000-111000000, |
| | chr12: 111000000-111500000, chr12: 112000000-112500000, chr12: 112500000-113000000, |
| | chr12: 113000000-113500000, chr12: 116000000-116500000, chr12: 116500000-117000000, |
| | chr12: 118000000-118500000, chr12: 120000000-120500000, chr12: 120500000-121000000, |
| | chr12: 121000000-121500000, chr12: 121500000-122000000, chr12: 122000000-122500000, |
| | chr12: 123500000-124000000, chr12: 127500000-128000000 |
| 13 | chr13: 16000000-16500000, chr13: 18000000-18500000, chr13: 18500000-19000000, |
| | chr13: 19000000-19500000, chr13: 21500000-22000000, chr13: 27000000-27500000, |
| | chr13: 28000000-28500000, chr13: 30000000-30500000, chr13: 35500000-36000000, |
| | chr13: 36000000-36500000, chr13: 40000000-40500000, chr13: 41000000-41500000, |
| | chr13: 44000000-44500000, chr13: 46000000-46500000, chr13: 49500000-50000000, |
| | chr13: 56500000-57000000, chr13: 57000000-57500000, chr13: 60500000-61000000, |
| | chr13: 66500000-67000000, chr13: 73000000-73500000, chr13: 90500000-91000000, |
| | chr13: 92500000-93000000, chr13: 97500000-98000000, chr13: 112000000-112500000 |
| 14 | chr14: 18000000-18500000, chr14: 18500000-19000000, chr14: 19000000-19500000, |
| | chr14: 19500000-20000000, chr14: 21000000-21500000, chr14: 22500000-23000000, |
| | chr14: 23000000-23500000, chr14: 25500000-26000000, chr14: 28000000-28500000, |
| | chr14: 30000000-30500000, chr14: 34500000-35000000, chr14: 35000000-35500000, |
| | chr14: 39000000-39500000, chr14: 39500000-40000000, chr14: 43000000-43500000, |
| | chr14: 49500000-50000000, chr14: 50500000-51000000, chr14: 51500000-52000000, |
| | chr14: 55000000-55500000, chr14: 55500000-56000000, chr14: 59000000-59500000, |
| | chr14: 60000000-60500000, chr14: 64500000-65000000, chr14: 65000000-65500000, |
| | chr14: 70500000-71000000, chr14: 73000000-73500000, chr14: 74000000-74500000, |
| | chr14: 75000000-75500000, chr14: 77000000-77500000, chr14: 86500000-87000000, |
| | chr14: 88500000-89000000, chr14: 89000000-89500000, chr14: 91000000-91500000, |
| | chr14: 97000000-97500000, chr14: 97500000-98000000 |
| 15 | chr15: 17000000-17500000, chr15: 19500000-20000000, chr15: 20000000-20500000, |
| | chr15: 20500000-21000000, chr15: 21000000-21500000, chr15: 21500000-22000000, |
| | chr15: 22000000-22500000, chr15: 23000000-23500000, chr15: 24000000-24500000, |
| | chr15: 24500000-25000000, chr15: 26500000-27000000, chr15: 28500000-29000000, |
| | chr15: 30000000-30500000, chr15: 30500000-31000000, chr15: 32000000-32500000, |
| | chr15: 34000000-34500000, chr15: 35000000-35500000, chr15: 36000000-36500000, |
| | chr15: 41000000-41500000, chr15: 41500000-42000000, chr15: 43000000-43500000, |
| | chr15: 46000000-46500000, chr15: 47000000-47500000, chr15: 48500000-49000000, |
| | chr15: 50000000-50500000, chr15: 53000000-53500000, chr15: 55000000-55500000, |
| | chr15: 57000000-57500000, chr15: 58000000-58500000, chr15: 60000000-60500000, |
| | chr15: 63000000-63500000, chr15: 63500000-64000000, chr15: 64000000-64500000, |
| | chr15: 64500000-65000000, chr15: 65500000-66000000, chr15: 69000000-69500000, |
| | chr15: 70000000-70500000, chr15: 74500000-75000000, chr15: 81000000-81500000, |
| | chr15: 81500000-82000000, chr15: 82000000-82500000, chr15: 84000000-84500000, |
| | chr15: 85500000-86000000, chr15: 90000000-90500000, chr15: 90500000-91000000, |
| | chr15: 91500000-92000000, chr15: 94000000-94500000 |
| 16 | chr16: 4500000-5000000, chr16: 8500000-9000000, chr16: 10500000-11000000, chr16: 11500000- |
| | 12000000, chr16: 14500000-15000000, chr16: 15000000-15500000, chr16: 15500000-16000000, |
| | chr16: 17000000-17500000, chr16: 18000000-18500000, chr16: 22000000-22500000, |
| | chr16: 23500000-24000000, chr16: 24000000-24500000, chr16: 24500000-25000000, |
| | chr16: 26500000-27000000, chr16: 27000000-27500000, chr16: 29000000-29500000, |
| | chr16: 29500000-30000000, chr16: 30500000-31000000, chr16: 31000000-31500000, |
| | chr16: 32000000-32500000, chr16: 32500000-33000000, chr16: 33000000-33500000, |
| | chr16: 33500000-34000000, chr16: 34000000-34500000, chr16: 34500000-35000000, |
| | chr16: 35000000-35500000, chr16: 46000000-46500000, chr16: 53000000-53500000, |
| | chr16: 53500000-54000000, chr16: 54000000-54500000, chr16: 57000000-57500000, |
| | chr16: 67500000-68000000, chr16: 68000000-68500000, chr16: 69000000-69500000, |
| | chr16: 71500000-72000000, chr16: 72000000-72500000, chr16: 74500000-75000000, |
| | chr16: 79000000-79500000, chr16: 81000000-81500000, chr16: 81500000-82000000, |
| | chr16: 84500000-85000000, chr16: 90000000-90338345 |
| 17 | chr17: 0-500000, chr17: 1000000-1500000, chr17: 1500000-2000000, chr17: 2000000-2500000, |
| | chr17: 2500000-3000000, chr17: 4000000-4500000, chr17: 4500000-5000000, chr17: 5000000- |
| | 5500000, chr17: 7000000-7500000, chr17: 7500000-8000000, chr17: 8000000-8500000, |
| | chr17: 8500000-9000000, chr17: 14000000-14500000, chr17: 16000000-16500000, |
| | chr17: 19000000-19500000, chr17: 19500000-20000000, chr17: 20500000-21000000, |
| | chr17: 21000000-21500000, chr17: 21500000-22000000, chr17: 22000000-22500000, |
| | chr17: 22500000-23000000, chr17: 26500000-27000000, chr17: 28500000-29000000, |
| | chr17: 30000000-30500000, chr17: 31000000-31500000, chr17: 35000000-35500000, |
| | chr17: 35500000-36000000, chr17: 36000000-36500000, chr17: 36500000-37000000, |
| | chr17: 37000000-37500000, chr17: 38000000-38500000, chr17: 38500000-39000000, |
| | chr17: 39000000-39500000, chr17: 39500000-40000000, chr17: 40000000-40500000, |
| | chr17: 42000000-42500000, chr17: 42500000-43000000, chr17: 43500000-44000000, |
| | chr17: 44000000-44500000, chr17: 44500000-45000000, chr17: 45000000-45500000, |
| | chr17: 47500000-48000000, chr17: 48500000-49000000, chr17: 49000000-49500000, |
| | chr17: 49500000-50000000, chr17: 50000000-50500000, chr17: 50500000-51000000, |
| | chr17: 54000000-54500000, chr17: 56500000-57000000, chr17: 57000000-57500000, |
| | chr17: 58000000-58500000, chr17: 59500000-60000000, chr17: 62500000-63000000, |
| | chr17: 63500000-64000000, chr17: 64000000-64500000, chr17: 65000000-65500000, |
| | chr17: 66000000-66500000, chr17: 67000000-67500000, chr17: 68000000-68500000, |
| | chr17: 73500000-74000000, chr17: 74500000-75000000, chr17: 75000000-75500000, |
| | chr17: 75500000-76000000, chr17: 76000000-76500000, chr17: 77000000-77500000, |
| | chr17: 78500000-79000000, chr17: 79500000-80000000, chr17: 80000000-80500000 |
| 18 | chr18: 3000000-3500000, chr18: 5500000-6000000, chr18: 6500000-7000000, chr18: 11000000- |
| | 11500000, chr18: 14000000-14500000, chr18: 14500000-15000000, chr18: 15000000-15500000, |
| | chr18: 21000000-21500000, chr18: 23500000-24000000, chr18: 27500000-28000000, |
| | chr18: 35000000-35500000, chr18: 46000000-46500000, chr18: 51000000-51500000, |
| | chr18: 61000000-61500000, chr18: 78000000-78500000 |
| 19 | chr19: 0-500000, chr19: 500000-1000000, chr19: 1500000-2000000, chr19: 2000000-2500000, |
| | chr19: 2500000-3000000, chr19: 3000000-3500000, chr19: 4000000-4500000, chr19: 4500000- |
| | 5000000, chr19: 5500000-6000000, chr19: 6000000-6500000, chr19: 6500000-7000000, |
| | chr19: 7000000-7500000, chr19: 7500000-8000000, chr19: 8000000-8500000, chr19: 9500000- |
| | 10000000, chr19: 10000000-10500000, chr19: 10500000-11000000, chr19: 11000000-11500000, |
| | chr19: 11500000-12000000, chr19: 12500000-13000000, chr19: 13000000-13500000, |
| | chr19: 13500000-14000000, chr19: 14000000-14500000, chr19: 14500000-15000000, |
| | chr19: 15500000-16000000, chr19: 16000000-16500000, chr19: 16500000-17000000, |
| | chr19: 17000000-17500000, chr19: 17500000-18000000, chr19: 18000000-18500000, |
| | chr19: 18500000-19000000, chr19: 19000000-19500000, chr19: 20500000-21000000, |
| | chr19: 22000000-22500000, chr19: 22500000-23000000, chr19: 27500000-28000000, |
| | chr19: 34000000-34500000, chr19: 35000000-35500000, chr19: 35500000-36000000, |
| | chr19: 38000000-38500000, chr19: 38500000-39000000, chr19: 39000000-39500000, |
| | chr19: 39500000-40000000, chr19: 40000000-40500000, chr19: 40500000-41000000, |
| | chr19: 41000000-41500000, chr19: 41500000-42000000, chr19: 42000000-42500000, |
| | chr19: 42500000-43000000, chr19: 43500000-44000000, chr19: 44500000-45000000, |
| | chr19: 45000000-45500000, chr19: 45500000-46000000, chr19: 46500000-47000000, |
| | chr19: 47000000-47500000, chr19: 48000000-48500000, chr19: 48500000-49000000, |
| | chr19: 49000000-49500000, chr19: 49500000-50000000, chr19: 50500000-51000000, |
| | chr19: 51000000-51500000, chr19: 51500000-52000000, chr19: 54000000-54500000, |
| | chr19: 55500000-56000000 |
| 20 | chr20: 1000000-1500000, chr20: 1500000-2000000, chr20: 2500000-3000000, chr20: 3000000- |
| | 3500000, chr20: 3500000-4000000, chr20: 4500000-5000000, chr20: 5000000-5500000, |
| | chr20: 5500000-6000000, chr20: 8000000-8500000, chr20: 21500000-22000000, chr20: 25500000- |
| | 26000000, chr20: 26000000-26500000, chr20: 28500000-29000000, chr20: 29000000-29500000, |
| | chr20: 29500000-30000000, chr20: 30000000-30500000, chr20: 30500000-31000000, |
| | chr20: 31500000-32000000, chr20: 32000000-32500000, chr20: 33000000-33500000, |
| | chr20: 33500000-34000000, chr20: 34000000-34500000, chr20: 34500000-35000000, |
| | chr20: 36500000-37000000, chr20: 37000000-37500000, chr20: 38000000-38500000, |
| | chr20: 38500000-39000000, chr20: 40500000-41000000, chr20: 43500000-44000000, |
| | chr20: 44000000-44500000, chr20: 44500000-45000000, chr20: 45000000-45500000, |
| | chr20: 45500000-46000000, chr20: 46000000-46500000, chr20: 47000000-47500000, |
| | chr20: 48500000-49000000, chr20: 49500000-50000000, chr20: 50000000-50500000, |
| | chr20: 51500000-52000000, chr20: 52000000-52500000, chr20: 53500000-54000000, |
| | chr20: 57000000-57500000, chr20: 62500000-63000000, chr20: 63000000-63500000 |
| 21 | chr21: 5000000-5500000, chr21: 5500000-6000000, chr21: 6000000-6500000, chr21: 6500000- |
| | 7000000, chr21: 7000000-7500000, chr21: 7500000-8000000, chr21: 8000000-8500000, |
| | chr21: 8500000-9000000, chr21: 9000000-9500000, chr21: 9500000-10000000, chr21: 10000000- |
| | 10500000, chr21: 10500000-11000000, chr21: 13000000-13500000, chr21: 13500000-14000000, |
| | chr21: 17000000-17500000, chr21: 25500000-26000000, chr21: 29000000-29500000, |
| | chr21: 31000000-31500000, chr21: 33000000-33500000, chr21: 34500000-35000000, |
| | chr21: 36000000-36500000, chr21: 40000000-40500000, chr21: 42000000-42500000 |
| 22 | chr22: 10500000-11000000, chr22: 11000000-11500000, chr22: 11500000-12000000, |
| | chr22: 12000000-12500000, chr22: 12500000-13000000, chr22: 15000000-15500000, |
| | chr22: 15500000-16000000, chr22: 16000000-16500000, chr22: 16500000-17000000, |
| | chr22: 18000000-18500000, chr22: 18500000-19000000, chr22: 21000000-21500000, |
| | chr22: 21500000-22000000, chr22: 27000000-27500000, chr22: 29500000-30000000, |
| | chr22: 30000000-30500000, chr22: 31000000-31500000, chr22: 31500000-32000000, |
| | chr22: 35000000-35500000, chr22: 35500000-36000000, chr22: 36000000-36500000, |
| | chr22: 36500000-37000000, chr22: 37000000-37500000, chr22: 37500000-38000000, |
| | chr22: 38000000-38500000, chr22: 40000000-40500000, chr22: 40500000-41000000, |
| | chr22: 41000000-41500000, chr22: 41500000-42000000, chr22: 48500000-49000000 |

TABLE 17

| | |
|---|---|
| 1 | chr1: 500000-1000000, chr1: 1500000-2000000, chr1: 2500000-3000000, chr1: 8000000-8500000, |
| | chr1: 10000000-10500000, chr1: 12500000-13000000, chr1: 13000000-13500000, chr1: 16000000- |
| | 16500000, chr1: 16500000-17000000, chr1: 18000000-18500000, chr1: 19000000-19500000, |
| | chr1: 20500000-21000000, chr1: 21000000-21500000, chr1: 23000000-23500000, chr1: 23500000- |
| | 24000000, chr1: 26000000-26500000, chr1: 26500000-27000000, chr1: 27000000-27500000, |
| | chr1: 27500000-28000000, chr1: 28000000-28500000, chr1: 31500000-32000000, chr1: 32000000- |
| | 32500000, chr1: 35500000-36000000, chr1: 40000000-40500000, chr1: 41000000-41500000, |
| | chr1: 44500000-45000000, chr1: 45500000-46000000, chr1: 48000000-48500000, chr1: 50000000- |
| | 50500000, chr1: 51500000-52000000, chr1: 52500000-53000000, chr1: 61000000-61500000, |
| | chr1: 63500000-64000000, chr1: 64500000-65000000, chr1: 65000000-65500000, chr1: 66500000- |
| | 67000000, chr1: 67000000-67500000, chr1: 83000000-83500000, chr1: 84000000-84500000, |
| | chr1: 88500000-89000000, chr1: 91500000-92000000, chr1: 92500000-93000000, chr1: 94500000- |
| | 95000000, chr1: 97500000-98000000, chr1: 98000000-98500000, chr1: 98500000-99000000, |
| | chr1: 107500000-108000000, chr1: 108500000-109000000, chr1: 111500000-112000000, |
| | chr1: 112500000-113000000, chr1: 116000000-116500000, chr1: 116500000-117000000, |
| | chr1: 120000000-120500000, chr1: 120500000-121000000, chr1: 121000000-121500000, |
| | chr1: 125000000-125500000, chr1: 143000000-143500000, chr1: 143500000-144000000, |
| | chr1: 144000000-144500000, chr1: 144500000-145000000, chr1: 145000000-145500000, |
| | chr1: 146000000-146500000, chr1: 146500000-147000000, chr1: 147500000-148000000, |
| | chr1: 148000000-148500000, chr1: 148500000-149000000, chr1: 149000000-149500000, |
| | chr1: 150000000-150500000, chr1: 150500000-151000000, chr1: 151000000-151500000, |
| | chr1: 151500000-152000000, chr1: 153500000-154000000, chr1: 154000000-154500000, |
| | chr1: 154500000-155000000, chr1: 156000000-156500000, chr1: 156500000-157000000, |
| | chr1: 157000000-157500000, chr1: 159000000-159500000, chr1: 160000000-160500000, |
| | chr1: 161000000-161500000, chr1: 161500000-162000000, chr1: 165500000-166000000, |
| | chr1: 167500000-168000000, chr1: 168500000-169000000, chr1: 169500000-170000000, |
| | chr1: 171000000-171500000, chr1: 171500000-172000000, chr1: 172500000-173000000, |
| | chr1: 173500000-174000000, chr1: 174000000-174500000, chr1: 176500000-177000000, |
| | chr1: 177500000-178000000, chr1: 181000000-181500000, chr1: 182500000-183000000, |
| | chr1: 183000000-183500000, chr1: 185000000-185500000, chr1: 193000000-193500000, |
| | chr1: 197000000-197500000, chr1: 198500000-199000000, chr1: 199000000-199500000, |
| | chr1: 200000000-200500000, chr1: 203000000-203500000, chr1: 204000000-204500000, |
| | chr1: 206500000-207000000, chr1: 210000000-210500000, chr1: 213500000-214000000, |
| | chr1: 220000000-220500000, chr1: 222500000-223000000, chr1: 227000000-227500000, |
| | chr1: 233000000-233500000, chr1: 233500000-234000000, chr1: 235000000-235500000, |
| | chr1: 235500000-236000000, chr1: 236000000-236500000, chr1: 243500000-244000000, |
| | chr1: 246500000-247000000, chr1: 247000000-247500000 |
| 2 | chr2: 1500000-2000000, chr2: 8500000-9000000, chr2: 20000000-20500000, chr2: 21000000- |
| | 21500000, chr2: 24500000-25000000, chr2: 25000000-25500000, chr2: 27500000-28000000, |
| | chr2: 36000000-36500000, chr2: 36500000-37000000, chr2: 37000000-37500000, chr2: 37500000- |
| | 38000000, chr2: 39000000-39500000, chr2: 42000000-42500000, chr2: 43000000-43500000, |
| | chr2: 45500000-46000000, chr2: 47000000-47500000, chr2: 48000000-48500000, chr2: 50000000- |
| | 50500000, chr2: 61500000-62000000, chr2: 68500000-69000000, chr2: 69500000-70000000, |
| | chr2: 72500000-73000000, chr2: 75000000-75500000, chr2: 80000000-80500000, chr2: 84500000- |
| | 85000000, chr2: 87000000-87500000, chr2: 87500000-88000000, chr2: 89500000-90000000, |
| | chr2: 90000000-90500000, chr2: 91000000-91500000, chr2: 91500000-92000000, chr2: 92000000- |
| | 92500000, chr2: 94500000-95000000, chr2: 95500000-96000000, chr2: 98500000-99000000, |
| | chr2: 102000000-102500000, chr2: 107000000-107500000, chr2: 109500000-110000000, |
| | chr2: 112500000-113000000, chr2: 113000000-113500000, chr2: 113500000-114000000, |
| | chr2: 119500000-120000000, chr2: 129000000-129500000, chr2: 130000000-130500000, |
| | chr2: 131500000-132000000, chr2: 132000000-132500000, chr2: 132500000-133000000, |
| | chr2: 134000000-134500000, chr2: 135500000-136000000, chr2: 136000000-136500000, |
| | chr2: 139500000-140000000, chr2: 143000000-143500000, chr2: 143500000-144000000, |
| | chr2: 144500000-145000000, chr2: 148500000-149000000, chr2: 156000000-156500000, |
| | chr2: 157000000-157500000, chr2: 158000000-158500000, chr2: 174500000-175000000, |
| | chr2: 177000000-177500000, chr2: 178000000-178500000, chr2: 181000000-181500000, |
| | chr2: 181500000-182000000, chr2: 189000000-189500000, chr2: 189500000-190000000, |
| | chr2: 190000000-190500000, chr2: 190500000-191000000, chr2: 191000000-191500000, |
| | chr2: 191500000-192000000, chr2: 196000000-196500000, chr2: 197000000-197500000, |
| | chr2: 200500000-201000000, chr2: 201000000-201500000, chr2: 201500000-202000000, |
| | chr2: 203000000-203500000, chr2: 203500000-204000000, chr2: 207500000-208000000, |
| | chr2: 208500000-209000000, chr2: 210000000-210500000, chr2: 216000000-216500000, |
| | chr2: 218000000-218500000, chr2: 224500000-225000000, chr2: 235000000-235500000, |
| | chr2: 235500000-236000000, chr2: 240500000-241000000 |
| 3 | chr3: 2500000-3000000, chr3: 3000000-3500000, chr3: 4500000-5000000, chr3: 10000000- |
| | 10500000, chr3: 12500000-13000000, chr3: 14000000-14500000, chr3: 17000000-17500000, |
| | chr3: 17500000-18000000, chr3: 26000000-26500000, chr3: 27500000-28000000, chr3: 28500000- |
| | 29000000, chr3: 30500000-31000000, chr3: 32000000-32500000, chr3: 33500000-34000000, |
| | chr3: 36000000-36500000, chr3: 38000000-38500000, chr3: 39000000-39500000, chr3: 47000000- |
| | 47500000, chr3: 47500000-48000000, chr3: 48500000-49000000, chr3: 54500000-55000000, |
| | chr3: 55500000-56000000, chr3: 56500000-57000000, chr3: 57500000-58000000, chr3: 59000000- |
| | 59500000, chr3: 64500000-65000000, chr3: 70000000-70500000, chr3: 71000000-71500000, |
| | chr3: 75000000-75500000, chr3: 75500000-76000000, chr3: 88000000-88500000, chr3: 91000000- |
| | 91500000, chr3: 100000000-100500000, chr3: 101000000-101500000, chr3: 101500000- |
| | 102000000, chr3: 105500000-106000000, chr3: 106000000-106500000, chr3: 107500000- |
| | 108000000, chr3: 112500000-113000000, chr3: 122000000-122500000, chr3: 125000000- |
| | 125500000, chr3: 125500000-126000000, chr3: 127000000-127500000, chr3: 129500000- |
| | 130000000, chr3: 134000000-134500000, chr3: 135500000-136000000, chr3: 136500000- |
| | 137000000, chr3: 143000000-143500000, chr3: 150000000-150500000, chr3: 151000000- |
| | 151500000, chr3: 151500000-152000000, chr3: 152000000-152500000, chr3: 153000000- |
| | 153500000, chr3: 156500000-157000000, chr3: 157000000-157500000, chr3: 158500000- |
| | 159000000, chr3: 160500000-161000000, chr3: 161000000-161500000, chr3: 165500000- |
| | 166000000, chr3: 169000000-169500000, chr3: 170000000-170500000, chr3: 171000000- |
| | 171500000, chr3: 172000000-172500000, chr3: 179000000-179500000, chr3: 182500000- |
| | 183000000, chr3: 184500000-185000000, chr3: 186500000-187000000, chr3: 198000000- |
| | 198295559 |
| 4 | chr4: 5500000-6000000, chr4: 8500000-9000000, chr4: 9000000-9500000, chr4: 18000000- |
| | 18500000, chr4: 21000000-21500000, chr4: 26500000-27000000, chr4: 36000000-36500000, |
| | chr4: 38500000-39000000, chr4: 39000000-39500000, chr4: 39500000-40000000, chr4: 40000000- |
| | 40500000, chr4: 48500000-49000000, chr4: 49000000-49500000, chr4: 49500000-50000000, |
| | chr4: 54500000-55000000, chr4: 69500000-70000000, chr4: 70500000-71000000, chr4: 71500000- |
| | 72000000, chr4: 75500000-76000000, chr4: 77500000-78000000, chr4: 78500000-79000000, |
| | chr4: 81500000-82000000, chr4: 82000000-82500000, chr4: 83000000-83500000, chr4: 89500000- |
| | 90000000, chr4: 98000000-98500000, chr4: 99500000-100000000, chr4: 107500000-108000000, |
| | chr4: 109500000-110000000, chr4: 113500000-114000000, chr4: 128500000-129000000, |
| | chr4: 139000000-139500000, chr4: 142500000-143000000, chr4: 150500000-151000000, |
| | chr4: 152000000-152500000, chr4: 152500000-153000000, chr4: 153000000-153500000, |
| | chr4: 153500000-154000000, chr4: 158500000-159000000, chr4: 159000000-159500000, |
| | chr4: 173000000-173500000, chr4: 176000000-176500000, chr4: 181500000-182000000, |
| | chr4: 189500000-190000000, chr4: 190000000-190214555 |
| 5 | chr5: 9000000-9500000, chr5: 9500000-10000000, chr5: 14000000-14500000, chr5: 15500000- |
| | 16000000, chr5: 17500000-18000000, chr5: 21000000-21500000, chr5: 23500000-24000000, |
| | chr5: 34000000-34500000, chr5: 39000000-39500000, chr5: 50500000-51000000, chr5: 51000000- |
| | 51500000, chr5: 54500000-55000000, chr5: 56000000-56500000, chr5: 56500000-57000000, |
| | chr5: 59000000-59500000, chr5: 59500000-60000000, chr5: 62000000-62500000, chr5: 65500000- |
| | 66000000, chr5: 68000000-68500000, chr5: 69000000-69500000, chr5: 69500000-70000000, |
| | chr5: 70000000-70500000, chr5: 70500000-71000000, chr5: 71000000-71500000, chr5: 75000000- |
| | 75500000, chr5: 75500000-76000000, chr5: 80000000-80500000, chr5: 85000000-85500000, |
| | chr5: 87000000-87500000, chr5: 91000000-91500000, chr5: 94500000-95000000, chr5: 96000000- |
| | 96500000, chr5: 96500000-97000000, chr5: 109500000-110000000, chr5: 111000000-111500000, |
| | chr5: 112000000-112500000, chr5: 112500000-113000000, chr5: 114000000-114500000, |
| | chr5: 115500000-116000000, chr5: 119000000-119500000, chr5: 124500000-125000000, |
| | chr5: 126500000-127000000, chr5: 131000000-131500000, chr5: 138000000-138500000, |
| | chr5: 140000000-140500000, chr5: 140500000-141000000, chr5: 142000000-142500000, |
| | chr5: 142500000-143000000, chr5: 143000000-143500000, chr5: 145000000-145500000, |
| | chr5: 146000000-146500000, chr5: 157500000-158000000, chr5: 159000000-159500000, |
| | chr5: 167500000-168000000, chr5: 169000000-169500000, chr5: 172000000-172500000, |
| | chr5: 177000000-177500000 |
| 6 | chr6: 11000000-11500000, chr6: 13500000-14000000, chr6: 14500000-15000000, chr6: 15000000- |
| | 15500000, chr6: 16500000-17000000, chr6: 17500000-18000000, chr6: 25500000-26000000, |
| | chr6: 26000000-26500000, chr6: 29500000-30000000, chr6: 30000000-30500000, chr6: 30500000- |
| | 31000000, chr6: 31000000-31500000, chr6: 31500000-32000000, chr6: 32000000-32500000, |
| | chr6: 32500000-33000000, chr6: 33000000-33500000, chr6: 33500000-34000000, chr6: 36000000- |
| | 36500000, chr6: 36500000-37000000, chr6: 38500000-39000000, chr6: 41000000-41500000, |
| | chr6: 41500000-42000000, chr6: 42000000-42500000, chr6: 42500000-43000000, chr6: 43000000- |
| | 43500000, chr6: 44000000-44500000, chr6: 45000000-45500000, chr6: 53000000-53500000, |
| | chr6: 57500000-58000000, chr6: 58000000-58500000, chr6: 60000000-60500000, chr6: 60500000- |
| | 61000000, chr6: 61000000-61500000, chr6: 63500000-64000000, chr6: 73000000-73500000, |
| | chr6: 87000000-87500000, chr6: 87500000-88000000, chr6: 89500000-90000000, chr6: 90500000- |
| | 91000000, chr6: 105000000-105500000, chr6: 106000000-106500000, chr6: 108500000- |
| | 109000000, chr6: 110500000-111000000, chr6: 111500000-112000000, chr6: 116000000- |
| | 116500000, chr6: 118500000-119000000, chr6: 119000000-119500000, chr6: 119500000- |
| | 120000000, chr6: 127500000-128000000, chr6: 128000000-128500000, chr6: 129000000- |
| | 129500000, chr6: 129500000-130000000, chr6: 131000000-131500000, chr6: 135000000- |
| | 135500000, chr6: 135500000-136000000, chr6: 137000000-137500000, chr6: 137500000- |
| | 138000000, chr6: 139000000-139500000, chr6: 142000000-142500000, chr6: 142500000- |
| | 143000000, chr6: 143500000-144000000, chr6: 144500000-145000000, chr6: 149000000- |
| | 149500000, chr6: 149500000-150000000, chr6: 154000000-154500000, chr6: 156500000- |
| | 157000000, chr6: 157000000-157500000, chr6: 158500000-159000000, chr6: 166500000- |
| | 167000000, chr6: 167500000-168000000 |
| 7 | chr7: 1000000-1500000, chr7: 3000000-3500000, chr7: 6000000-6500000, chr7: 6500000-7000000, |
| | chr7: 7000000-7500000, chr7: 7500000-8000000, chr7: 12000000-12500000, chr7: 12500000- |
| | 13000000, chr7: 17000000-17500000, chr7: 18000000-18500000, chr7: 21000000-21500000, |
| | chr7: 36500000-37000000, chr7: 37000000-37500000, chr7: 37500000-38000000, chr7: 38000000- |
| | 38500000, chr7: 38500000-39000000, chr7: 40000000-40500000, chr7: 45500000-46000000, |
| | chr7: 51000000-51500000, chr7: 51500000-52000000, chr7: 56500000-57000000, chr7: 57500000- |
| | 58000000, chr7: 58000000-58500000, chr7: 63000000-63500000, chr7: 63500000-64000000, |
| | chr7: 71500000-72000000, chr7: 72000000-72500000, chr7: 72500000-73000000, chr7: 76500000- |
| | 77000000, chr7: 77500000-78000000, chr7: 91000000-91500000, chr7: 92500000-93000000, |
| | chr7: 93500000-94000000, chr7: 98000000-98500000, chr7: 100000000-100500000, |
| | chr7: 100500000-101000000, chr7: 102500000-103000000, chr7: 103000000-103500000, |
| | chr7: 105000000-105500000, chr7: 106000000-106500000, chr7: 107000000-107500000, |
| | chr7: 115000000-115500000, chr7: 127500000-128000000, chr7: 130000000-130500000, |
| | chr7: 130500000-131000000, chr7: 133000000-133500000, chr7: 133500000-134000000, |
| | chr7: 135500000-136000000, chr7: 139000000-139500000, chr7: 141500000-142000000, |
| | chr7: 142500000-143000000, chr7: 143000000-143500000, chr7: 144500000-145000000, |
| | chr7: 154000000-154500000 |
| 8 | chr8: 3000000-3500000, chr8: 6500000-7000000, chr8: 7000000-7500000, chr8: 7500000-8000000, |
| | chr8: 8000000-8500000, chr8: 8500000-9000000, chr8: 12000000-12500000, chr8: 12500000- |
| | 13000000, chr8: 17500000-18000000, chr8: 27000000-27500000, chr8: 29000000-29500000, |
| | chr8: 30000000-30500000, chr8: 30500000-31000000, chr8: 33000000-33500000, chr8: 35500000- |
| | 36000000, chr8: 37500000-38000000, chr8: 55000000-55500000, chr8: 60500000-61000000, |
| | chr8: 61000000-61500000, chr8: 65500000-66000000, chr8: 66500000-67000000, chr8: 69000000- |
| | 69500000, chr8: 80500000-81000000, chr8: 81500000-82000000, chr8: 84000000-84500000, |
| | chr8: 85500000-86000000, chr8: 86000000-86500000, chr8: 91000000-91500000, chr8: 91500000- |
| | 92000000, chr8: 97000000-97500000, chr8: 100000000-100500000, chr8: 120500000-121000000, |
| | chr8: 124000000-124500000, chr8: 125000000-125500000, chr8: 129500000-130000000, |
| | chr8: 130500000-131000000, chr8: 135000000-135500000, chr8: 135500000-136000000, |
| | chr8: 137500000-138000000, chr8: 139500000-140000000, chr8: 140500000-141000000 |
| 9 | chr9: 0-500000, chr9: 2500000-3000000, chr9: 6500000-7000000, chr9: 8000000-8500000, |
| | chr9: 13500000-14000000, chr9: 15000000-15500000, chr9: 17500000-18000000, chr9: 21500000- |
| | 22000000, chr9: 22000000-22500000, chr9: 28000000-28500000, chr9: 32000000-32500000, |
| | chr9: 33000000-33500000, chr9: 34000000-34500000, chr9: 35500000-36000000, chr9: 36000000- |
| | 36500000, chr9: 39000000-39500000, chr9: 39500000-40000000, chr9: 40000000-40500000, |
| | chr9: 40500000-41000000, chr9: 41000000-41500000, chr9: 41500000-42000000, chr9: 42000000- |
| | 42500000, chr9: 42500000-43000000, chr9: 60500000-61000000, chr9: 61000000-61500000, |
| | chr9: 61500000-62000000, chr9: 62000000-62500000, chr9: 62500000-63000000, chr9: 63000000- |
| | 63500000, chr9: 63500000-64000000, chr9: 64000000-64500000, chr9: 64500000-65000000, |
| | chr9: 65000000-65500000, chr9: 65500000-66000000, chr9: 66000000-66500000, chr9: 66500000- |
| | 67000000, chr9: 67000000-67500000, chr9: 67500000-68000000, chr9: 71500000-72000000, |
| | chr9: 72000000-72500000, chr9: 73500000-74000000, chr9: 75500000-76000000, chr9: 77500000- |
| | 78000000, chr9: 78500000-79000000, chr9: 80500000-81000000, chr9: 85000000-85500000, |
| | chr9: 89000000-89500000, chr9: 89500000-90000000, chr9: 92500000-93000000, chr9: 97500000- |
| | 98000000, chr9: 98000000-98500000, chr9: 105000000-105500000, chr9: 107500000-108000000, |
| | chr9: 112000000-112500000, chr9: 125500000-126000000, chr9: 126000000-126500000, |
| | chr9: 128000000-128500000, chr9: 129500000-130000000, chr9: 132000000-132500000, |
| | chr9: 135000000-135500000 |
| 10 | chr10: 3500000-4000000, chr10: 6000000-6500000, chr10: 7500000-8000000, chr10: 8000000- |
| | 8500000, chr10: 8500000-9000000, chr10: 9000000-9500000, chr10: 12000000-12500000, |
| | chr10: 14500000-15000000, chr10: 17000000-17500000, chr10: 21500000-22000000, |
| | chr10: 24000000-24500000, chr10: 27000000-27500000, chr10: 28000000-28500000, |
| | chr10: 32000000-32500000, chr10: 35000000-35500000, chr10: 36500000-37000000, |
| | chr10: 37000000-37500000, chr10: 38000000-38500000, chr10: 38500000-39000000, |
| | chr10: 42000000-42500000, chr10: 44000000-44500000, chr10: 46500000-47000000, |
| | chr10: 47500000-48000000, chr10: 49000000-49500000, chr10: 53500000-54000000, |
| | chr10: 58000000-58500000, chr10: 59000000-59500000, chr10: 62000000-62500000, |
| | chr10: 62500000-63000000, chr10: 63000000-63500000, chr10: 68000000-68500000, |
| | chr10: 68500000-69000000, chr10: 69500000-70000000, chr10: 72000000-72500000, |
| | chr10: 73000000-73500000, chr10: 82000000-82500000, chr10: 87500000-88000000, |
| | chr10: 88500000-89000000, chr10: 89500000-90000000, chr10: 92500000-93000000, |
| | chr10: 94500000-95000000, chr10: 95500000-96000000, chr10: 97000000-97500000, |
| | chr10: 100000000-100500000, chr10: 101000000-101500000, chr10: 101500000-102000000, |
| | chr10: 102000000-102500000, chr10: 102500000-103000000, chr10: 104000000-104500000, |
| | chr10: 107000000-107500000, chr10: 108000000-108500000, chr10: 109000000-109500000, |
| | chr10: 109500000-110000000, chr10: 110500000-111000000, chr10: 111500000-112000000, |
| | chr10: 116000000-116500000, chr10: 122000000-122500000, chr10: 123500000-124000000, |
| | chr10: 127000000-127500000, chr10: 128500000-129000000, chr10: 129500000-130000000, |
| | chr10: 133500000-133797422 |
| 11 | chr11: 0-500000, chr11: 3500000-4000000, chr11: 6500000-7000000, chr11: 11500000-12000000, |
| | chr11: 12000000-12500000, chr11: 18000000-18500000, chr11: 33000000-33500000, |
| | chr11: 34500000-35000000, chr11: 39500000-40000000, chr11: 46000000-46500000, |
| | chr11: 47500000-48000000, chr11: 48500000-49000000, chr11: 49000000-49500000, |
| | chr11: 49500000-50000000, chr11: 50000000-50500000, chr11: 58500000-59000000, |
| | chr11: 61000000-61500000, chr11: 62000000-62500000, chr11: 62500000-63000000, |
| | chr11: 64500000-65000000, chr11: 65000000-65500000, chr11: 65500000-66000000, |
| | chr11: 66000000-66500000, chr11: 67000000-67500000, chr11: 72000000-72500000, |
| | chr11: 72500000-73000000, chr11: 73000000-73500000, chr11: 76000000-76500000, |
| | chr11: 77500000-78000000, chr11: 78000000-78500000, chr11: 80500000-81000000, |
| | chr11: 86000000-86500000, chr11: 88000000-88500000, chr11: 89500000-90000000, |
| | chr11: 92000000-92500000, chr11: 92500000-93000000, chr11: 95000000-95500000, |
| | chr11: 95500000-96000000, chr11: 100000000-100500000, chr11: 102000000-102500000, |
| | chr11: 103000000-103500000, chr11: 107500000-108000000, chr11: 111500000-112000000, |
| | chr11: 114000000-114500000, chr11: 116000000-116500000, chr11: 117000000-117500000, |
| | chr11: 118500000-119000000, chr11: 119000000-119500000, chr11: 121000000-121500000, |
| | chr11: 128500000-129000000, chr11: 133500000-134000000 |
| 12 | chr12: 500000-1000000, chr12: 6500000-7000000, chr12: 8500000-9000000, chr12: 9500000- |
| | 10000000, chr12: 12000000-12500000, chr12: 12500000-13000000, chr12: 14500000-15000000, |
| | chr12: 15500000-16000000, chr12: 21500000-22000000, chr12: 22000000-22500000, |
| | chr12: 23500000-24000000, chr12: 24500000-25000000, chr12: 25000000-25500000, |
| | chr12: 25500000-26000000, chr12: 30000000-30500000, chr12: 31500000-32000000, |
| | chr12: 34500000-35000000, chr12: 40000000-40500000, chr12: 47000000-47500000, |
| | chr12: 48500000-49000000, chr12: 49500000-50000000, chr12: 50000000-50500000, |
| | chr12: 53000000-53500000, chr12: 53500000-54000000, chr12: 54000000-54500000, |
| | chr12: 54500000-55000000, chr12: 55500000-56000000, chr12: 56000000-56500000, |
| | chr12: 57000000-57500000, chr12: 57500000-58000000, chr12: 62000000-62500000, |
| | chr12: 64500000-65000000, chr12: 65000000-65500000, chr12: 66000000-66500000, |
| | chr12: 68000000-68500000, chr12: 71500000-72000000, chr12: 75000000-75500000, |
| | chr12: 75500000-76000000, chr12: 76000000-76500000, chr12: 89500000-90000000, |
| | chr12: 91000000-91500000, chr12: 91500000-92000000, chr12: 96000000-96500000, |
| | chr12: 98500000-99000000, chr12: 101500000-102000000, chr12: 105000000-105500000, |
| | chr12: 107000000-107500000, chr12: 108500000-109000000, chr12: 110000000-110500000, |
| | chr12: 110500000-111000000, chr12: 111000000-111500000, chr12: 111500000-112000000, |
| | chr12: 112000000-112500000, chr12: 112500000-113000000, chr12: 113000000-113500000, |
| | chr12: 116000000-116500000, chr12: 116500000-117000000, chr12: 118000000-118500000, |
| | chr12: 120000000-120500000, chr12: 121500000-122000000, chr12: 122000000-122500000, |
| | chr12: 123000000-123500000, chr12: 127500000-128000000 |
| 13 | chr13: 18000000-18500000, chr13: 18500000-19000000, chr13: 19000000-19500000, |
| | chr13: 21500000-22000000, chr13: 22000000-22500000, chr13: 27000000-27500000, |
| | chr13: 28000000-28500000, chr13: 32500000-33000000, chr13: 35500000-36000000, |
| | chr13: 36000000-36500000, chr13: 40000000-40500000, chr13: 41000000-41500000, |
| | chr13: 42000000-42500000, chr13: 45000000-45500000, chr13: 48000000-48500000, |
| | chr13: 49500000-50000000, chr13: 73500000-74000000, chr13: 74000000-74500000, |
| | chr13: 100000000-100500000, chr13: 106000000-106500000, chr13: 108000000-108500000 |
| 14 | chr14: 18000000-18500000, chr14: 18500000-19000000, chr14: 19000000-19500000, |
| | chr14: 21000000-21500000, chr14: 22500000-23000000, chr14: 25500000-26000000, |
| | chr14: 31500000-32000000, chr14: 34500000-35000000, chr14: 35000000-35500000, |
| | chr14: 39500000-40000000, chr14: 49500000-50000000, chr14: 50500000-51000000, |
| | chr14: 55000000-55500000, chr14: 60000000-60500000, chr14: 66000000-66500000, |
| | chr14: 70500000-71000000, chr14: 71000000-71500000, chr14: 74000000-74500000, |
| | chr14: 75000000-75500000, chr14: 77000000-77500000, chr14: 80500000-81000000, |
| | chr14: 84000000-84500000, chr14: 86500000-87000000, chr14: 88500000-89000000, |
| | chr14: 89500000-90000000, chr14: 91000000-91500000, chr14: 106500000-107000000 |
| 15 | chr15: 17000000-17500000, chr15: 19500000-20000000, chr15: 20000000-20500000, |
| | chr15: 20500000-21000000, chr15: 21000000-21500000, chr15: 21500000-22000000, |
| | chr15: 23000000-23500000, chr15: 23500000-24000000, chr15: 24000000-24500000, |
| | chr15: 28500000-29000000, chr15: 30000000-30500000, chr15: 30500000-31000000, |
| | chr15: 32000000-32500000, chr15: 34000000-34500000, chr15: 35000000-35500000, |
| | chr15: 36500000-37000000, chr15: 41000000-41500000, chr15: 43000000-43500000, |
| | chr15: 45500000-46000000, chr15: 46000000-46500000, chr15: 47000000-47500000, |
| | chr15: 50000000-50500000, chr15: 51500000-52000000, chr15: 53000000-53500000, |
| | chr15: 60500000-61000000, chr15: 61500000-62000000, chr15: 64500000-65000000, |
| | chr15: 65500000-66000000, chr15: 72000000-72500000, chr15: 73000000-73500000, |
| | chr15: 81500000-82000000, chr15: 84000000-84500000, chr15: 85500000-86000000, |
| | chr15: 88000000-88500000, chr15: 90000000-90500000, chr15: 90500000-91000000, |
| | chr15: 91000000-91500000, chr15: 92500000-93000000, chr15: 94000000-94500000, |
| | chr15: 100000000-100500000 |
| 16 | chr16: 3000000-3500000, chr16: 5000000-5500000, chr16: 8500000-9000000, chr16: 11500000- |
| | 12000000, chr16: 15000000-15500000, chr16: 15500000-16000000, chr16: 16000000-16500000, |
| | chr16: 18000000-18500000, chr16: 23000000-23500000, chr16: 23500000-24000000, |
| | chr16: 24500000-25000000, chr16: 27500000-28000000, chr16: 29000000-29500000, |
| | chr16: 29500000-30000000, chr16: 30500000-31000000, chr16: 32000000-32500000, |
| | chr16: 32500000-33000000, chr16: 33000000-33500000, chr16: 33500000-34000000, |
| | chr16: 34000000-34500000, chr16: 34500000-35000000, chr16: 35000000-35500000, |
| | chr16: 46000000-46500000, chr16: 51000000-51500000, chr16: 52000000-52500000, |
| | chr16: 54000000-54500000, chr16: 57000000-57500000, chr16: 57500000-58000000, |
| | chr16: 67500000-68000000, chr16: 72000000-72500000, chr16: 74500000-75000000, |
| | chr16: 81000000-81500000, chr16: 83500000-84000000, chr16: 90000000-90338345 |
| 17 | chr17: 0-500000, chr17: 1000000-1500000, chr17: 2000000-2500000, chr17: 2500000-3000000, |
| | chr17: 4000000-4500000, chr17: 4500000-5000000, chr17: 5000000-5500000, chr17: 7000000- |
| | 7500000, chr17: 7500000-8000000, chr17: 8000000-8500000, chr17: 8500000-9000000, |
| | chr17: 11500000-12000000, chr17: 16000000-16500000, chr17: 19000000-19500000, |
| | chr17: 19500000-20000000, chr17: 20000000-20500000, chr17: 21500000-22000000, |
| | chr17: 22000000-22500000, chr17: 22500000-23000000, chr17: 26500000-27000000, |
| | chr17: 28500000-29000000, chr17: 29000000-29500000, chr17: 31000000-31500000, |
| | chr17: 34500000-35000000, chr17: 35000000-35500000, chr17: 37000000-37500000, |
| | chr17: 38500000-39000000, chr17: 39000000-39500000, chr17: 39500000-40000000, |
| | chr17: 40000000-40500000, chr17: 40500000-41000000, chr17: 42000000-42500000, |
| | chr17: 43500000-44000000, chr17: 44000000-44500000, chr17: 44500000-45000000, |
| | chr17: 47500000-48000000, chr17: 48000000-48500000, chr17: 48500000-49000000, |
| | chr17: 49000000-49500000, chr17: 50000000-50500000, chr17: 50500000-51000000, |
| | chr17: 59500000-60000000, chr17: 61500000-62000000, chr17: 63500000-64000000, |
| | chr17: 64000000-64500000, chr17: 65000000-65500000, chr17: 65500000-66000000, |
| | chr17: 66000000-66500000, chr17: 68000000-68500000, chr17: 69000000-69500000, |
| | chr17: 71500000-72000000, chr17: 74500000-75000000, chr17: 75000000-75500000, |
| | chr17: 76000000-76500000, chr17: 76500000-77000000 |
| 18 | chr18: 3000000-3500000, chr18: 5500000-6000000, chr18: 7000000-7500000, chr18: 11000000- |
| | 11500000, chr18: 12500000-13000000, chr18: 14000000-14500000, chr18: 15000000-15500000, |
| | chr18: 21000000-21500000, chr18: 24000000-24500000, chr18: 31500000-32000000, |
| | chr18: 35000000-35500000, chr18: 42000000-42500000, chr18: 48500000-49000000, |
| | chr18: 73000000-73500000, chr18: 78000000-78500000 |
| 19 | chr19: 0-500000, chr19: 1000000-1500000, chr19: 2000000-2500000, chr19: 3000000-3500000, |
| | chr19: 4000000-4500000, chr19: 4500000-5000000, chr19: 6500000-7000000, chr19: 7000000- |
| | 7500000, chr19: 7500000-8000000, chr19: 10000000-10500000, chr19: 10500000-11000000, |
| | chr19: 11000000-11500000, chr19: 12500000-13000000, chr19: 13000000-13500000, |
| | chr19: 13500000-14000000, chr19: 14000000-14500000, chr19: 14500000-15000000, |
| | chr19: 15000000-15500000, chr19: 15500000-16000000, chr19: 16500000-17000000, |
| | chr19: 17000000-17500000, chr19: 18000000-18500000, chr19: 18500000-19000000, |
| | chr19: 19000000-19500000, chr19: 20500000-21000000, chr19: 21000000-21500000, |
| | chr19: 21500000-22000000, chr19: 22000000-22500000, chr19: 22500000-23000000, |
| | chr19: 28000000-28500000, chr19: 34000000-34500000, chr19: 35000000-35500000, |
| | chr19: 38000000-38500000, chr19: 38500000-39000000, chr19: 39000000-39500000, |
| | chr19: 40500000-41000000, chr19: 41000000-41500000, chr19: 42000000-42500000, |
| | chr19: 42500000-43000000, chr19: 43500000-44000000, chr19: 44500000-45000000, |
| | chr19: 45000000-45500000, chr19: 45500000-46000000, chr19: 46500000-47000000, |
| | chr19: 47000000-47500000, chr19: 48500000-49000000, chr19: 49500000-50000000, |
| | chr19: 51000000-51500000, chr19: 54000000-54500000, chr19: 55000000-55500000, |
| | chr19: 58500000-58617616 |
| 20 | chr20: 1500000-2000000, chr20: 3500000-4000000, chr20: 4500000-5000000, chr20: 5000000- |
| | 5500000, chr20: 8000000-8500000, chr20: 10000000-10500000, chr20: 11500000-12000000, |
| | chr20: 19000000-19500000, chr20: 20000000-20500000, chr20: 21500000-22000000, |
| | chr20: 24500000-25000000, chr20: 25500000-26000000, chr20: 26000000-26500000, |
| | chr20: 28500000-29000000, chr20: 29000000-29500000, chr20: 29500000-30000000, |
| | chr20: 30000000-30500000, chr20: 30500000-31000000, chr20: 31500000-32000000, |
| | chr20: 33000000-33500000, chr20: 34500000-35000000, chr20: 38500000-39000000, |
| | chr20: 41000000-41500000, chr20: 45000000-45500000, chr20: 45500000-46000000, |
| | chr20: 48500000-49000000, chr20: 49000000-49500000, chr20: 49500000-50000000, |
| | chr20: 50000000-50500000 |
| 21 | chr21: 5000000-5500000, chr21: 5500000-6000000, chr21: 6000000-6500000, chr21: 6500000- |
| | 7000000, chr21: 7000000-7500000, chr21: 7500000-8000000, chr21: 8000000-8500000, |
| | chr21: 8500000-9000000, chr21: 9000000-9500000, chr21: 9500000-10000000, chr21: 10000000- |
| | 10500000, chr21: 10500000-11000000, chr21: 13000000-13500000, chr21: 13500000-14000000, |
| | chr21: 25500000-26000000, chr21: 29000000-29500000, chr21: 34500000-35000000, |
| | chr21: 42000000-42500000 |
| 22 | chr22: 10500000-11000000, chr22: 11000000-11500000, chr22: 11500000-12000000, |
| | chr22: 12000000-12500000, chr22: 12500000-13000000, chr22: 15000000-15500000, |
| | chr22: 15500000-16000000, chr22: 16000000-16500000, chr22: 16500000-17000000, |
| | chr22: 18000000-18500000, chr22: 18500000-19000000, chr22: 21000000-21500000, |
| | chr22: 23000000-23500000, chr22: 31000000-31500000, chr22: 31500000-32000000, |
| | chr22: 35500000-36000000, chr22: 36000000-36500000, chr22: 36500000-37000000, |
| | chr22: 39500000-40000000, chr22: 41500000-42000000 |

TABLE 18

| | |
|---|---|
| 1 | chr1: 500000-1000000, chr1: 1500000-2000000, chr1: 3000000-3500000, chr1: 8000000-8500000, |
| | chr1: 8500000-9000000, chr1: 9500000-10000000, chr1: 10000000-10500000, chr1: 11500000- |
| | 12000000, chr1: 12000000-12500000, chr1: 13000000-13500000, chr1: 15500000-16000000, |
| | chr1: 16000000-16500000, chr1: 16500000-17000000, chr1: 19000000-19500000, chr1: 19500000- |
| | 20000000, chr1: 20000000-20500000, chr1: 20500000-21000000, chr1: 21000000-21500000, |
| | chr1: 22000000-22500000, chr1: 23000000-23500000, chr1: 24500000-25000000, chr1: 25500000- |
| | 26000000, chr1: 26500000-27000000, chr1: 27000000-27500000, chr1: 27500000-28000000, |
| | chr1: 28000000-28500000, chr1: 28500000-29000000, chr1: 30500000-31000000, chr1: 31000000- |
| | 31500000, chr1: 31500000-32000000, chr1: 32000000-32500000, chr1: 32500000-33000000, |
| | chr1: 33000000-33500000, chr1: 35000000-35500000, chr1: 35500000-36000000, chr1: 36000000- |
| | 36500000, chr1: 36500000-37000000, chr1: 37500000-38000000, chr1: 39000000-39500000, |
| | chr1: 39500000-40000000, chr1: 40000000-40500000, chr1: 40500000-41000000, chr1: 42000000- |
| | 42500000, chr1: 42500000-43000000, chr1: 44500000-45000000, chr1: 45500000-46000000, |
| | chr1: 46000000-46500000, chr1: 49000000-49500000, chr1: 51500000-52000000, chr1: 52000000- |
| | 52500000, chr1: 52500000-53000000, chr1: 53500000-54000000, chr1: 54000000-54500000, |
| | chr1: 56500000-57000000, chr1: 58500000-59000000, chr1: 63500000-64000000, chr1: 65000000- |
| | 65500000, chr1: 66000000-66500000, chr1: 67500000-68000000, chr1: 68500000-69000000, |
| | chr1: 70000000-70500000, chr1: 73000000-73500000, chr1: 77000000-77500000, chr1: 77500000- |
| | 78000000, chr1: 79000000-79500000, chr1: 81500000-82000000, chr1: 83000000-83500000, |
| | chr1: 88500000-89000000, chr1: 89500000-90000000, chr1: 90000000-90500000, chr1: 91500000- |
| | 92000000, chr1: 93000000-93500000, chr1: 94500000-95000000, chr1: 98500000-99000000, |
| | chr1: 101000000-101500000, chr1: 104500000-105000000, chr1: 107500000-108000000, |
| | chr1: 108500000-109000000, chr1: 109000000-109500000, chr1: 109500000-110000000, |
| | chr1: 110000000-110500000, chr1: 110500000-111000000, chr1: 111500000-112000000, |
| | chr1: 112500000-113000000, chr1: 114000000-114500000, chr1: 116000000-116500000, |
| | chr1: 116500000-117000000, chr1: 118500000-119000000, chr1: 120000000-120500000, |
| | chr1: 120500000-121000000, chr1: 121000000-121500000, chr1: 125000000-125500000, |
| | chr1: 143000000-143500000, chr1: 143500000-144000000, chr1: 144000000-144500000, |
| | chr1: 144500000-145000000, chr1: 145000000-145500000, chr1: 146000000-146500000, |
| | chr1: 146500000-147000000, chr1: 147500000-148000000, chr1: 148500000-149000000, |
| | chr1: 149000000-149500000, chr1: 149500000-150000000, chr1: 150000000-150500000, |
| | chr1: 150500000-151000000, chr1: 151000000-151500000, chr1: 151500000-152000000, |
| | chr1: 152000000-152500000, chr1: 153500000-154000000, chr1: 154500000-155000000, |
| | chr1: 155000000-155500000, chr1: 155500000-156000000, chr1: 156000000-156500000, |
| | chr1: 156500000-157000000, chr1: 158000000-158500000, chr1: 158500000-159000000, |
| | chr1: 159000000-159500000, chr1: 160000000-160500000, chr1: 160500000-161000000, |
| | chr1: 161000000-161500000, chr1: 162000000-162500000, chr1: 163500000-164000000, |
| | chr1: 165500000-166000000, chr1: 167000000-167500000, chr1: 169500000-170000000, |
| | chr1: 171500000-172000000, chr1: 172500000-173000000, chr1: 173500000-174000000, |
| | chr1: 175000000-175500000, chr1: 177500000-178000000, chr1: 179000000-179500000, |
| | chr1: 181000000-181500000, chr1: 182000000-182500000, chr1: 182500000-183000000, |
| | chr1: 183000000-183500000, chr1: 183500000-184000000, chr1: 184500000-185000000, |
| | chr1: 186000000-186500000, chr1: 192500000-193000000, chr1: 198500000-199000000, |
| | chr1: 200000000-200500000, chr1: 200500000-201000000, chr1: 201500000-202000000, |
| | chr1: 202500000-203000000, chr1: 203000000-203500000, chr1: 203500000-204000000, |
| | chr1: 204000000-204500000, chr1: 205000000-205500000, chr1: 206500000-207000000, |
| | chr1: 207000000-207500000, chr1: 208000000-208500000, chr1: 211500000-212000000, |
| | chr1: 212500000-213000000, chr1: 214000000-214500000, chr1: 221500000-222000000, |
| | chr1: 222500000-223000000, chr1: 224000000-224500000, chr1: 224500000-225000000, |
| | chr1: 225500000-226000000, chr1: 226000000-226500000, chr1: 229000000-229500000, |
| | chr1: 230500000-231000000, chr1: 231500000-232000000, chr1: 232000000-232500000, |
| | chr1: 233000000-233500000, chr1: 235000000-235500000, chr1: 235500000-236000000, |
| | chr1: 242000000-242500000, chr1: 242500000-243000000, chr1: 243500000-244000000, |
| | chr1: 244000000-244500000, chr1: 244500000-245000000, chr1: 246500000-247000000 |
| 2 | chr2: 9500000-10000000, chr2: 13500000-14000000, chr2: 14500000-15000000, chr2: 15500000- |
| | 16000000, chr2: 21500000-22000000, chr2: 24500000-25000000, chr2: 25500000-26000000, |
| | chr2: 26000000-26500000, chr2: 27000000-27500000, chr2: 27500000-28000000, chr2: 28500000- |
| | 29000000, chr2: 30000000-30500000, chr2: 36500000-37000000, chr2: 37000000-37500000, |
| | chr2: 37500000-38000000, chr2: 38500000-39000000, chr2: 39000000-39500000, chr2: 42000000- |
| | 42500000, chr2: 45500000-46000000, chr2: 47500000-48000000, chr2: 48000000-48500000, |
| | chr2: 53000000-53500000, chr2: 54000000-54500000, chr2: 60500000-61000000, chr2: 61000000- |
| | 61500000, chr2: 61500000-62000000, chr2: 62000000-62500000, chr2: 64000000-64500000, |
| | chr2: 64500000-65000000, chr2: 65000000-65500000, chr2: 68000000-68500000, chr2: 69000000- |
| | 69500000, chr2: 69500000-70000000, chr2: 70000000-70500000, chr2: 70500000-71000000, |
| | chr2: 71000000-71500000, chr2: 71500000-72000000, chr2: 72000000-72500000, chr2: 74500000- |
| | 75000000, chr2: 76000000-76500000, chr2: 76500000-77000000, chr2: 78500000-79000000, |
| | chr2: 79000000-79500000, chr2: 81000000-81500000, chr2: 84000000-84500000, chr2: 84500000- |
| | 85000000, chr2: 85500000-86000000, chr2: 87000000-87500000, chr2: 87500000-88000000, |
| | chr2: 89000000-89500000, chr2: 89500000-90000000, chr2: 90000000-90500000, chr2: 91000000- |
| | 91500000, chr2: 91500000-92000000, chr2: 92000000-92500000, chr2: 94500000-95000000, |
| | chr2: 96000000-96500000, chr2: 96500000-97000000, chr2: 97000000-97500000, chr2: 97500000- |
| | 98000000, chr2: 99000000-99500000, chr2: 101500000-102000000, chr2: 102000000-102500000, |
| | chr2: 103000000-103500000, chr2: 105500000-106000000, chr2: 107500000-108000000, |
| | chr2: 109500000-110000000, chr2: 112500000-113000000, chr2: 113500000-114000000, |
| | chr2: 114500000-115000000, chr2: 116000000-116500000, chr2: 117000000-117500000, |
| | chr2: 119500000-120000000, chr2: 122000000-122500000, chr2: 125000000-125500000, |
| | chr2: 126500000-127000000, chr2: 128000000-128500000, chr2: 129000000-129500000, |
| | chr2: 129500000-130000000, chr2: 130000000-130500000, chr2: 130500000-131000000, |
| | chr2: 131500000-132000000, chr2: 132000000-132500000, chr2: 133000000-133500000, |
| | chr2: 134000000-134500000, chr2: 135500000-136000000, chr2: 136000000-136500000, |
| | chr2: 143000000-143500000, chr2: 143500000-144000000, chr2: 144000000-144500000, |
| | chr2: 144500000-145000000, chr2: 145500000-146000000, chr2: 147500000-148000000, |
| | chr2: 148500000-149000000, chr2: 150000000-150500000, chr2: 150500000-151000000, |
| | chr2: 151000000-151500000, chr2: 153000000-153500000, chr2: 156000000-156500000, |
| | chr2: 158000000-158500000, chr2: 161000000-161500000, chr2: 162000000-162500000, |
| | chr2: 165500000-166000000, chr2: 168000000-168500000, chr2: 168500000-169000000, |
| | chr2: 171000000-171500000, chr2: 171500000-172000000, chr2: 174500000-175000000, |
| | chr2: 176000000-176500000, chr2: 177000000-177500000, chr2: 178000000-178500000, |
| | chr2: 181000000-181500000, chr2: 181500000-182000000, chr2: 183500000-184000000, |
| | chr2: 189500000-190000000, chr2: 190000000-190500000, chr2: 190500000-191000000, |
| | chr2: 196000000-196500000, chr2: 197000000-197500000, chr2: 198000000-198500000, |
| | chr2: 200500000-201000000, chr2: 201000000-201500000, chr2: 204000000-204500000, |
| | chr2: 207000000-207500000, chr2: 207500000-208000000, chr2: 208500000-209000000, |
| | chr2: 215500000-216000000, chr2: 216000000-216500000, chr2: 217500000-218000000, |
| | chr2: 218000000-218500000, chr2: 218500000-219000000, chr2: 219000000-219500000, |
| | chr2: 220000000-220500000, chr2: 224500000-225000000, chr2: 225000000-225500000, |
| | chr2: 226000000-226500000, chr2: 226500000-227000000, chr2: 227000000-227500000, |
| | chr2: 228000000-228500000, chr2: 230000000-230500000, chr2: 230500000-231000000, |
| | chr2: 231000000-231500000, chr2: 231500000-232000000, chr2: 235500000-236000000, |
| | chr2: 238500000-239000000, chr2: 240500000-241000000 |
| 3 | chr3: 500000-1000000, chr3: 6500000-7000000, chr3: 9000000-9500000, chr3: 9500000-10000000, |
| | chr3: 10000000-10500000, chr3: 12500000-13000000, chr3: 13500000-14000000, chr3: 14000000- |
| | 14500000, chr3: 16500000-17000000, chr3: 17000000-17500000, chr3: 22000000-22500000, |
| | chr3: 23500000-24000000, chr3: 25500000-26000000, chr3: 30000000-30500000, chr3: 30500000- |
| | 31000000, chr3: 31500000-32000000, chr3: 32500000-33000000, chr3: 33000000-33500000, |
| | chr3: 36500000-37000000, chr3: 37000000-37500000, chr3: 39000000-39500000, chr3: 42000000- |
| | 42500000, chr3: 43500000-44000000, chr3: 45000000-45500000, chr3: 46000000-46500000, |
| | chr3: 47000000-47500000, chr3: 47500000-48000000, chr3: 48500000-49000000, chr3: 49000000- |
| | 49500000, chr3: 49500000-50000000, chr3: 51500000-52000000, chr3: 52000000-52500000, |
| | chr3: 56500000-57000000, chr3: 57500000-58000000, chr3: 58500000-59000000, chr3: 59000000- |
| | 59500000, chr3: 64500000-65000000, chr3: 65000000-65500000, chr3: 66000000-66500000, |
| | chr3: 67500000-68000000, chr3: 68500000-69000000, chr3: 69000000-69500000, chr3: 69500000- |
| | 70000000, chr3: 70000000-70500000, chr3: 71000000-71500000, chr3: 71500000-72000000, |
| | chr3: 72000000-72500000, chr3: 72500000-73000000, chr3: 73500000-74000000, chr3: 75000000- |
| | 75500000, chr3: 75500000-76000000, chr3: 76500000-77000000, chr3: 77500000-78000000, |
| | chr3: 78000000-78500000, chr3: 88500000-89000000, chr3: 89000000-89500000, chr3: 89500000- |
| | 90000000, chr3: 91000000-91500000, chr3: 95500000-96000000, chr3: 101500000-102000000, |
| | chr3: 107500000-108000000, chr3: 108000000-108500000, chr3: 112000000-112500000, |
| | chr3: 113000000-113500000, chr3: 116500000-117000000, chr3: 119500000-120000000, |
| | chr3: 122000000-122500000, chr3: 127000000-127500000, chr3: 128500000-129000000, |
| | chr3: 129000000-129500000, chr3: 135000000-135500000, chr3: 135500000-136000000, |
| | chr3: 141000000-141500000, chr3: 141500000-142000000, chr3: 143000000-143500000, |
| | chr3: 143500000-144000000, chr3: 147500000-148000000, chr3: 148000000-148500000, |
| | chr3: 150000000-150500000, chr3: 151000000-151500000, chr3: 152000000-152500000, |
| | chr3: 155500000-156000000, chr3: 156500000-157000000, chr3: 157000000-157500000, |
| | chr3: 161000000-161500000, chr3: 161500000-162000000, chr3: 162000000-162500000, |
| | chr3: 163000000-163500000, chr3: 166500000-167000000, chr3: 169500000-170000000, |
| | chr3: 170000000-170500000, chr3: 171000000-171500000, chr3: 171500000-172000000, |
| | chr3: 172000000-172500000, chr3: 179500000-180000000, chr3: 183000000-183500000, |
| | chr3: 184000000-184500000, chr3: 184500000-185000000, chr3: 186000000-186500000, |
| | chr3: 186500000-187000000, chr3: 187500000-188000000, chr3: 188500000-189000000, |
| | chr3: 189000000-189500000, chr3: 191500000-192000000, chr3: 192000000-192500000, |
| | chr3: 194500000-195000000, chr3: 197000000-197500000, chr3: 198000000-198295559 |
| 4 | chr4: 2500000-3000000, chr4: 6000000-6500000, chr4: 8500000-9000000, chr4: 9000000-9500000, |
| | chr4: 9500000-10000000, chr4: 10000000-10500000, chr4: 15000000-15500000, chr4: 25000000- |
| | 25500000, chr4: 26000000-26500000, chr4: 27000000-27500000, chr4: 27500000-28000000, |
| | chr4: 36000000-36500000, chr4: 37500000-38000000, chr4: 38000000-38500000, chr4: 38500000- |
| | 39000000, chr4: 39500000-40000000, chr4: 40000000-40500000, chr4: 40500000-41000000, |
| | chr4: 45000000-45500000, chr4: 47500000-48000000, chr4: 49000000-49500000, chr4: 49500000- |
| | 50000000, chr4: 52000000-52500000, chr4: 57500000-58000000, chr4: 58000000-58500000, |
| | chr4: 59000000-59500000, chr4: 60500000-61000000, chr4: 61500000-62000000, chr4: 62000000- |
| | 62500000, chr4: 65000000-65500000, chr4: 66500000-67000000, chr4: 67500000-68000000, |
| | chr4: 68000000-68500000, chr4: 68500000-69000000, chr4: 70500000-71000000, chr4: 74000000- |
| | 74500000, chr4: 77000000-77500000, chr4: 78500000-79000000, chr4: 80000000-80500000, |
| | chr4: 80500000-81000000, chr4: 83000000-83500000, chr4: 83500000-84000000, chr4: 84500000- |
| | 85000000, chr4: 88500000-89000000, chr4: 89000000-89500000, chr4: 98500000-99000000, |
| | chr4: 99500000-100000000, chr4: 101000000-101500000, chr4: 102000000-102500000, |
| | chr4: 103000000-103500000, chr4: 105000000-105500000, chr4: 108000000-108500000, |
| | chr4: 111000000-111500000, chr4: 111500000-112000000, chr4: 113500000-114000000, |
| | chr4: 117500000-118000000, chr4: 121500000-122000000, chr4: 126500000-127000000, |
| | chr4: 129500000-130000000, chr4: 130500000-131000000, chr4: 131500000-132000000, |
| | chr4: 133500000-134000000, chr4: 135000000-135500000, chr4: 140000000-140500000, |
| | chr4: 142000000-142500000, chr4: 151000000-151500000, chr4: 152500000-153000000, |
| | chr4: 153000000-153500000, chr4: 153500000-154000000, chr4: 156000000-156500000, |
| | chr4: 160000000-160500000, chr4: 164500000-165000000, chr4: 165500000-166000000, |
| | chr4: 167000000-167500000, chr4: 171500000-172000000, chr4: 172000000-172500000, |
| | chr4: 175000000-175500000, chr4: 178500000-179000000, chr4: 184000000-184500000, |
| | chr4: 184500000-185000000, chr4: 185500000-186000000, chr4: 187000000-187500000, |
| | chr4: 187500000-188000000, chr4: 188000000-188500000, chr4: 188500000-189000000, |
| | chr4: 189500000-190000000, chr4: 190000000-190214555 |
| 5 | chr5: 1500000-2000000, chr5: 2000000-2500000, chr5: 3000000-3500000, chr5: 4000000-4500000, |
| | chr5: 4500000-5000000, chr5: 7000000-7500000, chr5: 9000000-9500000, chr5: 9500000- |
| | 10000000, chr5: 10500000-11000000, chr5: 13500000-14000000, chr5: 14500000-15000000, |
| | chr5: 16000000-16500000, chr5: 17500000-18000000, chr5: 19000000-19500000, chr5: 20000000- |
| | 20500000, chr5: 21000000-21500000, chr5: 22500000-23000000, chr5: 23000000-23500000, |
| | chr5: 26000000-26500000, chr5: 31500000-32000000, chr5: 34000000-34500000, chr5: 36500000- |
| | 37000000, chr5: 39000000-39500000, chr5: 40500000-41000000, chr5: 43000000-43500000, |
| | chr5: 44000000-44500000, chr5: 45500000-46000000, chr5: 50500000-51000000, chr5: 51000000- |
| | 51500000, chr5: 56000000-56500000, chr5: 56500000-57000000, chr5: 59000000-59500000, |
| | chr5: 59500000-60000000, chr5: 60500000-61000000, chr5: 61000000-61500000, chr5: 63500000- |
| | 64000000, chr5: 65000000-65500000, chr5: 66000000-66500000, chr5: 67000000-67500000, |
| | chr5: 67500000-68000000, chr5: 68000000-68500000, chr5: 69500000-70000000, chr5: 70000000- |
| | 70500000, chr5: 70500000-71000000, chr5: 71000000-71500000, chr5: 74500000-75000000, |
| | chr5: 78000000-78500000, chr5: 79500000-80000000, chr5: 80500000-81000000, chr5: 84000000- |
| | 84500000, chr5: 89000000-89500000, chr5: 91000000-91500000, chr5: 94500000-95000000, |
| | chr5: 95500000-96000000, chr5: 96500000-97000000, chr5: 97500000-98000000, chr5: 99500000- |
| | 100000000, chr5: 100500000-101000000, chr5: 102500000-103000000, chr5: 109500000- |
| | 110000000, chr5: 110000000-110500000, chr5: 112500000-113000000, chr5: 113000000- |
| | 113500000, chr5: 119000000-119500000, chr5: 124000000-124500000, chr5: 125000000- |
| | 125500000, chr5: 131000000-131500000, chr5: 131500000-132000000, chr5: 132000000- |
| | 132500000, chr5: 132500000-133000000, chr5: 134500000-135000000, chr5: 136500000- |
| | 137000000, chr5: 137500000-138000000, chr5: 138000000-138500000, chr5: 138500000- |
| | 139000000, chr5: 139000000-139500000, chr5: 139500000-140000000, chr5: 140500000 |
| | 141000000, chr5: 141000000-141500000, chr5: 141500000-142000000, chr5: 142000000- |
| | 142500000, chr5: 142500000-143000000, chr5: 143000000-143500000, chr5: 146000000- |
| | 146500000, chr5: 148500000-149000000, chr5: 149000000-149500000, chr5: 149500000- |
| | 150000000, chr5: 151000000-151500000, chr5: 151500000-152000000, chr5: 157000000- |
| | 157500000, chr5: 157500000-158000000, chr5: 158000000-158500000, chr5: 159000000 |
| | 159500000, chr5: 160000000-160500000, chr5: 161500000-162000000, chr5: 162500000- |
| | 163000000, chr5: 166500000-167000000, chr5: 169500000-170000000, chr5: 170000000- |
| | 170500000, chr5: 171500000-172000000, chr5: 172500000-173000000, chr5: 177000000- |
| | 177500000, chr5: 180000000-180500000, chr5: 181000000-181500000 |
| 6 | chr6: 2500000-3000000, chr6: 4000000-4500000, chr6: 6500000-7000000, chr6: 8500000-9000000, |
| | chr6: 9000000-9500000, chr6: 10500000-11000000, chr6: 11000000-11500000, chr6: 12000000- |
| | 12500000, chr6: 13500000-14000000, chr6: 14000000-14500000, chr6: 14500000-15000000, |
| | chr6: 15000000-15500000, chr6: 16000000-16500000, chr6: 17500000-18000000, chr6: 18000000- |
| | 18500000, chr6: 19500000-20000000, chr6: 20000000-20500000, chr6: 25000000-25500000, |
| | chr6: 25500000-26000000, chr6: 26000000-26500000, chr6: 27500000-28000000, chr6: 29500000- |
| | 30000000, chr6: 30000000-30500000, chr6: 30500000-31000000, chr6: 31000000-31500000, |
| | chr6: 31500000-32000000, chr6: 32500000-33000000, chr6: 33000000-33500000, chr6: 33500000- |
| | 34000000, chr6: 34500000-35000000, chr6: 35000000-35500000, chr6: 36000000-36500000, |
| | chr6: 36500000-37000000, chr6: 37000000-37500000, chr6: 37500000-38000000, chr6: 40500000- |
| | 41000000, chr6: 41000000-41500000, chr6: 41500000-42000000, chr6: 42500000-43000000, |
| | chr6: 43000000-43500000, chr6: 44000000-44500000, chr6: 47000000-47500000, chr6: 49500000- |
| | 50000000, chr6: 51000000-51500000, chr6: 53000000-53500000, chr6: 53500000-54000000, |
| | chr6: 55500000-56000000, chr6: 57500000-58000000, chr6: 58000000-58500000, chr6: 58500000- |
| | 59000000, chr6: 60000000-60500000, chr6: 60500000-61000000, chr6: 62000000-62500000, |
| | chr6: 63500000-64000000, chr6: 68500000-69000000, chr6: 70500000-71000000, chr6: 74000000- |
| | 74500000, chr6: 74500000-75000000, chr6: 77000000-77500000, chr6: 87500000-88000000, |
| | chr6: 88500000-89000000, chr6: 89000000-89500000, chr6: 92500000-93000000, chr6: 94500000- |
| | 95000000, chr6: 97000000-97500000, chr6: 104000000-104500000, chr6: 105500000-106000000, |
| | chr6: 106000000-106500000, chr6: 108500000-109000000, chr6: 110500000-111000000, |
| | chr6: 111000000-111500000, chr6: 111500000-112000000, chr6: 112000000-112500000, |
| | chr6: 112500000-113000000, chr6: 119000000-119500000, chr6: 119500000-120000000, |
| | chr6: 128000000-128500000, chr6: 129500000-130000000, chr6: 130000000-130500000, |
| | chr6: 130500000-131000000, chr6: 133000000-133500000, chr6: 134000000-134500000, |
| | chr6: 134500000-135000000, chr6: 135000000-135500000, chr6: 135500000-136000000, |
| | chr6: 137000000-137500000, chr6: 137500000-138000000, chr6: 138000000-138500000, |
| | chr6: 139000000-139500000, chr6: 143500000-144000000, chr6: 144000000-144500000, |
| | chr6: 147000000-147500000, chr6: 149000000-149500000, chr6: 149500000-150000000, |
| | chr6: 154000000-154500000, chr6: 159500000-160000000, chr6: 162000000-162500000, |
| | chr6: 165000000-165500000, chr6: 168000000-168500000 |
| 7 | chr7: 6000000-6500000, chr7: 7500000-8000000, chr7: 26000000-26500000, chr7: 27000000- |
| | 27500000, chr7: 30000000-30500000, chr7: 32000000-32500000, chr7: 37000000-37500000, |
| | chr7: 39500000-40000000, chr7: 45500000-46000000, chr7: 46000000-46500000, chr7: 48000000- |
| | 48500000, chr7: 51500000-52000000, chr7: 53000000-53500000, chr7: 53500000-54000000, |
| | chr7: 54000000-54500000, chr7: 56000000-56500000, chr7: 56500000-57000000, chr7: 57000000- |
| | 57500000, chr7: 57500000-58000000, chr7: 58000000-58500000, chr7: 63000000-63500000, |
| | chr7: 63500000-64000000, chr7: 65000000-65500000, chr7: 66500000-67000000, chr7: 68000000- |
| | 68500000, chr7: 72000000-72500000, chr7: 73500000-74000000, chr7: 74500000-75000000, |
| | chr7: 75500000-76000000, chr7: 77500000-78000000, chr7: 88000000-88500000, chr7: 89500000- |
| | 90000000, chr7: 97500000-98000000, chr7: 98000000-98500000, chr7: 99000000-99500000, |
| | chr7: 100000000-100500000, chr7: 100500000-101000000, chr7: 101000000-101500000, |
| | chr7: 103000000-103500000, chr7: 105500000-106000000, chr7: 106000000-106500000, |
| | chr7: 107000000-107500000, chr7: 109500000-110000000, chr7: 112000000-112500000, |
| | chr7: 112500000-113000000, chr7: 114500000-115000000, chr7: 116000000-116500000, |
| | chr7: 127500000-128000000, chr7: 128000000-128500000, chr7: 129000000-129500000, |
| | chr7: 129500000-130000000, chr7: 130000000-130500000, chr7: 130500000-131000000, |
| | chr7: 131000000-131500000, chr7: 131500000-132000000, chr7: 137000000-137500000, |
| | chr7: 139000000-139500000, chr7: 140000000-140500000, chr7: 147500000-148000000, |
| | chr7: 148500000-149000000, chr7: 149000000-149500000, chr7: 151000000-151500000, |
| | chr7: 151500000-152000000, chr7: 152500000-153000000, chr7: 153500000-154000000, |
| | chr7: 154000000-154500000 |
| 8 | chr8: 0-500000, chr8: 3000000-3500000, chr8: 4000000-4500000, chr8: 7000000-7500000, |
| | chr8: 7500000-8000000, chr8: 8000000-8500000, chr8: 11000000-11500000, chr8: 12000000- |
| | 12500000, chr8: 12500000-13000000, chr8: 18000000-18500000, chr8: 19500000-20000000, |
| | chr8: 20000000-20500000, chr8: 20500000-21000000, chr8: 21000000-21500000, chr8: 22500000- |
| | 23000000, chr8: 23500000-24000000, chr8: 25000000-25500000, chr8: 27000000-27500000, |
| | chr8: 28500000-29000000, chr8: 30000000-30500000, chr8: 30500000-31000000, chr8: 32500000- |
| | 33000000, chr8: 35000000-35500000, chr8: 35500000-36000000, chr8: 36000000-36500000, |
| | chr8: 37500000-38000000, chr8: 42000000-42500000, chr8: 42500000-43000000, chr8: 43500000- |
| | 44000000, chr8: 47500000-48000000, chr8: 49000000-49500000, chr8: 55000000-55500000, |
| | chr8: 58500000-59000000, chr8: 60500000-61000000, chr8: 62000000-62500000, chr8: 64500000- |
| | 65000000, chr8: 66000000-66500000, chr8: 66500000-67000000, chr8: 68500000-69000000, |
| | chr8: 69000000-69500000, chr8: 70000000-70500000, chr8: 72000000-72500000, chr8: 73500000- |
| | 74000000, chr8: 74500000-75000000, chr8: 77000000-77500000, chr8: 79500000-80000000, |
| | chr8: 85500000-86000000, chr8: 87000000-87500000, chr8: 87500000-88000000, chr8: 88000000- |
| | 88500000, chr8: 91500000-92000000, chr8: 94500000-95000000, chr8: 95000000-95500000, |
| | chr8: 96500000-97000000, chr8: 101000000-101500000, chr8: 104000000-104500000, |
| | chr8: 106000000-106500000, chr8: 115000000-115500000, chr8: 120500000-121000000, |
| | chr8: 121500000-122000000, chr8: 122500000-123000000, chr8: 123500000-124000000, |
| | chr8: 124000000-124500000, chr8: 124500000-125000000, chr8: 125000000-125500000, |
| | chr8: 127500000-128000000, chr8: 128000000-128500000, chr8: 128500000-129000000, |
| | chr8: 130000000-130500000, chr8: 130500000-131000000, chr8: 133000000-133500000, |
| | chr8: 134000000-134500000, chr8: 138000000-138500000, chr8: 139500000-140000000, |
| | chr8: 140000000-140500000, chr8: 140500000-141000000 |
| 9 | chr9: 3500000-4000000, chr9: 4500000-5000000, chr9: 5500000-6000000, chr9: 10500000- |
| | 11000000, chr9: 15000000-15500000, chr9: 17000000-17500000, chr9: 19000000-19500000, |
| | chr9: 21000000-21500000, chr9: 23500000-24000000, chr9: 33000000-33500000, chr9: 34000000- |
| | 34500000, chr9: 34500000-35000000, chr9: 35000000-35500000, chr9: 35500000-36000000, |
| | chr9: 36000000-36500000, chr9: 37000000-37500000, chr9: 37500000-38000000, chr9: 39000000- |
| | 39500000, chr9: 39500000-40000000, chr9: 40000000-40500000, chr9: 40500000-41000000, |
| | chr9: 41000000-41500000, chr9: 41500000-42000000, chr9: 42000000-42500000, chr9: 42500000- |
| | 43000000, chr9: 43000000-43500000, chr9: 60500000-61000000, chr9: 61500000-62000000, |
| | chr9: 62000000-62500000, chr9: 62500000-63000000, chr9: 63000000-63500000, chr9: 63500000- |
| | 64000000, chr9: 64000000-64500000, chr9: 64500000-65000000, chr9: 65000000-65500000, |
| | chr9: 65500000-66000000, chr9: 66000000-66500000, chr9: 66500000-67000000, chr9: 67000000- |
| | 67500000, chr9: 67500000-68000000, chr9: 75000000-75500000, chr9: 78000000-78500000, |
| | chr9: 80500000-81000000, chr9: 83500000-84000000, chr9: 84000000-84500000, chr9: 85500000- |
| | 86000000, chr9: 86500000-87000000, chr9: 87000000-87500000, chr9: 89000000-89500000, |
| | chr9: 99000000-99500000, chr9: 102000000-102500000, chr9: 103000000-103500000, |
| | chr9: 105000000-105500000, chr9: 106000000-106500000, chr9: 109000000-109500000, |
| | chr9: 113500000-114000000, chr9: 114500000-115000000, chr9: 117000000-117500000, |
| | chr9: 117500000-118000000, chr9: 120500000-121000000, chr9: 122000000-122500000, |
| | chr9: 124500000-125000000, chr9: 125000000-125500000, chr9: 125500000-126000000, |
| | chr9: 126500000-127000000, chr9: 128000000-128500000, chr9: 128500000-129000000, |
| | chr9: 129000000-129500000, chr9: 129500000-130000000, chr9: 132000000-132500000, |
| | chr9: 135000000-135500000 |
| 10 | chr10: 0-500000, chr10: 2000000-2500000, chr10: 3000000-3500000, chr10: 3500000-4000000, |
| | chr10: 4000000-4500000, chr10: 6000000-6500000, chr10: 7000000-7500000, chr10: 8000000- |
| | 8500000, chr10: 9000000-9500000, chr10: 13500000-14000000, chr10: 16500000-17000000, |
| | chr10: 19000000-19500000, chr10: 20500000-21000000, chr10: 23000000-23500000, |
| | chr10: 27500000-28000000, chr10: 28000000-28500000, chr10: 30000000-30500000, |
| | chr10: 31500000-32000000, chr10: 35000000-35500000, chr10: 35500000-36000000, |
| | chr10: 36500000-37000000, chr10: 37000000-37500000, chr10: 38000000-38500000, |
| | chr10: 38500000-39000000, chr10: 42000000-42500000, chr10: 42500000-43000000, |
| | chr10: 43000000-43500000, chr10: 44000000-44500000, chr10: 46500000-47000000, |
| | chr10: 47500000-48000000, chr10: 52000000-52500000, chr10: 52500000-53000000, |
| | chr10: 55500000-56000000, chr10: 58000000-58500000, chr10: 62000000-62500000, |
| | chr10: 63000000-63500000, chr10: 64000000-64500000, chr10: 68000000-68500000, |
| | chr10: 69000000-69500000, chr10: 72000000-72500000, chr10: 73000000-73500000, |
| | chr10: 73500000-74000000, chr10: 74500000-75000000, chr10: 76500000-77000000, |
| | chr10: 79000000-79500000, chr10: 84500000-85000000, chr10: 87000000-87500000, |
| | chr10: 88000000-88500000, chr10: 89000000-89500000, chr10: 92500000-93000000, |
| | chr10: 94500000-95000000, chr10: 96500000-97000000, chr10: 97000000-97500000, |
| | chr10: 97500000-98000000, chr10: 99000000-99500000, chr10: 100000000-100500000, |
| | chr10: 100500000-101000000, chr10: 101000000-101500000, chr10: 102000000-102500000, |
| | chr10: 102500000-103000000, chr10: 104000000-104500000, chr10: 108000000-108500000, |
| | chr10: 109000000-109500000, chr10: 109500000-110000000, chr10: 110000000-110500000, |
| | chr10: 111500000-112000000, chr10: 117000000-117500000, chr10: 119000000-119500000, |
| | chr10: 119500000-120000000, chr10: 120500000-121000000, chr10: 125500000-126000000, |
| | chr10: 126500000-127000000, chr10: 127000000-127500000, chr10: 127500000-128000000, |
| | chr10: 128500000-129000000, chr10: 129500000-130000000, chr10: 130000000-130500000, |
| | chr10: 130500000-131000000, chr10: 133500000-133797422 |
| 11 | chr11: 2500000-3000000, chr11: 4500000-5000000, chr11: 5500000-6000000, chr11: 6000000- |
| | 6500000, chr11: 6500000-7000000, chr11: 9000000-9500000, chr11: 10500000-11000000, |
| | chr11: 13000000-13500000, chr11: 18000000-18500000, chr11: 20500000-21000000, |
| | chr11: 23000000-23500000, chr11: 24000000-24500000, chr11: 25500000-26000000, |
| | chr11: 27500000-28000000, chr11: 29000000-29500000, chr11: 33000000-33500000, |
| | chr11: 33500000-34000000, chr11: 35000000-35500000, chr11: 37500000-38000000, |
| | chr11: 38000000-38500000, chr11: 39500000-40000000, chr11: 40000000-40500000, |
| | chr11: 40500000-41000000, chr11: 41500000-42000000, chr11: 43500000-44000000, |
| | chr11: 44500000-45000000, chr11: 46000000-46500000, chr11: 47000000-47500000, |
| | chr11: 47500000-48000000, chr11: 48500000-49000000, chr11: 49000000-49500000, |
| | chr11: 49500000-50000000, chr11: 50000000-50500000, chr11: 55500000-56000000, |
| | chr11: 56500000-57000000, chr11: 57500000-58000000, chr11: 59500000-60000000, |
| | chr11: 61000000-61500000, chr11: 61500000-62000000, chr11: 62500000-63000000, |
| | chr11: 63500000-64000000, chr11: 64000000-64500000, chr11: 64500000-65000000, |
| | chr11: 65000000-65500000, chr11: 65500000-66000000, chr11: 66000000-66500000, |
| | chr11: 67000000-67500000, chr11: 68500000-69000000, chr11: 72000000-72500000, |
| | chr11: 72500000-73000000, chr11: 77000000-77500000, chr11: 77500000-78000000, |
| | chr11: 78000000-78500000, chr11: 80000000-80500000, chr11: 85500000-86000000, |
| | chr11: 86000000-86500000, chr11: 89000000-89500000, chr11: 89500000-90000000, |
| | chr11: 92000000-92500000, chr11: 93000000-93500000, chr11: 93500000-94000000, |
| | chr11: 95000000-95500000, chr11: 95500000-96000000, chr11: 96500000-97000000, |
| | chr11: 99500000-100000000, chr11: 100000000-100500000, chr11: 101000000-101500000, |
| | chr11: 101500000-102000000, chr11: 102000000-102500000, chr11: 104000000-104500000, |
| | chr11: 104500000-105000000, chr11: 106500000-107000000, chr11: 107000000-107500000, |
| | chr11: 108500000-109000000, chr11: 109000000-109500000, chr11: 111500000-112000000, |
| | chr11: 114000000-114500000, chr11: 116500000-117000000, chr11: 117500000-118000000, |
| | chr11: 118000000-118500000, chr11: 118500000-119000000, chr11: 119000000-119500000, |
| | chr11: 121000000-121500000, chr11: 122500000-123000000, chr11: 125500000-126000000, |
| | chr11: 127000000-127500000, chr11: 128500000-129000000, chr11: 130000000-130500000, |
| | chr11: 133500000-134000000, chr11: 135000000-135086622 |
| 12 | chr12: 500000-1000000, chr12: 1500000-2000000, chr12: 6000000-6500000, chr12: 6500000- |
| | 7000000, chr12: 7500000-8000000, chr12: 8500000-9000000, chr12: 9500000-10000000, |
| | chr12: 10500000-11000000, chr12: 11000000-11500000, chr12: 11500000-12000000, |
| | chr12: 12000000-12500000, chr12: 12500000-13000000, chr12: 13000000-13500000, |
| | chr12: 17000000-17500000, chr12: 18000000-18500000, chr12: 25500000-26000000, |
| | chr12: 26000000-26500000, chr12: 27000000-27500000, chr12: 33000000-33500000, |
| | chr12: 34500000-35000000, chr12: 37000000-37500000, chr12: 38500000-39000000, |
| | chr12: 40500000-41000000, chr12: 41000000-41500000, chr12: 42000000-42500000, |
| | chr12: 42500000-43000000, chr12: 47500000-48000000, chr12: 48500000-49000000, |
| | chr12: 49000000-49500000, chr12: 49500000-50000000, chr12: 50000000-50500000, |
| | chr12: 50500000-51000000, chr12: 51000000-51500000, chr12: 53000000-53500000, |
| | chr12: 53500000-54000000, chr12: 54000000-54500000, chr12: 56000000-56500000, |
| | chr12: 57000000-57500000, chr12: 57500000-58000000, chr12: 58000000-58500000, |
| | chr12: 58500000-59000000, chr12: 60500000-61000000, chr12: 61000000-61500000, |
| | chr12: 62000000-62500000, chr12: 64500000-65000000, chr12: 66000000-66500000, |
| | chr12: 67500000-68000000, chr12: 68000000-68500000, chr12: 70500000-71000000, |
| | chr12: 71500000-72000000, chr12: 73000000-73500000, chr12: 75500000-76000000, |
| | chr12: 76000000-76500000, chr12: 76500000-77000000, chr12: 83500000-84000000, |
| | chr12: 84000000-84500000, chr12: 88000000-88500000, chr12: 89000000-89500000, |
| | chr12: 91500000-92000000, chr12: 93000000-93500000, chr12: 93500000-94000000, |
| | chr12: 94500000-95000000, chr12: 96000000-96500000, chr12: 98500000-99000000, |
| | chr12: 101500000-102000000, chr12: 102500000-103000000, chr12: 104000000-104500000, |
| | chr12: 104500000-105000000, chr12: 105000000-105500000, chr12: 106500000-107000000, |
| | chr12: 108500000-109000000, chr12: 109500000-110000000, chr12: 110000000-110500000, |
| | chr12: 110500000-111000000, chr12: 111000000-111500000, chr12: 112000000-112500000, |
| | chr12: 112500000-113000000, chr12: 113000000-113500000, chr12: 116000000-116500000, |
| | chr12: 116500000-117000000, chr12: 118000000-118500000, chr12: 120000000-120500000, |
| | chr12: 120500000-121000000, chr12: 121000000-121500000, chr12: 121500000-122000000, |
| | chr12: 122000000-122500000, chr12: 122500000-123000000, chr12: 127500000-128000000 |
| 13 | chr13: 16000000-16500000, chr13: 18000000-18500000, chr13: 18500000-19000000, |
| | chr13: 19000000-19500000, chr13: 20500000-21000000, chr13: 27000000-27500000, |
| | chr13: 28000000-28500000, chr13: 30000000-30500000, chr13: 30500000-31000000, |
| | chr13: 34500000-35000000, chr13: 35500000-36000000, chr13: 37500000-38000000, |
| | chr13: 38500000-39000000, chr13: 40000000-40500000, chr13: 41000000-41500000, |
| | chr13: 44000000-44500000, chr13: 45000000-45500000, chr13: 46000000-46500000, |
| | chr13: 48500000-49000000, chr13: 49500000-50000000, chr13: 50000000-50500000, |
| | chr13: 54000000-54500000, chr13: 56500000-57000000, chr13: 57000000-57500000, |
| | chr13: 60500000-61000000, chr13: 66500000-67000000, chr13: 67000000-67500000, |
| | chr13: 67500000-68000000, chr13: 68000000-68500000, chr13: 73000000-73500000, |
| | chr13: 75500000-76000000, chr13: 85000000-85500000, chr13: 90500000-91000000, |
| | chr13: 92500000-93000000, chr13: 93500000-94000000, chr13: 94000000-94500000, |
| | chr13: 97500000-98000000, chr13: 99000000-99500000, chr13: 100000000-100500000, |
| | chr13: 102000000-102500000, chr13: 102500000-103000000, chr13: 104000000-104500000, |
| | chr13: 107000000-107500000, chr13: 112000000-112500000 |
| 14 | chr14: 18000000-18500000, chr14: 18500000-19000000, chr14: 19000000-19500000, |
| | chr14: 19500000-20000000, chr14: 21000000-21500000, chr14: 22500000-23000000, |
| | chr14: 23000000-23500000, chr14: 25500000-26000000, chr14: 27000000-27500000, |
| | chr14: 28000000-28500000, chr14: 29000000-29500000, chr14: 29500000-30000000, |
| | chr14: 30500000-31000000, chr14: 34500000-35000000, chr14: 35000000-35500000, |
| | chr14: 35500000-36000000, chr14: 36500000-37000000, chr14: 39000000-39500000, |
| | chr14: 39500000-40000000, chr14: 43000000-43500000, chr14: 45500000-46000000, |
| | chr14: 47000000-47500000, chr14: 47500000-48000000, chr14: 48000000-48500000, |
| | chr14: 49500000-50000000, chr14: 50500000-51000000, chr14: 51500000-52000000, |
| | chr14: 55000000-55500000, chr14: 55500000-56000000, chr14: 58000000-58500000, |
| | chr14: 59000000-59500000, chr14: 61000000-61500000, chr14: 63500000-64000000, |
| | chr14: 64500000-65000000, chr14: 65000000-65500000, chr14: 67500000-68000000, |
| | chr14: 68500000-69000000, chr14: 69000000-69500000, chr14: 69500000-70000000, |
| | chr14: 70500000-71000000, chr14: 73000000-73500000, chr14: 74000000-74500000, |
| | chr14: 75000000-75500000, chr14: 77000000-77500000, chr14: 79500000-80000000, |
| | chr14: 80500000-81000000, chr14: 82000000-82500000, chr14: 83000000-83500000, |
| | chr14: 86000000-86500000, chr14: 86500000-87000000, chr14: 87000000-87500000, |
| | chr14: 88500000-89000000, chr14: 89000000-89500000, chr14: 91000000-91500000, |
| | chr14: 92000000-92500000, chr14: 94000000-94500000, chr14: 97000000-97500000, |
| | chr14: 97500000-98000000, chr14: 98000000-98500000, chr14: 100000000-100500000, |
| | chr14: 101000000-101500000 |
| 15 | chr15: 17000000-17500000, chr15: 19500000-20000000, chr15: 20000000-20500000, |
| | chr15: 20500000-21000000, chr15: 21000000-21500000, chr15: 21500000-22000000, |
| | chr15: 22000000-22500000, chr15: 23000000-23500000, chr15: 24000000-24500000, |
| | chr15: 24500000-25000000, chr15: 25000000-25500000, chr15: 26500000-27000000, |
| | chr15: 27500000-28000000, chr15: 28000000-28500000, chr15: 28500000-29000000, |
| | chr15: 30000000-30500000, chr15: 32000000-32500000, chr15: 34000000-34500000, |
| | chr15: 36000000-36500000, chr15: 39500000-40000000, chr15: 40500000-41000000, |
| | chr15: 41000000-41500000, chr15: 41500000-42000000, chr15: 42000000-42500000, |
| | chr15: 42500000-43000000, chr15: 43000000-43500000, chr15: 43500000-44000000, |
| | chr15: 46000000-46500000, chr15: 48500000-49000000, chr15: 50000000-50500000, |
| | chr15: 53000000-53500000, chr15: 55000000-55500000, chr15: 57000000-57500000, |
| | chr15: 58000000-58500000, chr15: 60000000-60500000, chr15: 62000000-62500000, |
| | chr15: 63000000-63500000, chr15: 63500000-64000000, chr15: 64000000-64500000, |
| | chr15: 64500000-65000000, chr15: 65500000-66000000, chr15: 68500000-69000000, |
| | chr15: 69000000-69500000, chr15: 69500000-70000000, chr15: 70000000-70500000, |
| | chr15: 72500000-73000000, chr15: 73000000-73500000, chr15: 78000000-78500000, |
| | chr15: 79000000-79500000, chr15: 81000000-81500000, chr15: 82000000-82500000, |
| | chr15: 84000000-84500000, chr15: 85500000-86000000, chr15: 89000000-89500000, |
| | chr15: 90000000-90500000, chr15: 90500000-91000000, chr15: 91500000-92000000, |
| | chr15: 92500000-93000000, chr15: 94000000-94500000, chr15: 96500000-97000000, |
| | chr15: 97500000-98000000 |
| 6 | chr16: 4000000-4500000, chr16: 4500000-5000000, chr16: 9000000-9500000, chr16: 10500000- |
| | 11000000, chr16: 11500000-12000000, chr16: 12000000-12500000, chr16: 12500000-13000000, |
| | chr16: 15500000-16000000, chr16: 17000000-17500000, chr16: 18000000-18500000, |
| | chr16: 22000000-22500000, chr16: 23500000-24000000, chr16: 24000000-24500000, |
| | chr16: 24500000-25000000, chr16: 26500000-27000000, chr16: 27000000-27500000, |
| | chr16: 28500000-29000000, chr16: 29000000-29500000, chr16: 29500000-30000000, |
| | chr16: 30000000-30500000, chr16: 30500000-31000000, chr16: 31000000-31500000, |
| | chr16: 32000000-32500000, chr16: 32500000-33000000, chr16: 33000000-33500000, |
| | chr16: 33500000-34000000, chr16: 34000000-34500000, chr16: 34500000-35000000, |
| | chr16: 35000000-35500000, chr16: 46000000-46500000, chr16: 46500000-47000000, |
| | chr16: 48000000-48500000, chr16: 53000000-53500000, chr16: 53500000-54000000, |
| | chr16: 57000000-57500000, chr16: 58000000-58500000, chr16: 61000000-61500000, |
| | chr16: 63000000-63500000, chr16: 67500000-68000000, chr16: 68000000-68500000, |
| | chr16: 69000000-69500000, chr16: 71500000-72000000, chr16: 74500000-75000000, |
| | chr16: 79000000-79500000, chr16: 81000000-81500000, chr16: 81500000-82000000, |
| | chr16: 84500000-85000000, chr16: 85000000-85500000, chr16: 90000000-90338345 |
| 17 | chr17: 0-500000, chr17: 1000000-1500000, chr17: 1500000-2000000, chr17: 2000000-2500000, |
| | chr17: 2500000-3000000, chr17: 4500000-5000000, chr17: 7000000-7500000, chr17: 7500000- |
| | 8000000, chr17: 8000000-8500000, chr17: 8500000-9000000, chr17: 10000000-10500000, |
| | chr17: 11000000-11500000, chr17: 13500000-14000000, chr17: 14000000-14500000, |
| | chr17: 17500000-18000000, chr17: 19000000-19500000, chr17: 20500000-21000000, |
| | chr17: 21000000-21500000, chr17: 21500000-22000000, chr17: 22000000-22500000, |
| | chr17: 22500000-23000000, chr17: 26500000-27000000, chr17: 28000000-28500000, |
| | chr17: 28500000-29000000, chr17: 29500000-30000000, chr17: 30000000-30500000, |
| | chr17: 32000000-32500000, chr17: 35000000-35500000, chr17: 35500000-36000000, |
| | chr17: 36000000-36500000, chr17: 36500000-37000000, chr17: 38000000-38500000, |
| | chr17: 38500000-39000000, chr17: 39000000-39500000, chr17: 39500000-40000000, |
| | chr17: 40000000-40500000, chr17: 41500000-42000000, chr17: 42000000-42500000, |
| | chr17: 42500000-43000000, chr17: 43500000-44000000, chr17: 44000000-44500000, |
| | chr17: 45000000-45500000, chr17: 47500000-48000000, chr17: 48500000-49000000, |
| | chr17: 49000000-49500000, chr17: 49500000-50000000, chr17: 50000000-50500000, |
| | chr17: 50500000-51000000, chr17: 51000000-51500000, chr17: 53500000-54000000, |
| | chr17: 54000000-54500000, chr17: 55000000-55500000, chr17: 56500000-57000000, |
| | chr17: 57000000-57500000, chr17: 58000000-58500000, chr17: 58500000-59000000, |
| | chr17: 59000000-59500000, chr17: 59500000-60000000, chr17: 60000000-60500000, |
| | chr17: 61500000-62000000, chr17: 62500000-63000000, chr17: 63500000-64000000, |
| | chr17: 64000000-64500000, chr17: 65000000-65500000, chr17: 66000000-66500000, |
| | chr17: 67000000-67500000, chr17: 67500000-68000000, chr17: 68000000-68500000, |
| | chr17: 73500000-74000000, chr17: 74000000-74500000, chr17: 74500000-75000000, |
| | chr17: 75000000-75500000, chr17: 75500000-76000000, chr17: 76000000-76500000, |
| | chr17: 77000000-77500000, chr17: 78000000-78500000, chr17: 78500000-79000000, |
| | chr17: 79500000-80000000, chr17: 80000000-80500000, chr17: 82500000-83000000 |
| 18 | chr18: 1000000-1500000, chr18: 3000000-3500000, chr18: 5500000-6000000, chr18: 6500000- |
| | 7000000, chr18: 8000000-8500000, chr18: 9000000-9500000, chr18: 10000000-10500000, |
| | chr18: 11000000-11500000, chr18: 12000000-12500000, chr18: 12500000-13000000, |
| | chr18: 14000000-14500000, chr18: 14500000-15000000, chr18: 15000000-15500000, |
| | chr18: 20500000-21000000, chr18: 21000000-21500000, chr18: 23500000-24000000, |
| | chr18: 25000000-25500000, chr18: 27000000-27500000, chr18: 27500000-28000000, |
| | chr18: 35000000-35500000, chr18: 46000000-46500000, chr18: 47000000-47500000, |
| | chr18: 48500000-49000000, chr18: 49000000-49500000, chr18: 51000000-51500000, |
| | chr18: 53000000-53500000, chr18: 59500000-60000000, chr18: 61000000-61500000, |
| | chr18: 63000000-63500000, chr18: 64500000-65000000, chr18: 74500000-75000000, |
| | chr18: 76000000-76500000, chr18: 78000000-78500000 |
| 19 | chr19: 0-500000, chr19: 1500000-2000000, chr19: 2000000-2500000, chr19: 2500000-3000000, |
| | chr19: 3000000-3500000, chr19: 4000000-4500000, chr19: 4500000-5000000, chr19: 5500000- |
| | 6000000, chr19: 6000000-6500000, chr19: 6500000-7000000, chr19: 7000000-7500000, |
| | chr19: 7500000-8000000, chr19: 8000000-8500000, chr19: 9500000-10000000, chr19: 10000000- |
| | 10500000, chr19: 10500000-11000000, chr19: 11000000-11500000, chr19: 11500000-12000000, |
| | chr19: 12500000-13000000, chr19: 13000000-13500000, chr19: 13500000-14000000, |
| | chr19: 14000000-14500000, chr19: 14500000-15000000, chr19: 15500000-16000000, |
| | chr19: 16000000-16500000, chr19: 16500000-17000000, chr19: 17000000-17500000, |
| | chr19: 17500000-18000000, chr19: 18000000-18500000, chr19: 18500000-19000000, |
| | chr19: 19000000-19500000, chr19: 20500000-21000000, chr19: 22000000-22500000, |
| | chr19: 22500000-23000000, chr19: 27500000-28000000, chr19: 34000000-34500000, |
| | chr19: 35000000-35500000, chr19: 35500000-36000000, chr19: 38000000-38500000, |
| | chr19: 38500000-39000000, chr19: 39000000-39500000, chr19: 39500000-40000000, |
| | chr19: 40000000-40500000, chr19: 40500000-41000000, chr19: 41000000-41500000, |
| | chr19: 41500000-42000000, chr19: 42000000-42500000, chr19: 43500000-44000000, |
| | chr19: 44500000-45000000, chr19: 45000000-45500000, chr19: 45500000-46000000, |
| | chr19: 46500000-47000000, chr19: 47000000-47500000, chr19: 48000000-48500000, |
| | chr19: 48500000-49000000, chr19: 49000000-49500000, chr19: 49500000-50000000, |
| | chr19: 51000000-51500000, chr19: 51500000-52000000, chr19: 53500000-54000000, |
| | chr19: 54000000-54500000, chr19: 55000000-55500000, chr19: 58500000-58617616 |
| 20 | chr20: 0-500000, chr20: 1000000-1500000, chr20: 1500000-2000000, chr20: 2500000-3000000, |
| | chr20: 3500000-4000000, chr20: 4500000-5000000, chr20: 5000000-5500000, chr20: 5500000- |
| | 6000000, chr20: 8000000-8500000, chr20: 12500000-13000000, chr20: 16000000-16500000, |
| | chr20: 21000000-21500000, chr20: 21500000-22000000, chr20: 28500000-29000000, |
| | chr20: 29000000-29500000, chr20: 29500000-30000000, chr20: 30000000-30500000, |
| | chr20: 30500000-31000000, chr20: 31500000-32000000, chr20: 32000000-32500000, |
| | chr20: 33000000-33500000, chr20: 33500000-34000000, chr20: 34000000-34500000, |
| | chr20: 36500000-37000000, chr20: 37000000-37500000, chr20: 37500000-38000000, |
| | chr20: 38000000-38500000, chr20: 39500000-40000000, chr20: 40500000-41000000, |
| | chr20: 42500000-43000000, chr20: 43500000-44000000, chr20: 44000000-44500000, |
| | chr20: 44500000-45000000, chr20: 45000000-45500000, chr20: 45500000-46000000, |
| | chr20: 46000000-46500000, chr20: 47000000-47500000, chr20: 47500000-48000000, |
| | chr20: 48500000-49000000, chr20: 49000000-49500000, chr20: 49500000-50000000, |
| | chr20: 50000000-50500000, chr20: 50500000-51000000, chr20: 51000000-51500000, |
| | chr20: 51500000-52000000, chr20: 52000000-52500000, chr20: 52500000-53000000, |
| | chr20: 53500000-54000000, chr20: 55000000-55500000, chr20: 55500000-56000000, |
| | chr20: 59000000-59500000, chr20: 62000000-62500000, chr20: 63000000-63500000 |
| 21 | chr21: 5000000-5500000, chr21: 5500000-6000000, chr21: 6000000-6500000, chr21: 6500000- |
| | 7000000, chr21: 7000000-7500000, chr21: 7500000-8000000, chr21: 8000000-8500000, |
| | chr21: 8500000-9000000, chr21: 9000000-9500000, chr21: 9500000-10000000, chr21: 10000000- |
| | 10500000, chr21: 10500000-11000000, chr21: 13000000-13500000, chr21: 13500000-14000000, |
| | chr21: 14000000-14500000, chr21: 15500000-16000000, chr21: 16500000-17000000, |
| | chr21: 17000000-17500000, chr21: 17500000-18000000, chr21: 23500000-24000000, |
| | chr21: 25500000-26000000, chr21: 31000000-31500000, chr21: 31500000-32000000, |
| | chr21: 33000000-33500000, chr21: 33500000-34000000, chr21: 34500000-35000000, |
| | chr21: 35000000-35500000, chr21: 36000000-36500000, chr21: 37000000-37500000, |
| | chr21: 40000000-40500000, chr21: 40500000-41000000, chr21: 42000000-42500000, |
| | chr21: 44000000-44500000 |
| 22 | chr22: 10500000-11000000, chr22: 11000000-11500000, chr22: 11500000-12000000, |
| | chr22: 12000000-12500000, chr22: 12500000-13000000, chr22: 15000000-15500000, |
| | chr22: 15500000-16000000, chr22: 16000000-16500000, chr22: 16500000-17000000, |
| | chr22: 18000000-18500000, chr22: 18500000-19000000, chr22: 20500000-21000000, |
| | chr22: 21500000-22000000, chr22: 23500000-24000000, chr22: 26000000-26500000, |
| | chr22: 27000000-27500000, chr22: 28500000-29000000, chr22: 29500000-30000000, |
| | chr22: 30000000-30500000, chr22: 31000000-31500000, chr22: 31500000-32000000, |
| | chr22: 32500000-33000000, chr22: 35000000-35500000, chr22: 35500000-36000000, |
| | chr22: 36000000-36500000, chr22: 36500000-37000000, chr22: 37000000-37500000, |
| | chr22: 37500000-38000000, chr22: 38000000-38500000, chr22: 39000000-39500000, |
| | chr22: 40000000-40500000, chr22: 40500000-41000000, chr22: 41000000-41500000, |
| | chr22: 41500000-42000000, chr22: 48500000-49000000 |

TABLE 19

| | |
|---|---|
| 1 | chr1: 500000-1000000, chr1: 1500000-2000000, chr1: 4000000-4500000, chr1: 10000000-10500000, |
| | chr1: 16500000-17000000, chr1: 18000000-18500000, chr1: 19500000-20000000, chr1: 20000000- |
| | 20500000, chr1: 21500000-22000000, chr1: 22500000-23000000, chr1: 23000000-23500000, |
| | chr1: 25500000-26000000, chr1: 26000000-26500000, chr1: 27000000-27500000, chr1: 27500000- |
| | 28000000, chr1: 28000000-28500000, chr1: 28500000-29000000, chr1: 29000000-29500000, |
| | chr1: 29500000-30000000, chr1: 30500000-31000000, chr1: 31500000-32000000, chr1: 32000000- |
| | 32500000, chr1: 32500000-33000000, chr1: 33000000-33500000, chr1: 33500000-34000000, |
| | chr1: 34500000-35000000, chr1: 35500000-36000000, chr1: 36000000-36500000, chr1: 36500000- |
| | 37000000, chr1: 37000000-37500000, chr1: 38500000-39000000, chr1: 45500000-46000000, |
| | chr1: 49500000-50000000, chr1: 50000000-50500000, chr1: 56500000-57000000, chr1: 57000000- |
| | 57500000, chr1: 58500000-59000000, chr1: 59500000-60000000, chr1: 60000000-60500000, |
| | chr1: 64000000-64500000, chr1: 74500000-75000000, chr1: 87500000-88000000, chr1: 94500000- |
| | 95000000, chr1: 97000000-97500000, chr1: 107500000-108000000, chr1: 109500000-110000000, |
| | chr1: 110000000-110500000, chr1: 115000000-115500000, chr1: 120000000-120500000, |
| | chr1: 120500000-121000000, chr1: 121000000-121500000, chr1: 125000000-125500000, |
| | chr1: 143000000-143500000, chr1: 144000000-144500000, chr1: 144500000-145000000, |
| | chr1: 145000000-145500000, chr1: 146000000-146500000, chr1: 146500000-147000000, |
| | chr1: 148000000-148500000, chr1: 148500000-149000000, chr1: 149000000-149500000, |
| | chr1: 150000000-150500000, chr1: 150500000-151000000, chr1: 151000000-151500000, |
| | chr1: 151500000-152000000, chr1: 153000000-153500000, chr1: 153500000-154000000, |
| | chr1: 154500000-155000000, chr1: 155500000-156000000, chr1: 156000000-156500000, |
| | chr1: 156500000-157000000, chr1: 158000000-158500000, chr1: 159500000-160000000, |
| | chr1: 160000000-160500000, chr1: 161000000-161500000, chr1: 162000000-162500000, |
| | chr1: 175500000-176000000, chr1: 176500000-177000000, chr1: 181000000-181500000, |
| | chr1: 183000000-183500000, chr1: 186000000-186500000, chr1: 190000000-190500000, |
| | chr1: 192500000-193000000, chr1: 200500000-201000000, chr1: 201500000-202000000, |
| | chr1: 204000000-204500000, chr1: 205000000-205500000, chr1: 208000000-208500000, |
| | chr1: 209500000-210000000, chr1: 214000000-214500000, chr1: 219000000-219500000, |
| | chr1: 221000000-221500000, chr1: 226000000-226500000, chr1: 229500000-230000000, |
| | chr1: 230500000-231000000, chr1: 234000000-234500000, chr1: 240500000-241000000, |
| | chr1: 241500000-242000000, chr1: 245000000-245500000, chr1: 247000000-247500000, |
| | chr1: 248000000-248500000 |
| 2 | chr2: 3000000-3500000, chr2: 8000000-8500000, chr2: 11500000-12000000, chr2: 22500000- |
| | 23000000, chr2: 27000000-27500000, chr2: 28500000-29000000, chr2: 36500000-37000000, |
| | chr2: 37000000-37500000, chr2: 37500000-38000000, chr2: 39000000-39500000, chr2: 44000000- |
| | 44500000, chr2: 47000000-47500000, chr2: 48500000-49000000, chr2: 49000000-49500000, |
| | chr2: 57500000-58000000, chr2: 59500000-60000000, chr2: 61500000-62000000, chr2: 64000000- |
| | 64500000, chr2: 64500000-65000000, chr2: 65000000-65500000, chr2: 73500000-74000000, |
| | chr2: 75000000-75500000, chr2: 87000000-87500000, chr2: 87500000-88000000, chr2: 88500000- |
| | 89000000, chr2: 89500000-90000000, chr2: 90000000-90500000, chr2: 91000000-91500000, |
| | chr2: 91500000-92000000, chr2: 92000000-92500000, chr2: 95500000-96000000, chr2: 97000000- |
| | 97500000, chr2: 102000000-102500000, chr2: 106000000-106500000, chr2: 109500000- |
| | 110000000, chr2: 110000000-110500000, chr2: 110500000-111000000, chr2: 112500000- |
| | 113000000, chr2: 113000000-113500000, chr2: 118000000-118500000, chr2: 125000000- |
| | 125500000, chr2: 126500000-127000000, chr2: 127500000-128000000, chr2: 130000000- |
| | 130500000, chr2: 132000000-132500000, chr2: 132500000-133000000, chr2: 134500000- |
| | 135000000, chr2: 136000000-136500000, chr2: 144500000-145000000, chr2: 145000000- |
| | 145500000, chr2: 148500000-149000000, chr2: 159000000-159500000, chr2: 162000000- |
| | 162500000, chr2: 165500000-166000000, chr2: 171500000-172000000, chr2: 173500000- |
| | 174000000, chr2: 188500000-189000000, chr2: 190500000-191000000, chr2: 191000000- |
| | 191500000, chr2: 197000000-197500000, chr2: 200500000-201000000, chr2: 201500000- |
| | 202000000, chr2: 206000000-206500000, chr2: 207000000-207500000, chr2: 216000000- |
| | 216500000, chr2: 218000000-218500000, chr2: 219500000-220000000, chr2: 221000000- |
| | 221500000, chr2: 227500000-228000000, chr2: 229500000-230000000, chr2: 236500000- |
| | 237000000 |
| 3 | chr3: 4500000-5000000, chr3: 5000000-5500000, chr3: 7000000-7500000, chr3: 9000000-9500000, |
| | chr3: 9500000-10000000, chr3: 10000000-10500000, chr3: 10500000-11000000, chr3: 12500000- |
| | 13000000, chr3: 13500000-14000000, chr3: 21000000-21500000, chr3: 21500000-22000000, |
| | chr3: 32500000-33000000, chr3: 34000000-34500000, chr3: 36000000-36500000, chr3: 37000000- |
| | 37500000, chr3: 43500000-44000000, chr3: 44000000-44500000, chr3: 45000000-45500000, |
| | chr3: 46000000-46500000, chr3: 47000000-47500000, chr3: 48500000-49000000, chr3: 49000000- |
| | 49500000, chr3: 49500000-50000000, chr3: 52000000-52500000, chr3: 63500000-64000000, |
| | chr3: 72000000-72500000, chr3: 75500000-76000000, chr3: 79000000-79500000, chr3: 91000000- |
| | 91500000, chr3: 96500000-97000000, chr3: 99500000-100000000, chr3: 108500000-109000000, |
| | chr3: 115000000-115500000, chr3: 127500000-128000000, chr3: 128500000-129000000, |
| | chr3: 129500000-130000000, chr3: 132500000-133000000, chr3: 138500000-139000000, |
| | chr3: 145500000-146000000, chr3: 149000000-149500000, chr3: 152500000-153000000, |
| | chr3: 157500000-158000000, chr3: 170000000-170500000, chr3: 171500000-172000000, |
| | chr3: 172000000-172500000, chr3: 184500000-185000000, chr3: 185500000-186000000, |
| | chr3: 186000000-186500000, chr3: 188000000-188500000, chr3: 194500000-195000000, |
| | chr3: 195000000-195500000, chr3: 196500000-197000000, chr3: 198000000-198295559 |
| 4 | chr4: 2000000-2500000, chr4: 4500000-5000000, chr4: 5500000-6000000, chr4: 7500000-8000000, |
| | chr4: 9500000-10000000, chr4: 15000000-15500000, chr4: 16500000-17000000, chr4: 32500000- |
| | 33000000, chr4: 37500000-38000000, chr4: 40500000-41000000, chr4: 49000000-49500000, |
| | chr4: 49500000-50000000, chr4: 64000000-64500000, chr4: 67500000-68000000, chr4: 70500000- |
| | 71000000, chr4: 73500000-74000000, chr4: 78000000-78500000, chr4: 80000000-80500000, |
| | chr4: 88500000-89000000, chr4: 103000000-103500000, chr4: 112500000-113000000, |
| | chr4: 125500000-126000000, chr4: 145000000-145500000, chr4: 146000000-146500000, |
| | chr4: 152000000-152500000, chr4: 161500000-162000000, chr4: 164000000-164500000, |
| | chr4: 175500000-176000000, chr4: 176000000-176500000, chr4: 184500000-185000000, |
| | chr4: 185500000-186000000, chr4: 186000000-186500000, chr4: 187000000-187500000, |
| | chr4: 190000000-190214555 |
| 5 | chr5: 10500000-11000000, chr5: 11000000-11500000, chr5: 11500000-12000000, chr5: 13500000- |
| | 14000000, chr5: 21000000-21500000, chr5: 31500000-32000000, chr5: 34000000-34500000, |
| | chr5: 38000000-38500000, chr5: 43500000-44000000, chr5: 54000000-54500000, chr5: 56000000- |
| | 56500000, chr5: 61000000-61500000, chr5: 69500000-70000000, chr5: 70000000-70500000, |
| | chr5: 70500000-71000000, chr5: 71000000-71500000, chr5: 80000000-80500000, chr5: 81500000- |
| | 82000000, chr5: 91000000-91500000, chr5: 94000000-94500000, chr5: 112500000-113000000, |
| | chr5: 119500000-120000000, chr5: 124000000-124500000, chr5: 138000000-138500000, |
| | chr5: 139500000-140000000, chr5: 140500000-141000000, chr5: 148000000-148500000, |
| | chr5: 148500000-149000000, chr5: 157500000-158000000, chr5: 158000000-158500000, |
| | chr5: 158500000-159000000, chr5: 171500000-172000000, chr5: 172000000-172500000, |
| | chr5: 172500000-173000000, chr5: 173500000-174000000, chr5: 174500000-175000000, |
| | chr5: 176000000-176500000, chr5: 176500000-177000000 |
| 6 | chr6: 2500000-3000000, chr6: 6500000-7000000, chr6: 13000000-13500000, chr6: 15000000- |
| | 15500000, chr6: 17500000-18000000, chr6: 25000000-25500000, chr6: 26000000-26500000, |
| | chr6: 26500000-27000000, chr6: 33000000-33500000, chr6: 33500000-34000000, chr6: 36000000- |
| | 36500000, chr6: 36500000-37000000, chr6: 37000000-37500000, chr6: 40000000-40500000, |
| | chr6: 41000000-41500000, chr6: 41500000-42000000, chr6: 42000000-42500000, chr6: 42500000- |
| | 43000000, chr6: 43000000-43500000, chr6: 43500000-44000000, chr6: 57500000-58000000, |
| | chr6: 58000000-58500000, chr6: 60000000-60500000, chr6: 60500000-61000000, chr6: 77500000- |
| | 78000000, chr6: 79500000-80000000, chr6: 81000000-81500000, chr6: 86000000-86500000, |
| | chr6: 88500000-89000000, chr6: 107000000-107500000, chr6: 109000000-109500000, |
| | chr6: 112000000-112500000, chr6: 114000000-114500000, chr6: 119500000-120000000, |
| | chr6: 121000000-121500000, chr6: 124000000-124500000, chr6: 129000000-129500000, |
| | chr6: 130500000-131000000, chr6: 154000000-154500000, chr6: 160000000-160500000, |
| | chr6: 163500000-164000000, chr6: 168500000-169000000, chr6: 170000000-170500000, |
| | chr6: 170500000-170805979 |
| 7 | chr7: 5500000-6000000, chr7: 6000000-6500000, chr7: 25500000-26000000, chr7: 26000000- |
| | 26500000, chr7: 26500000-27000000, chr7: 30500000-31000000, chr7: 32500000-33000000, |
| | chr7: 36000000-36500000, chr7: 36500000-37000000, chr7: 39500000-40000000, chr7: 41500000- |
| | 42000000, chr7: 44000000-44500000, chr7: 44500000-45000000, chr7: 54000000-54500000, |
| | chr7: 55500000-56000000, chr7: 56500000-57000000, chr7: 57500000-58000000, chr7: 58000000- |
| | 58500000, chr7: 63000000-63500000, chr7: 63500000-64000000, chr7: 64000000-64500000, |
| | chr7: 65000000-65500000, chr7: 65500000-66000000, chr7: 66000000-66500000, chr7: 66500000- |
| | 67000000, chr7: 73500000-74000000, chr7: 74000000-74500000, chr7: 74500000-75000000, |
| | chr7: 75500000-76000000, chr7: 76000000-76500000, chr7: 76500000-77000000, chr7: 95500000- |
| | 96000000, chr7: 97500000-98000000, chr7: 100000000-100500000, chr7: 100500000-101000000, |
| | chr7: 101500000-102000000, chr7: 105000000-105500000, chr7: 108000000-108500000, |
| | chr7: 112000000-112500000, chr7: 125000000-125500000, chr7: 127500000-128000000, |
| | chr7: 128000000-128500000, chr7: 128500000-129000000, chr7: 131500000-132000000, |
| | chr7: 133000000-133500000, chr7: 143000000-143500000, chr7: 145000000-145500000 |
| 8 | chr8: 0-500000, chr8: 8000000-8500000, chr8: 8500000-9000000, chr8: 12000000-12500000, |
| | chr8: 15000000-15500000, chr8: 17500000-18000000, chr8: 20000000-20500000, chr8: 20500000- |
| | 21000000, chr8: 22500000-23000000, chr8: 27000000-27500000, chr8: 29500000-30000000, |
| | chr8: 37500000-38000000, chr8: 38000000-38500000, chr8: 41500000-42000000, chr8: 42000000- |
| | 42500000, chr8: 42500000-43000000, chr8: 46000000-46500000, chr8: 48000000-48500000, |
| | chr8: 57000000-57500000, chr8: 57500000-58000000, chr8: 58500000-59000000, chr8: 60500000- |
| | 61000000, chr8: 72000000-72500000, chr8: 73500000-74000000, chr8: 85500000-86000000, |
| | chr8: 91500000-92000000, chr8: 94500000-95000000, chr8: 96000000-96500000, chr8: 97000000- |
| | 97500000, chr8: 98000000-98500000, chr8: 101000000-101500000, chr8: 113000000-113500000, |
| | chr8: 114500000-115000000, chr8: 117500000-118000000, chr8: 122500000-123000000, |
| | chr8: 125000000-125500000, chr8: 126000000-126500000, chr8: 128500000-129000000, |
| | chr8: 129000000-129500000, chr8: 130000000-130500000, chr8: 132000000-132500000, |
| | chr8: 133500000-134000000, chr8: 138500000-139000000, chr8: 144000000-144500000 |
| 9 | chr9: 5500000-6000000, chr9: 10000000-10500000, chr9: 21000000-21500000, chr9: 21500000- |
| | 22000000, chr9: 29000000-29500000, chr9: 33000000-33500000, chr9: 34000000-34500000, |
| | chr9: 34500000-35000000, chr9: 35500000-36000000, chr9: 36000000-36500000, chr9: 39000000- |
| | 39500000, chr9: 39500000-40000000, chr9: 40000000-40500000, chr9: 40500000-41000000, |
| | chr9: 41000000-41500000, chr9: 41500000-42000000, chr9: 42000000-42500000, chr9: 42500000- |
| | 43000000, chr9: 60500000-61000000, chr9: 61500000-62000000, chr9: 62000000-62500000, |
| | chr9: 62500000-63000000, chr9: 63000000-63500000, chr9: 63500000-64000000, chr9: 64000000- |
| | 64500000, chr9: 64500000-65000000, chr9: 65000000-65500000, chr9: 65500000-66000000, |
| | chr9: 66000000-66500000, chr9: 66500000-67000000, chr9: 67000000-67500000, chr9: 67500000- |
| | 68000000, chr9: 71500000-72000000, chr9: 72500000-73000000, chr9: 74500000-75000000, |
| | chr9: 77500000-78000000, chr9: 80500000-81000000, chr9: 84000000-84500000, chr9: 86000000- |
| | 86500000, chr9: 89500000-90000000, chr9: 92500000-93000000, chr9: 105000000-105500000, |
| | chr9: 114000000-114500000, chr9: 114500000-115000000, chr9: 116000000-116500000, |
| | chr9: 120500000-121000000, chr9: 121500000-122000000, chr9: 122000000-122500000, |
| | chr9: 123000000-123500000, chr9: 127500000-128000000, chr9: 129000000-129500000, |
| | chr9: 129500000-130000000, chr9: 131500000-132000000, chr9: 132000000-132500000 |
| 10 | chr10: 3500000-4000000, chr10: 6000000-6500000, chr10: 9000000-9500000, chr10: 21000000- |
| | 21500000, chr10: 22500000-23000000, chr10: 23500000-24000000, chr10: 38000000-38500000, |
| | chr10: 38500000-39000000, chr10: 42000000-42500000, chr10: 46000000-46500000, |
| | chr10: 46500000-47000000, chr10: 47500000-48000000, chr10: 50000000-50500000, |
| | chr10: 69000000-69500000, chr10: 71500000-72000000, chr10: 72000000-72500000, |
| | chr10: 77500000-78000000, chr10: 94000000-94500000, chr10: 94500000-95000000, |
| | chr10: 95000000-95500000, chr10: 97000000-97500000, chr10: 97500000-98000000, |
| | chr10: 98000000-98500000, chr10: 98500000-99000000, chr10: 100500000-101000000, |
| | chr10: 101000000-101500000, chr10: 104000000-104500000, chr10: 109500000-110000000, |
| | chr10: 110000000-110500000, chr10: 111500000-112000000, chr10: 116000000-116500000, |
| | chr10: 123500000-124000000, chr10: 133500000-133797422 |
| 11 | chr11: 0-500000, chr11: 1500000-2000000, chr11: 3500000-4000000, chr11: 4000000-4500000, |
| | chr11: 5500000-6000000, chr11: 8500000-9000000, chr11: 9500000-10000000, chr11: 12000000- |
| | 12500000, chr11: 15500000-16000000, chr11: 17500000-18000000, chr11: 28000000-28500000, |
| | chr11: 32000000-32500000, chr11: 44500000-45000000, chr11: 46000000-46500000, |
| | chr11: 47000000-47500000, chr11: 49000000-49500000, chr11: 49500000-50000000, |
| | chr11: 56500000-57000000, chr11: 57500000-58000000, chr11: 59500000-60000000, |
| | chr11: 62000000-62500000, chr11: 62500000-63000000, chr11: 64500000-65000000, |
| | chr11: 65500000-66000000, chr11: 66000000-66500000, chr11: 69500000-70000000, |
| | chr11: 72000000-72500000, chr11: 72500000-73000000, chr11: 73000000-73500000, |
| | chr11: 73500000-74000000, chr11: 78000000-78500000, chr11: 79000000-79500000, |
| | chr11: 81500000-82000000, chr11: 86000000-86500000, chr11: 89000000-89500000, |
| | chr11: 89500000-90000000, chr11: 99000000-99500000, chr11: 111000000-111500000, |
| | chr11: 111500000-112000000, chr11: 113500000-114000000, chr11: 117000000-117500000, |
| | chr11: 119000000-119500000, chr11: 120500000-121000000, chr11: 121000000-121500000, |
| | chr11: 122500000-123000000, chr11: 123500000-124000000, chr11: 126000000-126500000, |
| | chr11: 126500000-127000000, chr11: 134500000-135000000 |
| 12 | chr12: 0-500000, chr12: 500000-1000000, chr12: 3500000-4000000, chr12: 6000000-6500000, |
| | chr12: 9000000-9500000, chr12: 12500000-13000000, chr12: 17500000-18000000, |
| | chr12: 28000000-28500000, chr12: 37000000-37500000, chr12: 37500000-38000000, |
| | chr12: 38000000-38500000, chr12: 39500000-40000000, chr12: 42000000-42500000, |
| | chr12: 47500000-48000000, chr12: 49000000-49500000, chr12: 52500000-53000000, |
| | chr12: 53000000-53500000, chr12: 53500000-54000000, chr12: 54000000-54500000, |
| | chr12: 55500000-56000000, chr12: 56000000-56500000, chr12: 57000000-57500000, |
| | chr12: 61000000-61500000, chr12: 66500000-67000000, chr12: 69500000-70000000, |
| | chr12: 74500000-75000000, chr12: 76000000-76500000, chr12: 79500000-80000000, |
| | chr12: 80000000-80500000, chr12: 93000000-93500000, chr12: 93500000-94000000, |
| | chr12: 95500000-96000000, chr12: 105000000-105500000, chr12: 106500000-107000000, |
| | chr12: 109500000-110000000, chr12: 110500000-111000000, chr12: 111000000-111500000, |
| | chr12: 111500000-112000000, chr12: 112000000-112500000, chr12: 112500000-113000000, |
| | chr12: 113000000-113500000, chr12: 113500000-114000000, chr12: 116500000-117000000, |
| | chr12: 120000000-120500000, chr12: 120500000-121000000, chr12: 121000000-121500000, |
| | chr12: 121500000-122000000, chr12: 122000000-122500000, chr12: 123500000-124000000, |
| | chr12: 128500000-129000000, chr12: 129500000-130000000 |
| 13 | chr13: 18000000-18500000, chr13: 18500000-19000000, chr13: 21000000-21500000, |
| | chr13: 29000000-29500000, chr13: 39000000-39500000, chr13: 41000000-41500000, |
| | chr13: 47500000-48000000, chr13: 49500000-50000000, chr13: 51000000-51500000, |
| | chr13: 57000000-57500000, chr13: 60500000-61000000, chr13: 67500000-68000000, |
| | chr13: 75500000-76000000, chr13: 85000000-85500000, chr13: 85500000-86000000, |
| | chr13: 86500000-87000000, chr13: 92500000-93000000, chr13: 101500000-102000000, |
| | chr13: 113500000-114000000 |
| 14 | chr14: 18000000-18500000, chr14: 18500000-19000000, chr14: 19000000-19500000, |
| | chr14: 20000000-20500000, chr14: 21000000-21500000, chr14: 22500000-23000000, |
| | chr14: 23000000-23500000, chr14: 28000000-28500000, chr14: 30000000-30500000, |
| | chr14: 32500000-33000000, chr14: 35000000-35500000, chr14: 37000000-37500000, |
| | chr14: 39000000-39500000, chr14: 44500000-45000000, chr14: 55000000-55500000, |
| | chr14: 55500000-56000000, chr14: 60000000-60500000, chr14: 67000000-67500000, |
| | chr14: 68500000-69000000, chr14: 73000000-73500000, chr14: 75000000-75500000, |
| | chr14: 76000000-76500000, chr14: 77000000-77500000, chr14: 81000000-81500000, |
| | chr14: 88500000-89000000, chr14: 90500000-91000000, chr14: 92000000-92500000, |
| | chr14: 92500000-93000000, chr14: 94500000-95000000, chr14: 95500000-96000000, |
| | chr14: 96000000-96500000, chr14: 96500000-97000000, chr14: 97000000-97500000, |
| | chr14: 97500000-98000000, chr14: 99500000-100000000, chr14: 103000000-103500000, |
| | chr14: 104000000-104500000 |
| 15 | chr15: 17000000-17500000, chr15: 19500000-20000000, chr15: 20000000-20500000, |
| | chr15: 20500000-21000000, chr15: 21000000-21500000, chr15: 21500000-22000000, |
| | chr15: 23000000-23500000, chr15: 30000000-30500000, chr15: 30500000-31000000, |
| | chr15: 31500000-32000000, chr15: 34000000-34500000, chr15: 38000000-38500000, |
| | chr15: 39500000-40000000, chr15: 41000000-41500000, chr15: 46000000-46500000, |
| | chr15: 57500000-58000000, chr15: 60000000-60500000, chr15: 63000000-63500000, |
| | chr15: 63500000-64000000, chr15: 64500000-65000000, chr15: 65000000-65500000, |
| | chr15: 66500000-67000000, chr15: 69000000-69500000, chr15: 70000000-70500000, |
| | chr15: 74500000-75000000, chr15: 79500000-80000000, chr15: 81000000-81500000, |
| | chr15: 81500000-82000000, chr15: 84000000-84500000, chr15: 86500000-87000000, |
| | chr15: 90000000-90500000, chr15: 91500000-92000000, chr15: 94000000-94500000 |
| 16 | chr16: 6500000-7000000, chr16: 10500000-11000000, chr16: 11500000-12000000, |
| | chr16: 13000000-13500000, chr16: 13500000-14000000, chr16: 14000000-14500000, |
| | chr16: 14500000-15000000, chr16: 15000000-15500000, chr16: 16000000-16500000, |
| | chr16: 17500000-18000000, chr16: 18000000-18500000, chr16: 21500000-22000000, |
| | chr16: 23500000-24000000, chr16: 24500000-25000000, chr16: 26000000-26500000, |
| | chr16: 26500000-27000000, chr16: 27000000-27500000, chr16: 27500000-28000000, |
| | chr16: 29000000-29500000, chr16: 30500000-31000000, chr16: 31000000-31500000, |
| | chr16: 32000000-32500000, chr16: 32500000-33000000, chr16: 33000000-33500000, |
| | chr16: 33500000-34000000, chr16: 34000000-34500000, chr16: 34500000-35000000, |
| | chr16: 46000000-46500000, chr16: 49000000-49500000, chr16: 49500000-50000000, |
| | chr16: 53000000-53500000, chr16: 59500000-60000000, chr16: 66500000-67000000, |
| | chr16: 67500000-68000000, chr16: 71500000-72000000, chr16: 72000000-72500000, |
| | chr16: 74000000-74500000, chr16: 76000000-76500000, chr16: 79500000-80000000, |
| | chr16: 82500000-83000000, chr16: 84500000-85000000, chr16: 85500000-86000000, |
| | chr16: 90000000-90338345 |
| 17 | chr17: 500000-1000000, chr17: 1000000-1500000, chr17: 1500000-2000000, chr17: 2500000- |
| | 3000000, chr17: 4000000-4500000, chr17: 4500000-5000000, chr17: 5000000-5500000, |
| | chr17: 7000000-7500000, chr17: 7500000-8000000, chr17: 8000000-8500000, chr17: 8500000- |
| | 9000000, chr17: 10000000-10500000, chr17: 10500000-11000000, chr17: 13000000-13500000, |
| | chr17: 19500000-20000000, chr17: 21500000-22000000, chr17: 26500000-27000000, |
| | chr17: 27500000-28000000, chr17: 28500000-29000000, chr17: 31000000-31500000, |
| | chr17: 33000000-33500000, chr17: 33500000-34000000, chr17: 36500000-37000000, |
| | chr17: 38500000-39000000, chr17: 40000000-40500000, chr17: 41500000-42000000, |
| | chr17: 42500000-43000000, chr17: 43500000-44000000, chr17: 44000000-44500000, |
| | chr17: 45000000-45500000, chr17: 48500000-49000000, chr17: 49500000-50000000, |
| | chr17: 56000000-56500000, chr17: 56500000-57000000, chr17: 59000000-59500000, |
| | chr17: 59500000-60000000, chr17: 64000000-64500000, chr17: 65000000-65500000, |
| | chr17: 65500000-66000000, chr17: 66000000-66500000, chr17: 67000000-67500000, |
| | chr17: 70500000-71000000, chr17: 71000000-71500000, chr17: 72500000-73000000, |
| | chr17: 74000000-74500000, chr17: 75000000-75500000, chr17: 80000000-80500000 |
| 18 | chr18: 2500000-3000000, chr18: 3500000-4000000, chr18: 9500000-10000000, chr18: 14000000- |
| | 14500000, chr18: 15000000-15500000, chr18: 20500000-21000000, chr18: 24000000-24500000, |
| | chr18: 42500000-43000000, chr18: 50500000-51000000, chr18: 53500000-54000000, |
| | chr18: 67000000-67500000, chr18: 73500000-74000000 |
| 19 | chr19: 0-500000, chr19: 500000-1000000, chr19: 1000000-1500000, chr19: 1500000-2000000, |
| | chr19: 2500000-3000000, chr19: 3000000-3500000, chr19: 4500000-5000000, chr19: 5500000- |
| | 6000000, chr19: 6000000-6500000, chr19: 6500000-7000000, chr19: 7500000-8000000, |
| | chr19: 8000000-8500000, chr19: 8500000-9000000, chr19: 9500000-10000000, chr19: 10000000- |
| | 10500000, chr19: 10500000-11000000, chr19: 11000000-11500000, chr19: 12500000-13000000, |
| | chr19: 14500000-15000000, chr19: 15500000-16000000, chr19: 16000000-16500000, |
| | chr19: 16500000-17000000, chr19: 17000000-17500000, chr19: 17500000-18000000, |
| | chr19: 18000000-18500000, chr19: 19000000-19500000, chr19: 27500000-28000000, |
| | chr19: 35000000-35500000, chr19: 35500000-36000000, chr19: 38000000-38500000, |
| | chr19: 38500000-39000000, chr19: 39000000-39500000, chr19: 39500000-40000000, |
| | chr19: 40000000-40500000, chr19: 40500000-41000000, chr19: 41000000-41500000, |
| | chr19: 41500000-42000000, chr19: 42000000-42500000, chr19: 43000000-43500000, |
| | chr19: 44500000-45000000, chr19: 45000000-45500000, chr19: 45500000-46000000, |
| | chr19: 46500000-47000000, chr19: 47000000-47500000, chr19: 48000000-48500000, |
| | chr19: 49500000-50000000, chr19: 50500000-51000000, chr19: 51000000-51500000, |
| | chr19: 52000000-52500000, chr19: 55500000-56000000, chr19: 56000000-56500000 |
| 20 | chr20: 1500000-2000000, chr20: 2000000-2500000, chr20: 3000000-3500000, chr20: 5000000- |
| | 5500000, chr20: 5500000-6000000, chr20: 10000000-10500000, chr20: 10500000-11000000, |
| | chr20: 14500000-15000000, chr20: 20500000-21000000, chr20: 25000000-25500000, |
| | chr20: 25500000-26000000, chr20: 26000000-26500000, chr20: 28500000-29000000, |
| | chr20: 29000000-29500000, chr20: 29500000-30000000, chr20: 30000000-30500000, |
| | chr20: 30500000-31000000, chr20: 31500000-32000000, chr20: 32000000-32500000, |
| | chr20: 33000000-33500000, chr20: 35000000-35500000, chr20: 35500000-36000000, |
| | chr20: 36500000-37000000, chr20: 37000000-37500000, chr20: 38500000-39000000, |
| | chr20: 40500000-41000000, chr20: 44000000-44500000, chr20: 44500000-45000000, |
| | chr20: 46000000-46500000, chr20: 47000000-47500000, chr20: 48000000-48500000, |
| | chr20: 48500000-49000000, chr20: 50000000-50500000, chr20: 51500000-52000000, |
| | chr20: 53500000-54000000, chr20: 56500000-57000000, chr20: 57000000-57500000, |
| | chr20: 57500000-58000000, chr20: 58500000-59000000, chr20: 61000000-61500000, |
| | chr20: 62500000-63000000 |
| 21 | chr21: 5000000-5500000, chr21: 5500000-6000000, chr21: 6000000-6500000, chr21: 6500000- |
| | 7000000, chr21: 7000000-7500000, chr21: 7500000-8000000, chr21: 8000000-8500000, |
| | chr21: 8500000-9000000, chr21: 9000000-9500000, chr21: 9500000-10000000, chr21: 10000000- |
| | 10500000, chr21: 13000000-13500000, chr21: 13500000-14000000, chr21: 14500000-15000000, |
| | chr21: 15000000-15500000, chr21: 18000000-18500000, chr21: 24500000-25000000, |
| | chr21: 29000000-29500000, chr21: 31000000-31500000, chr21: 32500000-33000000, |
| | chr21: 33500000-34000000, chr21: 35500000-36000000, chr21: 36000000-36500000, |
| | chr21: 38000000-38500000, chr21: 39500000-40000000 |
| 22 | chr22: 10500000-11000000, chr22: 11000000-11500000, chr22: 11500000-12000000, |
| | chr22: 12000000-12500000, chr22: 12500000-13000000, chr22: 15000000-15500000, |
| | chr22: 15500000-16000000, chr22: 16000000-16500000, chr22: 18500000-19000000, |
| | chr22: 19000000-19500000, chr22: 23500000-24000000, chr22: 25000000-25500000, |
| | chr22: 27000000-27500000, chr22: 27500000-28000000, chr22: 29500000-30000000, |
| | chr22: 31500000-32000000, chr22: 32000000-32500000, chr22: 35000000-35500000, |
| | chr22: 35500000-36000000, chr22: 36500000-37000000, chr22: 37500000-38000000, |
| | chr22: 38000000-38500000, chr22: 38500000-39000000, chr22: 41500000-42000000, |
| | chr22: 42500000-43000000, chr22: 43000000-43500000, chr22: 48000000-48500000 |

TABLE 20

| | |
|---|---|
| 1 | 1_10000000_10500000, 1_101000000_101500000, 1_103000000_103500000, |
| | 1_104500000_105000000, 1_107500000_108000000, 1_108500000_109000000, |
| | 1_109000000_109500000, 1_109500000_110000000, 1_110000000_110500000, |
| | 1_110500000_111000000, 1_111500000_112000000, 1_112500000_113000000, |
| | 1_114000000_114500000, 1_11500000_12000000, 1_115000000_115500000, |
| | 1_116000000_116500000, 1_116500000_117000000, 1_118500000_119000000, |
| | 1_12000000_12500000, 1_120000000_120500000, 1_120500000_121000000, |
| | 1_121000000_121500000, 1_12500000_13000000, 1_125000000_125500000, |
| | 1_13000000_13500000, 1_143000000_143500000, 1_143500000_144000000, |
| | 1_144000000_144500000, 1_144500000_145000000, 1_145000000_145500000, |
| | 1_146000000_146500000, 1_146500000_147000000, 1_147500000_148000000, |
| | 1_148000000_148500000, 1_148500000_149000000, 1_149000000_149500000, |
| | 1_149500000_150000000, 1_1500000_2000000, 1_150000000_150500000, |
| | 1_150500000_151000000, 1_151000000_151500000, 1_151500000_152000000, |
| | 1_152000000_152500000, 1_153000000_153500000, 1_153500000_154000000, |
| | 1_154000000_154500000, 1_154500000_155000000, 1_15500000_16000000, |
| | 1_155000000_155500000, 1_155500000_156000000, 1_156000000_156500000, |
| | 1_156500000_157000000, 1_157000000_157500000, 1_158000000_158500000, |
| | 1_158500000_159000000, 1_159000000_159500000, 1_159500000_160000000, |
| | 1_16000000_16500000, 1_160000000_160500000, 1_160500000_161000000, |
| | 1_161000000_161500000, 1_161500000_162000000, 1_162000000_162500000, |
| | 1_163500000_164000000, 1_16500000_17000000, 1_165500000_166000000, |
| | 1_167000000_167500000, 1_167500000_168000000, 1_168500000_169000000, |
| | 1_169500000_170000000, 1_171000000_171500000, 1_171500000_172000000, |
| | 1_172500000_173000000, 1_173500000_174000000, 1_174000000_174500000, |
| | 1_175000000_175500000, 1_175500000_176000000, 1_176500000_177000000, |
| | 1_177500000_178000000, 1_179000000_179500000, 1_18000000_18500000, |
| | 1_181000000_181500000, 1_182000000_182500000, 1_182500000_183000000, |
| | 1_183000000_183500000, 1_183500000_184000000, 1_184500000_185000000, |
| | 1_185000000_185500000, 1_186000000_186500000, 1_188500000_189000000, |
| | 1_19000000_19500000, 1_190000000_190500000, 1_192500000_193000000, |
| | 1_193000000_193500000, 1_193500000_194000000, 1_19500000_20000000, |
| | 1_197000000_197500000, 1_198500000_199000000, 1_199000000_199500000, |
| | 1_20000000_20500000, 1_200000000_200500000, 1_200500000_201000000, |
| | 1_201500000_202000000, 1_202500000_203000000, 1_203000000_203500000, |
| | 1_203500000_204000000, 1_204000000_204500000, 1_20500000_21000000, |
| | 1_205000000_205500000, 1_206500000_207000000, 1_207000000_207500000, |
| | 1_208000000_208500000, 1_209500000_210000000, 1_21000000_21500000, |
| | 1_210000000_210500000, 1_211500000_212000000, 1_212500000_213000000, |
| | 1_213500000_214000000, 1_214000000_214500000, 1_21500000_22000000, |
| | 1_219000000_219500000, 1_22000000_22500000, 1_220000000_220500000, |
| | 1_221000000_221500000, 1_221500000_222000000, 1_222500000_223000000, |
| | 1_224000000_224500000, 1_224500000_225000000, 1_22500000_23000000, |
| | 1_225500000_226000000, 1_226000000_226500000, 1_227000000_227500000, |
| | 1_229000000_229500000, 1_229500000_230000000, 1_23000000_23500000, |
| | 1_230500000_231000000, 1_231500000_232000000, 1_232000000_232500000, |
| | 1_233000000_233500000, 1_233500000_234000000, 1_234000000_234500000, |
| | 1_23500000_24000000, 1_235000000_235500000, 1_235500000_236000000, |
| | 1_236000000_236500000, 1_239500000_240000000, 1_240500000_241000000, |
| | 1_241500000_242000000, 1_242000000_242500000, 1_242500000_243000000, |
| | 1_243500000_244000000, 1_244000000_244500000, 1_244500000_245000000, |
| | 1_24500000_25000000, 1_245000000_245500000, 1_246500000_247000000, |
| | 1_247000000_247500000, 1_248000000_248500000, 1_2500000_3000000, |
| | 1_25500000_26000000, 1_26000000_26500000, 1_26500000_27000000, |
| | 1_27000000_27500000, 1_27500000_28000000, 1_28000000_28500000, |
| | 1_28500000_29000000, 1_29000000_29500000, 1_29500000_30000000, |
| | 1_3000000_3500000, 1_30500000_31000000, 1_31000000_31500000, |
| | 1_31500000_32000000, 1_32000000_32500000, 1_32500000_33000000, |
| | 1_33000000_33500000, 1_33500000_34000000, 1_34500000_35000000, |
| | 1_35000000_35500000, 1_35500000_36000000, 1_36000000_36500000, |
| | 1_36500000_37000000, 1_37000000_37500000, 1_37500000_38000000, |
| | 1_38500000_39000000, 1_39000000_39500000, 1_39500000_40000000, |
| | 1_4000000_4500000, 1_40000000_40500000, 1_40500000_41000000, |
| | 1_41000000_41500000, 1_42000000_42500000, 1_42500000_43000000, |
| | 1_44500000_45000000, 1_45500000_46000000, 1_46000000_46500000, |
| | 1_48000000_48500000, 1_49000000_49500000, 1_49500000_50000000, |
| | 1_500000_1000000, 1_50000000_50500000, 1_51500000_52000000, |
| | 1_52000000_52500000, 1_52500000_53000000, 1_53500000_54000000, |
| | 1_54000000_54500000, 1_56500000_57000000, 1_57000000_57500000, |
| | 1_58500000_59000000, 1_59500000_60000000, 1_60000000_60500000, |
| | 1_61000000_61500000, 1_63500000_64000000, 1_64000000_64500000, |
| | 1_64500000_65000000, 1_65000000_65500000, 1_66000000_66500000, |
| | 1_66500000_67000000, 1_67000000_67500000, 1_67500000_68000000, |
| | 1_68500000_69000000, 1_70000000_70500000, 1_73000000_73500000, |
| | 1_74500000_75000000, 1_77000000_77500000, 1_77500000_78000000, |
| | 1_79000000_79500000, 1_8000000_8500000, 1_81500000_82000000, |
| | 1_83000000_83500000, 1_83500000_84000000, 1_84000000_84500000, |
| | 1_8500000_9000000, 1_87500000_88000000, 1_88500000_89000000, |
| | 1_89500000_90000000, 1_90000000_90500000, 1_91500000_92000000, |
| | 1_92500000_93000000, 1_93000000_93500000, 1_94500000_95000000, |
| | 1_9500000_10000000, 1_96500000_97000000, 1_97000000_97500000, |
| | 1_97500000_98000000, 1_98000000_98500000, 1_98500000_99000000 |
| 2 | 2_101500000_102000000, 2_102000000_102500000, 2_103000000_103500000, |
| | 2_105500000_106000000, 2_106000000_106500000, 2_107000000_107500000, |
| | 2_107500000_108000000, 2_109500000_110000000, 2_110000000_110500000, |
| | 2_110500000_111000000, 2_112500000_113000000, 2_113000000_113500000, |
| | 2_113500000_114000000, 2_114500000_115000000, 2_11500000_12000000, |
| | 2_116000000_116500000, 2_117000000_117500000, 2_118000000_118500000, |
| | 2_119500000_120000000, 2_122000000_122500000, 2_125000000_125500000, |
| | 2_126500000_127000000, 2_127500000_128000000, 2_128000000_128500000, |
| | 2_129000000_129500000, 2_129500000_130000000, 2_130000000_130500000, |
| | 2_130500000_131000000, 2_131500000_132000000, 2_132000000_132500000, |
| | 2_132500000_133000000, 2_133000000_133500000, 2_134000000_134500000, |
| | 2_134500000_135000000, 2_13500000_14000000, 2_135500000_136000000, |
| | 2_136000000_136500000, 2_139500000_140000000, 2_143000000_143500000, |
| | 2_143500000_144000000, 2_144000000_144500000, 2_144500000_145000000, |
| | 2_14500000_15000000, 2_145000000_145500000, 2_145500000_146000000, |
| | 2_147500000_148000000, 2_148500000_149000000, 2_1500000_2000000, |
| | 2_150000000_150500000, 2_150500000_151000000, 2_151000000_151500000, |
| | 2_152000000_152500000, 2_153000000_153500000, 2_15500000_16000000, |
| | 2_156000000_156500000, 2_157000000_157500000, 2_158000000_158500000, |
| | 2_159000000_159500000, 2_16000000_16500000, 2_161000000_161500000, |
| | 2_162000000_162500000, 2_165500000_166000000, 2_168000000_168500000, |
| | 2_168500000_169000000, 2_171000000_171500000, 2_171500000_172000000, |
| | 2_173500000_174000000, 2_174000000_174500000, 2_174500000_175000000, |
| | 2_176000000_176500000, 2_177000000_177500000, 2_178000000_178500000, |
| | 2_181000000_181500000, 2_181500000_182000000, 2_183500000_184000000, |
| | 2_185500000_186000000, 2_188500000_189000000, 2_189000000_189500000, |
| | 2_189500000_190000000, 2_190000000_190500000, 2_190500000_191000000, |
| | 2_191000000_191500000, 2_191500000_192000000, 2_195000000_195500000, |
| | 2_196000000_196500000, 2_197000000_197500000, 2_198000000_198500000, |
| | 2_20000000_20500000, 2_200500000_201000000, 2_201000000_201500000, |
| | 2_201500000_202000000, 2_203000000_203500000, 2_203500000_204000000, |
| | 2_204000000_204500000, 2_206000000_206500000, 2_207000000_207500000, |
| | 2_207500000_208000000, 2_208500000_209000000, 2_21000000_21500000, |
| | 2_210000000_210500000, 2_213000000_213500000, 2_21500000_22000000, |
| | 2_215500000_216000000, 2_216000000_216500000, 2_216500000_217000000, |
| | 2_217500000_218000000, 2_218000000_218500000, 2_218500000_219000000, |
| | 2_219000000_219500000, 2_219500000_220000000, 2_220000000_220500000, |
| | 2_221000000_221500000, 2_224500000_225000000, 2_22500000_23000000, |
| | 2_225000000_225500000, 2_226000000_226500000, 2_226500000_227000000, |
| | 2_227000000_227500000, 2_227500000_228000000, 2_228000000_228500000, |
| | 2_229500000_230000000, 2_230000000_230500000, 2_230500000_231000000, |
| | 2_231000000_231500000, 2_231500000_232000000, 2_235000000_235500000, |
| | 2_235500000_236000000, 2_236500000_237000000, 2_238500000_239000000, |
| | 2_240500000_241000000, 2_24500000_25000000, 2_25000000_25500000, |
| | 2_25500000_26000000, 2_26000000_26500000, 2_27000000_27500000, |
| | 2_27500000_28000000, 2_28500000_29000000, 2_3000000_3500000, |
| | 2_30000000_30500000, 2_32000000_32500000, 2_35000000_35500000, |
| | 2_36000000_36500000, 2_36500000_37000000, 2_37000000_37500000, |
| | 2_37500000_38000000, 2_38500000_39000000, 2_39000000_39500000, |
| | 2_42000000_42500000, 2_43000000_43500000, 2_44000000_44500000, |
| | 2_45500000_46000000, 2_47000000_47500000, 2_47500000_48000000, |
| | 2_48000000_48500000, 2_48500000_49000000, 2_49000000_49500000, |
| | 2_5000000_5500000, 2_50000000_50500000, 2_51500000_52000000, |
| | 2_53000000_53500000, 2_54000000_54500000, 2_57500000_58000000, |
| | 2_59500000_60000000, 2_60500000_61000000, 2_61000000_61500000, |
| | 2_61500000_62000000, 2_62000000_62500000, 2_64000000_64500000, |
| | 2_64500000_65000000, 2_65000000_65500000, 2_66000000_66500000, |
| | 2_68000000_68500000, 2_68500000_69000000, 2_69000000_69500000, |
| | 2_69500000_70000000, 2_70000000_70500000, 2_70500000_71000000, |
| | 2_71000000_71500000, 2_71500000_72000000, 2_72000000_72500000, |
| | 2_72500000_73000000, 2_73500000_74000000, 2_74500000_75000000, |
| | 2_75000000_75500000, 2_76000000_76500000, 2_76500000_77000000, |
| | 2_78500000_79000000, 2_79000000_79500000, 2_8000000_8500000, |
| | 2_80000000_80500000, 2_81000000_81500000, 2_84000000_84500000, |
| | 2_84500000_85000000, 2_8500000_9000000, 2_85500000_86000000, |
| | 2_87000000_87500000, 2_87500000_88000000, 2_88500000_89000000, |
| | 2_89000000_89500000, 2_89500000_90000000, 2_90000000_90500000, |
| | 2_91000000_91500000, 2_91500000_92000000, 2_92000000_92500000, |
| | 2_94500000_95000000, 2_9500000_10000000, 2_95500000_96000000, |
| | 2_96000000_96500000, 2_96500000_97000000, 2_97000000_97500000, |
| | 2_97500000_98000000, 2_98500000_99000000, 2_99000000_99500000 |
| 3 | 3_10000000_10500000, 3_100000000_100500000, 3_101000000_101500000, |
| | 3_101500000_102000000, 3_10500000_11000000, 3_105500000_106000000, |
| | 3_106000000_106500000, 3_107500000_108000000, 3_108000000_108500000, |
| | 3_108500000_109000000, 3_110000000_110500000, 3_112000000_112500000, |
| | 3_112500000_113000000, 3_113000000_113500000, 3_115000000_115500000, |
| | 3_116500000_117000000, 3_117000000_117500000, 3_119500000_120000000, |
| | 3_122000000_122500000, 3_12500000_13000000, 3_125000000_125500000, |
| | 3_125500000_126000000, 3_127000000_127500000, 3_127500000_128000000, |
| | 3_128500000_129000000, 3_129000000_129500000, 3_129500000_130000000, |
| | 3_132500000_133000000, 3_134000000_134500000, 3_13500000_14000000, |
| | 3_135000000_135500000, 3_135500000_136000000, 3_136500000_137000000, |
| | 3_138500000_139000000, 3_14000000_14500000, 3_141000000_141500000, |
| | 3_141500000_142000000, 3_143000000_143500000, 3_143500000_144000000, |
| | 3_145500000_146000000, 3_146000000_146500000, 3_147500000_148000000, |
| | 3_148000000_148500000, 3_149000000_149500000, 3_150000000_150500000, |
| | 3_151000000_151500000, 3_151500000_152000000, 3_152000000_152500000, |
| | 3_152500000_153000000, 3_153000000_153500000, 3_153500000_154000000, |
| | 3_155500000_156000000, 3_156500000_157000000, 3_157000000_157500000, |
| | 3_157500000_158000000, 3_158500000_159000000, 3_160500000_161000000, |
| | 3_161000000_161500000, 3_161500000_162000000, 3_162000000_162500000, |
| | 3_163000000_163500000, 3_16500000_17000000, 3_165500000_166000000, |
| | 3_166500000_167000000, 3_169000000_169500000, 3_169500000_170000000, |
| | 3_17000000_17500000, 3_170000000_170500000, 3_171000000_171500000, |
| | 3_171500000_172000000, 3_172000000_172500000, 3_17500000_18000000, |
| | 3_179000000_179500000, 3_179500000_180000000, 3_182500000_183000000, |
| | 3_183000000_183500000, 3_184000000_184500000, 3_184500000_185000000, |
| | 3_185500000_186000000, 3_186000000_186500000, 3_186500000_187000000, |
| | 3_187500000_188000000, 3_188000000_188500000, 3_188500000_189000000, |
| | 3_189000000_189500000, 3_191000000_191500000, 3_191500000_192000000, |
| | 3_192000000_192500000, 3_194500000_195000000, 3_195000000_195500000, |
| | 3_196500000_197000000, 3_197000000_197500000, 3_197500000_198000000, |
| | 3_198000000_198295559, 3_21000000_21500000, 3_21500000_22000000, |
| | 3_22000000_22500000, 3_23500000_24000000, 3_2500000_3000000, |
| | 3_25500000_26000000, 3_26000000_26500000, 3_27500000_28000000, |
| | 3_28500000_29000000, 3_3000000_3500000, 3_30000000_30500000, |
| | 3_30500000_31000000, 3_31500000_32000000, 3_32000000_32500000, |
| | 3_32500000_33000000, 3_33000000_33500000, 3_33500000_34000000, |
| | 3_34000000_34500000, 3_36000000_36500000, 3_36500000_37000000, |
| | 3_37000000_37500000, 3_38000000_38500000, 3_39000000_39500000, |
| | 3_42000000_42500000, 3_43500000_44000000, 3_44000000_44500000, |
| | 3_4500000_5000000, 3_45000000_45500000, 3_46000000_46500000, |
| | 3_47000000_47500000, 3_47500000_48000000, 3_48500000_49000000, |
| | 3_49000000_49500000, 3_49500000_50000000, 3_500000_1000000, |
| | 3_5000000_5500000, 3_51500000_52000000, 3_52000000_52500000, |
| | 3_54500000_55000000, 3_55500000_56000000, 3_56500000_57000000, |
| | 3_57500000_58000000, 3_58500000_59000000, 3_59000000_59500000, |
| | 3_63500000_64000000, 3_64500000_65000000, 3_6500000_7000000, |
| | 3_65000000_65500000, 3_66000000_66500000, 3_67500000_68000000, |
| | 3_68500000_69000000, 3_69000000_69500000, 3_69500000_70000000, |
| | 3_7000000_7500000, 3_70000000_70500000, 3_71000000_71500000, |
| | 3_71500000_72000000, 3_72000000_72500000, 3_72500000_73000000, |
| | 3_73500000_74000000, 3_75000000_75500000, 3_75500000_76000000, |
| | 3_76500000_77000000, 3_77500000_78000000, 3_78000000_78500000, |
| | 3_79000000_79500000, 3_88000000_88500000, 3_88500000_89000000, |
| | 3_89000000_89500000, 3_89500000_90000000, 3_9000000_9500000, |
| | 3_91000000_91500000, 3_9500000_10000000, 3_95500000_96000000, |
| | 3_96500000_97000000, 3_99500000_100000000 |
| 4 | 4_1000000_1500000, 4_10000000_10500000, 4_101000000_101500000, |
| | 4_102000000_102500000, 4_103000000_103500000, 4_105000000_105500000, |
| | 4_107500000_108000000, 4_108000000_108500000, 4_109000000_109500000, |
| | 4_109500000_110000000, 4_111000000_111500000, 4_111500000_112000000, |
| | 4_112500000_113000000, 4_113500000_114000000, 4_117500000_118000000, |
| | 4_121500000_122000000, 4_125500000_126000000, 4_126500000_127000000, |
| | 4_128500000_129000000, 4_129500000_130000000, 4_130500000_131000000, |
| | 4_131500000_132000000, 4_133500000_134000000, 4_135000000_135500000, |
| | 4_139000000_139500000, 4_140000000_140500000, 4_142000000_142500000, |
| | 4_142500000_143000000, 4_145000000_145500000, 4_146000000_146500000, |
| | 4_147000000_147500000, 4_15000000_15500000, 4_150500000_151000000, |
| | 4_151000000_151500000, 4_152000000_152500000, 4_152500000_153000000, |
| | 4_153000000_153500000, 4_153500000_154000000, 4_156000000_156500000, |
| | 4_158500000_159000000, 4_159000000_159500000, 4_160000000_160500000, |
| | 4_161500000_162000000, 4_164000000_164500000, 4_164500000_165000000, |
| | 4_16500000_17000000, 4_165500000_166000000, 4_166500000_167000000, |
| | 4_167000000_167500000, 4_171500000_172000000, 4_172000000_172500000, |
| | 4_173000000_173500000, 4_175000000_175500000, 4_175500000_176000000, |
| | 4_176000000_176500000, 4_178500000_179000000, 4_18000000_18500000, |
| | 4_181500000_182000000, 4_184000000_184500000, 4_184500000_185000000, |
| | 4_185500000_186000000, 4_186000000_186500000, 4_187000000_187500000, |
| | 4_187500000_188000000, 4_188000000_188500000, 4_188500000_189000000, |
| | 4_189500000_190000000, 4_190000000_190214555, 4_2000000_2500000, |
| | 4_21000000_21500000, 4_2500000_3000000, 4_25000000_25500000, |
| | 4_26000000_26500000, 4_26500000_27000000, 4_27000000_27500000, |
| | 4_27500000_28000000, 4_29000000_29500000, 4_29500000_30000000, |
| | 4_32500000_33000000, 4_34500000_35000000, 4_36000000_36500000, |
| | 4_36500000_37000000, 4_37500000_38000000, 4_38000000_38500000, |
| | 4_38500000_39000000, 4_39000000_39500000, 4_39500000_40000000, |
| | 4_40000000_40500000, 4_40500000_41000000, 4_4500000_5000000, |
| | 4_45000000_45500000, 4_47500000_48000000, 4_48500000_49000000, |
| | 4_49000000_49500000, 4_49500000_50000000, 4_52000000_52500000, |
| | 4_54500000_55000000, 4_5500000_6000000, 4_57500000_58000000, |
| | 4_58000000_58500000, 4_59000000_59500000, 4_6000000_6500000, |
| | 4_60500000_61000000, 4_61000000_61500000, 4_61500000_62000000, |
| | 4_62000000_62500000, 4_64000000_64500000, 4_65000000_65500000, |
| | 4_66000000_66500000, 4_66500000_67000000, 4_67000000_67500000, |
| | 4_67500000_68000000, 4_68000000_68500000, 4_68500000_69000000, |
| | 4_69500000_70000000, 4_70500000_71000000, 4_71500000_72000000, |
| | 4_72500000_73000000, 4_73500000_74000000, 4_74000000_74500000, |
| | 4_7500000_8000000, 4_75500000_76000000, 4_77000000_77500000, |
| | 4_77500000_78000000, 4_78000000_78500000, 4_78500000_79000000, |
| | 4_80000000_80500000, 4_80500000_81000000, 4_81500000_82000000, |
| | 4_82000000_82500000, 4_83000000_83500000, 4_83500000_84000000, |
| | 4_84500000_85000000, 4_8500000_9000000, 4_88500000_89000000, |
| | 4_89000000_89500000, 4_89500000_90000000, 4_9000000_9500000, |
| | 4_9500000_10000000, 4_98000000_98500000, 4_98500000_99000000, |
| | 4_99500000_100000000 |
| 5 | 5_100500000_101000000, 5_102500000_103000000, 5_10500000_11000000, |
| | 5_109500000_110000000, 5_11000000_11500000, 5_110000000_110500000, |
| | 5_110500000_111000000, 5_111000000_111500000, 5_112000000_112500000, |
| | 5_112500000_113000000, 5_113000000_113500000, 5_114000000_114500000, |
| | 5_11500000_12000000, 5_115500000_116000000, 5_119000000_119500000, |
| | 5_119500000_120000000, 5_124000000_124500000, 5_124500000_125000000, |
| | 5_125000000_125500000, 5_126500000_127000000, 5_131000000_131500000, |
| | 5_131500000_132000000, 5_132000000_132500000, 5_132500000_133000000, |
| | 5_134500000_135000000, 5_13500000_14000000, 5_136500000_137000000, |
| | 5_137500000_138000000, 5_138000000_138500000, 5_138500000_139000000, |
| | 5_139000000_139500000, 5_139500000_140000000, 5_14000000_14500000, |
| | 5_140000000_140500000, 5_140500000_141000000, 5_141000000_141500000, |
| | 5_141500000_142000000, 5_142000000_142500000, 5_142500000_143000000, |
| | 5_143000000_143500000, 5_14500000_15000000, 5_145000000_145500000, |
| | 5_146000000_146500000, 5_148000000_148500000, 5_148500000_149000000, |
| | 5_149000000_149500000, 5_149500000_150000000, 5_1500000_2000000, |
| | 5_151000000_151500000, 5_151500000_152000000, 5_152000000_152500000, |
| | 5_15500000_16000000, 5_157000000_157500000, 5_157500000_158000000, |
| | 5_158000000_158500000, 5_158500000_159000000, 5_159000000_159500000, |
| | 5_16000000_16500000, 5_160000000_160500000, 5_161500000_162000000, |
| | 5_162500000_163000000, 5_166500000_167000000, 5_167500000_168000000, |
| | 5_169000000_169500000, 5_169500000_170000000, 5_170000000_170500000, |
| | 5_171500000_172000000, 5_172000000_172500000, 5_172500000_173000000, |
| | 5_173500000_174000000, 5_174500000_175000000, 5_17500000_18000000, |
| | 5_176000000_176500000, 5_176500000_177000000, 5_177000000_177500000, |
| | 5_180000000_180500000, 5_181000000_181500000, 5_19000000_19500000, |
| | 5_2000000_2500000, 5_20000000_20500000, 5_21000000_21500000, |
| | 5_22500000_23000000, 5_23000000_23500000, 5_23500000_24000000, |
| | 5_24000000_24500000, 5_24500000_25000000, 5_26000000_26500000, |
| | 5_3000000_3500000, 5_31500000_32000000, 5_34000000_34500000, |
| | 5_36500000_37000000, 5_38000000_38500000, 5_39000000_39500000, |
| | 5_4000000_4500000, 5_40500000_41000000, 5_41000000_41500000, |
| | 5_43000000_43500000, 5_43500000_44000000, 5_44000000_44500000, |
| | 5_4500000_5000000, 5_45500000_46000000, 5_50000000_50500000, |
| | 5_50500000_51000000, 5_51000000_51500000, 5_54000000_54500000, |
| | 5_54500000_55000000, 5_55500000_56000000, 5_56000000_56500000, |
| | 5_56500000_57000000, 5_59000000_59500000, 5_59500000_60000000, |
| | 5_60500000_61000000, 5_61000000_61500000, 5_62000000_62500000, |
| | 5_63500000_64000000, 5_65000000_65500000, 5_65500000_66000000, |
| | 5_66000000_66500000, 5_67000000_67500000, 5_67500000_68000000, |
| | 5_68000000_68500000, 5_69000000_69500000, 5_69500000_70000000, |
| | 5_7000000_7500000, 5_70000000_70500000, 5_70500000_71000000, |
| | 5_71000000_71500000, 5_74500000_75000000, 5_75000000_75500000, |
| | 5_75500000_76000000, 5_78000000_78500000, 5_79500000_80000000, |
| | 5_80000000_80500000, 5_80500000_81000000, 5_81500000_82000000, |
| | 5_84000000_84500000, 5_85000000_85500000, 5_87000000_87500000, |
| | 5_88500000_89000000, 5_89000000_89500000, 5_89500000_90000000, |
| | 5_9000000_9500000, 5_91000000_91500000, 5_94000000_94500000, |
| | 5_94500000_95000000, 5_9500000_10000000, 5_95500000_96000000, |
| | 5_96000000_96500000, 5_96500000_97000000, 5_97500000_98000000, |
| | 5_99500000_100000000 |
| 6 | 6_104000000_104500000, 6_10500000_11000000, 6_105000000_105500000, |
| | 6_105500000_106000000, 6_106000000_106500000, 6_107000000_107500000, |
| | 6_108500000_109000000, 6_109000000_109500000, 6_11000000_11500000, |
| | 6_110500000_111000000, 6_111000000_111500000, 6_111500000_112000000, |
| | 6_112000000_112500000, 6_112500000_113000000, 6_114000000_114500000, |
| | 6_116000000_116500000, 6_118500000_119000000, 6_119000000_119500000, |
| | 6_119500000_120000000, 6_12000000_12500000, 6_121000000_121500000, |
| | 6_124000000_124500000, 6_127500000_128000000, 6_128000000_128500000, |
| | 6_129000000_129500000, 6_129500000_130000000, 6_13000000_13500000, |
| | 6_130000000_130500000, 6_130500000_131000000, 6_131000000_131500000, |
| | 6_132000000_132500000, 6_133000000_133500000, 6_134000000_134500000, |
| | 6_134500000_135000000, 6_13500000_14000000, 6_135000000_135500000, |
| | 6_135500000_136000000, 6_137000000_137500000, 6_137500000_138000000, |
| | 6_138000000_138500000, 6_139000000_139500000, 6_14000000_14500000, |
| | 6_142000000_142500000, 6_142500000_143000000, 6_143500000_144000000, |
| | 6_144000000_144500000, 6_144500000_145000000, 6_14500000_15000000, |
| | 6_145000000_145500000, 6_147000000_147500000, 6_149000000_149500000, |
| | 6_149500000_150000000, 6_15000000_15500000, 6_154000000_154500000, |
| | 6_156500000_157000000, 6_157000000_157500000, 6_158500000_159000000, |
| | 6_159500000_160000000, 6_16000000_16500000, 6_160000000_160500000, |
| | 6_162000000_162500000, 6_163500000_164000000, 6_16500000_17000000, |
| | 6_165000000_165500000, 6_166500000_167000000, 6_167500000_168000000, |
| | 6_168000000_168500000, 6_168500000_169000000, 6_170000000_170500000, |
| | 6_170500000_170805979, 6_17500000_18000000, 6_18000000_18500000, |
| | 6_19500000_20000000, 6_20000000_20500000, 6_2500000_3000000, |
| | 6_25000000_25500000, 6_25500000_26000000, 6_26000000_26500000, |
| | 6_26500000_27000000, 6_27500000_28000000, 6_29500000_30000000, |
| | 6_30000000_30500000, 6_30500000_31000000, 6_31000000_31500000, |
| | 6_31500000_32000000, 6_32000000_32500000, 6_32500000_33000000, |
| | 6_33000000_33500000, 6_33500000_34000000, 6_34500000_35000000, |
| | 6_35000000_35500000, 6_36000000_36500000, 6_36500000_37000000, |
| | 6_37000000_37500000, 6_37500000_38000000, 6_38500000_39000000, |
| | 6_39500000_40000000, 6_4000000_4500000, 6_40000000_40500000, |
| | 6_40500000_41000000, 6_41000000_41500000, 6_41500000_42000000, |
| | 6_42000000_42500000, 6_42500000_43000000, 6_43000000_43500000, |
| | 6_43500000_44000000, 6_44000000_44500000, 6_45000000_45500000, |
| | 6_46000000_46500000, 6_47000000_47500000, 6_49500000_50000000, |
| | 6_51000000_51500000, 6_53000000_53500000, 6_53500000_54000000, |
| | 6_55500000_56000000, 6_57500000_58000000, 6_58000000_58500000, |
| | 6_58500000_59000000, 6_60000000_60500000, 6_60500000_61000000, |
| | 6_61000000_61500000, 6_62000000_62500000, 6_63500000_64000000, |
| | 6_6500000_7000000, 6_68500000_69000000, 6_69500000_70000000, |
| | 6_70500000_71000000, 6_73000000_73500000, 6_74000000_74500000, |
| | 6_74500000_75000000, 6_77000000_77500000, 6_77500000_78000000, |
| | 6_79500000_80000000, 6_81000000_81500000, 6_8500000_9000000, |
| | 6_86000000_86500000, 6_87000000_87500000, 6_87500000_88000000, |
| | 6_88500000_89000000, 6_89000000_89500000, 6_89500000_90000000, |
| | 6_9000000_9500000, 6_90500000_91000000, 6_92500000_93000000, |
| | 6_94500000_95000000, 6_95000000_95500000, 6_97000000_97500000 |
| 7 | 7_66500000_67000000, 7_45500000_46000000, 7_18000000_18500000, |
| | 7_53500000_54000000, 7_101000000_101500000, 7_6000000_6500000, |
| | 7_130000000_130500000, 7_63500000_64000000, 7_32000000_32500000, |
| | 7_95500000_96000000, 7_26500000_27000000, 7_140000000_140500000, |
| | 7_36500000_37000000, 7_41500000_42000000, 7_44000000_44500000, |
| | 7_6500000_7000000, 7_129000000_129500000, 7_133500000_134000000, |
| | 7_56500000_57000000, 7_74500000_75000000, 7_74000000_74500000, |
| | 7_143000000_143500000, 7_145000000_145500000, 7_152500000_153000000, |
| | 7_139000000_139500000, 7_48000000_48500000, 7_100500000_101000000, |
| | 7_21000000_21500000, 7_127500000_128000000, 7_129500000_130000000, |
| | 7_7000000_7500000, 7_25500000_26000000, 7_26000000_26500000, |
| | 7_131500000_132000000, 7_36000000_36500000, 7_27000000_27500000, |
| | 7_12000000_12500000, 7_84000000_84500000, 7_108000000_108500000, |
| | 7_141500000_142000000, 7_102500000_103000000, 7_128000000_128500000, |
| | 7_151500000_152000000, 7_125000000_125500000, 7_7500000_8000000, |
| | 7_92500000_93000000, 7_112500000_113000000, 7_55500000_56000000, |
| | 7_44500000_45000000, 7_89500000_90000000, 7_103000000_103500000, |
| | 7_3000000_3500000, 7_98000000_98500000, 7_12500000_13000000, |
| | 7_57000000_57500000, 7_38500000_39000000, 7_114500000_115000000, |
| | 7_53000000_53500000, 7_40000000_40500000, 7_1000000_1500000, |
| | 7_135500000_136000000, 7_154000000_154500000, 7_58000000_58500000, |
| | 7_46000000_46500000, 7_30000000_30500000, 7_115000000_115500000, |
| | 7_51500000_52000000, 7_0_500000, 7_116000000_116500000, |
| | 7_75500000_76000000, 7_63000000_63500000, 7_77500000_78000000, |
| | 7_99000000_99500000, 7_71500000_72000000, 7_105000000_105500000, |
| | 7_5500000_6000000, 7_76000000_76500000, 7_54000000_54500000, |
| | 7_17000000_17500000, 7_131000000_131500000, 7_133000000_133500000, |
| | 7_37000000_37500000, 7_39500000_40000000, 7_101500000_102000000, |
| | 7_130500000_131000000, 7_66000000_66500000, 7_147500000_148000000, |
| | 7_57500000_58000000, 7_149000000_149500000, 7_105500000_106000000, |
| | 7_142500000_143000000, 7_65000000_65500000, 7_92000000_92500000, |
| | 7_64000000_64500000, 7_91000000_91500000, 7_143500000_144000000, |
| | 7_112000000_112500000, 7_151000000_151500000, 7_30500000_31000000, |
| | 7_144500000_145000000, 7_148500000_149000000, 7_88000000_88500000, |
| | 7_76500000_77000000, 7_56000000_56500000, 7_72500000_73000000, |
| | 7_109500000_110000000, 7_97500000_98000000, 7_72000000_72500000, |
| | 7_37500000_38000000, 7_73500000_74000000, 7_46500000_47000000, |
| | 7_32500000_33000000, 7_137000000_137500000, 7_93500000_94000000, |
| | 7_68000000_68500000, 7_106000000_106500000, 7_111500000_112000000, |
| | 7_51000000_51500000, 7_100000000_100500000, 7_128500000_129000000, |
| | 7_65500000_66000000, 7_38000000_38500000, 7_107000000_107500000, |
| | 7_153500000_154000000 |
| 8 | 8_0_500000, 8_100000000_100500000, 8_101000000_101500000, |
| | 8_104000000_104500000, 8_106000000_106500000, 8_11000000_11500000, |
| | 8_113000000_113500000, 8_113500000_114000000, 8_114500000_115000000, |
| | 8_115000000_115500000, 8_117500000_118000000, 8_12000000_12500000, |
| | 8_120500000_121000000, 8_121500000_122000000, 8_122500000_123000000, |
| | 8_123500000_124000000, 8_124000000_124500000, 8_124500000_125000000, |
| | 8_12500000_13000000, 8_125000000_125500000, 8_126000000_126500000, |
| | 8_127500000_128000000, 8_128000000_128500000, 8_128500000_129000000, |
| | 8_129000000_129500000, 8_129500000_130000000, 8_130000000_130500000, |
| | 8_130500000_131000000, 8_132000000_132500000, 8_133000000_133500000, |
| | 8_133500000_134000000, 8_134000000_134500000, 8_135000000_135500000, |
| | 8_135500000_136000000, 8_137000000_137500000, 8_137500000_138000000, |
| | 8_138000000_138500000, 8_138500000_139000000, 8_139500000_140000000, |
| | 8_140000000_140500000, 8_140500000_141000000, 8_144000000_144500000, |
| | 8_15000000_15500000, 8_17500000_18000000, 8_18000000_18500000, |
| | 8_19500000_20000000, 8_20000000_20500000, 8_20500000_21000000, |
| | 8_21000000_21500000, 8_22500000_23000000, 8_23500000_24000000, |
| | 8_25000000_25500000, 8_27000000_27500000, 8_28500000_29000000, |
| | 8_29000000_29500000, 8_29500000_30000000, 8_3000000_3500000, |
| | 8_30000000_30500000, 8_30500000_31000000, 8_32500000_33000000, |
| | 8_33000000_33500000, 8_35000000_35500000, 8_35500000_36000000, |
| | 8_36000000_36500000, 8_37500000_38000000, 8_38000000_38500000, |
| | 8_4000000_4500000, 8_41500000_42000000, 8_42000000_42500000, |
| | 8_42500000_43000000, 8_43500000_44000000, 8_46000000_46500000, |
| | 8_47500000_48000000, 8_48000000_48500000, 8_49000000_49500000, |
| | 8_55000000_55500000, 8_57000000_57500000, 8_57500000_58000000, |
| | 8_58500000_59000000, 8_60500000_61000000, 8_61000000_61500000, |
| | 8_62000000_62500000, 8_64500000_65000000, 8_6500000_7000000, |
| | 8_65500000_66000000, 8_66000000_66500000, 8_66500000_67000000, |
| | 8_68500000_69000000, 8_69000000_69500000, 8_7000000_7500000, |
| | 8_70000000_70500000, 8_72000000_72500000, 8_73500000_74000000, |
| | 8_74500000_75000000, 8_7500000_8000000, 8_77000000_77500000, |
| | 8_79500000_80000000, 8_8000000_8500000, 8_80500000_81000000, |
| | 8_81500000_82000000, 8_84000000_84500000, 8_8500000_9000000, |
| | 8_85500000_86000000, 8_86000000_86500000, 8_86500000_87000000, |
| | 8_87000000_87500000, 8_87500000_88000000, 8_88000000_88500000, |
| | 8_91000000_91500000, 8_91500000_92000000, 8_93000000_93500000, |
| | 8_94500000_95000000, 8_95000000_95500000, 8_96000000_96500000, |
| | 8_96500000_97000000, 8_97000000_97500000, 8_98000000_98500000, |
| 9 | 9_0_500000, 9_1000000_1500000, 9_10000000_10500000, |
| | 9_102000000_102500000, 9_103000000_103500000, 9_103500000_104000000, |
| | 9_10500000_11000000, 9_105000000_105500000, 9_106000000_106500000, |
| | 9_107500000_108000000, 9_109000000_109500000, 9_112000000_112500000, |
| | 9_113500000_114000000, 9_114000000_114500000, 9_114500000_115000000, |
| | 9_116000000_116500000, 9_117000000_117500000, 9_117500000_118000000, |
| | 9_120500000_121000000, 9_121500000_122000000, 9_122000000_122500000, |
| | 9_123000000_123500000, 9_124500000_125000000, 9_125000000_125500000, |
| | 9_125500000_126000000, 9_126000000_126500000, 9_126500000_127000000, |
| | 9_127500000_128000000, 9_128000000_128500000, 9_128500000_129000000, |
| | 9_129000000_129500000, 9_129500000_130000000, 9_13000000_13500000, |
| | 9_131500000_132000000, 9_132000000_132500000, 9_13500000_14000000, |
| | 9_135000000_135500000, 9_1500000_2000000, 9_15000000_15500000, |
| | 9_17000000_17500000, 9_17500000_18000000, 9_19000000_19500000, |
| | 9_20500000_21000000, 9_21000000_21500000, 9_21500000_22000000, |
| | 9_22000000_22500000, 9_23500000_24000000, 9_2500000_3000000, |
| | 9_28000000_28500000, 9_29000000_29500000, 9_32000000_32500000, |
| | 9_33000000_33500000, 9_34000000_34500000, 9_34500000_35000000, |
| | 9_3500000_4000000, 9_35000000_35500000, 9_35500000_36000000, |
| | 9_36000000_36500000, 9_37000000_37500000, 9_37500000_38000000, |
| | 9_39000000_39500000, 9_39500000_40000000, 9_40000000_40500000, |
| | 9_40500000_41000000, 9_41000000_41500000, 9_41500000_42000000, |
| | 9_42000000_42500000, 9_42500000_43000000, 9_43000000_43500000, |
| | 9_4500000_5000000, 9_5500000_6000000, 9_60500000_61000000, |
| | 9_61000000_61500000, 9_61500000_62000000, 9_62000000_62500000, |
| | 9_62500000_63000000, 9_63000000_63500000, 9_63500000_64000000, |
| | 9_64000000_64500000, 9_64500000_65000000, 9_6500000_7000000, |
| | 9_65000000_65500000, 9_65500000_66000000, 9_66000000_66500000, |
| | 9_66500000_67000000, 9_67000000_67500000, 9_67500000_68000000, |
| | 9_71500000_72000000, 9_72000000_72500000, 9_72500000_73000000, |
| | 9_73500000_74000000, 9_74500000_75000000, 9_75000000_75500000, |
| | 9_75500000_76000000, 9_77500000_78000000, 9_78000000_78500000, |
| | 9_78500000_79000000, 9_8000000_8500000, 9_80500000_81000000, |
| | 9_83500000_84000000, 9_84000000_84500000, 9_85000000_85500000, |
| | 9_85500000_86000000, 9_86000000_86500000, 9_86500000_87000000, |
| | 9_87000000_87500000, 9_89000000_89500000, 9_89500000_90000000, |
| | 9_92500000_93000000, 9_97500000_98000000, 9_98000000_98500000, |
| | 9_99000000_99500000, |
| 10 | 10_0_500000, 10_100000000_100500000, 10_100500000_101000000, |
| | 10_101000000_101500000, 10_101500000_102000000, 10_102000000_102500000, |
| | 10_102500000_103000000, 10_104000000_104500000, 10_107000000_107500000, |
| | 10_108000000_108500000, 10_109000000_109500000, 10_109500000_110000000, |
| | 10_110000000_110500000, 10_110500000_111000000, 10_111500000_112000000, |
| | 10_116000000_116500000, 10_117000000_117500000, 10_119000000_119500000, |
| | 10_119500000_120000000, 10_12000000_12500000, 10_120500000_121000000, |
| | 10_122000000_122500000, 10_123500000_124000000, 10_125500000_126000000, |
| | 10_126500000_127000000, 10_127000000_127500000, 10_127500000_128000000, |
| | 10_128500000_129000000, 10_129000000_129500000, 10_129500000_130000000, |
| | 10_130000000_130500000, 10_130500000_131000000, 10_133500000_133797422, |
| | 10_13500000_14000000, 10_14500000_15000000, 10_1500000_2000000, |
| | 10_16500000_17000000, 10_17000000_17500000, 10_19000000_19500000, |
| | 10_2000000_2500000, 10_20500000_21000000, 10_21000000_21500000, |
| | 10_21500000_22000000, 10_22000000_22500000, 10_22500000_23000000, |
| | 10_23000000_23500000, 10_23500000_24000000, 10_24000000_24500000, |
| | 10_27000000_27500000, 10_27500000_28000000, 10_28000000_28500000, |
| | 10_3000000_3500000, 10_30000000_30500000, 10_31500000_32000000, |
| | 10_32000000_32500000, 10_3500000_4000000, 10_35000000_35500000, |
| | 10_35500000_36000000, 10_36500000_37000000, 10_37000000_37500000, |
| | 10_38000000_38500000, 10_38500000_39000000, 10_4000000_4500000, |
| | 10_42000000_42500000, 10_42500000_43000000, 10_43000000_43500000, |
| | 10_44000000_44500000, 10_46000000_46500000, 10_46500000_47000000, |
| | 10_47500000_48000000, 10_49000000_49500000, 10_50000000_50500000, |
| | 10_52000000_52500000, 10_52500000_53000000, 10_53500000_54000000, |
| | 10_54500000_55000000, 10_55500000_56000000, 10_58000000_58500000, |
| | 10_59000000_59500000, 10_6000000_6500000, 10_62000000_62500000, |
| | 10_62500000_63000000, 10_63000000_63500000, 10_64000000_64500000, |
| | 10_68000000_68500000, 10_68500000_69000000, 10_69000000_69500000, |
| | 10_69500000_70000000, 10_7000000_7500000, 10_71500000_72000000, |
| | 10_72000000_72500000, 10_73000000_73500000, 10_73500000_74000000, |
| | 10_74500000_75000000, 10_7500000_8000000, 10_76500000_77000000, |
| | 10_77500000_78000000, 10_79000000_79500000, 10_8000000_8500000, |
| | 10_82000000_82500000, 10_82500000_83000000, 10_84500000_85000000, |
| | 10_8500000_9000000, 10_87000000_87500000, 10_87500000_88000000, |
| | 10_88000000_88500000, 10_88500000_89000000, 10_89000000_89500000, |
| | 10_89500000_90000000, 10_9000000_9500000, 10_92500000_93000000, |
| | 10_94000000_94500000, 10_94500000_95000000, 10_95000000_95500000, |
| | 10_95500000_96000000, 10_96500000_97000000, 10_97000000_97500000, |
| | 10_97500000_98000000, 10_98000000_98500000, 10_98500000_99000000, |
| | 10_99000000_99500000 |
| 11 | 11_0_500000, 11_100000000_100500000, 11_101000000_101500000, |
| | 11_101500000_102000000, 11_102000000_102500000, 11_103000000_103500000, |
| | 11_104000000_104500000, 11_104500000_105000000, 11_10500000_11000000, |
| | 11_106500000_107000000, 11_107000000_107500000, 11_107500000_108000000, |
| | 11_108000000_108500000, 11_108500000_109000000, 11_109000000_109500000, |
| | 11_111000000_111500000, 11_111500000_112000000, 11_113500000_114000000, |
| | 11_114000000_114500000, 11_11500000_12000000, 11_116000000_116500000, |
| | 11_116500000_117000000, 11_117000000_117500000, 11_117500000_118000000, |
| | 11_118000000_118500000, 11_118500000_119000000, 11_119000000_119500000, |
| | 11_12000000_12500000, 11_120500000_121000000, 11_121000000_121500000, |
| | 11_122500000_123000000, 11_123500000_124000000, 11_125500000_126000000, |
| | 11_126000000_126500000, 11_126500000_127000000, 11_127000000_127500000, |
| | 11_128500000_129000000, 11_13000000_13500000, 11_130000000_130500000, |
| | 11_133500000_134000000, 11_134500000_135000000, 11_135000000_135086622, |
| | 11_1500000_2000000, 11_15500000_16000000, 11_17500000_18000000, |
| | 11_18000000_18500000, 11_20500000_21000000, 11_23000000_23500000, |
| | 11_24000000_24500000, 11_2500000_3000000, 11_25500000_26000000, |
| | 11_27500000_28000000, 11_28000000_28500000, 11_29000000_29500000, |
| | 11_32000000_32500000, 11_33000000_33500000, 11_33500000_34000000, |
| | 11_34500000_35000000, 11_3500000_4000000, 11_35000000_35500000, |
| | 11_37500000_38000000, 11_38000000_38500000, 11_38500000_39000000, |
| | 11_39500000_40000000, 11_4000000_4500000, 11_40000000_40500000, |
| | 11_40500000_41000000, 11_41500000_42000000, 11_43500000_44000000, |
| | 11_44500000_45000000, 11_4500000_5000000, 11_46000000_46500000, |
| | 11_47000000_47500000, 11_47500000_48000000, 11_48500000_49000000, |
| | 11_49000000_49500000, 11_49500000_50000000, 11_50000000_50500000, |
| | 11_5500000_6000000, 11_55500000_56000000, 11_56500000_57000000, |
| | 11_57500000_58000000, 11_58500000_59000000, 11_59500000_60000000, |
| | 11_6000000_6500000, 11_61000000_61500000, 11_61500000_62000000, |
| | 11_62000000_62500000, 11_62500000_63000000, 11_63500000_64000000, |
| | 11_64000000_64500000, 11_64500000_65000000, 11_6500000_7000000, |
| | 11_65000000_65500000, 11_65500000_66000000, 11_66000000_66500000, |
| | 11_67000000_67500000, 11_68500000_69000000, 11_69500000_70000000, |
| | 11_72000000_72500000, 11_72500000_73000000, 11_73000000_73500000, |
| | 11_73500000_74000000, 11_76000000_76500000, 11_77000000_77500000, |
| | 11_77500000_78000000, 11_78000000_78500000, 11_79000000_79500000, |
| | 11_80000000_80500000, 11_80500000_81000000, 11_81500000_82000000, |
| | 11_8500000_9000000, 11_85500000_86000000, 11_86000000_86500000, |
| | 11_88000000_88500000, 11_89000000_89500000, 11_89500000_90000000, |
| | 11_9000000_9500000, 11_92000000_92500000, 11_92500000_93000000, |
| | 11_93000000_93500000, 11_93500000_94000000, 11_9500000_10000000, |
| | 11_95000000_95500000, 11_95500000_96000000, 11_96500000_97000000, |
| | 11_99000000_99500000, 11_99500000_100000000, |
| 12 | 12_0_500000, 12_101500000_102000000, 12_102500000_103000000, |
| | 12_104000000_104500000, 12_104500000_105000000, 12_10500000_11000000, |
| | 12_105000000_105500000, 12_106500000_107000000, 12_107000000_107500000, |
| | 12_108500000_109000000, 12_109500000_110000000, 12_11000000_11500000, |
| | 12_110000000_110500000, 12_110500000_111000000, 12_111000000_111500000, |
| | 12_111500000_112000000, 12_112000000_112500000, 12_112500000_113000000, |
| | 12_113000000_113500000, 12_113500000_114000000, 12_11500000_12000000, |
| | 12_116000000_116500000, 12_116500000_117000000, 12_118000000_118500000, |
| | 12_12000000_12500000, 12_120000000_120500000, 12_120500000_121000000, |
| | 12_121000000_121500000, 12_121500000_122000000, 12_122000000_122500000, |
| | 12_122500000_123000000, 12_123000000_123500000, 12_123500000_124000000, |
| | 12_12500000_13000000, 12_127500000_128000000, 12_128500000_129000000, |
| | 12_129500000_130000000, 12_13000000_13500000, 12_14500000_15000000, |
| | 12_1500000_2000000, 12_15500000_16000000, 12_17000000_17500000, |
| | 12_17500000_18000000, 12_18000000_18500000, 12_21500000_22000000, |
| | 12_22000000_22500000, 12_22500000_23000000, 12_23500000_24000000, |
| | 12_24500000_25000000, 12_25000000_25500000, 12_25500000_26000000, |
| | 12_26000000_26500000, 12_27000000_27500000, 12_28000000_28500000, |
| | 12_30000000_30500000, 12_31500000_32000000, 12_33000000_33500000, |
| | 12_34500000_35000000, 12_3500000_4000000, 12_37000000_37500000, |
| | 12_37500000_38000000, 12_38000000_38500000, 12_38500000_39000000, |
| | 12_39500000_40000000, 12_40000000_40500000, 12_40500000_41000000, |
| | 12_41000000_41500000, 12_42000000_42500000, 12_42500000_43000000, |
| | 12_47000000_47500000, 12_47500000_48000000, 12_48500000_49000000, |
| | 12_49000000_49500000, 12_49500000_50000000, 12_500000_1000000, |
| | 12_50000000_50500000, 12_50500000_51000000, 12_51000000_51500000, |
| | 12_52500000_53000000, 12_53000000_53500000, 12_53500000_54000000, |
| | 12_54000000_54500000, 12_54500000_55000000, 12_55500000_56000000, |
| | 12_56000000_56500000, 12_57000000_57500000, 12_57500000_58000000, |
| | 12_58000000_58500000, 12_58500000_59000000, 12_6000000_6500000, |
| | 12_60500000_61000000, 12_61000000_61500000, 12_62000000_62500000, |
| | 12_64500000_65000000, 12_6500000_7000000, 12_65000000_65500000, |
| | 12_65500000_66000000, 12_66000000_66500000, 12_66500000_67000000, |
| | 12_67_500000_68000000, 12_68000000_68500000, 12_69500000_70000000, |
| | 12_70500000_71000000, 12_71500000_72000000, 12_73000000_73500000, |
| | 12_74500000_75000000, 12_7500000_8000000, 12_75000000_75500000, |
| | 12_75500000_76000000, 12_76000000_76500000, 12_76500000_77000000, |
| | 12_79500000_80000000, 12_80000000_80500000, 12_83500000_84000000, |
| | 12_84000000_84500000, 12_8500000_9000000, 12_85000000_85500000, |
| | 12_88000000_88500000, 12_89000000_89500000, 12_89500000_90000000, |
| | 12_9000000_9500000, 12_91000000_91500000, 12_91500000_92000000, |
| | 12_93000000_93500000, 12_93500000_94000000, 12_94500000_95000000, |
| | 12_9500000_10000000, 12_95500000_96000000, 12_96000000_96500000, |
| | 12_98500000_99000000, |
| 13 | 13_100000000_100500000, 13_101500000_102000000, 13_102000000_102500000, |
| | 13_102500000_103000000, 13_104000000_104500000, 13_106000000_106500000, |
| | 13_106500000_107000000, 13_107000000_107500000, 13_108000000_108500000, |
| | 13_112000000_112500000, 13_113500000_114000000, 13_16000000_16500000, |
| | 13_18000000_18500000, 13_18500000_19000000, 13_19000000_19500000, |
| | 13_20500000_21000000, 13_21000000_21500000, 13_21500000_22000000, |
| | 13_22000000_22500000, 13_27000000_27500000, 13_28000000_28500000, |
| | 13_29000000_29500000, 13_30000000_30500000, 13_30500000_31000000, |
| | 13_32500000_33000000, 13_34500000_35000000, 13_35500000_36000000, |
| | 13_36000000_36500000, 13_37500000_38000000, 13_38500000_39000000, |
| | 13_39000000_39500000, 13_40000000_40500000, 13_40500000_41000000, |
| | 13_41000000_41500000, 13_42000000_42500000, 13_44000000_44500000, |
| | 13_45000000_45500000, 13_46000000_46500000, 13_47500000_48000000, |
| | 13_48000000_48500000, 13_48500000_49000000, 13_49500000_50000000, |
| | 13_50000000_50500000, 13_51000000_51500000, 13_54000000_54500000, |
| | 13_56500000_57000000, 13_57000000_57500000, 13_60500000_61000000, |
| | 13_64500000_65000000, 13_66500000_67000000, 13_67000000_67500000, |
| | 13_67500000_68000000, 13_68000000_68500000, 13_73000000_73500000, |
| | 13_73500000_74000000, 13_74000000_74500000, 13_75500000_76000000, |
| | 13_84500000_85000000, 13_85000000_85500000, 13_85500000_86000000, |
| | 13_86500000_87000000, 13_90500000_91000000, 13_92500000_93000000, |
| | 13_93500000_94000000, 13_94000000_94500000, 13_97500000_98000000, |
| | 13_99000000_99500000 |
| 14 | 14_100000000_100500000, 14_101000000_101500000, 14_103000000_103500000, |
| | 14_104000000_104500000, 14_106500000_107000000, 14_18000000_18500000, |
| | 14_18500000_19000000, 14_19000000_19500000, 14_19500000_20000000, |
| | 14_20000000_20500000, 14_21000000_21500000, 14_22000000_22500000, |
| | 14_22500000_23000000, 14_23000000_23500000, 14_25500000_26000000, |
| | 14_27000000_27500000, 14_28000000_28500000, 14_29000000_29500000, |
| | 14_29500000_30000000, 14_30000000_30500000, 14_30500000_31000000, |
| | 14_31500000_32000000, 14_32500000_33000000, 14_34500000_35000000, |
| | 14_35000000_35500000, 14_35500000_36000000, 14_36500000_37000000, |
| | 14_37000000_37500000, 14_39000000_39500000, 14_39500000_40000000, |
| | 14_43000000_43500000, 14_44500000_45000000, 14_45500000_46000000, |
| | 14_47000000_47500000, 14_47500000_48000000, 14_48000000_48500000, |
| | 14_49500000_50000000, 14_50500000_51000000, 14_51500000_52000000, |
| | 14_55000000_55500000, 14_55500000_56000000, 14_58000000_58500000, |
| | 14_59000000_59500000, 14_60000000_60500000, 14_61000000_61500000, |
| | 14_63500000_64000000, 14_64500000_65000000, 14_65000000_65500000, |
| | 14_66000000_66500000, 14_67000000_67500000, 14_67500000_68000000, |
| | 14_68500000_69000000, 14_69000000_69500000, 14_69500000_70000000, |
| | 14_70500000_71000000, 14_71000000_71500000, 14_73000000_73500000, |
| | 14_74000000_74500000, 14_75000000_75500000, 14_76000000_76500000, |
| | 14_77000000_77500000, 14_79500000_80000000, 14_80500000_81000000, |
| | 14_81000000_81500000, 14_82000000_82500000, 14_83000000_83500000, |
| | 14_84000000_84500000, 14_86000000_86500000, 14_86500000_87000000, |
| | 14_87000000_87500000, 14_88500000_89000000, 14_89000000_89500000, |
| | 14_89500000_90000000, 14_90500000_91000000, 14_91000000_91500000, |
| | 14_92000000_92500000, 14_92500000_93000000, 14_94000000_94500000, |
| | 14_94500000_95000000, 14_95500000_96000000, 14_96000000_96500000, |
| | 14_96500000_97000000, 14_97000000_97500000, 14_97500000_98000000, |
| | 14_98000000_98500000, 14_99500000_100000000 |
| 15 | 15_100000000_100500000, 15_17000000_17500000, 15_19500000_20000000, |
| | 15_20000000_20500000, 15_20500000_21000000, 15_21000000_21500000, |
| | 15_21500000_22000000, 15_22000000_22500000, 15_23000000_23500000, |
| | 15_23500000_24000000, 15_24000000_24500000, 15_24500000_25000000, |
| | 15_25000000_25500000, 15_26500000_27000000, 15_27500000_28000000, |
| | 15_28000000_28500000, 15_28500000_29000000, 15_30000000_30500000, |
| | 15_30500000_31000000, 15_31500000_32000000, 15_32000000_32500000, |
| | 15_34000000_34500000, 15_35000000_35500000, 15_36000000_36500000, |
| | 15_36500000_37000000, 15_38000000_38500000, 15_39500000_40000000, |
| | 15_40500000_41000000, 15_41000000_41500000, 15_41500000_42000000, |
| | 15_42000000_42500000, 15_42500000_43000000, 15_43000000_43500000, |
| | 15_43500000_44000000, 15_45500000_46000000, 15_46000000_46500000, |
| | 15_47000000_47500000, 15_48500000_49000000, 15_50000000_50500000, |
| | 15_51500000_52000000, 15_53000000_53500000, 15_53500000_54000000, |
| | 15_55000000_55500000, 15_57000000_57500000, 15_57500000_58000000, |
| | 15_58000000_58500000, 15_60000000_60500000, 15_60500000_61000000, |
| | 15_61500000_62000000, 15_62000000_62500000, 15_63000000_63500000, |
| | 15_63500000_64000000, 15_64000000_64500000, 15_64500000_65000000, |
| | 15_65000000_65500000, 15_65500000_66000000, 15_66500000_67000000, |
| | 15_68500000_69000000, 15_69000000_69500000, 15_69500000_70000000, |
| | 15_70000000_70500000, 15_72000000_72500000, 15_72500000_73000000, |
| | 15_73000000_73500000, 15_74500000_75000000, 15_78000000_78500000, |
| | 15_79000000_79500000, 15_79500000_80000000, 15_81000000_81500000, |
| | 15_81500000_82000000, 15_82000000_82500000, 15_84000000_84500000, |
| | 15_85500000_86000000, 15_86500000_87000000, 15_88000000_88500000, |
| | 15_89000000_89500000, 15_90000000_90500000, 15_90500000_91000000, |
| | 15_91000000_91500000, 15_91500000_92000000, 15_92500000_93000000, |
| | 15_94000000_94500000, 15_96500000_97000000, 15_97500000_98000000 |
| 16 | 16_10500000_11000000, 16_11500000_12000000, 16_12000000_12500000, |
| | 16_12500000_13000000, 16_13000000_13500000, 16_13500000_14000000, |
| | 16_14000000_14500000, 16_14500000_15000000, 16_15000000_15500000, |
| | 16_15500000_16000000, 16_16000000_16500000, 16_17000000_17500000, |
| | 16_17500000_18000000, 16_18000000_18500000, 16_19500000_20000000, |
| | 16_21500000_22000000, 16_22000000_22500000, 16_23000000_23500000, |
| | 16_23500000_24000000, 16_24000000_24500000, 16_24500000_25000000, |
| | 16_26000000_26500000, 16_26500000_27000000, 16_27000000_27500000, |
| | 16_27500000_28000000, 16_28500000_29000000, 16_29000000_29500000, |
| | 16_29500000_30000000, 16_3000000_3500000, 16_30000000_30500000, |
| | 16_30500000_31000000, 16_31000000_31500000, 16_32000000_32500000, |
| | 16_32500000_33000000, 16_33000000_33500000, 16_33500000_34000000, |
| | 16_34000000_34500000, 16_34500000_35000000, 16_35000000_35500000, |
| | 16_4000000_4500000, 16_4500000_5000000, 16_46000000_46500000, |
| | 16_46500000_47000000, 16_48000000_48500000, 16_49000000_49500000, |
| | 16_49500000_50000000, 16_5000000_5500000, 16_51000000_51500000, |
| | 16_52000000_52500000, 16_53000000_53500000, 16_53500000_54000000, |
| | 16_54000000_54500000, 16_57000000_57500000, 16_57500000_58000000, |
| | 16_58000000_58500000, 16_59500000_60000000, 16_61000000_61500000, |
| | 16_63000000_63500000, 16_6500000_7000000, 16_66500000_67000000, |
| | 16_67500000_68000000, 16_68000000_68500000, 16_69000000_69500000, |
| | 16_71500000_72000000, 16_72000000_72500000, 16_74000000_74500000, |
| | 16_74500000_75000000, 16_76000000_76500000, 16_77500000_78000000, |
| | 16_79000000_79500000, 16_79500000_80000000, 16_81000000_81500000, |
| | 16_81500000_82000000, 16_82500000_83000000, 16_83500000_84000000, |
| | 16_84500000_85000000, 16_8500000_9000000, 16_85000000_85500000, |
| | 16_85500000_86000000, 16_9000000_9500000, 16_90000000_90338345, |
| 17 | 17_0_500000, 17_1000000_1500000, 17_10000000_10500000, |
| | 17_10500000_11000000, 17_11000000_11500000, 17_11500000_12000000, |
| | 17_13000000_13500000, 17_13500000_14000000, 17_14000000_14500000, |
| | 17_1500000_2000000, 17_16000000_16500000, 17_17500000_18000000, |
| | 17_19000000_19500000, 17_19500000_20000000, 17_2000000_2500000, |
| | 17_20000000_20500000, 17_20500000_21000000, 17_21000000_21500000, |
| | 17_21500000_22000000, 17_22000000_22500000, 17_22500000_23000000, |
| | 17_2500000_3000000, 17_26500000_27000000, 17_27500000_28000000, |
| | 17_28000000_28500000, 17_28500000_29000000, 17_29000000_29500000, |
| | 17_29500000_30000000, 17_30000000_30500000, 17_31000000_31500000, |
| | 17_32000000_32500000, 17_33000000_33500000, 17_33500000_34000000, |
| | 17_34500000_35000000, 17_35000000_35500000, 17_35500000_36000000, |
| | 17_36000000_36500000, 17_36500000_37000000, 17_37000000_37500000, |
| | 17_38000000_38500000, 17_38500000_39000000, 17_39000000_39500000, |
| | 17_39500000_40000000, 17_4000000_4500000, 17_40000000_40500000, |
| | 17_40500000_41000000, 17_41500000_42000000, 17_42000000_42500000, |
| | 17_42500000_43000000, 17_43500000_44000000, 17_44000000_44500000, |
| | 17_44500000_45000000, 17_4500000_5000000, 17_45000000_45500000, |
| | 17_47500000_48000000, 17_48000000_48500000, 17_48500000_49000000, |
| | 17_49000000_49500000, 17_49500000_50000000, 17_500000_1000000, |
| | 17_5000000_5500000, 17_50000000_50500000, 17_50500000_51000000, |
| | 17_51000000_51500000, 17_53500000_54000000, 17_54000000_54500000, |
| | 17_55000000_55500000, 17_56000000_56500000, 17_56500000_57000000, |
| | 17_57000000_57500000, 17_58000000_58500000, 17_58500000_59000000, |
| | 17_59000000_59500000, 17_59500000_60000000, 17_60000000_60500000, |
| | 17_61500000_62000000, 17_62500000_63000000, 17_63500000_64000000, |
| | 17_64000000_64500000, 17_65000000_65500000, 17_65500000_66000000, |
| | 17_66000000_66500000, 17_67000000_67500000, 17_67500000_68000000, |
| | 17_68000000_68500000, 17_69000000_69500000, 17_7000000_7500000, |
| | 17_70500000_71000000, 17_71000000_71500000, 17_71500000_72000000, |
| | 17_72500000_73000000, 17_73500000_74000000, 17_74000000_74500000, |
| | 17_74500000_75000000, 17_7500000_8000000, 17_75000000_75500000, |
| | 17_75500000_76000000, 17_76000000_76500000, 17_76500000_77000000, |
| | 17_77000000_77500000, 17_78000000_78500000, 17_78500000_79000000, |
| | 17_79500000_80000000, 17_8000000_8500000, 17_80000000_80500000, |
| | 17_82500000_83000000, 17_8500000_9000000, |
| 18 | 18_1000000_1500000, 18_10000000_10500000, 18_11000000_11500000, |
| | 18_12000000_12500000, 18_12500000_13000000, 18_14000000_14500000, |
| | 18_14500000_15000000, 18_15000000_15500000, 18_20500000_21000000, |
| | 18_21000000_21500000, 18_23500000_24000000, 18_24000000_24500000, |
| | 18_2500000_3000000, 18_25000000_25500000, 18_27000000_27500000, |
| | 18_27500000_28000000, 18_3000000_3500000, 18_30500000_31000000, |
| | 18_31500000_32000000, 18_3500000_4000000, 18_35000000_35500000, |
| | 18_39000000_39500000, 18_42000000_42500000, 18_42500000_43000000, |
| | 18_46000000_46500000, 18_47000000_47500000, 18_48500000_49000000, |
| | 18_49000000_49500000, 18_5000000_5500000, 18_50500000_51000000, |
| | 18_51000000_51500000, 18_53000000_53500000, 18_53500000_54000000, |
| | 18_5500000_6000000, 18_59500000_60000000, 18_61000000_61500000, |
| | 18_63000000_63500000, 18_64500000_65000000, 18_6500000_7000000, |
| | 18_65000000_65500000, 18_67000000_67500000, 18_7000000_7500000, |
| | 18_73000000_73500000, 18_73500000_74000000, 18_74500000_75000000, |
| | 18_76000000_76500000, 18_78000000_78500000, 18_8000000_8500000, |
| | 18_9000000_9500000, 18_9500000_10000000 |
| 19 | 19_0_500000, 19_1000000_1500000, 19_10000000_10500000, |
| | 19_10500000_11000000, 19_11000000_11500000, 19_11500000_12000000, |
| | 19_12500000_13000000, 19_13000000_13500000, 19_13500000_14000000, |
| | 19_14000000_14500000, 19_14500000_15000000, 19_1500000_2000000, |
| | 19_15000000_15500000, 19_15500000_16000000, 19_16000000_16500000, |
| | 19_16500000_17000000, 19_17000000_17500000, 19_17500000_18000000, |
| | 19_18000000_18500000, 19_18500000_19000000, 19_19000000_19500000, |
| | 19_2000000_2500000, 19_20500000_21000000, 19_21000000_21500000, |
| | 19_21500000_22000000, 19_22000000_22500000, 19_22500000_23000000, |
| | 19_2500000_3000000, 19_27500000_28000000, 19_28000000_28500000, |
| | 19_3000000_3500000, 19_34000000_34500000, 19_35000000_35500000, |
| | 19_35500000_36000000, 19_38000000_38500000, 19_38500000_39000000, |
| | 19_39000000_39500000, 19_39500000_40000000, 19_4000000_4500000, |
| | 19_40000000_40500000, 19_40500000_41000000, 19_41000000_41500000, |
| | 19_41500000_42000000, 19_42000000_42500000, 19_42500000_43000000, |
| | 19_43000000_43500000, 19_43500000_44000000, 19_44500000_45000000, |
| | 19_4500000_5000000, 19_45000000_45500000, 19_45500000_46000000, |
| | 19_46500000_47000000, 19_47000000_47500000, 19_48000000_48500000, |
| | 19_48500000_49000000, 19_49000000_49500000, 19_49500000_50000000, |
| | 19_500000_1000000, 19_50500000_51000000, 19_51000000_51500000, |
| | 19_51500000_52000000, 19_52000000_52500000, 19_53500000_54000000, |
| | 19_54000000_54500000, 19_5500000_6000000, 19_55000000_55500000, |
| | 19_55500000_56000000, 19_56000000_56500000, 19_58500000_58617616, |
| | 19_6000000_6500000, 19_6500000_7000000, 19_7000000_7500000, |
| | 19_7500000_8000000, 19_8000000_8500000, 19_8500000_9000000, |
| | 19_9500000_10000000 |
| 20 | 20_0_500000, 20_1000000_1500000, 20_10000000_10500000, |
| | 20_10500000_11000000, 20_11500000_12000000, 20_12500000_13000000, |
| | 20_14500000_15000000, 20_1500000_2000000, 20_16000000_16500000, |
| | 20_19000000_19500000, 20_2000000_2500000, 20_20000000_20500000, |
| | 20_20500000_21000000, 20_21000000_21500000, 20_21500000_22000000, |
| | 20_24500000_25000000, 20_2500000_3000000, 20_25000000_25500000, |
| | 20_25500000_26000000, 20_26000000_26500000, 20_28500000_29000000, |
| | 20_29000000_29500000, 20_29500000_30000000, 20_3000000_3500000, |
| | 20_30000000_30500000, 20_30500000_31000000, 20_31500000_32000000, |
| | 20_32000000_32500000, 20_33000000_33500000, 20_33500000_34000000, |
| | 20_34000000_34500000, 20_34500000_35000000, 20_3500000_4000000, |
| | 20_35000000_35500000, 20_35500000_36000000, 20_36500000_37000000, |
| | 20_37000000_37500000, 20_37500000_38000000, 20_38000000_38500000, |
| | 20_38500000_39000000, 20_39500000_40000000, 20_40500000_41000000, |
| | 20_41000000_41500000, 20_42500000_43000000, 20_43500000_44000000, |
| | 20_44000000_44500000, 20_44500000_45000000, 20_4500000_5000000, |
| | 20_45000000_45500000, 20_45500000_46000000, 20_46000000_46500000, |
| | 20_47000000_47500000, 20_47500000_48000000, 20_48000000_48500000, |
| | 20_48500000_49000000, 20_49000000_49500000, 20_49500000_50000000, |
| | 20_5000000_5500000, 20_50000000_50500000, 20_50500000_51000000, |
| | 20_51000000_51500000, 20_51500000_52000000, 20_52000000_52500000, |
| | 20_52500000_53000000, 20_53500000_54000000, 20_5500000_6000000, |
| | 20_55000000_55500000, 20_55500000_56000000, 20_56500000_57000000, |
| | 20_57000000_57500000, 20_57500000_58000000, 20_58500000_59000000, |
| | 20_59000000_59500000, 20_61000000_61500000, 20_62000000_62500000, |
| | 20_62500000_63000000, 20_63000000_63500000, 20_8000000_8500000 |
| 21 | 21_10000000_10500000, 21_10500000_11000000, 21_13000000_13500000, |
| | 21_13500000_14000000, 21_14000000_14500000, 21_14500000_15000000, |
| | 21_15000000_15500000, 21_15500000_16000000, 21_16500000_17000000, |
| | 21_17000000_17500000, 21_17500000_18000000, 21_18000000_18500000, |
| | 21_23500000_24000000, 21_24500000_25000000, 21_25500000_26000000, |
| | 21_29000000_29500000, 21_31000000_31500000, 21_31500000_32000000, |
| | 21_32500000_33000000, 21_33000000_33500000, 21_33500000_34000000, |
| | 21_34500000_35000000, 21_35000000_35500000, 21_35500000_36000000, |
| | 21_36000000_36500000, 21_37000000_37500000, 21_38000000_38500000, |
| | 21_39500000_40000000, 21_40000000_40500000, 21_40500000_41000000, |
| | 21_42000000_42500000, 21_44000000_44500000, 21_44500000_45000000, |
| | 21_5000000_5500000, 21_5500000_6000000, 21_6000000_6500000, |
| | 21_6500000_7000000, 21_7000000_7500000, 21_7500000_8000000, |
| | 21_8000000_8500000, 21_8500000_9000000, 21_9000000_9500000, |
| | 21_9500000_10000000, |
| 22 | 22_10500000_11000000, 22_11000000_11500000, 22_11500000_12000000, |
| | 22_12000000_12500000, 22_12500000_13000000, 22_15000000_15500000, |
| | 22_15500000_16000000, 22_16000000_16500000, 22_16500000_17000000, |
| | 22_18000000_18500000, 22_18500000_19000000, 22_19000000_19500000, |
| | 22_20500000_21000000, 22_21000000_21500000, 22_21500000_22000000, |
| | 22_23000000_23500000, 22_23500000_24000000, 22_25000000_25500000, |
| | 22_26000000_26500000, 22_27000000_27500000, 22_27500000_28000000, |
| | 22_28500000_29000000, 22_29500000_30000000, 22_30000000_30500000, |
| | 22_31000000_31500000, 22_31500000_32000000, 22_32000000_32500000, |
| | 22_32500000_33000000, 22_35000000_35500000, 22_35500000_36000000, |
| | 22_36000000_36500000, 22_36500000_37000000, 22_37000000_37500000, |
| | 22_37500000_38000000, 22_38000000_38500000, 22_38500000_39000000, |
| | 22_39000000_39500000, 22_39500000_40000000, 22_40000000_40500000, |
| | 22_40500000_41000000, 22_41000000_41500000, 22_41500000_42000000, |
| | 22_42500000_43000000, 22_43000000_43500000, 22_48000000_48500000, |
| | 22_48500000_49000000 |

### Example 16. Characterization of the rbcDNA Signature in Healthy Donors and Cancer Patients

Genome-wide sequencing profiles revealed rbcDNA signals distribute across autosomal chromosomes with specific patterns distinct from those of the corresponding genomic DNA (gDNA) (FIG. 15A). The mean genomic coverage of rbcDNA is higher in healthy donors compared to that of cancer patients, while no significant difference in coverage is observed among patients of different cancer types (FIGS. 15B and 15C). Nonetheless, genome-wide analysis showed pronounced signal enrichment of intergenic, intronic and exonic regions in rbcDNA of cancer patients versus healthy donors. A modest differential enrichment of rbcDNA signals was detected in intergenic and intronic regions of CRC patients compared to patients of other cancer types (FIG. 15D).

It can be clearly seen from the above examples that the inventors have successfully isolated the micronuclei DNA of peripheral red blood cells, and constructed a classifier for cancer detection by using the micronuclei DNA of peripheral red blood cells, thus realizing the effective detection of cancer, which is of great significance for clinical screening, diagnosis, classification and staging of cancer.

Although the specific embodiments of the present invention have been described in detail, it will be understood by those skilled in the art that various modifications and variations can be made to the details in light of all the teachings disclosed, and these changes are within the scope of protection of the present invention. The full scope of the invention is provided by the appended claims and any equivalents thereof.

1.-38. (canceled)

39. A method of treating a cancer in a subject in need thereof, the method comprising:
a) obtaining a peripheral blood sample from the subject;
b) isolating red blood cells from the peripheral blood sample;
c) extracting micronuclei DNA from the isolated red blood cells;
d) detecting presence and/or levels of a gene classifier in the micronuclei DNA;
e) comparing the detected gene classifier or levels thereof in the sample with a control to thereby diagnose or detect the cancer in the subject; and
f) treating the subject determined to have or diagnosed with cancer with an anti-cancer agent.

40. The method according to claim 39, wherein step (b) comprises (i) subjecting the peripheral blood sample to density gradient centrifugation to obtain a mononuclear cell layer and a red blood cell layer, (ii) collecting the red blood cells from the bottom of the red blood cell layer, and (iii) filtrating the collected red blood cells two or more times through 10 μm cell strainers.

41. The method according to claim 39, wherein step (c) comprises (i) treating the isolated red blood cells with a red blood cell lysis buffer, (ii) treating the lysed red blood cells with an extraction buffer comprising proteinase K, and (iii) extracting the micronuclei DNA from the extraction buffer.

42. The method of claim 41, further comprising sequencing the micronuclei DNA to thereby detect the presence and/or levels of a gene classifier.

43. The method according to claim 42, further comprising amplifying the micronuclei DNA prior to performing the sequencing, wherein the amplifying uses a method selected from the group consisting of multiple displacement amplification (MDA), Multiple Annealing and Looping-based Amplification Cycles (MALBAC) and degenerate oligonucleotide primer PCR (DOP-PCR).

44. The method according to claim 42, wherein the sequencing is high-throughput sequencing.

45. The method according to claim 39, further comprising before step f), removing sequence reads that are more enriched by peripheral blood mononuclear cells relative to erythrocyte micronuclei DNA.

46. The method according to claim 39, wherein the gene classifier comprises the genes shown in Tables 2, 4, 6, 8 and 10.

47. The method according to claim 39, wherein the cancer is selected from the group consisting of lung cancer, cervical cancer, hepatocellular carcinoma, and colorectal cancer.

48. The method according to claim 39, further comprising determining type and/or stage of cancer by analyzing the gene classifier.

49. A method for constructing a system for early detection or diagnosis of cancer, the method comprising:
a) obtaining a peripheral blood sample from healthy subjects and subjects diagnosed with cancer;
b) isolating red blood cells from the peripheral blood sample from each subject;
c) extracting micronuclei DNA from the isolated red blood cells;
d) performing sequencing on the micronuclei DNA to obtain fragment sequence information of the micronuclei DNA;
e) comparing the fragment sequence information of micronuclei DNA from peripheral red blood cells in healthy subjects and subjects diagnosed with cancer;
f) classifying the signature DNA fragment set for a specific cancer according to the differences in the distribution of the fragment in healthy subjects and subjects diagnosed with cancer;
g) obtaining a gene classifier for cancer detection or diagnosis,
wherein the gene classifier is used in the system for early detection or diagnosis of cancer.

50. The method according to claim 49, further comprising before step e), obtaining an enriched fragment in a specific chromosome region of the peripheral red blood cell micronuclei DNA of the subject relative to sequenced reads of the peripheral blood mononuclear cell genome DNA of the subject, thereby obtaining specific cleavage fragments of the peripheral red blood cell of the subject.

51. The method according to claim 49, wherein in step f) the characteristic DNA fragment set for a specific cancer is classified through hierarchical cluster analysis.

52. The method according to claim 49, wherein the cancer is selected from the group consisting of lung cancer, cervical cancer, hepatocellular carcinoma, and colorectal cancer.

53. The method according claim 49, wherein the fragment sequence information of micronuclei DNA are further classified for type or stage of cancer.

54. A kit for early detection or diagnosis of cancer, the kit comprising:
a) reagents for detecting expression of a gene classifier comprising the genes shown in Tables 2, 4, 6, 8 and 10; and
b) instructions for use.

55. The kit of claim 54, wherein the cancer is selected from the group consisting of lung cancer, cervical cancer, hepatocellular carcinoma, and colorectal cancer.
